SEQUENCE LISTING (64 sequences)

SEQ ID NO: 1 - RNA, 1291 nt, Saccharum sp.
  source 1..1291: mol_type = other RNA
gctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtaggtattagttgatccagatgccccaagcccaagcaatccatcactgaggggtcagcaaccgatcctttggtcatggctcatgtcatacaggatgtgttggatccctttacaacaaccattccactaagaataacatacaacaataggctacttctggcaagtgctgagctaaagccatctgcggttgcaagtaaaccacgagtcgatgtcggtggcaatgacatgagggctttctacaccctgggtttgaatcagattcagatttcattgcatcacagagatccatctttactctaccgcttgctctaacttaacttgtaattgttttttatcatgcagccagggtgtagaaagccctcatgtcattgccaccgacatcgactcgtggtttacttgcaaccgcagatggctttagctcagcacttgccagaagtagcctattgttgtatgttattcttagtggaatggttgttgtaaagggatccaacacatcctgtatgacatgagccatgaccaaaggatcggttgctgacccctcagtgatggattgcttgggcttggggcatctggatcaactaatacctaccagtgtaaagaacaccctgaggtcggtgcccccaacatcaactcttggcttattcaccactgcggatggcttcagctcaacacctgctagaagtagcctattgttgtaggttattctgaggggtatagttggtgtaaatgaatccaacacatctcgtatcacatgaccatcacgagggtgtagaaggtcctcatgtcgggtcccccgacctcgaccctgggctggttcaccaccatggacggcttgagctcgcagccgttggatacggtcctggtgccgtagctgaccctgaggttggtggtccggacgaaggggtccagcacgtcgcccaccaccctaccaaccaccagc

SEQ ID NO: 2 - RNA, 719 nt, Saccharum sp.
  source 1..719: mol_type = other RNA
gaccgtatccaacggctgcgagctcaagccgtccatggtggtgaaccagcccagggtcgaggtcgggggacccgacatgaggaccttctacaccctcgtgatgggaagccatccgcagtggtgaataagccaagagttgatgttgggggcaccgacctcagggtgttctttacactggtaggtattagttgatccagatgccccaagccaaccattccactaagaataacatacaacaataggctacttctggcaagtgctgagctaaagccatctgcggttgcaagtaaaccacgagtcgatgtcggtggcgtttgaatcagattcagatttcattgcatcacagagatccatctttactctaccgcttgctctaacttaacttgtaattgttttttatcatgcaggccaccgacatcgactcgtggtttacttgcaaccgcagatggctttagctcagcacttgccagaagtagcctattgttgtatgttattcttagtggaatggttggcttggggcatctggatcaactaatacctaccagtgtaaagaacaccctgaggtcggtgcccccaacatcaactcttggcttattcaccactgcggatggcttcccatcacgagggtgtagaaggtcctcatgtcgggtcccccgacctcgaccctgggctggttcaccaccatggacggcttgagctcgcagccgttggatacggtc

SEQ ID NO: 3 - RNA, 20 nt, Saccharum sp.
  source 1..20: mol_type = other RNA
gccgtagctgaccctgaggt

SEQ ID NO: 4 - RNA, 20 nt, Saccharum sp.
  source 1..20: mol_type = other RNA
ggcttgggctcggagcatcc

SEQ ID NO: 5 - RNA, 20 nt, Saccharum sp.
  source 1..20: mol_type = other RNA
gagaagtagcctattgttgt

SEQ ID NO: 6 - RNA, 20 nt, Saccharum sp.
  source 1..20: mol_type = other RNA
gcccaagcaatccatcactg

SEQ ID NO: 7 - RNA, 20 nt, Saccharum sp.
  source 1..20: mol_type = other RNA
ggtaaaccacgagtcgatgt

SEQ ID NO: 8 - RNA, 20 nt, Saccharum sp.
  source 1..20: mol_type = other RNA
gtccaagccatccatcacta

SEQ ID NO: 9 - RNA, 11724 nt, Saccharum sp.
  source 1..11724: mol_type = other RNA
ggaactagggataacagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtaggtattagttgatccagatgccccaagcccaagcaatccatcactgaggggtcagcaaccgatcctttggtcatggctcatgtcatacaggatgtgttggatccctttacaacaaccattccactaagaataacatacaacaataggctacttctggcaagtgctgagctaaagccatctgcggttgcaagtaaaccacgagtcgatgtcggtggcaatgacatgagggctttctacaccctgggtttgaatcagattcagatttcattgcatcacagagatccatctttactctaccgcttgctctaacttaacttgtaattgttttttatcatgcagccagggtgtagaaagccctcatgtcattgccaccgacatcgactcgtggtttacttgcaaccgcagatggctttagctcagcacttgccagaagtagcctattgttgtatgttattcttagtggaatggttgttgtaaagggatccaacacatcctgtatgacatgagccatgaccaaaggatcggttgctgacccctcagtgatggattgcttgggcttggggcatctggatcaactaatacctaccagtgtaaagaacaccctgaggtcggtgcccccaacatcaactcttggcttattcaccactgcggatggcttcagctcaacacctgctagaagtagcctattgttgtaggttattctgaggggtatagttggtgtaaatgaatccaacacatctcgtatcacatgaccatcacgagggtgtagaaggtcctcatgtcgggtcccccgacctcgaccctgggctggttcaccaccatggacggcttgagctcgcagccgttggatacggtcctggtgccgtagctgaccctgaggttggtggtccggacgaaggggtccagcacgtcgcccaccaccctaccaaccaccagcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataacagggtaatgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 10 - RNA, 12852 nt, Saccharum sp.
  source 1..12852: mol_type = other RNA
ggaactagggataacagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggccaccaccgccgccgccgctgccgccgcgctagccggcgccactaccgctgcgcccaaggcgaggcgccgggcgcacctcctggccgcacggcgcgccctcgccgcgcccatcaggtgctcagcggcgccacccgccacgctgacggtgacggctcccccggccaccccgctccggccgtggggccccaccgatccccgcaagggcgccgacatcctcgtcgaggctcttgagcgctgcggcgtccgcgacgtcttcgcctaccccggcggcgcgtccatggagatccaccaggcactcacccgttcccccgtcatcgccaaccacctcttccgccacgagcaaggggaggccttcgccgcctctggcttcgcgcgctcctcgggccgcgtcggcgtctgcgtcgccacctccggccccggcgccaccaacctagtctccgcgctcgccgacgcgctgctcgactccgtccccatggtcgccatcacgggacaggttccgcggcgcatgattggcaccgacgccttccaggagacgcccatcgtcgaggtcacccgctccatcaccaaacataactacctggtcctcgacgtcgacgacatcccccgcgtcgtgcaggaggctttcttcctcgcctcctccggtcgcccgggaccggtgcttgtcgacatccccaaggacatccagcagcagatggccgtgccggtctgggacacgcccatgagtctgcctgggtacattgcgcgccttcccaagcctcctgcgactgaattgcttgagcaggtgctgcgtcttgttggtgaatcaaggcgccctgttctttatgttggtggtggctgcgcagcatctggcgaggagttgcgccgctttgtggagatgactggaatcccagtcacaactactcttatgggccttggcaatttccctggcgacgacccactgtctctgcgcatgcttggtatgcatggcacggtgtatgcaaattatgcagtggataaggcggatctgttgcttgcatttggtgtgcggtttgatgatcgtgtgacagggaagattgaggcttttgcaagcagggctaagattgtgcacattgatattgatcccgctgagattggcaagaacaagcagccacatgtgtccatctgtgcagacgttaagcttgctttgcagggcatgaatgctcttctggaaggaagcacatcaaagaagagctttgactttggctcatggcaagctgagttggatcagcagaagagagagttcccccttgggtataaaacttttgatgacgagatccagccacaatatgctattcaggttcttgatgagctgacaaaaggggaggccatcattgccacaggtgttgggcagcaccagatgtgggcggcacagtactacacttacaagcggccaaggcagtggttgtcttcagctggtcttggggctatgggatttggtttgccggctgctgctggcgctgctgtggccaacccaggtatcactgttgttgacatcgacggagatggtagcttcctcatgaacattcaggagctagctatgatccgaattgagaacctcccagtgaaggtctttgtgctaaacaaccagcacctggggatggtggtgcagttggaggacaggttctataaggccaatagagcacacacatacttgggaaacccagagaatgaaagtgagatatatccagatttcgtgacaattgccaaagggttcaacattccagcagtccgtgtgacaaagaagagcgaagtccatgcagcaatcaagaagatgcttgagactccagggccatacctcttggatataatcgtcccgcaccaggagcatgtgttgcctatgatccctagtggtggggctttcaaggatatgatcctggatggtgatggcaggactgtgtattgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtaggtattagttgatccagatgccccaagcccaagcaatccatcactgaggggtcagcaaccgatcctttggtcatggctcatgtcatacaggatgtgttggatccctttacaacaaccattccactaagaataacatacaacaataggctacttctggcaagtgctgagctaaagccatctgcggttgcaagtaaaccacgagtcgatgtcggtggcaatgacatgagggctttctacaccctgggtttgaatcagattcagatttcattgcatcacagagatccatctttactctaccgcttgctctaacttaacttgtaattgttttttatcatgcagccagggtgtagaaagccctcatgtcattgccaccgacatcgactcgtggtttacttgcaaccgcagatggctttagctcagcacttgccagaagtagcctattgttgtatgttattcttagtggaatggttgttgtaaagggatccaacacatcctgtatgacatgagccatgaccaaaggatcggttgctgacccctcagtgatggattgcttgggcttggggcatctggatcaactaatacctaccagtgtaaagaacaccctgaggtcggtgcccccaacatcaactcttggcttattcaccactgcggatggcttcagctcaacacctgctagaagtagcctattgttgtaggttattctgaggggtatagttggtgtaaatgaatccaacacatctcgtatcacatgaccatcacgagggtgtagaaggtcctcatgtcgggtcccccgacctcgaccctgggctggttcaccaccatggacggcttgagctcgcagccgttggatacggtcctggtgccgtagctgaccctgaggttggtggtccggacgaaggggtccagcacgtcgcccaccaccctaccaaccaccagcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataacagggtaatgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 11 - RNA, 11152 nt, Saccharum sp.
  source 1..11152: mol_type = other RNA
ggaactagggataacagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggaccgtatccaacggctgcgagctcaagccgtccatggtggtgaaccagcccagggtcgaggtcgggggacccgacatgaggaccttctacaccctcgtgatgggaagccatccgcagtggtgaataagccaagagttgatgttgggggcaccgacctcagggtgttctttacactggtaggtattagttgatccagatgccccaagccaaccattccactaagaataacatacaacaataggctacttctggcaagtgctgagctaaagccatctgcggttgcaagtaaaccacgagtcgatgtcggtggcgtttgaatcagattcagatttcattgcatcacagagatccatctttactctaccgcttgctctaacttaacttgtaattgttttttatcatgcaggccaccgacatcgactcgtggtttacttgcaaccgcagatggctttagctcagcacttgccagaagtagcctattgttgtatgttattcttagtggaatggttggcttggggcatctggatcaactaatacctaccagtgtaaagaacaccctgaggtcggtgcccccaacatcaactcttggcttattcaccactgcggatggcttcccatcacgagggtgtagaaggtcctcatgtcgggtcccccgacctcgaccctgggctggttcaccaccatggacggcttgagctcgcagccgttggatacggtcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataacagggtaatgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 12 - RNA, 12280 nt, Saccharum sp.
  source 1..12280: mol_type = other RNA
ggaactagggataacagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggccaccaccgccgccgccgctgccgccgcgctagccggcgccactaccgctgcgcccaaggcgaggcgccgggcgcacctcctggccgcacggcgcgccctcgccgcgcccatcaggtgctcagcggcgccacccgccacgctgacggtgacggctcccccggccaccccgctccggccgtggggccccaccgatccccgcaagggcgccgacatcctcgtcgaggctcttgagcgctgcggcgtccgcgacgtcttcgcctaccccggcggcgcgtccatggagatccaccaggcactcacccgttcccccgtcatcgccaaccacctcttccgccacgagcaaggggaggccttcgccgcctctggcttcgcgcgctcctcgggccgcgtcggcgtctgcgtcgccacctccggccccggcgccaccaacctagtctccgcgctcgccgacgcgctgctcgactccgtccccatggtcgccatcacgggacaggttccgcggcgcatgattggcaccgacgccttccaggagacgcccatcgtcgaggtcacccgctccatcaccaaacataactacctggtcctcgacgtcgacgacatcccccgcgtcgtgcaggaggctttcttcctcgcctcctccggtcgcccgggaccggtgcttgtcgacatccccaaggacatccagcagcagatggccgtgccggtctgggacacgcccatgagtctgcctgggtacattgcgcgccttcccaagcctcctgcgactgaattgcttgagcaggtgctgcgtcttgttggtgaatcaaggcgccctgttctttatgttggtggtggctgcgcagcatctggcgaggagttgcgccgctttgtggagatgactggaatcccagtcacaactactcttatgggccttggcaatttccctggcgacgacccactgtctctgcgcatgcttggtatgcatggcacggtgtatgcaaattatgcagtggataaggcggatctgttgcttgcatttggtgtgcggtttgatgatcgtgtgacagggaagattgaggcttttgcaagcagggctaagattgtgcacattgatattgatcccgctgagattggcaagaacaagcagccacatgtgtccatctgtgcagacgttaagcttgctttgcagggcatgaatgctcttctggaaggaagcacatcaaagaagagctttgactttggctcatggcaagctgagttggatcagcagaagagagagttcccccttgggtataaaacttttgatgacgagatccagccacaatatgctattcaggttcttgatgagctgacaaaaggggaggccatcattgccacaggtgttgggcagcaccagatgtgggcggcacagtactacacttacaagcggccaaggcagtggttgtcttcagctggtcttggggctatgggatttggtttgccggctgctgctggcgctgctgtggccaacccaggtatcactgttgttgacatcgacggagatggtagcttcctcatgaacattcaggagctagctatgatccgaattgagaacctcccagtgaaggtctttgtgctaaacaaccagcacctggggatggtggtgcagttggaggacaggttctataaggccaatagagcacacacatacttgggaaacccagagaatgaaagtgagatatatccagatttcgtgacaattgccaaagggttcaacattccagcagtccgtgtgacaaagaagagcgaagtccatgcagcaatcaagaagatgcttgagactccagggccatacctcttggatataatcgtcccgcaccaggagcatgtgttgcctatgatccctagtggtggggctttcaaggatatgatcctggatggtgatggcaggactgtgtattgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggaccgtatccaacggctgcgagctcaagccgtccatggtggtgaaccagcccagggtcgaggtcgggggacccgacatgaggaccttctacaccctcgtgatgggaagccatccgcagtggtgaataagccaagagttgatgttgggggcaccgacctcagggtgttctttacactggtaggtattagttgatccagatgccccaagccaaccattccactaagaataacatacaacaataggctacttctggcaagtgctgagctaaagccatctgcggttgcaagtaaaccacgagtcgatgtcggtggcgtttgaatcagattcagatttcattgcatcacagagatccatctttactctaccgcttgctctaacttaacttgtaattgttttttatcatgcaggccaccgacatcgactcgtggtttacttgcaaccgcagatggctttagctcagcacttgccagaagtagcctattgttgtatgttattcttagtggaatggttggcttggggcatctggatcaactaatacctaccagtgtaaagaacaccctgaggtcggtgcccccaacatcaactcttggcttattcaccactgcggatggcttcccatcacgagggtgtagaaggtcctcatgtcgggtcccccgacctcgaccctgggctggttcaccaccatggacggcttgagctcgcagccgttggatacggtcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataacagggtaatgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 13 - RNA, 13810 nt, Saccharum sp.
  source 1..13810: mol_type = other RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattaactataacggtcctaaggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgccgtagctgaccctgaggtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggcttgggctcggagcatccgttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaaggtagcgaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - RNA, 13810 nt, Saccharum sp.
  source 1..13810: mol_type = other RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattaactataacggtcctaaggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgagaagtagcctattgttgtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgcccaagcaatccatcactggttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaaggtagcgaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - RNA, 13810 nt, Saccharum sp.
  source 1..13810: mol_type = other RNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattaactataacggtcctaaggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggtaaaccacgagtcgatgtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgtccaagccatccatcactagttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaaggtagcgaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - RNA, 9025 nt, Saccharum sp.
  source 1..9025: mol_type = other RNA
cagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtaggtattagttgatccagatgccccaagcccaagcaatccatcactgaggggtcagcaaccgatcctttggtcatggctcatgtcatacaggatgtgttggatccctttacaacaaccattccactaagaataacatacaacaataggctacttctggcaagtgctgagctaaagccatctgcggttgcaagtaaaccacgagtcgatgtcggtggcaatgacatgagggctttctacaccctgggtttgaatcagattcagatttcattgcatcacagagatccatctttactctaccgcttgctctaacttaacttgtaattgttttttatcatgcagccagggtgtagaaagccctcatgtcattgccaccgacatcgactcgtggtttacttgcaaccgcagatggctttagctcagcacttgccagaagtagcctattgttgtatgttattcttagtggaatggttgttgtaaagggatccaacacatcctgtatgacatgagccatgaccaaaggatcggttgctgacccctcagtgatggattgcttgggcttggggcatctggatcaactaatacctaccagtgtaaagaacaccctgaggtcggtgcccccaacatcaactcttggcttattcaccactgcggatggcttcagctcaacacctgctagaagtagcctattgttgtaggttattctgaggggtatagttggtgtaaatgaatccaacacatctcgtatcacatgaccatcacgagggtgtagaaggtcctcatgtcgggtcccccgacctcgaccctgggctggttcaccaccatggacggcttgagctcgcagccgttggatacggtcctggtgccgtagctgaccctgaggttggtggtccggacgaaggggtccagcacgtcgcccaccaccctaccaaccaccagcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataa

SEQ ID NO: 17 - RNA, 10153 nt, Saccharum sp.
  source 1..10153: mol_type = other RNA
cagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggccaccaccgccgccgccgctgccgccgcgctagccggcgccactaccgctgcgcccaaggcgaggcgccgggcgcacctcctggccgcacggcgcgccctcgccgcgcccatcaggtgctcagcggcgccacccgccacgctgacggtgacggctcccccggccaccccgctccggccgtggggccccaccgatccccgcaagggcgccgacatcctcgtcgaggctcttgagcgctgcggcgtccgcgacgtcttcgcctaccccggcggcgcgtccatggagatccaccaggcactcacccgttcccccgtcatcgccaaccacctcttccgccacgagcaaggggaggccttcgccgcctctggcttcgcgcgctcctcgggccgcgtcggcgtctgcgtcgccacctccggccccggcgccaccaacctagtctccgcgctcgccgacgcgctgctcgactccgtccccatggtcgccatcacgggacaggttccgcggcgcatgattggcaccgacgccttccaggagacgcccatcgtcgaggtcacccgctccatcaccaaacataactacctggtcctcgacgtcgacgacatcccccgcgtcgtgcaggaggctttcttcctcgcctcctccggtcgcccgggaccggtgcttgtcgacatccccaaggacatccagcagcagatggccgtgccggtctgggacacgcccatgagtctgcctgggtacattgcgcgccttcccaagcctcctgcgactgaattgcttgagcaggtgctgcgtcttgttggtgaatcaaggcgccctgttctttatgttggtggtggctgcgcagcatctggcgaggagttgcgccgctttgtggagatgactggaatcccagtcacaactactcttatgggccttggcaatttccctggcgacgacccactgtctctgcgcatgcttggtatgcatggcacggtgtatgcaaattatgcagtggataaggcggatctgttgcttgcatttggtgtgcggtttgatgatcgtgtgacagggaagattgaggcttttgcaagcagggctaagattgtgcacattgatattgatcccgctgagattggcaagaacaagcagccacatgtgtccatctgtgcagacgttaagcttgctttgcagggcatgaatgctcttctggaaggaagcacatcaaagaagagctttgactttggctcatggcaagctgagttggatcagcagaagagagagttcccccttgggtataaaacttttgatgacgagatccagccacaatatgctattcaggttcttgatgagctgacaaaaggggaggccatcattgccacaggtgttgggcagcaccagatgtgggcggcacagtactacacttacaagcggccaaggcagtggttgtcttcagctggtcttggggctatgggatttggtttgccggctgctgctggcgctgctgtggccaacccaggtatcactgttgttgacatcgacggagatggtagcttcctcatgaacattcaggagctagctatgatccgaattgagaacctcccagtgaaggtctttgtgctaaacaaccagcacctggggatggtggtgcagttggaggacaggttctataaggccaatagagcacacacatacttgggaaacccagagaatgaaagtgagatatatccagatttcgtgacaattgccaaagggttcaacattccagcagtccgtgtgacaaagaagagcgaagtccatgcagcaatcaagaagatgcttgagactccagggccatacctcttggatataatcgtcccgcaccaggagcatgtgttgcctatgatccctagtggtggggctttcaaggatatgatcctggatggtgatggcaggactgtgtattgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtaggtattagttgatccagatgccccaagcccaagcaatccatcactgaggggtcagcaaccgatcctttggtcatggctcatgtcatacaggatgtgttggatccctttacaacaaccattccactaagaataacatacaacaataggctacttctggcaagtgctgagctaaagccatctgcggttgcaagtaaaccacgagtcgatgtcggtggcaatgacatgagggctttctacaccctgggtttgaatcagattcagatttcattgcatcacagagatccatctttactctaccgcttgctctaacttaacttgtaattgttttttatcatgcagccagggtgtagaaagccctcatgtcattgccaccgacatcgactcgtggtttacttgcaaccgcagatggctttagctcagcacttgccagaagtagcctattgttgtatgttattcttagtggaatggttgttgtaaagggatccaacacatcctgtatgacatgagccatgaccaaaggatcggttgctgacccctcagtgatggattgcttgggcttggggcatctggatcaactaatacctaccagtgtaaagaacaccctgaggtcggtgcccccaacatcaactcttggcttattcaccactgcggatggcttcagctcaacacctgctagaagtagcctattgttgtaggttattctgaggggtatagttggtgtaaatgaatccaacacatctcgtatcacatgaccatcacgagggtgtagaaggtcctcatgtcgggtcccccgacctcgaccctgggctggttcaccaccatggacggcttgagctcgcagccgttggatacggtcctggtgccgtagctgaccctgaggttggtggtccggacgaaggggtccagcacgtcgcccaccaccctaccaaccaccagcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataa

SEQ ID NO: 18 - RNA, 8453 nt, Saccharum sp.
  source 1..8453: mol_type = other RNA
cagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggaccgtatccaacggctgcgagctcaagccgtccatggtggtgaaccagcccagggtcgaggtcgggggacccgacatgaggaccttctacaccctcgtgatgggaagccatccgcagtggtgaataagccaagagttgatgttgggggcaccgacctcagggtgttctttacactggtaggtattagttgatccagatgccccaagccaaccattccactaagaataacatacaacaataggctacttctggcaagtgctgagctaaagccatctgcggttgcaagtaaaccacgagtcgatgtcggtggcgtttgaatcagattcagatttcattgcatcacagagatccatctttactctaccgcttgctctaacttaacttgtaattgttttttatcatgcaggccaccgacatcgactcgtggtttacttgcaaccgcagatggctttagctcagcacttgccagaagtagcctattgttgtatgttattcttagtggaatggttggcttggggcatctggatcaactaatacctaccagtgtaaagaacaccctgaggtcggtgcccccaacatcaactcttggcttattcaccactgcggatggcttcccatcacgagggtgtagaaggtcctcatgtcgggtcccccgacctcgaccctgggctggttcaccaccatggacggcttgagctcgcagccgttggatacggtcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataa

SEQ ID NO: 19 - RNA, 9581 nt, Saccharum sp.
  source 1..9581: mol_type = other RNA
cagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggccaccaccgccgccgccgctgccgccgcgctagccggcgccactaccgctgcgcccaaggcgaggcgccgggcgcacctcctggccgcacggcgcgccctcgccgcgcccatcaggtgctcagcggcgccacccgccacgctgacggtgacggctcccccggccaccccgctccggccgtggggccccaccgatccccgcaagggcgccgacatcctcgtcgaggctcttgagcgctgcggcgtccgcgacgtcttcgcctaccccggcggcgcgtccatggagatccaccaggcactcacccgttcccccgtcatcgccaaccacctcttccgccacgagcaaggggaggccttcgccgcctctggcttcgcgcgctcctcgggccgcgtcggcgtctgcgtcgccacctccggccccggcgccaccaacctagtctccgcgctcgccgacgcgctgctcgactccgtccccatggtcgccatcacgggacaggttccgcggcgcatgattggcaccgacgccttccaggagacgcccatcgtcgaggtcacccgctccatcaccaaacataactacctggtcctcgacgtcgacgacatcccccgcgtcgtgcaggaggctttcttcctcgcctcctccggtcgcccgggaccggtgcttgtcgacatccccaaggacatccagcagcagatggccgtgccggtctgggacacgcccatgagtctgcctgggtacattgcgcgccttcccaagcctcctgcgactgaattgcttgagcaggtgctgcgtcttgttggtgaatcaaggcgccctgttctttatgttggtggtggctgcgcagcatctggcgaggagttgcgccgctttgtggagatgactggaatcccagtcacaactactcttatgggccttggcaatttccctggcgacgacccactgtctctgcgcatgcttggtatgcatggcacggtgtatgcaaattatgcagtggataaggcggatctgttgcttgcatttggtgtgcggtttgatgatcgtgtgacagggaagattgaggcttttgcaagcagggctaagattgtgcacattgatattgatcccgctgagattggcaagaacaagcagccacatgtgtccatctgtgcagacgttaagcttgctttgcagggcatgaatgctcttctggaaggaagcacatcaaagaagagctttgactttggctcatggcaagctgagttggatcagcagaagagagagttcccccttgggtataaaacttttgatgacgagatccagccacaatatgctattcaggttcttgatgagctgacaaaaggggaggccatcattgccacaggtgttgggcagcaccagatgtgggcggcacagtactacacttacaagcggccaaggcagtggttgtcttcagctggtcttggggctatgggatttggtttgccggctgctgctggcgctgctgtggccaacccaggtatcactgttgttgacatcgacggagatggtagcttcctcatgaacattcaggagctagctatgatccgaattgagaacctcccagtgaaggtctttgtgctaaacaaccagcacctggggatggtggtgcagttggaggacaggttctataaggccaatagagcacacacatacttgggaaacccagagaatgaaagtgagatatatccagatttcgtgacaattgccaaagggttcaacattccagcagtccgtgtgacaaagaagagcgaagtccatgcagcaatcaagaagatgcttgagactccagggccatacctcttggatataatcgtcccgcaccaggagcatgtgttgcctatgatccctagtggtggggctttcaaggatatgatcctggatggtgatggcaggactgtgtattgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatcactctgtggtctcaaatggaccgtatccaacggctgcgagctcaagccgtccatggtggtgaaccagcccagggtcgaggtcgggggacccgacatgaggaccttctacaccctcgtgatgggaagccatccgcagtggtgaataagccaagagttgatgttgggggcaccgacctcagggtgttctttacactggtaggtattagttgatccagatgccccaagccaaccattccactaagaataacatacaacaataggctacttctggcaagtgctgagctaaagccatctgcggttgcaagtaaaccacgagtcgatgtcggtggcgtttgaatcagattcagatttcattgcatcacagagatccatctttactctaccgcttgctctaacttaacttgtaattgttttttatcatgcaggccaccgacatcgactcgtggtttacttgcaaccgcagatggctttagctcagcacttgccagaagtagcctattgttgtatgttattcttagtggaatggttggcttggggcatctggatcaactaatacctaccagtgtaaagaacaccctgaggtcggtgcccccaacatcaactcttggcttattcaccactgcggatggcttcccatcacgagggtgtagaaggtcctcatgtcgggtcccccgacctcgaccctgggctggttcaccaccatggacggcttgagctcgcagccgttggatacggtcgctttgagaccacgaagtgttcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataa

SEQ ID NO: 20 - RNA, 11158 nt, Saccharum sp.
  source 1..11158: mol_type = other RNA
ggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgccgtagctgaccctgaggtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggcttgggctcggagcatccgttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaa

SEQ ID NO: 21 - RNA, 11158 nt, Saccharum sp.
  source 1..11158: mol_type = other RNA
ggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgagaagtagcctattgttgtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgcccaagcaatccatcactggttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaa

SEQ ID NO: 22 - RNA, 11158 nt, Saccharum sp.
  source 1..11158: mol_type = other RNA
ggtagcgaggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagatatcgcccgggctttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttggtaaaccacgagtcgatgtgttttagagctagaaatagcaagttaaaataaggctagtccgttatcaacttgaaaaagtggcaccgagtcggtgctttttttttaagctttttgtgaaagttgaattacggcatagccgaaggaataacagaatcgtttcacactttcgtaacaaagatcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgtccaagccatccatcactagttttagagctagaaatagcaagttaaaataaggctagtccgttatcaacttgaaaaagtggcaccgagtcggtgctttttttttgcggccgcctgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggccggccatggatcctaaaaagaaacgtaaggtcgacaagaagtacagcattggtctggacatcgggacgaattccgttggctgggccgtgatcaccgatgagtacaaggtcccttccaagaagttcaaggttctggggaacaccgatcggcacagcatcaagaagaacctcattggagccctcctgttcgactcaggcgagaccgccgaagcaacaaggctcaagagaaccgcaaggagacggtataccagaaggaagaacaggatctgctacctgcaggagatcttcagcaacgaaatggcgaaggtggacgattcgttcttccacagattggaagagagtttcctcgtcgaggaagacaagaagcacgagaggcatccgatcttcggcaacatcgtcgacgaggttgcctatcacgagaagtaccccaccatctaccatctgcggaagaagcttgtggactcgactgacaaggcggaccttaggttgatctacctcgctctggcccacatgatcaagttcaggggccatttcctgatcgagggggatctgaacccggacaacagcgatgtggacaagttgttcatccagctcgtccaaacctacaaccagctctttgaggagaacccgatcaacgcttcaggcgtcgacgccaaggcgatcctgtctgcacgcctgtcaaagtctcgccggcttgagaacttgatcgctcaactcccgggcgagaagaagaacggcttgttcgggaatctcattgcactttcgttggggctcaccccgaacttcaagagcaacttcgatctcgctgaggacgcaaagctgcagctttccaaggacacttacgacgatgacctggataacctgttggcccaaatcggcgatcagtacgcggacttgttcctcgccgcgaagaacttgtcggacgcgatcctcctgagtgacattctccgcgtgaacaccgagatcaccaaggccccgctctcggcgagcatgatcaagcggtatgacgagcaccatcaggatctgaccctgttgaaggctttggtccggcagcaactcccagagaagtacaaggagatcttcttcgatcagtccaagaacggctacgctggctacatcgacggcggggcatcgcaggaggagttctacaagttcatcaagccaatcctggagaagatggatggcacagaggaactcctggtgaagctcaacagggaggacctgttgcggaagcaaaggactttcgataacggcagcatccctcaccagatccatctcggggagctgcacgccatcctgagaaggcaggaagacttctaccccttccttaaggataaccgggagaagatcgagaagatcctgacgttcagaatcccgtactacgtcggaccactcgcccggggtaattccagattcgcgtggatgaccagaaagagcgaggaaaccatcaccccttggaacttcgaggaagtggtcgataagggcgcttccgcacagagcttcatcgagcgcatgaccaacttcgacaagaacctgcctaacgagaaggtccttcccaagcattccctcctgtacgagtacttcactgtgtacaacgaactcacgaaggtgaagtacgtgaccgagggaatgcgcaagcccgccttcctgagcggcgagcagaagaaggcgatcgtggacctgttgttcaagaccaatcggaaggtcacagtgaagcagctcaaggaggactacttcaagaagatcgaatgcttcgattccgttgagatcagcggcgtggaagacaggtttaacgcctcactggggacttaccacgatctcctgaagatcatcaaggataaggacttcttggacaacgaggagaatgaggacatcctcgaagacattgtcctgactcttacgttgttcgaggatagggagatgatcgaggaacgcttgaagacgtatgcccatctcttcgatgacaaggtgatgaagcagctcaagagaagaaggtacaccggatggggaaggctgtcccgcaagctgatcaacggcatcagagacaagcaatcagggaagacaatccttgacttcttgaagtctgatggcttcgcgaacaggaacttcatgcagctcattcacgatgactcactgaccttcaaggaggacatccagaaggctcaagtgtcgggacaaggtgacagtctgcacgagcatatcgccaaccttgcgggatctcctgccatcaagaagggtatcctgcagacagtcaaggttgtggatgagcttgtgaaggtcatgggacggcataagcccgagaacatcgttatcgagatggccagagagaatcagaccacccagaagggtcagaagaactcgagggagcgcatgaagcgcatcgaggaaggcattaaggagctggggagtcagatccttaaggagcacccggtggagaacacgcagttgcagaatgagaagctctatctgtactacctgcagaatggcagggacatgtacgtggaccaggagttggacatcaaccgcctctcggattacgacgtcgatcacatcgttcctcagtccttccttaaggatgacagcattgacaacaaggttctcaccaggtccgacaagaaccgcgggaagtccgataacgtgcccagcgaggaagtcgtgaagaagatgaagaactactggaggcaactgctgaatgccaagttgatcacccagaggaagttcgacaacctcactaaggccgagcgcggaggtctcagcgaactggacaaggcgggcttcatcaagcggcaactggttgagactagacagatcacgaagcacgtcgcgcagattctcgattcacgcatgaacacgaagtacgatgagaatgacaagctgatccgggaagtgaaggtcatcaccttgaagtcaaagctcgtttctgacttcaggaaggatttccagttctacaaggtgcgcgagatcaacaactaccaccatgctcatgacgcatacctcaacgctgtggtcggaacagcactgatcaagaagtacccgaagctcgagtccgaattcgtgtacggtgactacaaggtttacgatgtgcgcaagatgatcgccaagtcagagcaggaaattggcaaggccactgcgaagtacttcttctactccaacatcatgaacttcttcaagactgagatcacgctggctaatggcgaaatccggaagagaccactgattgagaccaacggcgagacaggggaaatcgtctgggacaaggggagggatttcgccacagtccgcaaggttctctctatgcctcaagtgaacatcgtcaagaagactgaagtccagacgggcgggttctcaaaggagtctatcctgcccaagcggaactcggataagcttatcgccaggaagaaggactgggatccgaagaagtacggaggtttcgactcaccaacggtggcttactctgtcctggttgtggcaaaggtggagaagggaaagtcgaagaagctcaagtctgtcaaggagctgctgggtatcaccattatggagaggtccagcttcgagaagaacccgatcgacttcctcgaggcgaagggctacaaggaagtgaagaaggacctgatcatcaagcttccgaagtacagtctgttcgagttggagaacggcaggaagcgcatgttggcttccgcaggagagctccagaagggtaacgagcttgctttgccgtccaagtacgtgaacttcctctatctggcatcccactacgagaagctcaagggcagcccagaggataacgaacagaagcaactgtttgtggagcaacacaagcattaccttgacgagatcatcgaacagatctcggagttcagtaagcgcgtcatcctcgccgacgcgaatttggataaggttctctcagcctacaacaagcaccgggacaagcctatcagagagcaggcggagaacatcatccatctcttcaccctgacaaaccttggggctcccgctgccttcaagtacttcgacactacgatcgaccggaagaggtacacttctacgaaggaggtgctggatgcaacccttatccaccaatcgatcactggcctctacgagacgcggatcgacttgagtcagctcggtggcgataagagacccgcagcaaccaagaaggcagggcaagcaaagaagaagaagtgagcgatcgcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagggtacgtaccgcgatcgcataacttcgtatagcatacattatacgaagttatgaattcggcaatcaagaggtaaaggataataatcccagcagaaaccgtgatcaaagaaacaattcattgcaatatagactaggccatgcatacgcaaatccaagcgtgcatccactgactgcataacataaacccccaggtactacgactaagccgaactgatacaaaatgcactagctattggataatcagatcctggtccatgctgctgtcattccatcgcaatcaccctggtagcgacgacaggatcatttgcttccagaaccgaactgtgcgcctagctcggcgcgcctaactataacggtcctaa

SEQ ID NO: 23 - RNA, 741 nt, Sorghum sp.
  source 1..741: mol_type = mRNA
atgtcagggccagcatgcggtgtgtgcggtgcagccgcctgctgccggcacctcttccacaccggcgacgagaacgacgacttcaacagccggcgggccttgttctctgtgttccctgcggcggttcaccatcatgagcccagccccagcagcatgcagcagcagcctccggcggggtgcctgcacgagttccagttctttggccatcaggacaacgatgaccaccaagaaagcatcgcctggctcttcgaccacccgccgccacctgcgcatgatgtcgacgacgacgaccggtccccagctgagaaccagcagcctcatcaccgggcgtttgacccgtttgggacggagggaaacgggctcacctttgaggttgatgcccggctgggcctcggcagcgggggcgccgcccggcaaacagcagagacagcagcagcaagcgccaccatcatgtcattctgtgggagcacattcacagacgccgcaagctcgaggctcaaggagccaaccctgactgacgacagtcagctgcaaatgccggtaggtcagtcaacggagagggaggctaagttgatgaggtacaaggagaagaggatgatgaggtgttatgagaagcagataagatatgcatccaggaaagcctatgcgcaggtgaggccccgggtgaaaggtcgctttgccaaggtaaccgaagcctgctccgccacagcagacaatgttggcaacgaccacctgctctga

SEQ ID NO: 24 - RNA, 11145 nt, Sorghum sp.
  source 1..11145: mol_type = other RNA
ggaactagggataacagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatgccgccgccatgtcagggccagcatgcggtgtgtgcggtgcagccgcctgctgccggcacctcttccacaccggcgacgagaacgacgacttcaacagccggcgggccttgttctctgtgttccctgcggcggttcaccatcatgagcccagccccagcagcatgcagcagcagcctccggcggggtgcctgcacgagttccagttctttggccatcaggacaacgatgaccaccaagaaagcatcgcctggctcttcgaccacccgccgccacctgcgcatgatgtcgacgacgacgaccggtccccagctgagaaccagcagcctcatcaccgggcgtttgacccgtttgggacggagggaaacgggctcacctttgaggttgatgcccggctgggcctcggcagcgggggcgccgcccggcaaacagcagagacagcagcagcaagcgccaccatcatgtcattctgtgggagcacattcacagacgccgcaagctcgaggctcaaggagccaaccctgactgacgacagtcagctgcaaatgccggtaggtcagtcaacggagagggaggctaagttgatgaggtacaaggagaagaggatgatgaggtgttatgagaagcagataagatatgcatccaggaaagcctatgcgcaggtgaggccccgggtgaaaggtcgctttgccaaggtaaccgaagcctgctccgccacagcagacaatgttggcaacgaccacctgctctgattcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataacagggtaatgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtctcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggat

SEQ ID NO: 25 - RNA, 8446 nt, Sorghum sp.
  source 1..8446: mol_type = other RNA
cagggtaatggcgcgccgttgttttagtgtccgaggtttggtttggtgaagtacatctgaactctgaaggttcttcgtgacatggttagacgtggtgttgcagtactgtgataaaacttatcagaataagttttcgcttctaatttgttttcttaaatcatgtgttgatcagcctatgagttgttcatatcgttgcctttcgtgttgaacatttgttttcttgccatactaatgagattcatctgcacaaactgtacgaagagaatcaagtttaaacactgcaggtcaacggatcacctatcaacatcccagctaaaaacagtaaaaagggggaaaacgtgggtgagttgagtctgtcttgtggaaaaaacgttttagtttctcctggaattaacaataaaaacagttgaacaagattgactgttcctccgggagggtttggaacatcgttacagatgtgagcgaaaggtgaggaaacagagcggagggcttggaggtgacctcggtagtcgacgccggagttgagcttgatgacgacaccgtactggcgtaccaggcctagtagtgaacaccgggcctgaagctgtcgccgccgctgctcatcttgtgggctgtgcccggtgtccctgttgcggattgcgggtggcagcctggcaggtgggtgcgacccgtttggactccctgatctgggccctttgtgtcagtaccgtctgtactccgatgacatgcacaccgtcgtccacagtcaagtccacaatctcccctctttttttaacggaatagttcaaaatctccttgacgcacgctatcgtgtaccagcgctcactggacaccacgtttgtaatccacgccgacacgtcggtcccacgtcgacaggccccaccgtccggtctgtagcgtgtacgtattcgggcgacggacgtgtcgtcgtcgtcttgcgagtcccattcccatcaccatctgagccacacatcctctgaacaaaagcagggaggcctctacgcacatccccctttctcccactccttgtccgtggcacccaccccaaaccctcgcgccgcctccgagacagccgccgcaaccatggccaccgcctgcaggaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcccctgcaggatggggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgatcgcagagccatgggggcggtgagagctcgcggcgtcggatctccggtttcagtttcagctacgcgacttgtgtgtgtgtgtgtgtgtcagtgcgtggttactctgagcttcggtatgtttggtgtgtactgttcagtgtttcatggtgcttcctagcagtgtctgtcgtgtactttgcctgtatgttgagctcggctcagtatgttctgggagtgaataaataaaaaaaaattaaccagatattgtcgcgtgtacttagtatactaatcacctgctcctgctcttgctctgttttatcacctttcgggcttgactgtctttgtggaggcctctcagtctgatctggaactctacctaagttttactagtcccgggaagcttctgcagtgcagcgtgacccggtcgtgcccctcactagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctctagagcggccgcatttaaatgccgccgccatgtcagggccagcatgcggtgtgtgcggtgcagccgcctgctgccggcacctcttccacaccggcgacgagaacgacgacttcaacagccggcgggccttgttctctgtgttccctgcggcggttcaccatcatgagcccagccccagcagcatgcagcagcagcctccggcggggtgcctgcacgagttccagttctttggccatcaggacaacgatgaccaccaagaaagcatcgcctggctcttcgaccacccgccgccacctgcgcatgatgtcgacgacgacgaccggtccccagctgagaaccagcagcctcatcaccgggcgtttgacccgtttgggacggagggaaacgggctcacctttgaggttgatgcccggctgggcctcggcagcgggggcgccgcccggcaaacagcagagacagcagcagcaagcgccaccatcatgtcattctgtgggagcacattcacagacgccgcaagctcgaggctcaaggagccaaccctgactgacgacagtcagctgcaaatgccggtaggtcagtcaacggagagggaggctaagttgatgaggtacaaggagaagaggatgatgaggtgttatgagaagcagataagatatgcatccaggaaagcctatgcgcaggtgaggccccgggtgaaaggtcgctttgccaaggtaaccgaagcctgctccgccacagcagacaatgttggcaacgaccacctgctctgattcgaattaattaaggggacaccgttgtcggagcagtgccagtgagctgggtggcaacctgcgggactctggtgaggaagagcaaatggtagaaagcagtagctgtgcttctgaacattgtgtgtgtttgtttcccttgttaccgccaatcatcctacctgatgtgtcgtctggggtttcgtttgaaccatgctgggtttggagctttgtgatgcaaagcagaatgtcctgatgctttctttctccatagaataaatctctcctgttacctaaaggccggccgatatccattcaagaggagagctcatttcctctcctctcatcacctttgcctaaaaacagtatgaaagtcaagctaaagactttaaactagctcacttgggaggaagtcccatgtctattcttgtatatacttttttcccttgttatttttgatgttttgtgtaagtatttcaggttttatagtccacggaagtcaacgatcgagtgatataaatcgagtcaaagaatcttagccgtgcgatagagtgacttaaggcgaagcatccagctaatccgagtcccacacaacctattccttggagctctaaacgggagagtatggacgacttcgtcaactcctatttcaccagatcactaaggtaacacaccacccccattgtatataccgttatcttttatttttctttgtttgttgtttgttttagtcttgaattctctctcagatttttaatacacaagggactgtgtaatttaagtgtgggggagggttcaagaaatatttgactttatgttctcttttcttatttcaaattttgcatcatctaaggcataaaaaaccataaaaatttaaaaacatttatgaaaatgatttcaccaaaacagagtgtcatgtagattgcattgcattttaggattgtatatagagtacttgcatttaggatttctcatttgcataggggataatgatgatttcgtaccgttaagcatgttttgattcaatgaagtaagttcaatgcccaggttgttagttctctgttgcataaccgattgtttctctgaagtaagactgcaccctgctttgaatttacaacttgactatacttctttgattcaaaactcgcttatctgatgccattccctatctattagaacctgaactgaatttgcaattatcatgtctatgcattgtttgtgaactcatggctaccatatacatacttgtattatcttattcacttcaccaccctctgttaatctaagtagctgactctcatttttagagcagtttcccatacccttagcctagccttatttcaagccaaagatcatttgtgtgtgattgtgaggtctttttcgattgagcttggtataacgtgtcaggtatgagccgacaagagcagtctttcatgtagacctagcccacgctttttgggttagctaggactaggtggagacttgtatttgggattggggttgtgtatggattgaatgggaaagagaatgagaatgaaaagaaaagaaaaaaagagagtaagagtttagagttcttaagggggatacaaaaagtgttagtctagtaaagggtttggaattcaaagaagttaggcattgtgatttaaagaaaagggtaaaacgacctaagattagaagaaaagccttaagtttgtcaaagtataaaaccaaacccactagactcttgaatcgttttaaagaagaaccccttagaacttgaaccaaaaagagaaagaatgagaaaaagggtagagttaggacatctatgggactggaaccatcttatgaactatcatactatatcattgggtagatgggagtctgttcttgtttgtgtccattggcttttacctttagcattctgctgacgctcaatcttttctttgagagtcccctgttactcgaccaaatcattaaagggaccatttttgtctcttagcctaagcctgaaaccaagtgagttcataaccattgcattgcttgatccattgttcgtgcttagtgaatgttaaaaagaattggttgatgtgaatgcttgaatagattaaggcaggttgttaggttgctttgtgacgagtatggctaaatttttaagtaagggtctatcattttgcaacctataattagctacctggacattgagcttacccgttctatatgcatgctacgggttttgaatccccaccttcaaacctctccttcaacttaagattttttgtttgcttgagggcaagcaagagataagtttgggggagttgataacactgtaatttactcattttgggcatgtatttatcatcatttagcttagtgatcactcattgtttcatatcattttgcatcacatttagcttattttgcacacttaggcatttagggtcatcttgcatacatattgcatgttatggtgtgttttcaggtgcaaaataagtttacaaggagtttactcgaccctctaccctattgccccagaggggaaccagaggagcagctactcgaccaattattcattgcctcagaggggaagcagcagagcagctactcgaccagttattcggcgcgcctagggataa

SEQ ID NO: 26 - DNA, 95 bp, Oryza sativa
  source 1..95: mol_type = genomic DNA
gtttgaatcagattcagatttcattgcatcacagagatccatctttactctaccgcttgctctaacttaacttgtaattgttttttatcatgcag

SEQ ID NO: 27 - RNA, 20 nt, Saccharum sp.
  source 1..20: mol_type = other RNA
ggcaccaggaccgtatccaa

SEQ ID NO: 28 - RNA, 20 nt, Saccharum sp.
  source 1..20: mol_type = other RNA
gtgtagaaggtcctcatgtc

SEQ ID NO: 29 - RNA, 20 nt, Saccharum sp.
  source 1..20: mol_type = other RNA
gtagctgaccctgaggttgg

SEQ ID NO: 30 - RNA, 20 nt, Saccharum sp.
  source 1..20: mol_type = other RNA
aacaataggctacttctagc

SEQ ID NO: 31 - RNA, 20 nt, Saccharum sp.
  source 1..20: mol_type = other RNA
gttattctgaggggtatagt

SEQ ID NO: 32 - RNA, 20 nt, Saccharum sp.
  source 1..20: mol_type = other RNA
ccctcagtgatggattgctt

SEQ ID NO: 33 - RNA, 20 nt, Saccharum sp.
  source 1..20: mol_type = other RNA
ggtttacttgcaaccgcaga

SEQ ID NO: 34 - RNA, 20 nt, Saccharum sp.
  source 1..20: mol_type = other RNA
ttgccaccgacatcgactcg

SEQ ID NO: 35 - RNA, 20 nt, Saccharum sp.
  source 1..20: mol_type = other RNA
aaaccacgagtcgatgtcgg

SEQ ID NO: 36 - RNA, 20 nt, Saccharum sp.
  source 1..20: mol_type = other RNA
ggtcatggctcatgtcatac

SEQ ID NO: 37 - DNA, 540 bp, Saccharum sp.
  source 1..540: mol_type = genomic DNA
atggccggcagcggcagggaaagggagacgctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcgacccggatgctccgagcccaagcgacccaaaccttagggagtatttgcactggctggtcacggatattccgggaactactggggcagcatttgggcaagaggtgatctgctacgagagtcctcggccgaccatggggatccaccgcttcgtgctggtgctgttccagcagctggtgcggcagacggtgtacgccccggggtggcgccagaacttcaacaccagggacttcgccgagctctacaacctgggccctcccgtggccgccgtctacttcaactgccagcgtgaggccggctctgggggaaggaggatgtactcatga

SEQ ID NO: 38 - DNA, 534 bp, Saccharum sp.
  source 1..534: mol_type = genomic DNA
atgtctcaggtggaaccgttggttctggttcatgtgatacgagatgtgttggattcatttacaccaactatacccctcagaataacctacaacaataggctacttctagcaggtgttgagctgaagccatccgcagtggtgaataagccaagagttgatgttgggggcaccgacctcagggtgttctttacactggtattagttgatccagatgccccaagcccaagcaatccatcactgagggaatatttgcactggatggtgatagatattccaggaacaactgcagccaactttggtcaggagctcatgttttacgaaaggccagaaccgagatctggtatacaccgcatggtatttgtgctgttccggcaacttggtagggggacagtttttgcaccagacatgcgacataacttcaactgcaagaactttgcacgtcagtaccacctggacattgtggctgccacatatttcaactgtcagagggaagcaggatctgggggcagaaggttcaggcccgaaagttcgtaa

SEQ ID NO: 39 - DNA, 529 bp, Saccharum sp.
  source 1..529: mol_type = genomic DNA
atgtcagcaaccgatcctttggtcatggctcatgtcatacaggatgtgttggatccctttacaccaaccattccactaagaataacatacaacaataggctacttctggcaagtgctgagctaaagccatctgcggttgcaagtaaaccacgagtcgatgtcggtggcaatgacatgagggctttctacaccctggtactgattgacccggacgctccaagtccaagccatccatcactaagggagtacttgcactggatggtgacagatattcctgaaacaactagtgtcaactttggcacaagagctagtattttatgagagaccggacccaagatctggcatccacaggctcgtatttgtgctgttccgtcaacttggcagggggacagtttttgcaccagaaatgcgccaccacttcaactgcagaagctttgcacggcaatatcacctcagcgttgccactgctacatatttcaactgtcaaagggaaggtggatcgggtggaagaaggtttagggaagagtag

SEQ ID NO: 40 - DNA, 3081 bp, Sorghum bicolor
  source 1..3081: mol_type = genomic DNA
acgtctagcgtctacgtgcagagtacgtgcgtgcggtctccaataggcaggatagctgctagctagctagatgctagccactgctttgggccgggggggtcgtcgtcaaaagcgcgcgcgcgagcgacactgatgcaatggccatggccgggggaatataatggccagcgatgccctccaacactatgcatatatacgtaccgcgcgcgagcgtgtcatcaggtccacacatgaagatagatcgagaaaaaaaaaggactcgagattctatgggcggccgggccctgtgctatcagactcagccagcaacaaaagctatagctagcgtagtatatggcgttgcacacagccacacagtagtaaaactctgctcatcagccaataacagtcgtcccggccggccacttccaaaaagttgattggtactgcatgctatgatcgcgagcttgacatactcctatataaacagggcatcagagcagtgtgtgggacaacaaaccaacaccaacaaccagtagctggccggtgcaagtttcctgcagccaagtacatatatatacaactaattagggtatagcaagcacatctatctatctcgtatactactcctgttctcgatcgagagttaataattaaggtcgtagggattggacgacgacatggccggcagcggcagggaaagggagacgctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtacgtatatgtatatatatatccatgcatcgttttctctagctagctcgtcacgaatattaattggaacagcaaaatgcatggcttattatataatatagtctgttgagctcgtacgtacgcgtgatgccatgcatgcatgctttccttgttgttcttcaggtgatggtcgacccggatgctccgagcccaagcgacccaaatcttagggagtatctgcactggtaaatatatgtgctcatcctgcagactctccatcccttcatcaccctatctatgttagtccattgtgtagcatgtaaatcttattatatgcatgggatcggagttgatatatatctcatgtggctgaatttttggaactttaattttcagtcaacaatcttacataattaattaatcaacaagtagtagtttagtacaactaaaaagaagagacaattcacaacttctagatgttgtctgaataataacaacaaaaatatttaggcatctaagctagatataggagaagtgaaatcttatatattatcatcatatatatgactggattattgatcatatcctcctctctctgtactgtcttcttcacgcaggcttgtcacggatattccaggaaccactggggcagcatttggtgagtgcatatatgtcactccaccctgtcataggctgtctatacaactcgatcgatctgtccagacaaactttttactacacggcagtatttcctatattgtgcagatcaaacgcatgcggcacaccaacatatatatatatagcgttacacttgtcggttggttatatagagacgcacaaattaattataagtcacaacacacataattaagctactaaatactctatctgtcccaaaatgaacacagtttctctgctctcaagggcatgtgccatcgcattgtgaaccattgaatttgcattgggatgtctctctcctgctgtgcagtgctaatccatccgttcgcaatgtgaggacacatgtcattgagggcaaactttacttttctattctaaaataagtgtccctacagtacgcgtaatgcatgaatacgggtgatatatgatgccaaaaaaaaaagagagagctgatggtggcatcgtcgtcgcatgcagggcaagaggtgatctgctacgagagccctcggccgaccatggggatccaccgcttcgtgctggtgctgttccagcagctggggcgtcagacggtgtacgccccggggtggcgccagaacttcaacaccagggacttcgccgagctctacaacctgggccctcccgtcgccgccgtctacttcaactgccagcgtgaggccggatctgggggaaggaggatgtactcatgaatcacacatgccactaacgtactcgatccatgagacatgcatgcatatatgagagcaacatatacatccatcccagtgacagaaaataagcacacactgctactactagctatatatatacttgcttgctactgctagcagcactggccaggcagcatgcatatatatgcgctatacttgcttggcaatactacatatcatatgcaggcatatgtatattgctagccgtattgtatgcttctatataggcgtcacttatgtgtgtgtatatatatacatatgatatgtaggtgcacaagcatgcataaatgtgtatgtatcatatgttggatatttagtcattttacaccatgcattattccataaaaacgacagccaatatactaacacaagtattatacaagcgacaacttcataattgctactgctactacttccagcaaaacattttatgcgaagtaatagaactacagtaacaaaataaagcgcatagagagtattagcattataccctagtgtggcacttccggtacccgcggaaagtgagccaccagtcatgttgacgtcgctaccgcctcctgcggtgttgtcgttggtggcgtcattcccggcagcaggggtacttctggtaccgacagccatcacagcagcagttgacgaaggtgaagcagtcgcagtcacgttgggacaacaggaagcagtcgcaccggaaggtgctccccaaaaacttaatttcccgcctaccccgagcagagtacgcttgacgggagaagttccggaggcctgctaccagagtcctctgtactcgcagaggctctagtcggagatgcagcacagcagctcgagctcaa

SEQ ID NO: 41 - DNA, 82 bp, Saccharum sp.
  source 1..82: mol_type = genomic DNA
atgtctcaggtggaaccgttggttctggctcatgtgatacgagatgtgttggattcattcctgaagccatccgcattggtga

SEQ ID NO: 42 - DNA, 82 bp, Saccharum sp.; identical to SEQ ID NO: 41
  source 1..82: mol_type = genomic DNA

SEQ ID NO: 43 - DNA, 82 bp, Saccharum sp.
  source 1..82: mol_type = genomic DNA
atgtctcaggtggaaccgttggttctggttcatgtgatacgagatgtgttggattcatttctgaagccatccgcagtggtga

SEQ ID NO: 44 - DNA, 82 bp, Saccharum sp.
  source 1..82: mol_type = genomic DNA
atgtctcaggtggaaccgttggttctggttcatgtgatacgagatgtgttggattcatttttgaagccatccgcagtggtga

SEQ ID NO: 45 - DNA, 123 bp, Saccharum sp.
  source 1..123: mol_type = genomic DNA
  misc_RNA 54^55: note = 241 nucleotides of the wt gene are omitted
ctaagccatctgcggttgcaagtaaaccacgagtcgatgtcggcaatgacatgatccaagtccaagccatccatcactaagggagtacttgcactggtaaatcaaatgaatatgtttcactct

SEQ ID NO: 46 - DNA, 79 bp, Saccharum sp.
  source 1..79: mol_type = genomic DNA
ctaaagccatctgcggttgcaagtaaaccacgagtcggtacttgcactggtaaatcaaatgcaaatatgttttcactct

SEQ ID NO: 47 - RNA, 21 nt, synthetic construct
  source 1..21: mol_type = other RNA
ctccacattcatcggcaactc

SEQ ID NO: 48 - RNA, 21 nt, synthetic construct
  source 1..21: mol_type = other RNA
tcctcctcttcttcctcctcg

SEQ ID NO: 49 - RNA, 25 nt, synthetic construct
  source 1..25: mol_type = other RNA
tcagattcagatttcattgcatcac

SEQ ID NO: 50 - RNA, 25 nt, synthetic construct
  source 1..25: mol_type = other RNA
ttacaagttaagttagagcaagcgg

SEQ ID NO: 51 - RNA, 20 nt, synthetic construct
  source 1..20: mol_type = other RNA
caacgaccacctgctctgat

SEQ ID NO: 52 - RNA, 20 nt, synthetic construct
  source 1..20: mol_type = other RNA
aaacgaaaccccagacgaca

SEQ ID NO: 53 - RNA, 20 nt, synthetic construct
  source 1..20: mol_type = other RNA
atgtgtcgtctggggtttcg

SEQ ID NO: 54 - RNA, 20 nt, synthetic construct
  source 1..20: mol_type = other RNA
gtgggactcggattagctgg

SEQ ID NO: 55 - RNA, 20 nt, synthetic construct
  source 1..20: mol_type = other RNA
tacctgcccattcgaccacc

SEQ ID NO: 56 - RNA, 20 nt, synthetic construct
  source 1..20: mol_type = other RNA
taaagcacgaggaagcggtc

SEQ ID NO: 57 - RNA, 19 nt, synthetic construct
  source 1..19: mol_type = other RNA
ggatyggacgacgacatgg

SEQ ID NO: 58 - RNA, 20 nt, synthetic construct
  source 1..20: mol_type = other RNA
ctccctaagrtttgggtcgc

SEQ ID NO: 59 - RNA, 22 nt, synthetic construct
  source 1..22: mol_type = other RNA
gcaayatgtcagcaaccratcc

SEQ ID NO: 60 - RNA, 25 nt, synthetic construct
  source 1..25: mol_type = other RNA
gtttcaggaatatctgtcaccatcc

SEQ ID NO: 61 - RNA, 23 nt, synthetic construct
  source 1..23: mol_type = other RNA
cgactcactatagggagagcggc

SEQ ID NO: 62 - RNA, 24 nt, synthetic construct
  source 1..24: mol_type = other RNA
aagaacatcgattttccatggcag

SEQ ID NO: 63 - RNA, 20 nt, synthetic construct
  source 1..20: mol_type = other RNA
aggtggagaagggaaagtcg

SEQ ID NO: 64 - RNA, 20 nt, synthetic construct
  source 1..20: mol_type = other RNA
agttcacgtacttggacggc

(19) United States
(12) Patent Application Publication
(10) Pub. No.: US 2025/0163446 A1
(43) Pub. Date: May 22, 2025

(54) Compositions and Methods for Suppression of Flowering in Sugarcane and Energycane

(71) Applicant: University of Florida Research Foundation, Incorporated, Gainesville, FL (US)

(72) Inventors: Fredy Altpeter, Gainesville, FL (US); Baskaran Kannan, Gainesville, FL (US)

(21) Appl. No.: 18/840,617

(22) PCT Filed: Feb. 23, 2023
(86) PCT No.: PCT/US2023/063133

§ 371 Date: Aug. 22, 2024

Related U.S. Application Data
(60) Provisional application No. 63/268,572, filed on Feb. 25, 2022.

(51) Int. Cl. C12N 15/82; A01H 6/46; C12N 9/22

(52) U.S. Cl. CPC C12N 15/827; A01H 6/46; C12N 9/22; C12N 2310/122; C12N 2310/20

(57) ABSTRACT

Described herein are compositions, including RNAi and/or CRISPR constructs, for modifying one or more Flowering Locus T (FT) genes in a plant and methods of using the compositions for producing plants having suppressed or delayed flowering time. In some embodiments, the plant is a Saccharum plant. The approach used herein involves methods for decreasing expression of one or more Flowering Locus T (FT) genes by RNAi inhibition or CRISPR/Cas9 targeted mutagenesis that results in suppressed or delayed flowering in Saccharum plants, such as sugarcane or energycane. Target genes were isolated from the sugarcane and energycane cultivar and a conserved sequence between these cultivars and closely related species was chosen to design intron-hairpin RNA constructs for RNAi suppression or sgRNA expression constructs for targeted mutagenesis. Recombinant DNA vectors were introduced into sugarcane by biolistic gene transfer and transgenic plants were regenerated and selected, vegetatively propagated for replicated field testing. These genetically modified plants produce significantly elevated biomass and recoverable sugar yield under replicated field conditions relative to a similar plant in which the FT4, FT8, and/or FT10 gene have not been disrupted, when grown under the same conditions.

### CROSS-REFERENCE TO RELATED APPLICATIONS

This application claims the benefit of and priority to U.S. Provisional Patent Application No. 63/268,572, filed on Feb. 25, 2022, the entire contents of which are incorporated herein by reference.

### STATEMENT REGARDING FEDERALLY FUNDED RESEARCH

This invention was made with government support under DE-SC0018420 and DE-SC0018254 awarded by the US Department of Energy Chicago. The government has certain rights in the invention.

### SEQUENCE LISTING

The Sequence Listing written in file ‘590619 T18724 Sequence Listing.xml’ is 239 kilobytes in size, was created Feb. 23, 2023, and is hereby incorporated by reference in its entirety.

### BACKGROUND

Sugar and biomass yield from crops like energycane or sugarcane are determined by the sugar concentration in the biomass, the total biomass yield and the extractability of the sugar from the biomass. Flowering of energycane and sugarcane adversely affects sugar yield and the extractability of sugar. Upon flower induction, vegetative growth ceases and sucrose that has accumulated in the stalks is remobilized for use in reproductive development. Often, flowering also leads to dehydration of the stalk tissues, which negatively affects stalk density, and also compromises sugar extraction.

The molecular genetic mechanisms governing flowering time response are well understood in model species like Arabidopsis and rice and are well conserved in other crops, offering many control points to interfere with flowering time via mutagenesis, transgenic or gene editing approaches. Sugarcane and energycane are highly polyploid interspecific hybrids from various Saccharum species and have the most complex genome of all the agriculturally used species/crops. Therefore, it is not obvious how flowering time control is regulated in sugarcane/energycane and reports of their manipulation are lacking. Controlling flowering time is more challenging in sugarcane/energycane due to the redundancy caused by the high number of functionally equivalent gene copies.

### SUMMARY

Regulation of flowering time is an important target trait in plant breeding and genetics. Described are three Flowering Locus T (FT) genes that control the expression of florigens, mobile flowering signal proteins in plants that impact plant reproduction. Disruption of these flowering genes results in suppression or delay of flowering in plants. Described are compositions and methods for generating plants having suppressed flowering and/or delayed flowering time. Also described are plants generated using the described compositions and methods.

Described are genes responsible for the expression of florigens in sugarcane and energycane. The genes include FT4, FT8, and FT10 which are homologous to Sorghum bicolor genes encoding proteins corresponding to GenBank protein accession nos: XP_002436509.1, XP 002456354.1, and XP_002456354.1, respectively. Inhibition of one of more of these genes in sugarcane or energycane results in suppressing flowering by at least 50%, at least 60%, at least 70%, at least 80%, at least 90%, or up to 100% or delayed flowering by at least 1 week, at least 2 weeks, at least 3 weeks, at least 4 weeks, at least 5 weeks, at least 6 weeks, at least 2 months, or at least 3 months. Described are plants in which expression of one or more of FT4, FT8, and FT10 is inhibited relative to sugarcane cultivar CP96-1252 (wild-type, 96-WT) and energycane cultivar UFCP84-1047 (wild-type, 84-WT). The plants exhibit suppressed or delayed flowering. In some embodiments, FT4 is inhibited. In some embodiments, FT8 is inhibited. In some embodiments, FT10 is inhibited. In some embodiments, FT4 and FT8 are inhibited. In some embodiments, FT4 and FT10 are inhibited. In some embodiments, FT8 and FT10 are inhibited. In some embodiments, FT4, FT8, and FT10 are inhibited. In some embodiments, one or more additional paralogs are inhibited.

Described are CRISPR constructs and systems that target one or more FT genes in sugarcane and energycane. CRISPR constructs in examples provided herein are for sugarcane; similar constructs can be used to disrupt FT genes in energycane as well. In some embodiments, the CRISPR constructs and systems target one or more of FT4, FT8, and FT10. The CRISPR constructs and systems can be used to generate disruptions or loss of function mutations in one or more the FT genes. In some embodiments, the CRISPR constructs and systems can be used to generate a disruption or loss of function mutation in the FT4 gene, the FT8 gene, or the FT10 gene. In some embodiments, the CRISPR constructs and systems can be used to generate disruptions or loss of function mutations in two or more of the FT4 gene, the FT8 gene, and the FT10 gene. In some embodiments, the CRISPR constructs and systems can be used to generate disruptions or loss of function mutations in the FT4 gene, the FT8 gene, and the FT10 gene. Disrupting one or more of the FT4, FT8, and/or FT10 genes can be used to generate sugarcane or energycane plants with suppressed flowering by at least 50%, at least 60%, at least 70%, at least 80%, at least 90%, or up to 100% or delayed flowering by at least 1 week, at least 2 weeks, at least 3 weeks, at least 4 weeks, at least 5 weeks, at least 6 weeks, at least 2 months, or at least 3 months. Methods of using the CRISPR constructs and systems to disrupt or introduced a loss of function in one or more FT genes are provided. Also described are sugarcane and energycane plants in which one or more FT genes has been disrupted using a CRISPR construct or system. Also described are CRISPR modified sugarcane and energycane plants with suppressed flowering by at least 50%, at least 60%, at least 70%, at least 80%, at least 90%, or up to 100% or delayed flowering by at least 1 week, at least 2 weeks, at least 3 weeks, at least 4 weeks, at least 5 weeks, at least 6 weeks, at least 2 months, or at least 3 months.

CRISPR constructs and systems for targeted disruption or introduction of loss of function mutations in one or more FT genes in Saccharum are described herein. In some embodiments, a CRISPR RNA or sgRNA of a CRISPR system is designed to target a sequence in a sugarcane or energycane FT gene having at least 90% identity to a corresponding sequence in a sorghum ortholog of the sugarcane or energycane FT gene. In some embodiments, CRISPR RNA or sgRNA of a CRISPR system is designed to target sequence in a FT gene that shares at least 90% identity between the sugarcane or energycane orthologs.

Described are RNAi constructs and vectors that target the FT4, FT8, and/or FT10 genes in sugarcane and energycane. The RNAi constructs and vectors can be used to inhibit or knock down expression of the FT4, FT8, and/or FT10 genes. Inhibiting expression of one or more of the FT4, FT8, and/or FT10 genes can be used to generate sugarcane or energycane plants with suppressed flowering by at least 50%, at least 60%, at least 70%, at least 80%, at least 90%, or up to 100% or delayed flowering by at least 1 week, at least 2 weeks, at least 3 weeks, at least 4 weeks, at least 5 weeks, at least 6 weeks, at least 2 months, or at least 3 months. The RNAi construct or vector can be, but is not limited to, a vector encoding a hairpin-RNA, an interference RNA, or a microRNA. In some embodiments, the RNAi constructs and systems can be used to inhibit or knockdown expression of the FT4 gene, the FT8 gene, or the FT10 gene. In some embodiments, the RNAi constructs and systems can be used to inhibit or knockdown expression of two or more of the FT4 gene, the FT8 gene, and the FT10 gene. In some embodiments, the RNAi constructs and systems can be used to inhibit or knockdown expression of the FT4 gene, the FT8 gene, and the FT10 gene. Methods of using the RNAi constructs and systems to generate plants having suppressed or delayed flowering are described. Also described are sugarcane and energycane plants in which expression of one or more FT genes in inhibited using any of the described RNAi constructs or systems. Also described are sugarcane and energycane plants engineered to express one or more RNAi constructs or systems, wherein flowering is suppressed by at least 50%, at least 60%, at least 70%, at least 80%, at least 90%, or up to 100% or delayed by at least 1 week, at least 2 weeks, at least 3 weeks, at least 4 weeks, at least 5 weeks, at least 6 weeks, at least 2 months, or at least 3 months.

In some embodiments, the RNAi construct or vector is or encodes a hairpin RNA. The hairpin RNA can be, but is not limited to, a long hairpin RNA. The double stranded region of the hairpin RNA (stem) can be about 20 to about 1000 nucleotides or more in length. The stem region contains sequence that is complementary to an mRNA of a target gene in the plant, e.g., a FT4 mRNA, a FT8 mRNA, and/or a FT10 mRNA. The stem region of a hairpin RNA can have sequences that are complementary to more than one target gene. For example, the hairpin RNA stem region can have sequences that that are complementary to, the FT4 gene and the FT8 gene, the FT4 gene and the FT10 gene, the FT8 gene and the FT10 gene, or all three of the FT4 gene, the FT8 gene, and the FT10 gene. In some embodiments, the RNAi construct can also target one or more additional genes, including one or more addition flowering genes. The stem sequences complementary to the FT4 gene, FT8 gene, and/or the FT10 gene can be selected from regions of these genes that are highly conserved across different species, such as sugarcane, sorghum and maize. In some embodiments, the hairpin RNA is designed to contain sequence corresponding to at least 20 contiguous nucleotides of the FT4 gene, FT8 gene, and/or FT10 gene that are at least 85%, at least 90%, or at least 95% identical between sugarcane and sorghum or between sugarcane and maize or between different sugarcane and energycane cultivars.

In some embodiments, a hairpin RNA contains a spacer or loop sequence that facilitates formation of the hairpin structure. This spacer sequence may be any nucleotide sequence that facilitates the formation or initiation of formation double stranded (stem) region of the hairpin. In some embodiments, the spacer is about 3 to about 250 nucleotides in length. In some embodiments, the spacer sequence is derived from an intron sequence, wherein the intron sequence promotes formation of the double stranded region. In some embodiments, the spacer is derived from the rice sucrose synthase 1 gene intron. In some embodiments, the spacer comprises or consists of SEQ ID NO: 26.

In some embodiments, methods are described for generating plants having suppressed or delayed flowering. The methods comprise introducing a described CRISPR or RNAi construct or system targeting one or more FT genes into a plant cell to form a transformed plant cell and generating a plant from the transformed plant cell. The plant cell can be prepared by somatic embryogenesis in leaf whorls followed by callus initiation. Following transformation, one or more plant calli are selected based on presence of the marker gene introduced with the CRISPR or RNAi construct or system, such as, but not limited to, resistance to the selective antibiotic corresponding to the co-expressed selectable marker gene. The selected calli are then regenerated to form a plant. The plant can be propagated using methods typical in the art. The selected calli or plants can then be screened for decreased expression of the one or more FT genes. The plants can be propagated vegetatively, by traditional plant breeding, or by genetic marker assisted plant breeding. The resulting plants exhibit suppressed flowering by at least 50%, at least 60%, at least 70%, at least 80%, at least 90%, or up to 100% or delayed flowering by at least 1 week, at least 2 weeks, at least 3 weeks, at least 4 weeks, at least 5 weeks, at least 6 weeks, at least 2 months, or at least 3 months. Reduced or delaying flowing can provide for increased sugar and biomass yield.

Described are Saccharum plants having suppressed or delayed flowering, wherein one or for more the FT4 gene, FT8 gene and FT10 gene contains a loss of function mutation. The plants can contain loss of function mutation in the FT4 gene, the FT8 gene, the FT10 gene, the FT4 gene and the FT8 gene, the FT4 gene and the FT10 gene, the FT8 gene and the FT10 gene, or all three of the FT4 gene, the FT8 gene, and the FT10 gene. The loss of function mutation can be, but is not limited to, a nonsense mutation, a deletion or all or a portion of the gene (disruption), a loss of function missense mutation, or an insertion. In some embodiments, loss of function mutation is introduced into the plant or a progenitor of the plant using a CRISPR system. The CRISPR system can comprise any of the CRISPR constructs, systems, or RNAs described herein. The Saccharum plant can be, but is not limited to, sugarcane or energycane. Flowering in the plants can be suppressed by at least 50%, at least 60%, at least 70%, at least 80%, at least 90%, or up to 100% in or delayed by at least 1 week, at least 2 weeks, at least 3 weeks, at least 4 weeks, at least 5 weeks, at least 6 weeks, at least 2 months, or at least 3 months.

Described are Saccharum plants having suppressed or delayed flowering, wherein expression of one or more of the FT4 gene, FT8 gene and FT10 gene is reduced. The plants can have reduced expression of the FT4 gene, the FT8 gene, the FT10 gene, the FT4 gene and the FT8 gene, the FT4 gene and the FT10 gene, the FT8 gene and the FT10 gene, or all three of the FT4 gene, the FT8 gene, and the FT10 gene. In some embodiments, expression of one or more of the FT4 gene, the FT8 gene, and the FT10 gene is reduced by expressing in the plant one or more RNAi constructs targeting one or more of the FT4 gene, the FT8 gene, and the FT10. The RNAi construct can comprise any of the RNAi constructs, vectors, or nucleic acids described herein. The Saccharum plant can be, but is not limited to, sugarcane or energycane. Flowering in the plants can be suppressed by at least 50%, at least 60%, at least 70%, at least 80%, at least 90%, or up to 100% or delayed by at least 1 week, at least 2 weeks, at least 3 weeks, at least 4 weeks, at least 5 weeks, at least 6 weeks, at least 2 months, or at least 3 months.

Plants in which expression of one or for more the FT4 gene, FT8 gene and FT10 gene is reduced by expressing in the plant one or more RNAi constructs targeting one or more of the FT4 gene, the FT8 gene, and the FT10 display significantly elevated biomass and recoverable sugar yield under replicated field conditions relative to a similar plant in which the FT4, FT8, and/or FT10 expression is not reduced, when grown under the same conditions.

### BRIEF DESCRIPTION OF THE DRAWINGS

FIG. 1. Transgenic energycane lines which are flowering or non-flowering compared to non-transgenic energycane (WT) in replicated field plots in November 2021, at Plant Science Research and Education Center (PSREU), Citra, FL.

FIG. 2. Sanger sequencing of the PCR amplicon of FT10 revealed that a segment of DNA sequence (WT, SEQ ID NO: 45; Transgenic Line, SEQ ID NO: 46) between two gRNA binding sites located on Exon 1 and Exon 2, respectively was deleted (292 bp deletion) indicating targeted mutagenesis and functionality of both gRNAs.

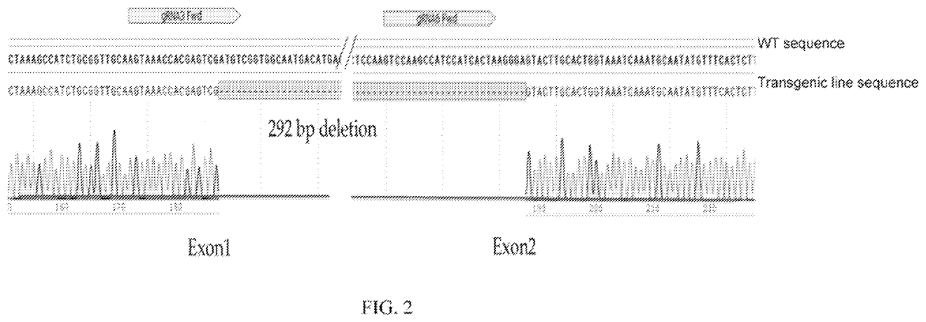

FIG. 3. Gene sequence of SbFT1 (S. bicolor, SEQ ID NO: 40) from sorghum and ScFT4 (S. cane, SEQ ID NO: 37) from sugarcane was retrieved from public domain and aligned to identify a conserved sequence. *-indicates matching nucleotides, sequence highlighted in light gray is starting position of exon 1 in ScFT4 and highlighted in dark gray is ending position of exon 1 in ScFT4.

FIG. 4. Transcriptome sequences of ScFT4_mRNA (SEQ ID NO: 37), ScFT8_mRNA (SEQ ID NO: 38), and ScFT10_mRNA (SEQ ID NO: 39). Sequences highlighted in light gray color were used for RNAi hairpin design. Nucleotides highlighted in dark gray are SNPs among the allelic variants (Table 5). Sequences with light gray color font are RNAi target identified by the RNAi target finder tools.

FIG. 5. Representative sequence of multiple alleles of ScFT4 flowering inducing gene (SEQ ID NOs: 41-44). One of the gRNA target sites is highlighted in light gray (without SNPs among the cloned amplicons). Gray color bold font indicates SNPs among the allelic variants.

FIG. 6. In-vitro cleavage assay to select superior sgRNAs for monocistronic vector construction.

FIG. 7. Generation and preliminary characterization of target mutagenesis of selected FT gene in transgenic sugarcane. Selected FT gene that was targeted for mutagenesis with sgRNAs (red arrows) (FIG. 7A). Schematic map of two monocistronic FT SgRNA expression cassettes for co-delivery with the Cas9 nuclease and selectable marker to sugarcane callus (FIG. 7B). Sanger sequencing of the PCR amplicons of ScFT10 gene from genomic DNA of transgenic sugarcane (L1, SEQ ID NO: 46) and non-modified sugarcane (WT, SEQ ID NO: 45) (FIG. 7C).

FIG. 8. Suppressed flowering of transgenic line (L1) with confirmed targeted mutagenesis of the flowering gene (ScFT10) in contrast to non-modified sugarcane cv. CP96-1252 (WT) which flowered on Nov. 24, 2022. Plants were grown under the same greenhouse conditions and natural flowering-inductive photoperiod in Gainesville, Florida.

### DETAILED DESCRIPTION

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (Ausbel et al., eds., John Wiley & Sons, Inc. 2001; Transgenic Plants: Methods and Protocols (Leandro Pena, ed., Humana Press, 1st edition, 2004); and, Agrobacterium Protocols (Wan, ed., Humana Press, 2nd edition, 2006). As appropriate, procedures involving the use of commercially available kits and reagents are generally carried out in accordance with manufacturer defined protocols and/or parameters unless otherwise noted.

The use of “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” indicates within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

The term “nucleic acid” refers to deoxyribonucleotides or ribonucleotides and polymers thereof (“polynucleotides”) in either single- or double-stranded form. Unless specifically limited, the term polynucleotide encompasses nucleic acids containing known analogues of natural nucleotides which have similar binding properties as the reference nucleic acid and are metabolized in a manner similar to naturally occurring nucleotides. Unless specifically limited, the term polynucleotide encompasses nucleic acids having one or more modified nucleotides. Modified nucleotides can modify binding properties or alter in vitro or in vivo stability. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985 J. Biol. Chem. 260:2605-2608; and Cassol et al., 1992; Rossolini et al., 1994, Mol. Cell. Probes 8:91-98). The term nucleic acid is used interchangeably with gene, cDNA, and mRNA encoded by a gene.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a sequence comparison algorithms, or by manual alignment and visual inspection.

The term “plant” includes whole plants, plant organs (e.g., leaves, stems, flowers, roots, reproductive organs, embryos and parts thereof, etc.), seedlings, seeds and plant cells and progeny thereof. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous.

The term “suppressed flowering” or “delayed flowering” refers to suppression or delay of the ability of the plant to exhibit flowering as compared to a matching control plant (e.g., a similar plant having the wild-type flowering phenotype). In some embodiments, suppressed or delayed flowering results in significantly elevated biomass and recoverable sugar yield under replicated field conditions. In some embodiments, the suppressed or delayed flowering phenotype can be achieved via targeted mutagenesis of FT genes using CRISPR/Cas9 systems and constructs. In some embodiments, the suppressed or delayed flowering phenotype can be achieved via inhibition or knockdown of expression of FT genes using RNAi systems and constructs. In some embodiments, the suppressed or delayed flowering phenotype can be achieved via genetic regulation of five pathways: photoperiod, vernalization, gibberellin, autonomy and age (Pin and Nilsson, 2012). The genetic networks underlying light and temperature-mediated flowering are not functionally validated in some crops, especially grasses with complex genome like sugarcane. However, major FT genes have significant effects on important agronomic traits including yield and yield component traits, and drought tolerance (Xue et al., 2008; Xu et al., 2014; Gol et al., 2017; Fang et al., 2019; Zhang et al., 2019). However, in addition to regulation of flowering, FT-like genes also have effects on plant architecture in grasses, growth cessation in trees, tuber formation in potato (Böhlenius et al., 2006; Danilevskaya et al., 2011; Abelenda et al., 2019).

The term “locus” refers to a position on the genome that corresponds to a measurable characteristic (e.g., a trait) or gene. A locus can be a genomic region or section of DNA (the locus) which correlates with a variation in a phenotype. A locus can comprise a single or multiple genes or other genetic information within a contiguous genomic region or linkage group.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs retain the same function in the course of evolution. “Paralogous” genes include genes related by duplication within a genome. Paralogs can evolve new functions in the course of evolution.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick or other non-traditional types. A percent complementarity indicates the percentage of bases, in a contiguous strand, in a first nucleic acid sequence which can form hydrogen bonds (e.g., Watson-Crick base pairing) with a second nucleic acid sequence (e, g., 5, 6, 7, 8, 9, 10 out of 10 being 50%, 60%, 70%, 80%, 90%, and 100% complementary). Percent complementarity is calculated in a similar manner to percent identify.

Compositions or methods “comprising” or “including” one or more recited elements may include other elements not specifically recited. For example, a composition that “comprises” or “includes” a marker may contain the marker alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”). The term “or” refers to any one member of a particular list and also includes any combination of members of that list.

The singular forms of the articles “a,” “an,” and “the” include plural references unless the context clearly dictates otherwise. For example, the term “a marker” or “at least one marker” can include a plurality of markers, including mixtures thereof.

The term “flowering locus T (FT) genes” refers to highly conserved flowering time genes that regulate flowering signals across various flowering plants. The adaptive evolution process contributes to the vast majority of phenotypic diversity in plant species that corresponds to diverse molecular mechanisms in their development. However, the majority of plant species share common transcription factors and retain ancestral gene families. Gene duplication events which occurred during plant speciation favor divergent gene functions (Flagel and Wendel, 2009). Duplication events can involve a segment of the genome or the entire genome. Whole genome duplication leads to polyploid species. For example, sugarcane is a highly polyploid species (2n=100-120; x=10-13) with typically more than 10 copies of each gene. The generation of several paralogous copies resulting from such duplication events allows divergent gene functions following mutation. Paralogous chromosomes may have redundant gene copies and increased protein dosage thereby increased gene function. This is because the duplicated genome not only retains the ancestral gene families but in addition may also develop non-, sub- or neo-functions corresponding to beneficial or deleterious mutations occurring during the evolution process. Deleterious mutations cause non-functional paralogs, whereas beneficial mutation may have sub- or neo-functional paralogs in response to environmental factors (Flagel and Wendel, 2009). Flowering time (FT) genes are conserved across dicot (model species Arabidopsis) and monocot (rice). However, functions of many homologues and their genetic relationship have not been identified in crops with complex genome like sugarcane. Flowering time locus T (FT), belongs to the FT-like genes and encodes proteins in plants that contain a phosphatidylethanolamine binding protein (PEBP) domain. Several PEBP encoding genes were identified in sugarcane and related C4 grass sorghum. PEBP encoding genes display homology between Arabidopsis and sorghum and some of those genes acts as florigens (Wolabu et al., 2016; Venail et al., 2021). Moreover, there is a synteny and microcolinearity between polyploid sugarcane and diploid sorghum genome (Wang et al., 2010). In sugarcane, 13 FT-like genes, 4 TERMINAL FLOWER (TFL)-like genes and 2 MOTHER OF FT AND TFL (MFT)-like genes, have been reported based on RNA-seq analysis and public database such as Sugarcane Genome Hub database, and the Sucest-Fun database searches (Coelho et al., 2013; Venail et al., 2021). These PEBP encoding genes display homology with corresponding genes in rice, sorghum, maize, and Arabidopsis.

A “florigen or flowering time (FT) protein” refers to a systemically mobile hormone-like molecule synthesized in the leaves and transported throughout the plant to the shoot apical meristem (SAM) through phloem in response to environmental signals such as light and temperature. Florigens functions as a hub to regulate flowering initiation in all plants where flowering time genes have been identified.

A “hairpin RNA” comprises an RNA sequence that folds back on itself to form the complementary double-stranded region (stem) and the hairpin loop structure. This double-stranded hairpin RNA is recognized by Endoribonuclease Dicer protein which cleaves double-stranded RNA into short double-stranded RNA fragments known as small interfering RNAs (siRNAs). These siRNAs associate with the RNA-induced silencing complex (RISC) and mediate knockdown of gene expression in a sequence-dependent manner. The binding of RNA-RISC complex to the target sequence triggers cleavage of the mRNA and inhibits expression of the encoded protein, thereby silencing the target gene. In some embodiments, even if the hairpin RNA is not a perfect match for the target mRNA sequence, the translation of the protein is stalled by the presence of the RISC complex physically blocking the ribosome from binding to the mRNA.

An “intron-hairpin RNA” comprises a hairpin RNA sequence wherein the hairpin loop spacer contains or comprises an intron sequence. The intron sequence promotes formation of the double-stranded region, thereby enhancing efficacy of the hairpin RNA construct. In some embodiments, the spacer sequence is derived from a rice sucrose synthase 1 gene intron. In some embodiments, the spacer comprises or consists of SEQ ID NO: 26.

An “RNA-guided DNA endonuclease” is an enzyme (endonuclease) that uses RNA-DNA complementarity to identify target sites for sequence-specific double-stranded DNA (dsDNA) cleavage. An RNA-guided DNA endonuclease may be, but is not limited to, a zCas9 nuclease, a Cas9 nuclease, type II Cas nuclease, an nCas9 nuclease, a type V Cas nuclease, a Cas12a nuclease, a Cas12b nuclease, a Cas12c nuclease, a CasY nuclease, a CasX nuclease, a Cas12i nuclease, or an engineered RNA-guided DNA endonuclease.

A “guide RNA or single guide RNA” (gRNA or sgRNA) comprises an RNA sequence (tracrRNA) bound by Cas and a spacer sequence (crRNA) that hybridizes to a target sequence and defines the genomic target to be modified. The tracrRNA and crRNA may be linked to form a “single chimeric guide RNA” (sgRNA).

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN [A/C/T] RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

The term “biolistic gene transformation” refers to the process of direct introduction of DNA or RNA into plant cells using high velocity microprojectiles that carry the DNA or RNA across cell walls and membranes. Because nucleic acids are being ‘shot’ into cells, the process represents biological ballistics, leading to the term “biolistics.” In some embodiments, these microprojectiles are gold or tungsten particles decorated with the DNA/RNA payload. In some embodiments, a gene gun is used to shoot these coated particles directly into cells thereby circumventing host-range limitations encountered with Agrobacteria delivery methods.

A “regenerant” is a plant produced from a plant tissue cell, such as a genetically modified plant tissue cell.

A “callus” is plant tissue that is formed to facilitate plant growth and give rise to roots, stems, and leaves. In some embodiments, the callus pieces of sugarcane and energycane cultivars were used for targeted gene transformation to introduce the desirable phenotypic modification.

A “plantlet” is a regenerated transformed callus that has undergone shoot development and rooting. Rooted plantlets are transferred to soil and grown under controlled conditions prior to being vegetatively propagated via cuttings and transplanted to the field site.

### II. Overview

Described are compositions, including RNAi and/or CRISPR constructs, for modifying one or more FT genes in a plant and methods of using the compositions for producing plants having suppressed flowering time. In some embodiments, the plant is a Saccharum plant.

The approach used herein involves methods for decreasing expression of one or more Flowering Locus T (FT) genes by RNAi inhibition or CRISPR/Cas9 targeted mutagenesis that results in suppressed or delayed flowering in Saccharum plants, such as sugarcane or energycane. For example, after planting on the same day, non-modified cultivar UFCP84-1047 flowered on Oct. 18, 2021, while genetically modified cultivar UFCP84-1047, with reduced expression of the FT4, FT8, and FT10 genes, did not flower until harvest on Jan. 4, 2022. A Florigen is a mobile flowering signal in plants that has a strong impact on plant reproduction and is considered one of the important targets for crop improvement. At the molecular level, a florigen is represented as a protein product encoded by the one or more FT genes.

Target genes were isolated from the sugarcane and energycane cultivar using PCR and a conserved sequence between these cultivars and closely related species was chosen to design intron-hairpin RNA constructs for RNAi suppression or sgRNA expression constructs for targeted mutagenesis. Recombinant DNA vectors were introduced into sugarcane by biolistic gene transfer and transgenic plants were regenerated and selected, vegetatively propagated for replicated field testing under natural photoperiod at the UF-IFAS research and education unit near Citra, FL. Emerging flowers were recorded weekly beginning in October and at the time of harvest biomass yield, tiller number, flower number and compressed plant circumference were determined. Juice was extracted from stems with a roller mill and soluble solids (BRIX) was determined from the extracted juice with a refractometer.

Since energycane is vegetatively propagated for establishment of plantings, suppression of flowering does not require an altered agronomic practice while improving the biomass and sugar accumulation and containment of the engineered crop.

### III. RNAi/CRISPR Systems

### A. RNA Interference

Described herein are nucleic acids for producing plants with suppressed or delayed flowering using a RNAi system. The described nucleic acids can be used to inhibit or knockdown the expression of one or more FT genes in a plant.

Sequence-selective, post-transcriptional inactivation of expression of a target gene can be achieved in a wide variety of eukaryotes by introducing double-stranded RNA (dsRNA) corresponding to the target gene, a phenomenon termed RNA interference (RNAi). RNAi occurs when an organism recognizes dsRNA molecules an processed the dsRNA into small RNA fragments of 15, 16, 17, 18, 19, 20, 21, 22, 23 or 24 nucleotides in length, called small interfering RNAs (siRNAs). The siRNAs then diffuse or are carried throughout the organism, including across cellular membranes, where associated with RISC and hybridize to mRNAs (or other RNAs), resulting in cleavage of the RNA. Most plant interfering RNAs (siRNAs and miRNAs) show extensive base pairing to, and guide cleavage of their target mRNAs (Jones-Rhoades et al. (2006) Annu. Rev. Plant Biol. 57, 19-53; Llave et al. (2002) Proc. Natl. Acad. Sci. USA 97, 13401-10406). In other instances, interfering RNAs may bind to target RNA molecules having imperfect complementarity, causing translational repression without mRNA degradation.

The term “RNAi” or “RNA interference” refers to the process of sequence-specific post-transcriptional gene silencing (e.g., in nematodes), mediated by double-stranded RNA (dsRNA). “DsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism (e.g., plants and/or crops), by, e.g., transformation, injection, spray, brush, mechanical abrasion, laser etching or immersion, etc. After introduction into the organism, the target gene-specific dsRNA is processed into relatively small fragments (siRNAs) and can subsequently become distributed throughout the organism, leading to a loss-of-function mutation having a phenotype that, over the period of a generation, may come to closely resemble the phenotype arising from a complete or partial deletion of the target gene.

This approach takes advantage of the discovery that siRNA can trigger the degradation of mRNA corresponding to the siRNA sequence. RNAi is a remarkably efficient process whereby dsRNA induces the sequence-specific degradation of homologous mRNA in animals and plant cells (Hutvagner and Zamore (2002), Curr. Opin. Genet. Dev., 12, 225-232; Sharp (2001), Genes Dev., 15, 485-490).

### B. RNAi Constructs Targeting FT Genes

In some embodiments, RNAi constructs, co-suppressing hairpin RNAs, are described that comprise an intron-hairpin RNA design to inhibit expression of FT4, FT8, and FT10 genes simultaneously. Since sugarcane and energycane are highly polyploid crops, multiple copies and alleles of the same FT gene exist. RNAi constructs that can co-suppress these multiple copies is advantageous in suppressing or delaying flowering in the plant. To achieve co-suppression of multiple copies and alleles, RNAi constructs were designed to target regions of the target Saccharum FT4, FT8, and FT10 genes that contain high sequence identity (at least 90%, at least 95%, or at least 98%) to orthologous genes in a related plants, such as sorghum or maize. The RNAi sequences for hairpin RNAs were designed by comparing different cloned PCR amplicons of the target genes from different sugarcane cultivars and by comparing sugarcane with closely related species like sorghum or maize. Regions of the genes having high sequence identify between these sequences is selected for inclusion in the co-suppression RNAi construct. In some embodiments, the co-suppressing hairpin RNA comprises SEQ ID NO: 1. In some embodiments, the co-suppressing hairpin RNA comprises SEQ ID NO: 2. SEQ ID NOs.: 1 and 2 each contain a 95 nucleotide loop sequence (spacer) derived from the rice sucrose synthase 1 gene. Other spacer sequences can be readily substituted for the 95 nucleotide spacer sequence derived from the rice sucrose synthase 1 gene. The hairpin loop spacer can be any nucleotide sequence that allows the hairpin to form. In some embodiments, the spacer is 3 to 250 nucleotides in length. In some embodiments, the spacer is derived from an intron sequence, wherein the intron sequence promotes formation of the double stranded region. In some embodiments, the spacer is derived from a rice sucrose synthase 1 (RSuS1) gene intron. In some embodiments, the spacer comprises or consists of SEQ ID NO: 26.

Additional RNAi constructs that target the FT4 gene, the FT8 gene, or the FT10 gene, or combinations thereof, are readily made using methods available in the art. An RNAi construct, such as a siRNA, hairpin RNA, short hairpin RNA, or long hairpin RNA, contains a dsRNA in which one strand (an antisense strand) contains a region that is complementary to a sequence in the target gene mRNA. The region can be a short as about 15-24 nucleotides as in a siRNA or up to several hundred nucleotides in length as in some long hairpin RNAs. An RNAi can contain a region that is complementary to a sequence in a single target gene. An RNAi construct can also contain a region or regions that are complementary to the same or different sequences present in multiple target genes. For RNAi constructs having a region or regions complementary to multiple target genes, a single region in the RNAi construct can be complementary to a common sequence present in each of one or more target genes, the RNAi construct can be chimeric in containing multiple regions that are complementary to different sequences in one or more target genes, or a combination thereof. Algorithms have been developed to identify sequences in target genes suitable for use in RNAi constructs (www.genscript.com/tools/sirna-target-finder and/or https://rnaidesigner.thermofisher.com/) and are readily adapted for identifying additional RNAi constructs for inhibiting one or more of FT4, FT8, and FT10.

Without wishing to be bound by theory, the double stranded hairpin RNA is believed to be recognized by the endoribonuclease Dicer which cleaves the double-stranded hairpin RNA into short double-stranded RNA fragments called small interfering RNAs (siRNAs), which are then used by the RNA induced silencing complex (RISC) in a homology search to target the specific endogenous genes for silencing.

### C. CRISPR/Cas9 Systems

Described herein are nucleic acids for producing plants with suppressed or delayed flowering using a CRISPR system. The described nucleic acids can be used to disrupt or introduce loss of function mutations in one or more FT genes in a plant.

A CRISPR system comprises an RNA-guided DNA endonuclease enzyme and a CRISPR RNA. In some embodiments, a CRISPR RNA is part of a guide RNA. The RNA-guided DNA endonuclease enzyme can be, but is not limited to, Cas9. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system can be delivered to a plant or plant cell via a bacterium. The bacterium can be, but is not limited to, Agrobacterium tumefaciens.

The CRISPR system is designed to target one or more of the described FT genes. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

### D. CRISPR/Cas9 Constructs Targeting FT Genes

Guide sequences suitable for forming gRNAs or crRNAs for CRISPR system mediated genetic modification of FT genes to achieve suppressed or delayed flowering are described. Suitable guide sequences include 17-20 nucleotide sequences in any of SEQ ID NOs: 3, 4, 5, 6, 7, and 8, or a complement thereof that are unique compared to the rest of the genome and immediately adjacent (5′) to a protospacer-adjacent motif (PAM) site. In some embodiments, the 17-20 nucleotide sequences are designed as gRNAs for FT4 (SEQ ID NOs: 4 and 5). In some embodiments, the 17-20 nucleotide sequences are designed as gRNAs for FT8 (SEQ ID NOs: 6 and 7). In some embodiments, the 17-20 nucleotide sequences are designed as gRNAs for FT10 (SEQ ID NOs: 8 and 9). For the RNA-guided DNA endonuclease enzyme zCas9, a PAM site is NGG. Thus, any unique 17-20 nucleotide sequence immediately 5′ of a 5′-NGG-3′ in the coding sequence of the sugarcane or energycane FT4 gene, the FT8 gene, or the FT10 gene (GenBank Accession Nos.: MT666096.1 (SEQ ID NO: 37), MT723926.1 (SEQ ID NO: 38), MT723928.1 (SEQ ID NO: 39), respectively) can be used in forming a gRNA. In some embodiments, the 17-20 nucleotide sequence immediately 5′ of a 5′-NGG-3′ in the coding sequence of the sugarcane or energycane FT4 gene, the FT8 gene, or the FT10 is at shares at least 90% to the corresponding ortholog in sorghum or maize. In some embodiments, the 17-20 nucleotide sequence immediately 5′ of a 5′-NGG-3′ in the coding sequence of the sugarcane or energycane FT4 gene, the FT8 gene, or the FT10 is at shares at least 90% to the corresponding ortholog other Saccharum plants or cultivars. Deletions or insertions in the flanking regions may alter expression of the FT gene leading to plants displaying a suppressed or delayed flowering phenotype. In some embodiments, the guide sequence is 100% complementary to the target sequence. In some embodiments, the guide sequence is at least 90% or at least 95% complementary to the target sequence. In some embodiments, the guide sequence contains 0, 1, or 2 mismatches when hybridized to the target sequence. In some embodiments, a mismatch, if present, is located distal to the PAM, in the 5′ end of the guide sequence.

CRISPR modification of a suppressed or delayed flowering phenotype is not limited to the CRISPR/zCas9 system. Other CRISPR systems using different nucleases and having different PAM sequence requirements are known in the art. PAM sequences vary by the species of RNA-guided DNA endonuclease. For example, Class 2 CRISPR-Cas type II endonuclease derived from S. pyogenes utilizes an NGG PAM sequence located on the immediate 3′ end of the guide sequence. Other PAM sequences include, but are not limited to, NNNNGATT (Neisseria meningitidis), NNAGAA (Streptococcus thermophilus), and NAAAAC (Treponema denticola). Guide sequences for CRISPR systems having nucleases with different PAM sequence requirements are identified as described above for zCas9, substituting the different PAM sequences.

Two or more guide RNAs can be used with the same RNA-guided DNA endonuclease (e.g., Cas nuclease) or different RNA-guided DNA endonucleases.

In some embodiments, two or more gRNAs targeting two or more different FT genes are used. The two or more gRNAs can be used with the same RNA-guided DNA endonuclease or different RNA-guided DNA endonucleases.

In some embodiments, three or more gRNAs targeting three or more different FT genes are used. The three or more gRNAs can be used with the same RNA-guided DNA endonuclease or different RNA-guided DNA endonucleases.

In some embodiments, two or more gRNAs targeting a single FT gene can be used. The two or more gRNAs can be used with the same RNA-guided DNA endonuclease (Cas nuclease) or different RNA-guided DNA endonucleases.

Any of the above-described guide RNAs can be provided as an RNA or a DNA encoding the RNA.

In some embodiments, a CRISPR system comprises one or more guide RNAs and a nucleic acid encoding an RNA-guided DNA endonuclease.

In some embodiments, a CRISPR system comprises one or more guide RNAs and a one or more nucleic acids encoding two or more different RNA-guided DNA endonucleases.

In some embodiments, a CRISPR system comprises a guide RNA and an RNA-guided DNA endonuclease in a complex. In some embodiments, a CRISPR system comprises a guide two or more RNAs each in a complex with an RNA-guided DNA endonuclease.

### IV. Methods of Transgenic Expression in Plants

Various methods for introducing the transgene expression vector constructs of the invention into a plant or plant cell are well known to those skilled in the art, and any method capable of transforming the target plant or plant cell may be utilized. Nucleic acids may be introduced (transformed) into a plant cell or cells using a number of methods known in the art, including, but not limited to, electroporation (U.S. Pat. No. 5,384,253, incorporated herein by reference), microprojectile bombardment or biolistic approaches (U.S. Pat. Nos. 5,550,318, 5,538,877, 5,538,880, 5,610,042, and PCT Application WO 94/09699; each incorporated herein by reference), various DNA-based vectors such as Agrobacterium tumefaciens vectors (U.S. Pat. Nos. 5,591,616 and 5,563,055; each incorporated herein by reference), and silicon carbide fiber transformation. In some embodiments, embryogenic callus, leaf whorls, whole plants, plant tissue culture cells, immature embryo, or friable tissue are transformed using one of the above methods. Additional methods include, but are not limited to, protoplast transformation of naked DNA by calcium, polyethylene glycol (PEG), or electroporation. Once a plant cell has been successfully transformed, it may be cultivated to regenerate a transgenic plant (regenerant).

In some embodiments, minimal transgene cassette is used to deliver the CRISPR or RNAi construct to a plant or plant cell using biolistic transformation, which is well described in the literature (Paszkowski et al., 1984, EMBO J. 3:2727-2722; Potrykus et al., 1985, Mol. Gen. Genet. 199:169-177). Alternative techniques for incorporating the constructs and vectors of the present invention into plant cells (for example by transduction, transfection or transformation) are well known to those skilled in the art. Such techniques include Agrobacterium mediated introduction, electroporation to tissues, cells and protoplasts, protoplast fusion, injection into reproductive organs, and injection into immature embryos (see, for example, Agrobacterium Protocols, Wan, ed., Humana Press, 2nd edition, 2006; Fromm et al., 1985, Proc. Nat. Acad. Sci. USA 82:5824-5828; Shimamoto et al., 1989, Nature, 338:274-276). The choice of technique will depend largely on the type of plant to be transformed. Cells incorporating the constructs and vectors of the present invention may be selected, as described above, and then cultured in an appropriate medium to regenerate transformed plants, using techniques well known in the art. The culture conditions, such as temperature, pH and the like, will be apparent to the person skilled in the art. The resulting plants may be reproduced, either sexually or asexually, using methods well known in the art, to produce successive generations of transformed plants.

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

T₀ transgenic plants (regenerants) may be used to generate subsequent generations (e.g., T1, T2, etc.) by selfing of primary or secondary transformants, or by sexual crossing of primary or secondary transformants with other plants (transformed or untransformed). Additional plants may also be made using available tissue culture methods for asexual propagation of plants.

### V. RNAi/CRISPR-Modified Plants

### A. RNAi-Modified Plants

Methods of producing plants with suppressed or delayed flowering and methods of genetically modifying a plant to produce a plant with suppressed or delayed flowering using a RNAi construct are described.

Described are methods of generating genetically modified plants with suppressed or delayed flowering comprising introducing into a plant, a plantlet, a plant tissue, a callus, or a plant cell, one or more RNAi constructs targeting one or more of the FT4 gene, the FT8 gene, and the FT10 gene. The RNAi constructs can comprise any of the described RNAi constructs. In some embodiments, genetically modified plants created using a RNAi construct are described. In some embodiments, the RNAi construct comprises or consists of an intron-hairpin RNA construct.

In some embodiments, methods are described for producing a sugarcane or energycane plant that displays suppressed or delayed flowering comprising introducing into the plant or plant cell one or more RNAi constructs targeting one or more of the FT4 gene, the FT8 gene, and the FT10 gene. The RNAi constructs can comprise any of the described RNAi constructs. In some embodiments, these RNAi constructs inhibit or knockdown the expression of one or more FT genes.

Plants produced using the described RNAi constructs (inhibiting or knocking down the expression of one or more FT genes) display suppressed or delayed flowering. These genetically modified plants produce significantly elevated biomass and recoverable sugar yield under replicated field conditions. In some embodiments, RNAi-modified energycane or sugarcane plants produced at least 5 kgs, at least 6 kgs, at least 7 kgs, at least 8 kgs, at least 9 kgs, at least 10 kgs more biomass per 5 plants relative to an unmodified WT cultivar or similar plant in which the FT4, FT8, and/or FT10 gene have not been inhibited, when grown under the same conditions.

### B. CRISPR-Modified Plants

Methods of producing plants with suppressed or delayed flowering and methods of genetically modifying a plant to produce a plant with suppressed or delayed flowering using a CRISPR system are described.

Described are methods of generating genetically modified plants with suppressed or delayed flowering comprising introducing into a plant, a plantlet, a plant tissue, a callus, or a plant cell, one or more CRISPR systems targeting one or more of the FT4 gene, the FT8 gene, and the FT10 gene. The CRISPR system can comprises any of the described CRISPR systems. In some embodiments, genetically modified plants created using a CRISPR system are described.

In some embodiments, methods are described for producing a sugarcane or energycane plant that displays suppressed or delayed flowering comprising introducing into the plant one or more CRISPR systems targeting one or more of the FT4 gene, the FT8 gene, and the FT10 gene The CRISPR system can comprise any of the described CRISPR systems. In some embodiments, these CRISPR systems disrupt or introduce a loss of function mutation in one or more FT genes.

In some embodiments, plants produced using the described CRISPR systems (disrupting or introducing one or more loss of function mutations to one or more FT genes) display suppressed or delayed flowering relative to a similar plant in which the FT4, FT8, and/or FT10 gene have not been disrupted when grown under the same conditions. These genetically modified plants produce significantly elevated biomass and recoverable sugar yield under replicated field conditions relative to a similar plant in which the FT4, FT8, and/or FT10 gene have not been disrupted, when grown under the same conditions.

### VI. Sequences

The nucleotide and amino acid sequences listed in the accompanying sequence listing are shown using standard letter abbreviations for nucleotide bases, and single-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

### VII. Detection of a Modified Gene

Modification of one or more FT genes using any of the described RNAi or CRISPR constructs can be detected or confirmed by any means known in the art for detecting genetic modifications.

In some embodiments, a modification can be detected in genomic DNA sample. Genomic DNA samples include, but are not limited to, genomic DNA isolated directly from a plant, cloned genomic DNA, or amplified genomic DNA.

Genetic analysis methods include, but are not limited to, polymerase chain reaction (PCR)-based detection methods (for example, TaqMan assays), microarray methods, mass spectrometry-based methods and/or nucleic acid sequencing methods, including whole genome sequencing. In some embodiments, the detection of genetic modification in a sample of DNA, RNA, or cDNA may be facilitated through the use of nucleic acid amplification methods. Such methods specifically increase the concentration of polynucleotides that span a target site, or include that site and sequences located either distal or proximal to it. Such amplified molecules can be readily detected by gel electrophoresis, fluorescence detection methods, or other means.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

The following examples are provided to illustrate certain particular features and/or embodiments. These examples should not be construed to limit the disclosure to the particular features or embodiments described.

### EXAMPLES

### Example 1. Identification of Flowering Locus T (FT) Homologs

Flowering in rice and sugarcane are induced under shortening photoperiod and rice is considered a facultative short-day plant (SD). In rice, there are several major FT-like genes involved in flowering, such as Hd3a, RFT1, FTL1, FT1, and FT2, which ensure flowering initiation through alternative pathways (Cao et al., 2021). Several FT paralogs were identified in rice and three paralogs such as Heading-date3a (Hd3a), Rice Flowering Locus T1 (RFT1) and Flowering Locus T-like (FTL) have been characterized for their functional roles in the regulation of flowering (Kojima et al., 2002; Komiya et al., 2008; Ishikawa et al., 2011). Hd3a is the rice ortholog of the Arabidopsis FT gene and promotes flowering under short day (SD) conditions. Heading date 1 (Hd1), a homolog of CO, promotes flowering in rice under SD through upregulation of Hd3a transcripts (Kojima et al., 2002; Ishikawa et al., 2011). RFT1 activates flowering under long-day (LD) conditions. Hd3a and RFT1 are florigens in rice and promote flowering under specific photoperiods (Komiya et al., 2008). Major flowering genes were functionally characterized in rice (Table 1). Transgenic approaches (RNAi mediated target gene suppression, over-expression and genome editing) were employed to suppress or to delay flowering in rice and are listed in the table below. The number of days delayed for flowering in transgenic rice lines varied from few days to no flowering. Although several FT homologs were tested in rice for the suppression of flowering, co-suppression of two flowering genes, RFT1 and Hd3a, by RNAi led to complete suppression of flowering in rice. RNAi suppression of only Hd3 a delayed flowering for 30 days. RNAi suppression of only RFT1 did not suppress the flowering (Komiya et al., 2008).

TABLE 1
Reports of transgenic or gene editing approaches for flowering suppression in
rice and resulting phenotypes

| | | | Number of days | | |
|---|---|---|---|---|---|
| | | Approach used | flowering | | |
| | | for flowering | delayed/no | | |
| Crop | Gene/genes | suppression | flowering | | References |
| Rice | Hd3a | RNAi | 30 | | Komiya et al., 2008 |
| | RFT1 and Hd3a | RNAi | No flowering | | Komiya et al., 2008 |
| | Hd1 | CRISPR | 12.5 | days | Zong et al., 2021 |
| | Hd2 uORFs | CRISPR | 4.6-11.2 | days | Liu et al., 2021 |
| | R domain of Edh1 | Over expression | 12-13 | days | Cho et al., 2016 |
| | Ghd7 | Over expression | Up to 71.1 | days | Xue et al., 2008 |
| | AtPRR5 | Over expression | 11 | days | Nakamichi et al., |
| | | | | | 2020 |
| | OsGI | Over expression | 90 | days | Hayama et al., 2003 |

| | | | |
|---|---|---|---|
| RC1/RCN2 | Over expression | 13 days to No | Nakagawa et al., |
| | | flowering | 2002 |

Sorghum also flowers under short-day. 19 PEBP encoding genes have been reported, which includes 13 FT-like genes, 4 TERMINAL FLOWER (TFL)-like and 2 MOTHER OF FT AND TFL (MFT)-like genes. Over expression of SbFT1, SbFT8, and SbFT10 induced expression of floral meristem identity genes AP1 and LFY and promoted flowering in Arabidopsis ft-1 mutants. SbFT1, SbFT8, and SbFT10 are expressed in leaves and apical meristems in different sorghum strains including those from grain, sweet and forage sorghum cultivars. These genes are considered candidates for florigens in sorghum (Wolabu et al., 2016). In contrast, SbFT2, SbFT6 and SbFT9 genes did not activate flowering when expressed in Arabidopsis ft-1 mutant background. Phylogenetic analysis of PEBP protein sequences revealed that SbFT1 has higher homology with the rice Hd3a, SbFT8 and SbFT10 with the maize ZCN8 (Wolabu et al., 2016).

A single amino acid change can result in contrasting activation or repression of flowering (Venail et al., 2021). For example, a single amino acid change in the Arabidopsis flowering repressor (Terminal flower 1, TFL1), converts it to flowering activator (Flowering locus T, FT) and vice versa (Hanzawa et al., 2005). The amino acid sequence of ScFT3 (sugar cane) and SbFT2 (sorghum) are identical except one amino acid change at position 120. Sugarcane ScFT3, but not sorghum sbFT2 was able to promote flowering when expressed in a Arabidopsis FT-mutant background (Venail et al., 2021). Unexpectedly, over-expression of ScFT1 in Arabidopsis, caused a delay in flowering despite its high similarity in expression pattern and sequence to the florigen-encoding FT. (Coelho et al. 2014). Such preliminary analysis of sugarcane FT gene family members suggests that their function in the floral transition has diverged from the predicted role of similar PEBP family members. Therefore, confirmation of sugarcane FT gene family members as flowering suppressor requires their suppression in sugarcane which has not been described previously.

### Example 2. RNAi or CRISPR/Cas9 Mediated Suppression of Flowering in Sugarcane and Energycane

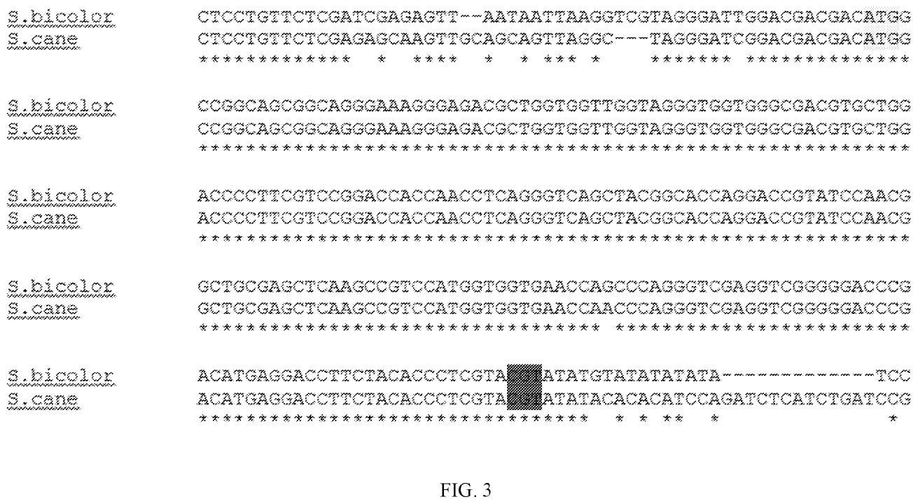

Three FT homologs encoding florigens in sorghum were earlier reported in the literature (Wolabu et al., 2016, doi.org/10.1111/nph.13834). Corresponding sugarcane transcriptome and genome sequences were retrieved from the public domain (https://blast.ncbi.nlm.nih.gov/Blast.cgi and http://bce.bioetanol.cnpem.br/ctbeblast/) and aligned with sorghum genome sequences (FIG. 3). Degenerate primers were designed for the conserved regions of ScFT4, ScFT8 and ScFT10 sequences to amplify all the alleles/copies of these genes from early flowering, elite sugarcane cultivar CP96-1252 and energycane cultivar UFCP84-1047. Cloned PCR amplicons were sequenced by the Sanger method and multiple sequence alignment tool (www.genome.jp/tools-bin/clustalw) was used to identify single nucleotide polymorphisms (SNPs) in exons of those florigens or allelic variants (Table 4). Highly conserved region of allelic variants was used to design RNAi hairpin for knockdown of florigens or sgRNA (single guide RNA) for knockout of florigens through CRISPR/Cas9 mediated genome editing.

TABLE 5
Number of SNPs identified in each candidate flowering
gene after multiple sequence alignment of allelic
variants in sugarcane and energycane.

| | SNP in | Exon 1 | SNP in | Exon 2 | SNP in | Exon 3 |
|---|---|---|---|---|---|---|
| Target | Exon 1 | (bp) | Exon 2 | (bp) | Exon 3 | (bp) |
| ScFT4 | 5 | 209 | 2 | 61 | — | — |
| ScFT8 | 4 | 198 | 1 | 64 | 1 | 43 |
| scFT10 | 4 | 197 | 2 | 64 | 0 | 43 |

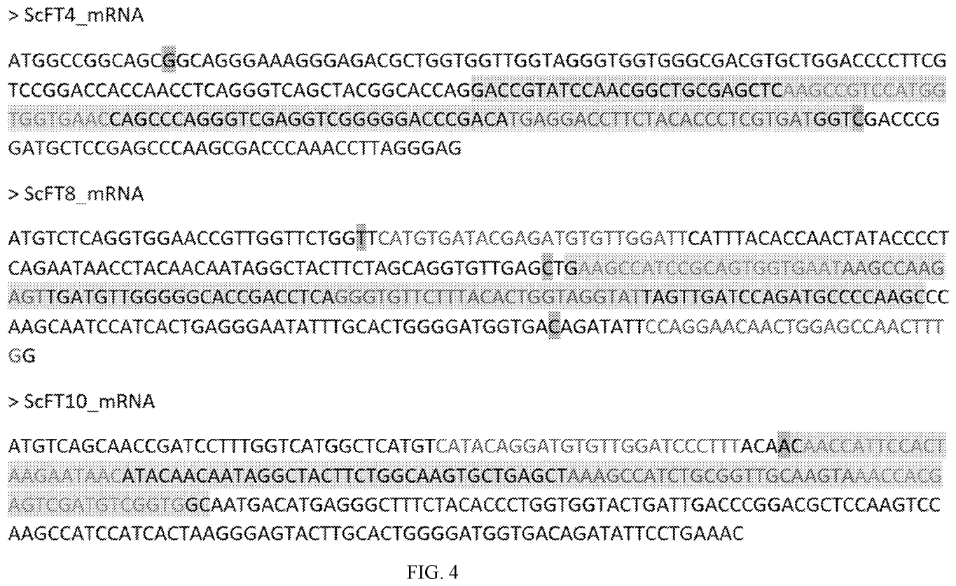

For RNAi mediated suppression of florigens, conserved regions of exon 1 and exon 2 from ScFT4, ScFT8 and ScFT10 sequences were selected (FIG. 4) encompassing multiple RNAi targets identified by RNAi finder tools (www.genscript.com/tools/sirna-target-finder or https://rnaidesigner.thermofisher.com/). One construct was 719 bp in length (short) while the other was 1291 bp in length (long). Sense and anti-sense orientation of 719 or 1291 bp transcriptome sequences with 95 bp of intron sequence from rice sucrose synthase1 (RSuS1) gene formed a RNAi hairpin design (SEQ ID NOs: 1 and 2). Hairpin sequences were generated by custom gene synthesis (www.genscript.com/gene_synthesis) and cloned under the transcription regulation of ZmUbi promoter (Zea mays Ubiquitin) and ZmHSP18 terminator (Zea mays Heat Shock protein 18). Selectable marker gene (Neomycin phosphotransferase II (NPTII) from Escherichia coli or Acetolactate synthase (ALS) from Zea mays) were cloned under the regulatory sequences of ZmALS2 promoter with ZmHSP70 intron (Heat shock protein 70 from Zea mays) and ZmHSP16.9 terminator. Both RNAi cassette and marker gene cassette were subcloned into multiple cloning site of pUC57 plasmid vector (SEQ ID NOs: 9-12). Before the biolistic gene transformation, pUC57 backbone was removed by restriction enzyme digestion, gel electrophoresis and gel elution. The minimal transgene cassette without vector backbone was used for biolistic gene transformation into sugarcane or energycane calli at a concentration of 1.5 ng DNA/kb/shot (SEQ ID NO: 16 to 19).

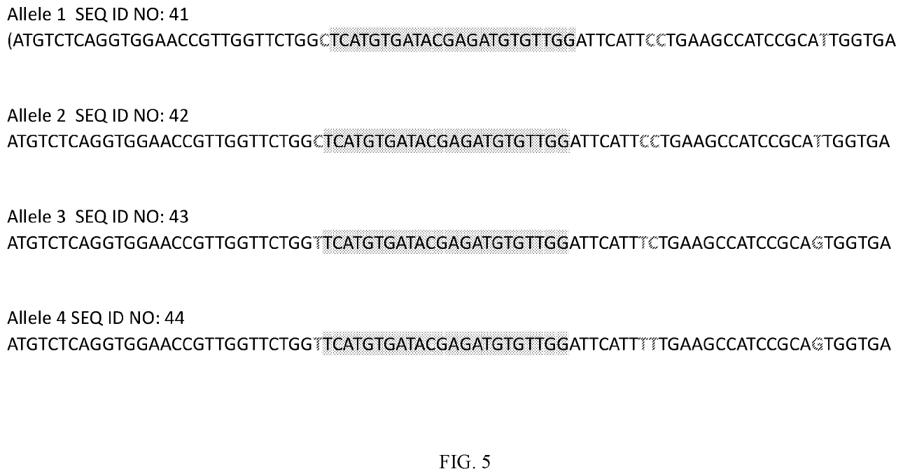

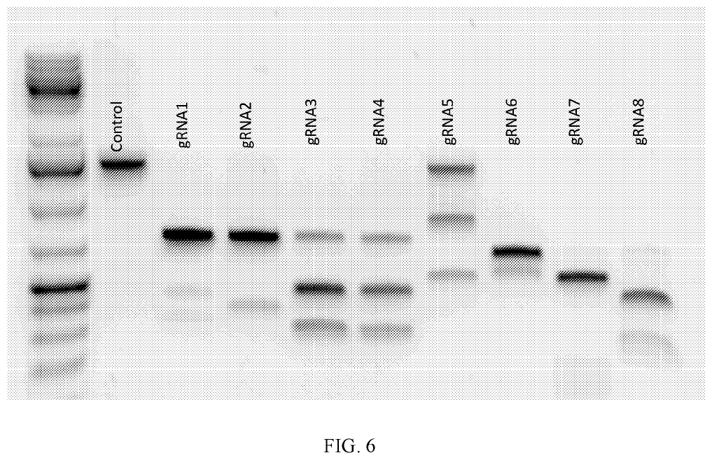

For CRISPR/Cas9 mediated co-suppression of florigens in sugarcane a partial sequence with highest conservation (without SNP's among all cloned amplicons) was selected for ScFT4, ScFT8 and ScFT10 sequences (FIG. 5) for sgRNA design using CRISPOR gRNA design tool (http://crispor.tefor.net/). Sixteen gRNAs were designed, 5 each for ScFT4 (SEQ ID NOs: 3, 4, and 27 to 29), ScFT8 (SEQ ID NOs: 5, 6, and 30 to 32) and 6 for ScFT10 (SEQ ID NOs: 7, 8, and 33 to 36). Multi-allelic cleavage efficiency of these gRNAs was tested by in vitro assays (FIG. 6). In vitro cleavage assays were performed using sgRNA, Cas9 protein and PCR amplified targets. Of the 16 gRNAs tested for in vitro cleavage assay, 14 of them cleaved the target with varying intensity. Two gRNAs were selected for each flower inducing gene, one each targeting exon 1 and exon 2 (SEQ ID NOs: 3 to 8). gRNAs for each target gene were synthesized and cloned under U6 promoter (Type III promoter) from Oryza sativa and CRISPR scaffold sequence (CRISPR RNA and tracr RNA) from Streptococcus pyogenes. sgRNAs cassette of each target gene was cloned into expression vector, which contains cassette for Cas9 (CRISPR associated 9) from Streptococcus pyogenes and selectable marker gene NPTII from E. coli. Cas9 was regulated by ZmUbi promoter with first intron and AtHSP18.2 terminator (Heat shock protein terminator 18.2 from Arabidopsis thaliana) and NPTII was under the transcriptional regulation of ZmALS2 promoter with HSP70 intron and ZmHSP16.9 terminator (SEQ ID NO: 13 to 15). As described for RNAi construct transformation, minimal cassette was used for CRISPR/Cas9 construct (concentration of 1.5 ng/kb/shot) transformation as well (SEQ ID NO: 20 to 22).

### Example 3: Description of Ghd7 (Repressor of Florigens) Construct

GRAIN NUMBER, PLANT HEIGHT AND HEADING DATE 7 (Ghd7) was reported as a repressor of florigens in sorghum (Yang et al., 2014) and in rice (Xue et al., 2008). Sorghum Ghd7 sequence was retrieved from the public domain phytozome (Sb06g000570) and commercially synthesized (SEQ ID NO: 23) to clone into expression vector. Expression vector components were the same as described above for the RNAi constructs except the target gene Ghd7 sequence (SEQ ID NO: 24). The minimal transgene cassette without vector backbone was used for biolistic gene transformation into sugarcane calli at a concentration of 1.5 ng DNA/kb/shot (SEQ ID NO: 25).

### Example 4: Quantitative Real-Time RT-PCR Analysis of RNAi Constructs or Ghd7 Expression

During a photoinductive period (photoperiod was 11 hr and 55 min), a 100 mg of top visible dewlap leaf was collected from RNAi lines of both sugarcane and energycane and wild-type grown under field conditions (3 replications) in an eppendorf tube and flash frozen immediately. Total RNA was isolated using TRIzol reagent (Invitrogen, Grand Island, NY) according to the manufacturer's instructions. Total RNA was treated with RNase-Free RQ1 DNase (Promega, San Luis Obispo, CA). cDNA was synthesized from 500 ng of DNase-treated total RNA using High capacity cDNA reverse transcription kit (Applied Biosystems™, Thermo Fisher Scientific, Madison, WI). The sugarcane Tubulin primers (Forward: 5′-CTCCACATTCATCGGCAACTC-3′ and reverse: 5′-TCCTCCTCTTCTTCCTCCTCG-3′, SEQ ID Nos. 47 and 48) were used to amplify a 237 bp fragment as a reference (housekeeping gene) for normalization of transcripts as described by Iskandar et al. (2004). RNAi construct transcriptome was amplified using hairpin specific primers (Forward: Rsus-in-F1 5′-TCAGATTCAGATTTCATTGCATCAC-3′ and reverse: Rsus-in-R1 5′-TTACAAGTTAAGTTAGAGCAAGCGG-3′, SEQ ID Nos. 49 and 50), and Ghd7 transcriptome was amplified using target gene specific primers (Forward: Ghd7-RT-F1 5′-CAACGACCACCTGCTCTGAT-3′ and reverse: Ghd7-RT-R1 5′-AAACGAAACCCCAGACGACA-3′, SEQ ID Nos. 51 and 52). Quantitative real-time PCR of the transcripts was performed in the CFX Connect Real-Time PCR (Bio-Rad, Hercules, CA) with SsoAdvanced SYBR Green Supermix (Bio-Rad, Hercules, CA) under the following conditions: 95° C. for 3 min denaturation, 40 cycles at 95° C. for 10 s and 58° C. for 45 s. Amplification specificity was verified by melt curve analysis from 55 to 95° C. RNAi construct hairpin expression levels of transgenic lines relative to wild-type plants were calculated using the 2-44Cl method (Livak and Schmittgen, 2001).

### Example 5: Complete Suppression of Flowering Achieved in Both Sugarcane and Energycane Using RNAi Mutagenesis

Complete suppression of flowering was achieved in both sugarcane and energycane cultivars and resulted in significantly elevated biomass and recoverable sugar yield under replicated field conditions.

TABLE 2
Biomass weight, agronomic traits and sugar yield of cv. UFCP84-1047 with expression
of RNAi suppression construct of ScFT4, ScFT8 and ScFT10 (L325; L326; L336; L342;
S430; S445) in comparison to non-modified Sugarcane cv. UFCP84-1047 (84-WT).

| | Average | RNAi construct | | Number of | | | |
|---|---|---|---|---|---|---|---|
| | Biomass | expression | | flowers at | | | |
| | weight of | (normalized to | | the time of | Number | Plant | Total soluble |
| Energycane | 5 plants | housekeeping | Date of first | harvest (Jan | of tillers/ | circumference | solids yield |
| Lines | (kg) | gene, Tubulin) | flowering | 4th)/ 5 plants | 5 plants | (cm) | (kg)/5 plants |
| 84-WT | 40.29 | 0.00 | Oct. 18 | 46 | 76 | 37.0 | 1.65 |
| L325 | 55.52 | 0.07 | Non-flowering | 0 | 98 | 45.7 | 1.98 |
| L326 | 54.51 | 0.10 | until the time | 0 | 110 | 47.4 | 2.39 |
| L336 | 49.14 | 0.11 | of harvest | 0 | 91 | 43.9 | 2.07 |
| L342 | 50.76 | 0.06 | (Jan. 4, 2022) | 0 | 96 | 40.0 | 2.24 |
| S430 | 55.30 | 0.03 | | 0 | 127 | 44.7 | 2.05 |
| S445 | 54.52 | 0.01 | | 0 | 89 | 47.0 | 2.66 |
| LSD: | 9.63 | | | | 18.9 | 6.97 | 0.60 |

Note:
LSD—Least square difference among the lines tested in replicated field plots

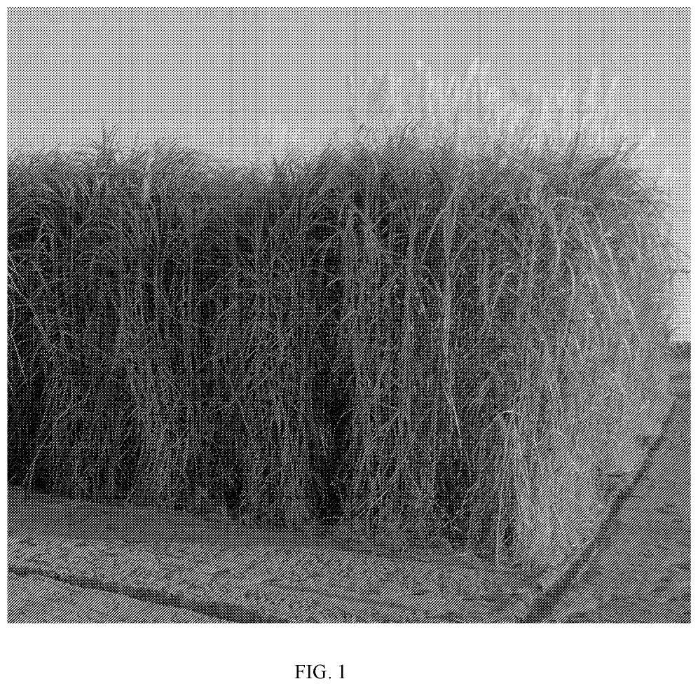

Energycane transgenic lines transformed with construct for suppression of flowering had RNAi construct expression of 0.03 to 0.11, normalized to housekeeping gene, Tubulin and these lines did not flower until the harvest on Jan. 4, 2022, whereas non-transformed energycane cultivar, UFCP84-1047, flowered on Oct. 18, 2021. Average biomass fresh weight of WT was 40.3 kg (average of 5 plants weight) and 6 transgenic lines produced significantly higher biomass fresh weight (49.1 kg to 55.5 kg; Table 2) than WT. Number of tillers produced by transgenic lines varied from 89 to 127 per 5 plants as compared to WT, which had 76 tillers (Table 2, FIG. 1). Plant circumference of WT was 37.0 cm, whereas 4 of these 6 transgenic lines had significantly higher plant circumference. Total soluble solids yield (calculated by multiplication of juice volume×percent of total soluble solids×sugarcane stalk weight per 5 plants) of non-transformed energycane was 1.7 kg per 5 plants. However, transgenic lines yielded up to 2.7 kg of total soluble solids per 5 plants (Table 2).

TABLE 3
Biomass weight, agronomic traits and sugar yield of Sugarcane cv. CP96-1252 with expression
of RNAi suppression construct of ScFT4, ScFT8 and ScFT10 (L19; L20; L3; L7; Li-B8;
L31; S157) in comparison to non-modified Sugarcane cv. CP96-1252 (96-WT).

| | Average | RNAi construct | | Number of | | | |
|---|---|---|---|---|---|---|---|
| | Biomass | expression | | flowers at | | | |
| | weight of | (normalized to | Date of | the time of | Number | Plant | Total soluble |
| Sugarcane | 5 plants | housekeeping | first | harvest (Jan | of tillers/ | circumference | solids yield |
| Lines | (kg) | gene, Tubulin) | flowering | 11th)/ 5 plants | 5 plants | (cm) | (kg)/5 plants |
| 96-WT | 50.76 | 0.00 | Dec. 6 | 2 | 41 | 39.2 | 4.57 |
| L19 | 59.94 | 0.11 | Non-flowering | 0 | 59 | 42.9 | 5.23 |
| L20 | 62.16 | 0.07 | until the time | 0 | 56 | 44.7 | 5.22 |
| L3 | 65.40 | 0.06 | of harvest | 0 | 65 | 46.1 | 5.82 |
| L7 | 61.77 | 0.06 | January 11th | 0 | 56 | 42.9 | 4.95 |
| Li-B8 | 56.86 | 0.15 | | 0 | 53 | 42.5 | 4.79 |
| L31 | 58.25 | 0.05 | | 0 | 65 | 38.9 | 5.37 |
| S157 | 58.43 | 0.10 | | 0 | 52 | 43.2 | 5.18 |
| LSD: | 8.65 | | | | 9.0 | 4.09 | 1.09 |

Note:
LSD—Least square difference among the lines tested in replicated field plots

Sugarcane cultivar CP96-1252 (wild-type, WT) yielded an average of 50.8 kg of fresh biomass weight (5 plants average) and flowered on Dec. 6, 2021, whereas sugarcane RNAi lines transformed with target gene suppression construct yielded up to 65.4 kg of biomass weight with no flag leaf/flowering until the harvest (Jan. 11, 2022) and 7 transgenic lines had significantly higher biomass yield than WT control. These transgenic lines had RNAi target construct expression of 0.05 to 0.15, normalized to housekeeping gene (Tubulin). Number of tillers produced by transgenic lines varied from 52 to 65, whereas WT had 41 tillers. Line L31 had similar plant circumference to WT (39.2 cm), however, it had significantly greater number of tillers (65), which contributed to higher biomass yield (Table 3). Total soluble solids yield of WT was 4.6 kg and transgenic lines recorded 4.8 kg to 5.8 kg per 5 plants (Table 3).

TABLE 4
Expression of Ghd7 flowering repressor construct in sugarcane cultivar CP96-1252 and corresponding biomass
weight, agronomic traits and sugar yield compared to non-modified sugarcane cultivar CP96-1252.

| | Average | Ghd7 construct | | Number of | | | |
|---|---|---|---|---|---|---|---|
| | Biomass | expression | | flowers at | | | |
| | weight of | (normalized to | Date of | the time of | Number | Plant | Total soluble |
| Sugarcane | 5 plants | housekeeping | first | harvest (Jan | of tillers/ | circumference | solids yield |
| Lines | (kg) | gene, Tubulin) | flowering | 13th)/ 5 plants | 5 plants | (cm) | (kg)/5 plants |
| 96-WT | 53.10 | 0.00 | Dec. 6 | 9 | 48 | 37.1 | 4.92 |
| Ghd-2 | 53.99 | 1.18 | Dec. 6 | 10 | 65 | 42.1 | 4.23 |
| Ghd-32 | 49.63 | 0.32 | Dec. 20 | 1 | 65 | 37.0 | 3.67 |
| Ghd-33 | 53.76 | 0.24 | Dec. 20 | 1 | 51 | 38.0 | 4.18 |
| Ghd-X2 | 45.62 | 0.64 | Dec. 20 | 1 | 64 | 34.9 | 3.21 |
| LSD: | 13.60 | | | | 14.5 | 5.38 | 1.69 |

Sugarcane transgenic lines harboring Ghd7 construct (repressor of florigens in sorghum) were also evaluated for transgene expression, biomass weight, agronomic traits and sugar yield (Table 4). Non-transformed sugarcane WT (CP96-1252) yielded an average of 53.1 kg fresh biomass weight (5 plants average) and flowered on Dec. 6, 2021, whereas transgenic lines over expressing sorghum Ghd7 construct had similar yield. Despite these transgenic lines had Ghd7 expression, which varied from 0.24 to 1.18 normalized to housekeeping gene (tubulin), all those transgenic lines displayed flowering (December 6th-December 20th) before the harvest. Plant circumference was similar between WT and transgenic lines. However, 3 transgenic lines (Ghd-2, Ghd-32 and Ghd-X2) had significantly more tillers than WT control. Total soluble solids yield was also similar between WT and transgenic lines, except Ghd-X2 which had significantly lower sugar yield (Table 4).

Sugarcane or energycane cultivars used for generation of transgenic lines: Elite sugarcane cv. CP96-1252 was used as source for leaf-whorl explants for generation of transgenic sugarcane RNAi lines and as non-modified control (designated wild-type; WT). The prefix “CP” stands for Canal Point, FL and this cultivar developed by a cooperative sugarcane cultivar development program of USDA-ARS, Canal Point, FL, UF/IFAS, FL and the Florida Sugar Cane League, Inc. CP96-1252 has characteristics of high cane yield and early and prolific flowering. CP96-1252 is the predominantly grown sugarcane cultivar in FL since 2014 (VanWeelden et al., 2019). In 2018, this cultivar alone occupied 35.8% of FL's total sugarcane acreage.

For energycane, cultivar UFCP84-1047 was used to generate energycane RNAi lines. Energycane is like sugarcane an interspecific hybrid in the genus Saccharum. In contrast to sugarcane, energycane has a high proportion of the ancestral species Saccharum spontaneum in its genome which contributes to higher tiller number, fiber content, biomass yield, early flowering and persistence in addition to a reduced stem diameter and sugar content. Energycane cv. UFCP84-1047 was released by USDA, ARS, Canal Point, FL and UF/EREC, Belle Glade, FL and derived from a cross between CP 78-0349 (commercial sugarcane hybrid×Mandalay (S. spontaneum clone originated from Myanmar (Gordon et al., 2016).

Generation of transgenic lines: Transgenic lines were regenerated through somatic embryogenesis following biolistic gene transfer of RNAi constructs or CRISPR/Cas9 constructs as described in Taparia et al., 2012. Briefly, 1-2 mm cross-sections of CP96-1252 or UFCP84-1047 leaf whorls were cultured on the Modified Murashige & Skoog medium with B5 vitamins (PhytoTech Labs, KS, USA) supplemented with 2,4-Dichloro phenoxy acetic acid (PhytoTech Labs, KS, USA) 3 mg/L to initiate callus. Cultures were maintained at 28° C. and 24 hr dark cycle and subcultured weekly. Eight weeks after callus induction, the callus pieces were used for target gene transformation using biolistic PDS-1000/He apparatus (Bio-Rad, Hercules, CA).

Transformed calli were transferred onto Modified MS media with B5 vitamins supplemented with Geneticin (20 mg/L, PhytoTech Labs, KS, USA), an antibiotic, for the selection of transgenic events. Four weeks after selection, calli were subcultured on to regeneration media (MS media with B5 vitamins, supplemented with α-Naphthaleneacetic acid/NAA (PhytoTech Labs, KS, USA) 1.86 mg/L and 6-Benzylaminopurine/BAP (PhytoTech Labs, KS, USA) 0.09 mg/L (sugarcane) or 0.045 mg/L (energycane). From regeneration phase onwards cultures were maintained at 28° C. temperature and 16/8 hr light (30 μmol m-2 s-1) and dark cycle. Regenerated calli were transferred to modified MS basal medium with Gamborg vitamins (PhytoTech Labs, KS, USA) 4.4 g/L for shoot development and rooting. Rooted plantlets were washed off media, roots dipped onto rooting hormone (Indole-3-butyric acid), transferred to soil (Jolly Gardener Pro-Line C/G mix) and acclimatized in a temperature-controlled growth chamber. Six months following transfer of plants to the soil under greenhouse conditions, plants were vegetatively propagated and transplanted to the field site.

PCR confirmation of transgenic lines: To confirm of the presence of transgene construct in the transgenic plants, PCR based screening was performed. A 100 mg sample of leaf tissue was collected from each putative transgenic line in a 2 ml Eppendorf tube and used for DNA isolation or stored in −80° C. freezer until used. DNA was isolated using modified cetyltrimethyl-ammonium bromide CTAB method (Murray and Thompson, 1980). Concentration of the isolated DNA was quantified using Nanodrop one spectrophotometer (Thermo Fisher Scientific, Madison, WI). PCR reaction was performed in 20 ul reaction volume using Standard Taq polymerase, dNTPs, Taq Polymerase (New England Biolabs Inc, Ipswich, MA) and construct specific primers (Forward: ZmHSPt_F3: 5′-ATGTGTCGTCTGGGGTTTCG-3′, Reverse: PhTBS_R3: 5′-GTGGGACTCGGATTAGCTGG-3′ and NPTII_F: 5′-TACCTGCCCATTCGACCACC-3′ and 5′-TAAAGCACGAGGAAGCGGTC-3′, SEQ ID Nos. 53, 54, 55, and 56, respectively), which yielded 405 bp and 345 bp amplicon, respectively on the 1.2% agarose gel visualized under UV transilluminator (Bio-Rad, Hercules, CA).

Detection of targeted mutations in genome editing reagents harboring transgenic lines: Transgenic lines harboring sgRNAs targeting ScFT4 and ScFT10 were screened for the targeted mutations such as SNAs or Insertions and deletions (InDels) using sanger method. PCR amplicons encompassing target region were amplified using gene specific primers (FT1-F3: 5′-GGATYGGACGACGACATGG-3′, FT1-F3: 5′-CTCCCTAAGRTTTGGGTCGC-3′ and FT10-F1: 5′-GCAAYATGTCAGCAACCRATCC-3′, FT10-R1: 5′-GTTTCAGGAATATCTGTCACCATCC-3′, SEQ ID Nos. 57, 58, 59, and 60, respectively) yielded 561 bp and 576 bp amplicons for ScFT1 and ScFT10, respectively. PCR amplicons were column purified using GeneJET PCR purification kit (Thermo Fisher Scientific, Madison, WI) and ligated into pJET1.2/blunt Cloning Vector (Thermo Fisher Scientific, Madison, WI) and transformed into E. coli DH10-beta competent cells (New England Biolabs Inc, Ipswich, MA) by heat shock transformation method. Transformed colonies were cultured on the LB (Luria-Bertani) liquid media at 37° C., 250 rpm shaker for 14-16 hrs and plasmids were purified using GeneJET plasmid miniprep kit (Thermo Fisher Scientific, Madison, WI). Sanger sequencing reactions were performed at Eurofins genomics facility (Eurofins Genomics, Louisville, KY) using forward sequencing primer (5′-CGACTCACTATAGGGAGAGCGGC-3′, SEQ ID No. 61) or reverse sequencing primer (5′-AAGAACATCGATTTTCCATGGCAG-3′, SEQ ID No. 62).

Propagation, transplanting and field trial establishment at PSREU, Citra, FL: After transfer to soil, transgenic plants were grown in a greenhouse for 6 months before vegetative propagation by stem segment cuttings. Four weeks after sprouting of vegetative buds in trays with potting mix, individual plants were moved to 3-L pot containing potting mix (Jolly Gardener Pro-Line C/G mix) for the establishment of shoots and roots for 3-4 weeks. Field experimental plots were established at the Plant Science Research and Education Unit (PSREU, Citra, FL), 29.409006 (latitude)-82.180473 (longitude) in March 2021. Field plots were laid out in a Randomized Complete Block Design (RCBD) with 2-3 replications in loamy sand soil and a total of 18 transgenic lines of energycane and 19 transgenic lines of sugarcane RNAi lines, and 4 transgenic lines of Ghd7 over expression and WT were planted. Each line was planted in one row plot with 5 plants per row. The spacings between rows and plants were 120 cm and 60 cm, respectively. At the time of planting, Osmocote® plus fertilizer were spot applied at a rate of 40 g/plant. Plots were fertilized with 68 kg/ha N, 23 kg/ha P and 68 kg/ha K two weeks after planting, followed by two additional applications at the same rate in an interval of 8 weeks. Plots were irrigated daily with a rate of 10 mm for two weeks following transplanting and three times a week to provide at least 25 mm of irrigation per week depending on rainfall during grand growth period. Weeds were removed mechanically during the plant establishment by using a mini-rototiller (Rear Tine Tiller, model: 100380, Champion Power Equipment Inc., CA, USA) between rows and by hoeing within rows. Bifenthrin (Brigade® 2EC), Imidacloprid (Admire® Pro) or Sulfoxaflor (Transform™) insecticides were applied at the labeled rate for the control of insects such as mealybugs, scales and aphids and Pyraclostrobin (Headline®) was applied at the labeled rate for the control of orange rust during the grand growth period.

Phenotyping of agronomic traits and biomass yield determination: Emerging of flowers in each transgenic line were recorded weekly beginning on Oct. 18, 2021 until the harvest (Jan. 4, 2022 for energycane and Jan. 11, 2022 for sugarcane RNAi lines, Jan. 13, 2022 for Ghd7 over expression lines). Compressed plant circumference was determined by measuring circumference of whole plant at 1 m height for 3 plants in a row plot using measuring tape and expressed in cm. Tiller number was determined at the time of harvest by counting individual tillers per 5 plants in a row plot. Biomass weight was determined at the time of harvest. For determining fresh biomass weight of each line, 5 plants per row were harvested 2″ above soil level using brush cutter (Makita®, EM2650UH, fitted with 9″ Tooth Saw Blade) and fresh biomass weight was measured using a hanging scale (Model: Transcell T1-500RF SS digital weight indicator fitted with BSA-2001b load cell, Central Carolina Scale, Inc. NC, USA). For determining biomass dry weight, a subsample of two tillers per line per replicate were cut, chopped into small pieces, fresh weight was measured, and the biomass was dried at 60° C. oven until constant weight was reached (4 weeks), and the dry weight was determined. Biomass dry weight of each accession was determined by multiplication of the biomass fresh weight with the ratio of each subsample dry weight to the fresh weight. For determining juice volume and Brix, two tiller per line for energycane or one tiller per line for sugarcane and replicate was cut, the stem weight was measured after removing leaves and tops and juice was extracted using a 4 roller sugarcane crusher (model L100B; ASC365 Ltd., EEZGlobal Inc., CA, USA). Brix (total soluble solids) was determined using digital handheld refractometer (Model: PAL-1, ATAGO®, Tokyo, Japan).

### Example 6: Complete Suppression of Flowering Achieved in Sugarcane Using CRISPR/Cas9 Targeted Mutagenesis

The data below show that targeted mutagenesis with designer nucleases like CRISPR/Cas9 of one or multiple of the FT-genes that were confirmed by RNAi will result in suppression of flowering in sugarcane (Saccharum spp. hybrid).

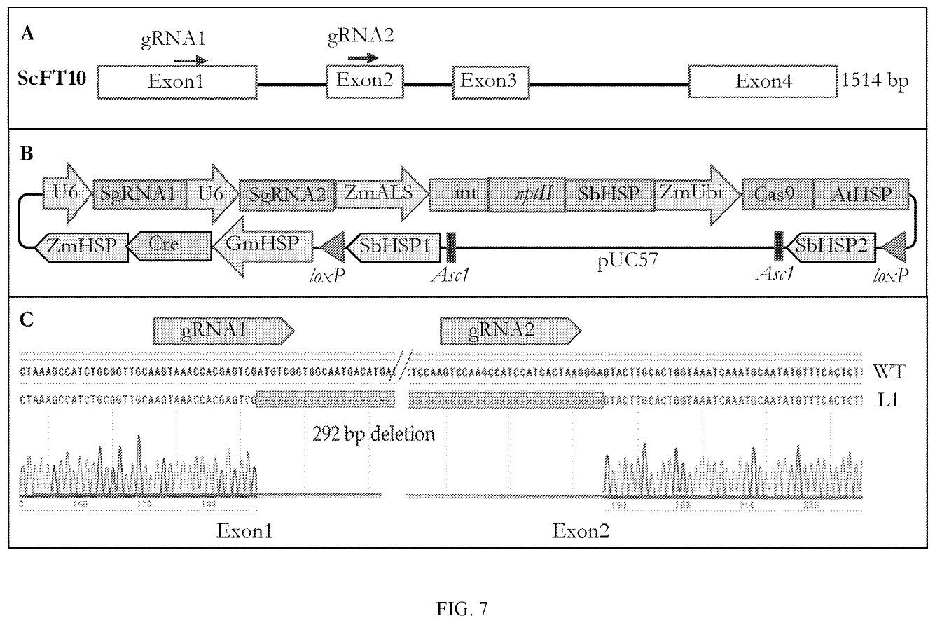

Generation of CRISPR/Cas9 transgenic lines targeting flowering genes: CRISPR/Cas mediated co-suppression of florigens in sugarcane used the most highly conserved region of the ScFT10 sequences for sgRNA design with CRISPOR. Two sgRNAs were selected (FIG. 7A), following in vitro cleavage assay. The two sgRNA's were synthesized and cloned under U6 promoter from Oryza sativa. The sgRNA cassettes were cloned into a multi-gene expression vector containing constitutive expression cassettes for Cas9, the nptII selectable marker and a sugarcane codon-optimized Cre recombinase following restriction digest with SrfI and NotI. Asc1 restriction sites located at either end of the multi-transgene construct allowed for isolation of the minimal cassette from the vector backbone (pUC57) by restriction enzyme digest, gel electrophoresis and purification prior to biolistic gene transfer (FIG. 7B). Transgenic lines were regenerated via somatic embryogenesis on the media containing antibiotic Geneticin following biolistic gene transfer of minimal expression constructs. Regenerated transgenic plants were transferred to soil and grown in a growth chamber for 6 weeks before transfer to a temperature controlled greenhouse under natural photoperiod in Gainesville, FL. Transgenic lines were identified by PCR analysis using construct specific primers (Forward: Cas9-F: 5′-AGGTGGAGAAGGGAAAGTCG-3′, Reverse: Cas9-R: 5′-AGTTCACGTACTTGGACGGC-3′ (SEQ ID Nos. 63 and 64 and NPTII_F: 5′-TACCTGCCCATTCGACCACC-3′ and NPTII R: 5′-TAAAGCACGAGGAAGCGGTC-3′, SEQ ID Nos. 55 and 56), which yielded 261 bp and 345 bp amplicons, respectively on the 1.2% agarose gel visualized under UV transilluminator (Bio-Rad, Hercules, CA) to confirm the presence of CRISPR/Cas9 vector.

Detection of targeted mutation in transgenic lines harboring genome editing reagents: Transgenic lines harboring sgRNAs targeting ScFT10 and Cas9 were screened for the targeted mutations such as SNPs or Insertions and deletions (InDels) using sanger method. PCR amplicons encompassing the target region, amplified using gene specific primers (FT10-F1: 5′-GCAAYATGTCAGCAACCRATCC-3′, FT10-R1: 5′-GTTTCAGGAATATCTGTCACCATCC-3′, SEQ ID Nos. 59 and 60) yielded 576 bp amplicons for ScFT10. PCR amplicons were column purified using GeneJET PCR purification kit (Thermo Fisher Scientific, Madison, WI), ligated into pJET1.2/blunt Cloning Vector (Thermo Fisher Scientific, Madison, WI), and transformed into E. coli DH10-beta competent cells (New England Biolabs Inc, Ipswich, MA) by heat shock transformation method. Transformed colonies were cultured on the LB (Luria-Bertani) liquid media at 37° C., 250 rpm shaker for 14-16 hours and plasmids were purified using GeneJET plasmid miniprep kit (Thermo Fisher Scientific, Madison, WI). Sanger sequencing reactions were performed at Eurofins genomics facility (Eurofins Genomics, Louisville, KY) using forward sequencing primer (5′-CGACTCACTATAGGGAGAGCGGC-3′) or reverse sequencing primer (5′-AAGAACATCGATTTTCCATGGCAG-3′, SEQ ID Nos. 61 and 62). One of the transgenic lines displayed a 292 bp deletion in the ScFT10 target region, which confirmed the functionality of both gRNAs targeted to FT10 (FIG. 7C).

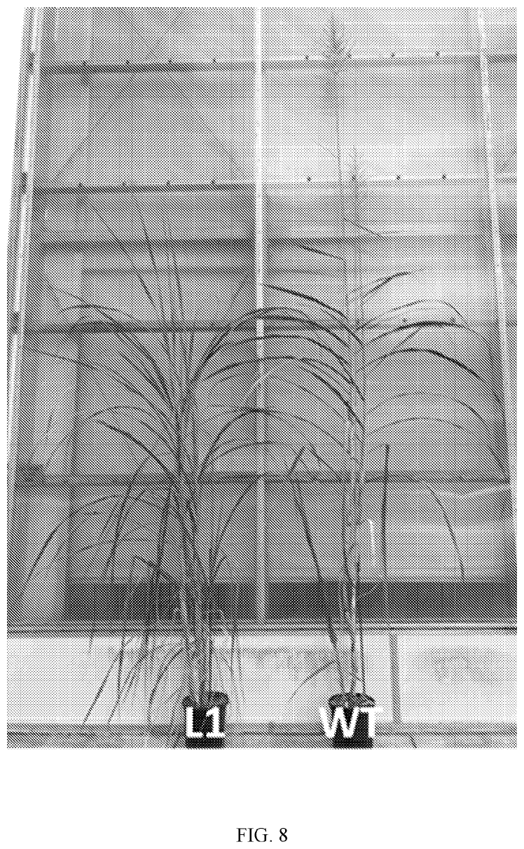

Flowering pattern of transgenic lines with targeted mutagenesis under greenhouse conditions: Line L1 carrying the gene editing vectors for ScFT10 and with a confirmed 292 bp deletion in the ScFT10 target region and non-modified sugarcane cv. CP96-1252 (WT) were transferred from a plant growth chamber to a temperature-controlled greenhouse with approximate 26° C. day and 22° C. night temperatures with natural photoperiod in Gainesville, FL and grown in 15-L pots containing potting mix (Jolly Gardener Pro-Line C/G mix). Pots were irrigated and fertilized by an automated drip-fertigation system to deliver twice daily 3 L irrigation including Miracle-Gro® water soluble lawn fertilizer (NPK: 36-0-6). Imidacloprid (Mallet® 2F T&O), Dinotefuran (Safari® 20SG) or S-Kinoprene (Enstar® AQ) insecticides were applied at the labeled rate for the control of insects such as mealybugs, scales and aphids and Azoxystrobin (Strobe® 50WG) was applied at the labeled rate for the control of orange and brown rust during the grand growth period. During photoinductive period (when natural photoperiod falls below 10 hrs 30 mins in Gainesville, FL) non-modified sugarcane cv. CP96-1252 (WT) flowered Nov. 24, 2022 (FIG. 8, right side), while line (L1) with confirmed targeted mutagenesis of the flowering gene (ScFT10) did not flower (FIG. 8 left side).

Complete suppression of flowering in sugarcane was achieved using CRISPR/Cas9 targeted mutagenesis of the flowering gene ScFT10.

SEQUENCE LISTING

SEQ ID NO: 1 (ScFT4, ScFT8, ScFT10 RNAi-hairpin-long sequence, 1291 bp)
GCTGGTGGTTGGTAGGGTGGTGGGCGACGTGCTGGACCCCTTCGTCCGGACCACCAACCTCAGGGTCAGCTACG
GCACCAGGACCGTATCCAACGGCTGCGAGCTCAAGCCGTCCATGGTGGTGAACCAGCCCAGGGTCGAGGTCGG
GGGACCCGACATGAGGACCTTCTACACCCTCGTGATGGTCATGTGATACGAGATGTGTTGGATTCATTTACACC
AACTATACCCCTCAGAATAACCTACAACAATAGGCTACTTCTAGCAGGTGTTGAGCTGAAGCCATCCGCAGTGG
TGAATAAGCCAAGAGTTGATGTTGGGGGCACCGACCTCAGGGTGTTCTTTACACTGGTAGGTATTAGTTGATCC
AGATGCCCCAAGCCCAAGCAATCCATCACTGAGGGGTCAGCAACCGATCCTTTGGTCATGGCTCATGTCATACA
GGATGTGTTGGATCCCTTTACAACAACCATTCCACTAAGAATAACATACAACAATAGGCTACTTCTGGCAAGTG
CTGAGCTAAAGCCATCTGCGGTTGCAAGTAAACCACGAGTCGATGTCGGTGGCAATGACATGAGGGCTTTCTAC
ACCCTGGGTTTGAATCAGATTCAGATTTCATTGCATCACAGAGATCCATCTTTACTCTACCGCTTGCTCTAACTT
AACTTGTAATTGTTTTTTATCATGCAGCCAGGGTGTAGAAAGCCCTCATGTCATTGCCACCGACATCGACTCGTG
GTTTACTTGCAACCGCAGATGGCTTTAGCTCAGCACTTGCCAGAAGTAGCCTATTGTTGTATGTTATTCTTAGTG
GAATGGTTGTTGTAAAGGGATCCAACACATCCTGTATGACATGAGCCATGACCAAAGGATCGGTTGCTGACCCC
TCAGTGATGGATTGCTTGGGCTTGGGGCATCTGGATCAACTAATACCTACCAGTGTAAAGAACACCCTGAGGTC
GGTGCCCCCAACATCAACTCTTGGCTTATTCACCACTGCGGATGGCTTCAGCTCAACACCTGCTAGAAGTAGCC
TATTGTTGTAGGTTATTCTGAGGGGTATAGTTGGTGTAAATGAATCCAACACATCTCGTATCACATGACCATCAC
GAGGGTGTAGAAGGTCCTCATGTCGGGTCCCCCGACCTCGACCCTGGGCTGGTTCACCACCATGGACGGCTTGA
GCTCGCAGCCGTTGGATACGGTCCTGGTGCCGTAGCTGACCCTGAGGTTGGTGGTCCGGACGAAGGGGTCCAGC
ACGTCGCCCACCACCCTACCAACCACCAGC
SEQ ID NO: 2 (ScFT4, ScFT8, ScFT10 RNAi-hairpin-short sequence, 719 bp)
GACCGTATCCAACGGCTGCGAGCTCAAGCCGTCCATGGTGGTGAACCAGCCCAGGGTCGAGGTCGGGGGACCC
GACATGAGGACCTTCTACACCCTCGTGATGGGAAGCCATCCGCAGTGGTGAATAAGCCAAGAGTTGATGTTGG
GGGCACCGACCTCAGGGTGTTCTTTACACTGGTAGGTATTAGTTGATCCAGATGCCCCAAGCCAACCATTCCAC
TAAGAATAACATACAACAATAGGCTACTTCTGGCAAGTGCTGAGCTAAAGCCATCTGCGGTTGCAAGTAAACC
ACGAGTCGATGTCGGTGGCGTTTGAATCAGATTCAGATTTCATTGCATCACAGAGATCCATCTTTACTCTACCGC
TTGCTCTAACTTAACTTGTAATTGTTTTTTATCATGCAGGCCACCGACATCGACTCGTGGTTTACTTGCAACCGC
AGATGGCTTTAGCTCAGCACTTGCCAGAAGTAGCCTATTGTTGTATGTTATTCTTAGTGGAATGGTTGGCTTGGG
GCATCTGGATCAACTAATACCTACCAGTGTAAAGAACACCCTGAGGTCGGTGCCCCCAACATCAACTCTTGGCT
TATTCACCACTGCGGATGGCTTCCCATCACGAGGGTGTAGAAGGTCCTCATGTCGGGTCCCCCGACCTCGACCC
TGGGCTGGTTCACCACCATGGACGGCTTGAGCTCGCAGCCGTTGGATACGGTC
SEQ ID NO: 3 (ScFT4g3: gRNA sequence, 20 bp)
GCCGTAGCTGACCCTGAGGT
SEQ ID NO: 4 (ScFT4g5: gRNA sequence, 20 bp)
GGCTTGGGCTCGGAGCATCC
SEQ ID NO: 5 (ScFT8g3: gRNA sequence, 20 bp)
GAGAAGTAGCCTATTGTTGT
SEQ ID NO: 6 (ScFT8g4: gRNA sequence, 20 bp)
GCCCAAGCAATCCATCACTG
SEQ ID NO: 7 (ScFT10g3: gRNA sequence, 20 bp)
GGTAAACCACGAGTCGATGT
SEQ ID NO: 8 (ScFT10g6: gRNA sequence, 20 bp)
GTCCAAGCCATCCATCACTA
SEQ ID NO: 9 (ScFT4, ScFT8, ScFT10 RNAi-hairpin-long expression vector, NPT marker,
11724 bp)
GGAACTAGGGATAACAGGGTAATGGCGCGCCGTTGTTTTAGTGTCCGAGGTTTGGTTTGGTGAAGTACATCTGA
ACTCTGAAGGTTCTTCGTGACATGGTTAGACGTGGTGTTGCAGTACTGTGATAAAACTTATCAGAATAAGTTTTC
GCTTCTAATTTGTTTTCTTAAATCATGTGTTGATCAGCCTATGAGTTGTTCATATCGTTGCCTTTCGTGTTGAACA
TTTGTTTTCTTGCCATACTAATGAGATTCATCTGCACAAACTGTACGAAGAGAATCAAGTTTAAACACTGCAGGT
CAACGGATCACCTATCAACATCCCAGCTAAAAACAGTAAAAAGGGGGAAAACGTGGGTGAGTTGAGTCTGTCT
TGTGGAAAAAACGTTTTAGTTTCTCCTGGAATTAACAATAAAAACAGTTGAACAAGATTGACTGTTCCTCCGGG
AGGGTTTGGAACATCGTTACAGATGTGAGCGAAAGGTGAGGAAACAGAGCGGAGGGCTTGGAGGTGACCTCGG
TAGTCGACGCCGGAGTTGAGCTTGATGACGACACCGTACTGGCGTACCAGGCCTAGTAGTGAACACCGGGCCT
GAAGCTGTCGCCGCCGCTGCTCATCTTGTGGGCTGTGCCCGGTGTCCCTGTTGCGGATTGCGGGTGGCAGCCTG
GCAGGTGGGTGCGACCCGTTTGGACTCCCTGATCTGGGCCCTTTGTGTCAGTACCGTCTGTACTCCGATGACATG
CACACCGTCGTCCACAGTCAAGTCCACAATCTCCCCTCTTTTTTTAACGGAATAGTTCAAAATCTCCTTGACGCA
CGCTATCGTGTACCAGCGCTCACTGGACACCACGTTTGTAATCCACGCCGACACGTCGGTCCCACGTCGACAGG
CCCCACCGTCCGGTCTGTAGCGTGTACGTATTCGGGCGACGGACGTGTCGTCGTCGTCTTGCGAGTCCCATTCCC
ATCACCATCTGAGCCACACATCCTCTGAACAAAAGCAGGGAGGCCTCTACGCACATCCCCCTTTCTCCCACTCC
TTGTCCGTGGCACCCACCCCAAACCCTCGCGCCGCCTCCGAGACAGCCGCCGCAACCATGGCCACCGCCTGCAG
GACCGTCTTCGGTACGCGCTCACTCCGCCCTCTGCCTTTGTTACTGCCACGTTTCTCTGAATGCTCTCTTGTGTGG
TGATTGCTGAGAGTGGTTTAGCTGGATCTAGAATTACACTCTGAAATCGTGTTCTGCCTGTGCTGATTACTTGCC
GTCCTTTGTAGCAGCAAAATATAGGGACATGGTAGTACGAAACGAAGATAGAACCTACACAGCAATACGAGAA
ATGTGTAATTTGGTGCTTAGCGGTATTTATTTAAGCACATGTTGGTGTTATAGGGCACTTGGATTCAGAAGTTTG
CTGTTAATTTAGGCACAGGCTTCATACTACATGGGTCAATAGTATAGGGATTCATATTATAGGCGATACTATAA
TAATTTGTTCGTCTGCAGAGCTTATTATTTGCCAAAATTAGATATTCCTATTCTGTTTTTGTTTGTGTGCTGTTAA
ATTGTTAACGCCTGAAGGAATAAATATAAATGACGAAATTTTGATGTTTATCTCTGCTCCTTTATTGTGACCATA
AGTCAAGATCAGATGCACTTGTTTTAAATATTGTTGTCTGAAGAAATAAGTACTGACAGTATTTTGATGCATTGA
TCTGCTTGTTTGTTGTAACAAAATTTAAAAATAAAGAGTTTCCTTTTTGTTGCTCTCCTTACCTCCTGATGGTATC
TAGTATCTACCAACTGACACTATATTGCTTCTCTTTACATACGTATCTTGCTCGATGCCTTCTCCCTAGTGTTGAC
CAGTGTTACTCACATAGTCTTTGCTCATTTCATTGTAATGCAGATACCAAGCGGCCCCTGCAGGATGGGGATTG
AACAAGATGGATTGCACGCAGGTTCTCCGGCCGCTTGGGTGGAGAGGCTATTCGGCTATGACTGGGCACAACA
GACAATCGGCTGCTCTGATGCCGCCGTGTTCCGGCTGTCAGCGCAGGGGCGCCCGGTTCTTTTTGTCAAGACCG
ACCTGTCCGGTGCCCTGAATGAACTCCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGTGCCGGGGCAGG
ATCTCCTGTCATCTCACCTTGCTCCTGCCGAGAAAGTATCCATCATGGCTGATGCAATGCGGCGGCTGCATACGC
TTGATCCGGCTACCTGCCCATTCGACCACCAAGCGAAACATCGCATCGAGCGAGCACGTACTCGGATGGAAGCC
GGTCTTGTCGATCAGGATGATCTGGACGAAGAGCATCAGGGGCTCGCGCCAGCCGAACTGTTCGCCAGGCTCA
AGGCGCGCATGCCCGACGGCGAGGATCTCGTCGTGACACATGGCGATGCCTGCTTGCCGAATATCATGGTGGA
AAATGGCCGCTTTTCTGGATTCATCGACTGTGGCCGGCTGGGTGTGGCGGACCGCTATCAGGACATAGCGTTGG
CTACCCGTGATATTGCTGAAGAGCTTGGCGGCGAATGGGCTGACCGCTTCCTCGTGCTTTACGGTATCGCCGCTC
CCGATTCGCAGCGCATCGCCTTCTATCGCCTTCTTGACGAGTTCTTCTGAGCGATCGCAGAGCCATGGGGGCGG
TGAGAGCTCGCGGCGTCGGATCTCCGGTTTCAGTTTCAGCTACGCGACTTGTGTGTGTGTGTGTGTGTCAGTGCG
TGGTTACTCTGAGCTTCGGTATGTTTGGTGTGTACTGTTCAGTGTTTCATGGTGCTTCCTAGCAGTGTCTGTCGTG
TACTTTGCCTGTATGTTGAGCTCGGCTCAGTATGTTCTGGGAGTGAATAAATAAAAAAAAATTAACCAGATATT
GTCGCGTGTACTTAGTATACTAATCACCTGCTCCTGCTCTTGCTCTGTTTTATCACCTTTCGGGCTTGACTGTCTT
TGTGGAGGCCTCTCAGTCTGATCTGGAACTCTACCTAAGTTTTACTAGTCCCGGGAAGCTTCTGCAGTGCAGCGT
GACCCGGTCGTGCCCCTCACTAGAGATAATGAGCATTGCATGTCTAAGTTATAAAAAATTACCACATATTTTTTT
TGTCACACTTGTTTGAAGTGCAGTTTATCTATCTTTATACATATATTTAAACTTTACTCTACGAATAATATAATCT
ATAGTACTACAATAATATCAGTGTTTTAGAGAATCATATAAATGAACAGTTAGACATGGTCTAAAGGACAATTG
AGTATTTTGACAACAGGACTCTACAGTTTTATCTTTTTAGTGTGCATGTGTTCTCCTTTTTTTTTGCAAATAGCTT
CACCTATATAATACTTCATCCATTTTATTAGTACATCCATTTAGGGTTTAGGGTTAATGGTTTTTATAGACTAATT
TTTTTAGTACATCTATTTTATTCTATTTTAGCCTCTAAATTAAGAAAACTAAAACTCTATTTTAGTTTTTTTATTTA
ATAATTTAGATATAAAATAGAATAAAATAAAGTGACTAAAAATTAAACAAATACCCTTTAAGAAATTAAAAAA
ACTAAGGAAACATTTTTCTTGTTTCGAGTAGATAATGCCAGCCTGTTAAACGCCGTCGACGAGTCTAACGGACA
CCAACCAGCGAACCAGCAGCGTCGCGTCGGGCCAAGCGAAGCAGACGGCACGGCATCTCTGTCGCTGCCTCTG
GACCCCTCTCGAGAGTTCCGCTCCACCGTTGGACTTGCTCCGCTGTCGGCATCCAGAAATTGCGTGGCGGAGCG
GCAGACGTGAGCCGGCACGGCAGGCGGCCTCCTCCTCCTCTCACGGCACGGCAGCTACGGGGGATTCCTTTCCC
ACCGCTCCTTCGCTTTCCCTTCCTCGCCCGCCGTAATAAATAGACACCCCCTCCACACCCTCTTTCCCCAACCTC
GTGTTGTTCGGAGCGCACACACACACAACCAGATCTCCCCCAAATCCACCCGTCGGCACCTCCGCTTCAAGGTA
CGCCGCTCGTCCTCCCCCCCCCCCCCTCTCTACCTTCTCTAGATCGGCGTTCCGGTCCATGGTTAGGGCCCGGTA
GTTCTACTTCTGTTCATGTTTGTGTTAGATCCGTGTTTGTGTTAGATCCGTGCTGCTAGCGTTCGTACACGGATGC
GACCTGTACGTCAGACACGTTCTGATTGCTAACTTGCCAGTGTTTCTCTTTGGGGAATCCTGGGATGGCTCTAGC
CGTTCCGCAGACGGGATCGATTTCATGATTTTTTTTGTTTCGTTGCATAGGGTTTGGTTTGCCCTTTTCCTTTATTT
CAATATATGCCGTGCACTTGTTTGTCGGGTCATCTTTTCATGCTTTTTTTTGTCTTGGTTGTGATGATGTGGTCTG
GTTGGGCGGTCGTTCTAGATCGGAGTAGAATTCTGTTTCAAACTACCTGGTGGATTTATTAATTTTGGATCTGTA
TGTGTGTGCCATACATATTCATAGTTACGAATTGAAGATGATGGATGGAAATATCGATCTAGGATAGGTATACA
TGTTGATGCGGGTTTTACTGATGCATATACAGAGATGCTTTTTGTTCGCTTGGTTGTGATGATGTGGTGTGGTTG
GGCGGTCGTTCATTCGTTCTAGATCGGAGTAGAATACTGTTTCAAACTACCTGGTGTATTTATTAATTTTGGAAC
TGTATGTGTGTGTCATACATCTTCATAGTTACGAGTTTAAGATGGATGGAAATATCGATCTAGGATAGGTATAC
ATGTTGATGTGGGTTTTACTGATGCATATACATGATGGCATATGCAGCATCTATTCATATGCTCTAACCTTGAGT
ACCTATCTATTATAATAAACAAGTATGTTTTATAATTATTTTGATCTTGATATACTTGGATGATGGCATATGCAG
CAGCTATATGTGGATTTTTTTAGCCCTGCCTTCATACGCTATTTATTTGCTTGGTACTGTTTCTTTTGTCGATGCTC
ACCCTGTTGTTTGGTGTTACTTCTCTAGAGCGGCCGCATTTAAATCACTCTGTGGTCTCAAATGGCTGGTGGTTG
GTAGGGTGGTGGGCGACGTGCTGGACCCCTTCGTCCGGACCACCAACCTCAGGGTCAGCTACGGCACCAGGAC
CGTATCCAACGGCTGCGAGCTCAAGCCGTCCATGGTGGTGAACCAGCCCAGGGTCGAGGTCGGGGGACCCGAC
ATGAGGACCTTCTACACCCTCGTGATGGTCATGTGATACGAGATGTGTTGGATTCATTTACACCAACTATACCCC
TCAGAATAACCTACAACAATAGGCTACTTCTAGCAGGTGTTGAGCTGAAGCCATCCGCAGTGGTGAATAAGCCA
AGAGTTGATGTTGGGGGCACCGACCTCAGGGTGTTCTTTACACTGGTAGGTATTAGTTGATCCAGATGCCCCAA
GCCCAAGCAATCCATCACTGAGGGGTCAGCAACCGATCCTTTGGTCATGGCTCATGTCATACAGGATGTGTTGG
ATCCCTTTACAACAACCATTCCACTAAGAATAACATACAACAATAGGCTACTTCTGGCAAGTGCTGAGCTAAAG
CCATCTGCGGTTGCAAGTAAACCACGAGTCGATGTCGGTGGCAATGACATGAGGGCTTTCTACACCCTGGGTTT
GAATCAGATTCAGATTTCATTGCATCACAGAGATCCATCTTTACTCTACCGCTTGCTCTAACTTAACTTGTAATT
GTTTTTTATCATGCAGCCAGGGTGTAGAAAGCCCTCATGTCATTGCCACCGACATCGACTCGTGGTTTACTTGCA
ACCGCAGATGGCTTTAGCTCAGCACTTGCCAGAAGTAGCCTATTGTTGTATGTTATTCTTAGTGGAATGGTTGTT
GTAAAGGGATCCAACACATCCTGTATGACATGAGCCATGACCAAAGGATCGGTTGCTGACCCCTCAGTGATGG
ATTGCTTGGGCTTGGGGCATCTGGATCAACTAATACCTACCAGTGTAAAGAACACCCTGAGGTCGGTGCCCCCA
ACATCAACTCTTGGCTTATTCACCACTGCGGATGGCTTCAGCTCAACACCTGCTAGAAGTAGCCTATTGTTGTAG
GTTATTCTGAGGGGTATAGTTGGTGTAAATGAATCCAACACATCTCGTATCACATGACCATCACGAGGGTGTAG
AAGGTCCTCATGTCGGGTCCCCCGACCTCGACCCTGGGCTGGTTCACCACCATGGACGGCTTGAGCTCGCAGCC
GTTGGATACGGTCCTGGTGCCGTAGCTGACCCTGAGGTTGGTGGTCCGGACGAAGGGGTCCAGCACGTCGCCCA
CCACCCTACCAACCACCAGCGCTTTGAGACCACGAAGTGTTCGAATTAATTAAGGGGACACCGTTGTCGGAGCA
GTGCCAGTGAGCTGGGTGGCAACCTGCGGGACTCTGGTGAGGAAGAGCAAATGGTAGAAAGCAGTAGCTGTGC
TTCTGAACATTGTGTGTGTTTGTTTCCCTTGTTACCGCCAATCATCCTACCTGATGTGTCGTCTGGGGTTTCGTTT
GAACCATGCTGGGTTTGGAGCTTTGTGATGCAAAGCAGAATGTCCTGATGCTTTCTTTCTCCATAGAATAAATCT
CTCCTGTTACCTAAAGGCCGGCCGATATCCATTCAAGAGGAGAGCTCATTTCCTCTCCTCTCATCACCTTTGCCT
AAAAACAGTATGAAAGTCAAGCTAAAGACTTTAAACTAGCTCACTTGGGAGGAAGTCCCATGTCTATTCTTGTA
TATACTTTTTTCCCTTGTTATTTTTGATGTTTTGTGTAAGTATTTCAGGTTTTATAGTCCACGGAAGTCAACGATC
GAGTGATATAAATCGAGTCAAAGAATCTTAGCCGTGCGATAGAGTGACTTAAGGCGAAGCATCCAGCTAATCC
GAGTCCCACACAACCTATTCCTTGGAGCTCTAAACGGGAGAGTATGGACGACTTCGTCAACTCCTATTTCACCA
GATCACTAAGGTAACACACCACCCCCATTGTATATACCGTTATCTTTTATTTTTCTTTGTTTGTTGTTTGTTTTAG
TCTTGAATTCTCTCTCAGATTTTTAATACACAAGGGACTGTGTAATTTAAGTGTGGGGGAGGGTTCAAGAAATA
TTTGACTTTATGTTCTCTTTTCTTATTTCAAATTTTGCATCATCTAAGGCATAAAAAACCATAAAAATTTAAAAA
CATTTATGAAAATGATTTCACCAAAACAGAGTGTCATGTAGATTGCATTGCATTTTAGGATTGTATATAGAGTA
CTTGCATTTAGGATTTCTCATTTGCATAGGGGATAATGATGATTTCGTACCGTTAAGCATGTTTTGATTCAATGA
AGTAAGTTCAATGCCCAGGTTGTTAGTTCTCTGTTGCATAACCGATTGTTTCTCTGAAGTAAGACTGCACCCTGC
TTTGAATTTACAACTTGACTATACTTCTTTGATTCAAAACTCGCTTATCTGATGCCATTCCCTATCTATTAGAACC
TGAACTGAATTTGCAATTATCATGTCTATGCATTGTTTGTGAACTCATGGCTACCATATACATACTTGTATTATCT
TATTCACTTCACCACCCTCTGTTAATCTAAGTAGCTGACTCTCATTTTTAGAGCAGTTTCCCATACCCTTAGCCTA
GCCTTATTTCAAGCCAAAGATCATTTGTGTGTGATTGTGAGGTCTTTTTCGATTGAGCTTGGTATAACGTGTCAG
GTATGAGCCGACAAGAGCAGTCTTTCATGTAGACCTAGCCCACGCTTTTTGGGTTAGCTAGGACTAGGTGGAGA
CTTGTATTTGGGATTGGGGTTGTGTATGGATTGAATGGGAAAGAGAATGAGAATGAAAAGAAAAGAAAAAAAG
AGAGTAAGAGTTTAGAGTTCTTAAGGGGGATACAAAAAGTGTTAGTCTAGTAAAGGGTTTGGAATTCAAAGAA
GTTAGGCATTGTGATTTAAAGAAAAGGGTAAAACGACCTAAGATTAGAAGAAAAGCCTTAAGTTTGTCAAAGT
ATAAAACCAAACCCACTAGACTCTTGAATCGTTTTAAAGAAGAACCCCTTAGAACTTGAACCAAAAAGAGAAA
GAATGAGAAAAAGGGTAGAGTTAGGACATCTATGGGACTGGAACCATCTTATGAACTATCATACTATATCATTG
GGTAGATGGGAGTCTGTTCTTGTTTGTGTCCATTGGCTTTTACCTTTAGCATTCTGCTGACGCTCAATCTTTTCTT
TGAGAGTCCCCTGTTACTCGACCAAATCATTAAAGGGACCATTTTTGTCTCTTAGCCTAAGCCTGAAACCAAGT
GAGTTCATAACCATTGCATTGCTTGATCCATTGTTCGTGCTTAGTGAATGTTAAAAAGAATTGGTTGATGTGAAT
GCTTGAATAGATTAAGGCAGGTTGTTAGGTTGCTTTGTGACGAGTATGGCTAAATTTTTAAGTAAGGGTCTATC
ATTTTGCAACCTATAATTAGCTACCTGGACATTGAGCTTACCCGTTCTATATGCATGCTACGGGTTTTGAATCCC
CACCTTCAAACCTCTCCTTCAACTTAAGATTTTTTGTTTGCTTGAGGGCAAGCAAGAGATAAGTTTGGGGGAGTT
GATAACACTGTAATTTACTCATTTTGGGCATGTATTTATCATCATTTAGCTTAGTGATCACTCATTGTTTCATATC
ATTTTGCATCACATTTAGCTTATTTTGCACACTTAGGCATTTAGGGTCATCTTGCATACATATTGCATGTTATGGT
GTGTTTTCAGGTGCAAAATAAGTTTACAAGGAGTTTACTCGACCCTCTACCCTATTGCCCCAGAGGGGAACCAG
AGGAGCAGCTACTCGACCAATTATTCATTGCCTCAGAGGGGAAGCAGCAGAGCAGCTACTCGACCAGTTATTCG
GCGCGCCTAGGGATAACAGGGTAATGTTCCATCCCAATGGCGCGCCGAGCTTGGCGTAATCATGGTCATAGCTG
TTTCCTGTGTGAAATTGTTATCCGCTCACAATTCCACACAACATACGAGCCGGAAGCATAAAGTGTAAAGCCTG
GGGTGCCTAATGAGTGAGCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATTGGGCGCTGTTCCGCTTCC
TCGCTCACTGACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAAATCGACGCTCAA
GTCAGAGGTGGCGAAACCCGACAGGACTATAAAGATACCAGGCGTTTCCCCCTGGAAGCTCCCTCGTGCGCTCT
CCTGTTCCGACCCTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGGGAGGGCTT
ACCATCTGGCCCCAGTGCTGCAATGATACCGCGAGAACCACGCTCACCGGCTCCAGATTTATCAGCAATAAACC
AGCCAGCCGGAAGGGCCGAGCGCAGAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGACACGGAAATG
TTGAATACTCATACTCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACCTGACGTCTA
AGAAACCATTATTATCATGACATTAACCTATAAAAATAGGCGTATCACGAGGCCCTTTTGTCTCGCGCGTTTCG
GTGATGACGGTGAAAACCTCTGACACATGCAGCTCCCGGAGACTGTCACAGCTTGTCTGTAAGCGGATGCCGGG
AGCAGACAAGCCCGTCAGGGCGCGTCAGCGGGTGTTGGCGGGTGTCGGGGCTGGCTTAACTATGCGGCATCAG
AGCAGATTGTACTGAGAGTGCACCATATGCGGTGTGAAATACCGCACAGATGCGTAAGGAGAAAATACCGCAT
CAGGCGCCATTCGCCATTCAGGCTGCGCAACTGTTGGGAAGGGCGATCGGTGCGGGCCTCTTCGCTATTACGCC
AGCTGGCGAAAGGGGGATGTGCTGCAAGGCGATTAAGTTGGGTAACGCCAGGGTTTTCCCAGTCACGACGTTG
TAAAACGACGGCCAGTGAATTGACGCGTATTGGGAT
SEQ ID NO: 10 (ScFT4, ScFT8, ScFT10 RNAi-hairpin-long expression vector, ALS marker,
12852 bp)
GGAACTAGGGATAACAGGGTAATGGCGCGCCGTTGTTTTAGTGTCCGAGGTTTGGTTTGGTGAAGTACATCTGA
ACTCTGAAGGTTCTTCGTGACATGGTTAGACGTGGTGTTGCAGTACTGTGATAAAACTTATCAGAATAAGTTTTC
GCTTCTAATTTGTTTTCTTAAATCATGTGTTGATCAGCCTATGAGTTGTTCATATCGTTGCCTTTCGTGTTGAACA
TTTGTTTTCTTGCCATACTAATGAGATTCATCTGCACAAACTGTACGAAGAGAATCAAGTTTAAACACTGCAGGT
CAACGGATCACCTATCAACATCCCAGCTAAAAACAGTAAAAAGGGGGAAAACGTGGGTGAGTTGAGTCTGTCT
TGTGGAAAAAACGTTTTAGTTTCTCCTGGAATTAACAATAAAAACAGTTGAACAAGATTGACTGTTCCTCCGGG
AGGGTTTGGAACATCGTTACAGATGTGAGCGAAAGGTGAGGAAACAGAGCGGAGGGCTTGGAGGTGACCTCGG
TAGTCGACGCCGGAGTTGAGCTTGATGACGACACCGTACTGGCGTACCAGGCCTAGTAGTGAACACCGGGCCT
GAAGCTGTCGCCGCCGCTGCTCATCTTGTGGGCTGTGCCCGGTGTCCCTGTTGCGGATTGCGGGTGGCAGCCTG
GCAGGTGGGTGCGACCCGTTTGGACTCCCTGATCTGGGCCCTTTGTGTCAGTACCGTCTGTACTCCGATGACATG
CACACCGTCGTCCACAGTCAAGTCCACAATCTCCCCTCTTTTTTTAACGGAATAGTTCAAAATCTCCTTGACGCA
CGCTATCGTGTACCAGCGCTCACTGGACACCACGTTTGTAATCCACGCCGACACGTCGGTCCCACGTCGACAGG
CCCCACCGTCCGGTCTGTAGCGTGTACGTATTCGGGCGACGGACGTGTCGTCGTCGTCTTGCGAGTCCCATTCCC
ATCACCATCTGAGCCACACATCCTCTGAACAAAAGCAGGGAGGCCTCTACGCACATCCCCCTTTCTCCCACTCC
TTGTCCGTGGCACCCACCCCAAACCCTCGCGCCGCCTCCGAGACAGCCGCCGCAACCATGGCCACCGCCTGCAG
GACCGTCTTCGGTACGCGCTCACTCCGCCCTCTGCCTTTGTTACTGCCACGTTTCTCTGAATGCTCTCTTGTGTGG
TGATTGCTGAGAGTGGTTTAGCTGGATCTAGAATTACACTCTGAAATCGTGTTCTGCCTGTGCTGATTACTTGCC
GTCCTTTGTAGCAGCAAAATATAGGGACATGGTAGTACGAAACGAAGATAGAACCTACACAGCAATACGAGAA
ATGTGTAATTTGGTGCTTAGCGGTATTTATTTAAGCACATGTTGGTGTTATAGGGCACTTGGATTCAGAAGTTTG
CTGTTAATTTAGGCACAGGCTTCATACTACATGGGTCAATAGTATAGGGATTCATATTATAGGCGATACTATAA
TAATTTGTTCGTCTGCAGAGCTTATTATTTGCCAAAATTAGATATTCCTATTCTGTTTTTGTTTGTGTGCTGTTAA
ATTGTTAACGCCTGAAGGAATAAATATAAATGACGAAATTTTGATGTTTATCTCTGCTCCTTTATTGTGACCATA
AGTCAAGATCAGATGCACTTGTTTTAAATATTGTTGTCTGAAGAAATAAGTACTGACAGTATTTTGATGCATTGA
TCTGCTTGTTTGTTGTAACAAAATTTAAAAATAAAGAGTTTCCTTTTTGTTGCTCTCCTTACCTCCTGATGGTATC
TAGTATCTACCAACTGACACTATATTGCTTCTCTTTACATACGTATCTTGCTCGATGCCTTCTCCCTAGTGTTGAC
CAGTGTTACTCACATAGTCTTTGCTCATTTCATTGTAATGCAGATACCAAGCGGCCCCTGCAGGATGGCCACCAC
CGCCGCCGCCGCTGCCGCCGCGCTAGCCGGCGCCACTACCGCTGCGCCCAAGGCGAGGCGCCGGGCGCACCTC
CTGGCCGCACGGCGCGCCCTCGCCGCGCCCATCAGGTGCTCAGCGGCGCCACCCGCCACGCTGACGGTGACGG
CTCCCCCGGCCACCCCGCTCCGGCCGTGGGGCCCCACCGATCCCCGCAAGGGCGCCGACATCCTCGTCGAGGCT
CTTGAGCGCTGCGGCGTCCGCGACGTCTTCGCCTACCCCGGCGGCGCGTCCATGGAGATCCACCAGGCACTCAC
CCGTTCCCCCGTCATCGCCAACCACCTCTTCCGCCACGAGCAAGGGGAGGCCTTCGCCGCCTCTGGCTTCGCGC
GCTCCTCGGGCCGCGTCGGCGTCTGCGTCGCCACCTCCGGCCCCGGCGCCACCAACCTAGTCTCCGCGCTCGCC
GACGCGCTGCTCGACTCCGTCCCCATGGTCGCCATCACGGGACAGGTTCCGCGGCGCATGATTGGCACCGACGC
CTTCCAGGAGACGCCCATCGTCGAGGTCACCCGCTCCATCACCAAACATAACTACCTGGTCCTCGACGTCGACG
ACATCCCCCGCGTCGTGCAGGAGGCTTTCTTCCTCGCCTCCTCCGGTCGCCCGGGACCGGTGCTTGTCGACATCC
CCAAGGACATCCAGCAGCAGATGGCCGTGCCGGTCTGGGACACGCCCATGAGTCTGCCTGGGTACATTGCGCG
CCTTCCCAAGCCTCCTGCGACTGAATTGCTTGAGCAGGTGCTGCGTCTTGTTGGTGAATCAAGGCGCCCTGTTCT
TTATGTTGGTGGTGGCTGCGCAGCATCTGGCGAGGAGTTGCGCCGCTTTGTGGAGATGACTGGAATCCCAGTCA
CAACTACTCTTATGGGCCTTGGCAATTTCCCTGGCGACGACCCACTGTCTCTGCGCATGCTTGGTATGCATGGCA
CGGTGTATGCAAATTATGCAGTGGATAAGGCGGATCTGTTGCTTGCATTTGGTGTGCGGTTTGATGATCGTGTG
ACAGGGAAGATTGAGGCTTTTGCAAGCAGGGCTAAGATTGTGCACATTGATATTGATCCCGCTGAGATTGGCAA
GAACAAGCAGCCACATGTGTCCATCTGTGCAGACGTTAAGCTTGCTTTGCAGGGCATGAATGCTCTTCTGGAAG
GAAGCACATCAAAGAAGAGCTTTGACTTTGGCTCATGGCAAGCTGAGTTGGATCAGCAGAAGAGAGAGTTCCC
CCTTGGGTATAAAACTTTTGATGACGAGATCCAGCCACAATATGCTATTCAGGTTCTTGATGAGCTGACAAAAG
GGGAGGCCATCATTGCCACAGGTGTTGGGCAGCACCAGATGTGGGCGGCACAGTACTACACTTACAAGCGGCC
AAGGCAGTGGTTGTCTTCAGCTGGTCTTGGGGCTATGGGATTTGGTTTGCCGGCTGCTGCTGGCGCTGCTGTGGC
CAACCCAGGTATCACTGTTGTTGACATCGACGGAGATGGTAGCTTCCTCATGAACATTCAGGAGCTAGCTATGA
TCCGAATTGAGAACCTCCCAGTGAAGGTCTTTGTGCTAAACAACCAGCACCTGGGGATGGTGGTGCAGTTGGAG
GACAGGTTCTATAAGGCCAATAGAGCACACACATACTTGGGAAACCCAGAGAATGAAAGTGAGATATATCCAG
ATTTCGTGACAATTGCCAAAGGGTTCAACATTCCAGCAGTCCGTGTGACAAAGAAGAGCGAAGTCCATGCAGC
AATCAAGAAGATGCTTGAGACTCCAGGGCCATACCTCTTGGATATAATCGTCCCGCACCAGGAGCATGTGTTGC
CTATGATCCCTAGTGGTGGGGCTTTCAAGGATATGATCCTGGATGGTGATGGCAGGACTGTGTATTGAGCGATC
GCAGAGCCATGGGGGCGGTGAGAGCTCGCGGCGTCGGATCTCCGGTTTCAGTTTCAGCTACGCGACTTGTGTGT
GTGTGTGTGTGTCAGTGCGTGGTTACTCTGAGCTTCGGTATGTTTGGTGTGTACTGTTCAGTGTTTCATGGTGCTT
CCTAGCAGTGTCTGTCGTGTACTTTGCCTGTATGTTGAGCTCGGCTCAGTATGTTCTGGGAGTGAATAAATAAAA
AAAAATTAACCAGATATTGTCGCGTGTACTTAGTATACTAATCACCTGCTCCTGCTCTTGCTCTGTTTTATCACCT
TTCGGGCTTGACTGTCTTTGTGGAGGCCTCTCAGTCTGATCTGGAACTCTACCTAAGTTTTACTAGTCCCGGGAA
GCTTCTGCAGTGCAGCGTGACCCGGTCGTGCCCCTCACTAGAGATAATGAGCATTGCATGTCTAAGTTATAAAA
AATTACCACATATTTTTTTTGTCACACTTGTTTGAAGTGCAGTTTATCTATCTTTATACATATATTTAAACTTTAC
TCTACGAATAATATAATCTATAGTACTACAATAATATCAGTGTTTTAGAGAATCATATAAATGAACAGTTAGAC
ATGGTCTAAAGGACAATTGAGTATTTTGACAACAGGACTCTACAGTTTTATCTTTTTAGTGTGCATGTGTTCTCC
TTTTTTTTTGCAAATAGCTTCACCTATATAATACTTCATCCATTTTATTAGTACATCCATTTAGGGTTTAGGGTTA
ATGGTTTTTATAGACTAATTTTTTTAGTACATCTATTTTATTCTATTTTAGCCTCTAAATTAAGAAAACTAAAACT
CTATTTTAGTTTTTTTATTTAATAATTTAGATATAAAATAGAATAAAATAAAGTGACTAAAAATTAAACAAATAC
CCTTTAAGAAATTAAAAAAACTAAGGAAACATTTTTCTTGTTTCGAGTAGATAATGCCAGCCTGTTAAACGCCG
TCGACGAGTCTAACGGACACCAACCAGCGAACCAGCAGCGTCGCGTCGGGCCAAGCGAAGCAGACGGCACGG
CATCTCTGTCGCTGCCTCTGGACCCCTCTCGAGAGTTCCGCTCCACCGTTGGACTTGCTCCGCTGTCGGCATCCA
GAAATTGCGTGGCGGAGCGGCAGACGTGAGCCGGCACGGCAGGCGGCCTCCTCCTCCTCTCACGGCACGGCAG
CTACGGGGGATTCCTTTCCCACCGCTCCTTCGCTTTCCCTTCCTCGCCCGCCGTAATAAATAGACACCCCCTCCA
CACCCTCTTTCCCCAACCTCGTGTTGTTCGGAGCGCACACACACACAACCAGATCTCCCCCAAATCCACCCGTCG
GCACCTCCGCTTCAAGGTACGCCGCTCGTCCTCCCCCCCCCCCCCTCTCTACCTTCTCTAGATCGGCGTTCCGGT
CCATGGTTAGGGCCCGGTAGTTCTACTTCTGTTCATGTTTGTGTTAGATCCGTGTTTGTGTTAGATCCGTGCTGCT
AGCGTTCGTACACGGATGCGACCTGTACGTCAGACACGTTCTGATTGCTAACTTGCCAGTGTTTCTCTTTGGGGA
ATCCTGGGATGGCTCTAGCCGTTCCGCAGACGGGATCGATTTCATGATTTTTTTTGTTTCGTTGCATAGGGTTTG
GTTTGCCCTTTTCCTTTATTTCAATATATGCCGTGCACTTGTTTGTCGGGTCATCTTTTCATGCTTTTTTTTGTCTT
GGTTGTGATGATGTGGTCTGGTTGGGCGGTCGTTCTAGATCGGAGTAGAATTCTGTTTCAAACTACCTGGTGGAT
TTATTAATTTTGGATCTGTATGTGTGTGCCATACATATTCATAGTTACGAATTGAAGATGATGGATGGAAATATC
GATCTAGGATAGGTATACATGTTGATGCGGGTTTTACTGATGCATATACAGAGATGCTTTTTGTTCGCTTGGTTG
TGATGATGTGGTGTGGTTGGGCGGTCGTTCATTCGTTCTAGATCGGAGTAGAATACTGTTTCAAACTACCTGGTG
TATTTATTAATTTTGGAACTGTATGTGTGTGTCATACATCTTCATAGTTACGAGTTTAAGATGGATGGAAATATC
GATCTAGGATAGGTATACATGTTGATGTGGGTTTTACTGATGCATATACATGATGGCATATGCAGCATCTATTCA
TATGCTCTAACCTTGAGTACCTATCTATTATAATAAACAAGTATGTTTTATAATTATTTTGATCTTGATATACTTG
GATGATGGCATATGCAGCAGCTATATGTGGATTTTTTTAGCCCTGCCTTCATACGCTATTTATTTGCTTGGTACT
GTTTCTTTTGTCGATGCTCACCCTGTTGTTTGGTGTTACTTCTCTAGAGCGGCCGCATTTAAATCACTCTGTGGTC
TCAAATGGCTGGTGGTTGGTAGGGTGGTGGGCGACGTGCTGGACCCCTTCGTCCGGACCACCAACCTCAGGGTC
AGCTACGGCACCAGGACCGTATCCAACGGCTGCGAGCTCAAGCCGTCCATGGTGGTGAACCAGCCCAGGGTCG
AGGTCGGGGGACCCGACATGAGGACCTTCTACACCCTCGTGATGGTCATGTGATACGAGATGTGTTGGATTCAT
TTACACCAACTATACCCCTCAGAATAACCTACAACAATAGGCTACTTCTAGCAGGTGTTGAGCTGAAGCCATCC
GCAGTGGTGAATAAGCCAAGAGTTGATGTTGGGGGCACCGACCTCAGGGTGTTCTTTACACTGGTAGGTATTAG
TTGATCCAGATGCCCCAAGCCCAAGCAATCCATCACTGAGGGGTCAGCAACCGATCCTTTGGTCATGGCTCATG
TCATACAGGATGTGTTGGATCCCTTTACAACAACCATTCCACTAAGAATAACATACAACAATAGGCTACTTCTG
GCAAGTGCTGAGCTAAAGCCATCTGCGGTTGCAAGTAAACCACGAGTCGATGTCGGTGGCAATGACATGAGGG
CTTTCTACACCCTGGGTTTGAATCAGATTCAGATTTCATTGCATCACAGAGATCCATCTTTACTCTACCGCTTGCT
CTAACTTAACTTGTAATTGTTTTTTATCATGCAGCCAGGGTGTAGAAAGCCCTCATGTCATTGCCACCGACATCG
ACTCGTGGTTTACTTGCAACCGCAGATGGCTTTAGCTCAGCACTTGCCAGAAGTAGCCTATTGTTGTATGTTATT
CTTAGTGGAATGGTTGTTGTAAAGGGATCCAACACATCCTGTATGACATGAGCCATGACCAAAGGATCGGTTGC
TGACCCCTCAGTGATGGATTGCTTGGGCTTGGGGCATCTGGATCAACTAATACCTACCAGTGTAAAGAACACCC
TGAGGTCGGTGCCCCCAACATCAACTCTTGGCTTATTCACCACTGCGGATGGCTTCAGCTCAACACCTGCTAGA
AGTAGCCTATTGTTGTAGGTTATTCTGAGGGGTATAGTTGGTGTAAATGAATCCAACACATCTCGTATCACATG
ACCATCACGAGGGTGTAGAAGGTCCTCATGTCGGGTCCCCCGACCTCGACCCTGGGCTGGTTCACCACCATGGA
CGGCTTGAGCTCGCAGCCGTTGGATACGGTCCTGGTGCCGTAGCTGACCCTGAGGTTGGTGGTCCGGACGAAGG
GGTCCAGCACGTCGCCCACCACCCTACCAACCACCAGCGCTTTGAGACCACGAAGTGTTCGAATTAATTAAGGG
GACACCGTTGTCGGAGCAGTGCCAGTGAGCTGGGTGGCAACCTGCGGGACTCTGGTGAGGAAGAGCAAATGGT
AGAAAGCAGTAGCTGTGCTTCTGAACATTGTGTGTGTTTGTTTCCCTTGTTACCGCCAATCATCCTACCTGATGT
GTCGTCTGGGGTTTCGTTTGAACCATGCTGGGTTTGGAGCTTTGTGATGCAAAGCAGAATGTCCTGATGCTTTCT
TTCTCCATAGAATAAATCTCTCCTGTTACCTAAAGGCCGGCCGATATCCATTCAAGAGGAGAGCTCATTTCCTCT
CCTCTCATCACCTTTGCCTAAAAACAGTATGAAAGTCAAGCTAAAGACTTTAAACTAGCTCACTTGGGAGGAAG
TCCCATGTCTATTCTTGTATATACTTTTTTCCCTTGTTATTTTTGATGTTTTGTGTAAGTATTTCAGGTTTTATAGT
CCACGGAAGTCAACGATCGAGTGATATAAATCGAGTCAAAGAATCTTAGCCGTGCGATAGAGTGACTTAAGGC
GAAGCATCCAGCTAATCCGAGTCCCACACAACCTATTCCTTGGAGCTCTAAACGGGAGAGTATGGACGACTTCG
TCAACTCCTATTTCACCAGATCACTAAGGTAACACACCACCCCCATTGTATATACCGTTATCTTTTATTTTTCTTT
GTTTGTTGTTTGTTTTAGTCTTGAATTCTCTCTCAGATTTTTAATACACAAGGGACTGTGTAATTTAAGTGTGGGG
GAGGGTTCAAGAAATATTTGACTTTATGTTCTCTTTTCTTATTTCAAATTTTGCATCATCTAAGGCATAAAAAAC
CATAAAAATTTAAAAACATTTATGAAAATGATTTCACCAAAACAGAGTGTCATGTAGATTGCATTGCATTTTAG
GATTGTATATAGAGTACTTGCATTTAGGATTTCTCATTTGCATAGGGGATAATGATGATTTCGTACCGTTAAGCA
TGTTTTGATTCAATGAAGTAAGTTCAATGCCCAGGTTGTTAGTTCTCTGTTGCATAACCGATTGTTTCTCTGAAG
TAAGACTGCACCCTGCTTTGAATTTACAACTTGACTATACTTCTTTGATTCAAAACTCGCTTATCTGATGCCATTC
CCTATCTATTAGAACCTGAACTGAATTTGCAATTATCATGTCTATGCATTGTTTGTGAACTCATGGCTACCATAT
ACATACTTGTATTATCTTATTCACTTCACCACCCTCTGTTAATCTAAGTAGCTGACTCTCATTTTTAGAGCAGTTT
CCCATACCCTTAGCCTAGCCTTATTTCAAGCCAAAGATCATTTGTGTGTGATTGTGAGGTCTTTTTCGATTGAGC
TTGGTATAACGTGTCAGGTATGAGCCGACAAGAGCAGTCTTTCATGTAGACCTAGCCCACGCTTTTTGGGTTAG
CTAGGACTAGGTGGAGACTTGTATTTGGGATTGGGGTTGTGTATGGATTGAATGGGAAAGAGAATGAGAATGA
AAAGAAAAGAAAAAAAGAGAGTAAGAGTTTAGAGTTCTTAAGGGGGATACAAAAAGTGTTAGTCTAGTAAAG
GGTTTGGAATTCAAAGAAGTTAGGCATTGTGATTTAAAGAAAAGGGTAAAACGACCTAAGATTAGAAGAAAAG
CCTTAAGTTTGTCAAAGTATAAAACCAAACCCACTAGACTCTTGAATCGTTTTAAAGAAGAACCCCTTAGAACT
TGAACCAAAAAGAGAAAGAATGAGAAAAAGGGTAGAGTTAGGACATCTATGGGACTGGAACCATCTTATGAA
CTATCATACTATATCATTGGGTAGATGGGAGTCTGTTCTTGTTTGTGTCCATTGGCTTTTACCTTTAGCATTCTGC
TGACGCTCAATCTTTTCTTTGAGAGTCCCCTGTTACTCGACCAAATCATTAAAGGGACCATTTTTGTCTCTTAGC
CTAAGCCTGAAACCAAGTGAGTTCATAACCATTGCATTGCTTGATCCATTGTTCGTGCTTAGTGAATGTTAAAAA
GAATTGGTTGATGTGAATGCTTGAATAGATTAAGGCAGGTTGTTAGGTTGCTTTGTGACGAGTATGGCTAAATT
TTTAAGTAAGGGTCTATCATTTTGCAACCTATAATTAGCTACCTGGACATTGAGCTTACCCGTTCTATATGCATG
CTACGGGTTTTGAATCCCCACCTTCAAACCTCTCCTTCAACTTAAGATTTTTTGTTTGCTTGAGGGCAAGCAAGA
GATAAGTTTGGGGGAGTTGATAACACTGTAATTTACTCATTTTGGGCATGTATTTATCATCATTTAGCTTAGTGA
TCACTCATTGTTTCATATCATTTTGCATCACATTTAGCTTATTTTGCACACTTAGGCATTTAGGGTCATCTTGCAT
ACATATTGCATGTTATGGTGTGTTTTCAGGTGCAAAATAAGTTTACAAGGAGTTTACTCGACCCTCTACCCTATT
GCCCCAGAGGGGAACCAGAGGAGCAGCTACTCGACCAATTATTCATTGCCTCAGAGGGGAAGCAGCAGAGCAG
CTACTCGACCAGTTATTCGGCGCGCCTAGGGATAACAGGGTAATGTTCCATCCCAATGGCGCGCCGAGCTTGGC
GTAATCATGGTCATAGCTGTTTCCTGTGTGAAATTGTTATCCGCTCACAATTCCACACAACATACGAGCCGGAA
GCATAAAGTGTAAAGCCTGGGGTGCCTAATGAGTGAGCTAACTCACATTAATTGCGTTGCGCTCACTGCCCGCT
TTCCAGTCGGGAAACCTGTCGTGCCAGCTGCATTAATGAATCGGCCAACGCGCGGGGAGAGGCGGTTTGCGTAT
TGGGCGCTGTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTACCAATGCTTAA
TCAGTGAGGCACCTATCTCAGCGATCTGTCTATTTCGTTCATCCATAGTTGCCTGACTCCCCGTCGTGTAGATAA
CTACGATACGGGAGGGCTTACCATCTGGCCCCAGTGCTGCAATGATACCGCGAGAACCACGCTCACCGGCTCCA
GATTTATCAGCAATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTTCCCAACGATCAAGGCG
AGTTACATGATCCCCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAACGTTCTTCGGGG
CGAAAACTCTCAAGGATCTTACCGCTGTTGAGATCCAGTTCGATGTAACCCACTCGTGCACCCAACTGATCTTC
AGCATCTTTTACTTTCACCAGCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAAGCATTTATCAGGGTTATTGT
CTCATGAGCGGATACATATTTGAATGTATTTAGAAAAATAAACAAATAGGGGTTCCGCGCACATTTCCCCGAAA
AGTGCCACCTGACGTCTAAGAAACCATTATTATCATGACATTAACCTATAAAAATAGGCGTATCACGAGGCCCT
TTTGTCTCGCGCGTTTCGGTGATGACGGTGAAAACCTCTGACACATGCAGCTCCCGGAGACTGTCACAGCTTGT
CTGTAAGCGGATGCCGGGAGCAGACAAGCCCGTCAGGGCGCGTCAGCGGGTGTTGGCGGGTGTCGGGGCTGGC
TTAACTATGCGGCATCAGAGCAGATTGTACTGAGAGTGCACCATATGCGGTGTGAAATACCGCACAGATGCGTA
AGGAGAAAATACCGCATCAGGCGCCATTCGCCATTCAGGCTGCGCAACTGTTGGGAAGGGCGATCGGTGCGGG
CCTCTTCGCTATTACGCCAGCTGGCGAAAGGGGGATGTGCTGCAAGGCGATTAAGTTGGGTAACGCCAGGGTTT
TCCCAGTCACGACGTTGTAAAACGACGGCCAGTGAATTGACGCGTATTGGGAT
AF
SEQ ID NO: 11 (ScFT4, ScFT8, ScFT10 RNAi-hairpin-short expression vector, NPT marker,
11152 bp)
GGAACTAGGGATAACAGGGTAATGGCGCGCCGTTGTTTTAGTGTCCGAGGTTTGGTTTGGTGAAGTACATCTGA
ACTCTGAAGGTTCTTCGTGACATGGTTAGACGTGGTGTTGCAGTACTGTGATAAAACTTATCAGAATAAGTTTTC
GCTTCTAATTTGTTTTCTTAAATCATGTGTTGATCAGCCTATGAGTTGTTCATATCGTTGCCTTTCGTGTTGAACA
TTTGTTTTCTTGCCATACTAATGAGATTCATCTGCACAAACTGTACGAAGAGAATCAAGTTTAAACACTGCAGGT
CAACGGATCACCTATCAACATCCCAGCTAAAAACAGTAAAAAGGGGGAAAACGTGGGTGAGTTGAGTCTGTCT
TGTGGAAAAAACGTTTTAGTTTCTCCTGGAATTAACAATAAAAACAGTTGAACAAGATTGACTGTTCCTCCGGG
AGGGTTTGGAACATCGTTACAGATGTGAGCGAAAGGTGAGGAAACAGAGCGGAGGGCTTGGAGGTGACCTCGG
TAGTCGACGCCGGAGTTGAGCTTGATGACGACACCGTACTGGCGTACCAGGCCTAGTAGTGAACACCGGGCCT
GAAGCTGTCGCCGCCGCTGCTCATCTTGTGGGCTGTGCCCGGTGTCCCTGTTGCGGATTGCGGGTGGCAGCCTG
GCAGGTGGGTGCGACCCGTTTGGACTCCCTGATCTGGGCCCTTTGTGTCAGTACCGTCTGTACTCCGATGACATG
CACACCGTCGTCCACAGTCAAGTCCACAATCTCCCCTCTTTTTTTAACGGAATAGTTCAAAATCTCCTTGACGCA
CGCTATCGTGTACCAGCGCTCACTGGACACCACGTTTGTAATCCACGCCGACACGTCGGTCCCACGTCGACAGG
CCCCACCGTCCGGTCTGTAGCGTGTACGTATTCGGGCGACGGACGTGTCGTCGTCGTCTTGCGAGTCCCATTCCC
ATCACCATCTGAGCCACACATCCTCTGAACAAAAGCAGGGAGGCCTCTACGCACATCCCCCTTTCTCCCACTCC
TTGTCCGTGGCACCCACCCCAAACCCTCGCGCCGCCTCCGAGACAGCCGCCGCAACCATGGCCACCGCCTGCAG
GACCGTCTTCGGTACGCGCTCACTCCGCCCTCTGCCTTTGTTACTGCCACGTTTCTCTGAATGCTCTCTTGTGTGG
TGATTGCTGAGAGTGGTTTAGCTGGATCTAGAATTACACTCTGAAATCGTGTTCTGCCTGTGCTGATTACTTGCC
GTCCTTTGTAGCAGCAAAATATAGGGACATGGTAGTACGAAACGAAGATAGAACCTACACAGCAATACGAGAA
ATGTGTAATTTGGTGCTTAGCGGTATTTATTTAAGCACATGTTGGTGTTATAGGGCACTTGGATTCAGAAGTTTG
CTGTTAATTTAGGCACAGGCTTCATACTACATGGGTCAATAGTATAGGGATTCATATTATAGGCGATACTATAA
TAATTTGTTCGTCTGCAGAGCTTATTATTTGCCAAAATTAGATATTCCTATTCTGTTTTTGTTTGTGTGCTGTTAA
ATTGTTAACGCCTGAAGGAATAAATATAAATGACGAAATTTTGATGTTTATCTCTGCTCCTTTATTGTGACCATA
AGTCAAGATCAGATGCACTTGTTTTAAATATTGTTGTCTGAAGAAATAAGTACTGACAGTATTTTGATGCATTGA
TCTGCTTGTTTGTTGTAACAAAATTTAAAAATAAAGAGTTTCCTTTTTGTTGCTCTCCTTACCTCCTGATGGTATC
TAGTATCTACCAACTGACACTATATTGCTTCTCTTTACATACGTATCTTGCTCGATGCCTTCTCCCTAGTGTTGAC
CAGTGTTACTCACATAGTCTTTGCTCATTTCATTGTAATGCAGATACCAAGCGGCCCCTGCAGGATGGGGATTG
AACAAGATGGATTGCACGCAGGTTCTCCGGCCGCTTGGGTGGAGAGGCTATTCGGCTATGACTGGGCACAACA
GACAATCGGCTGCTCTGATGCCGCCGTGTTCCGGCTGTCAGCGCAGGGGCGCCCGGTTCTTTTTGTCAAGACCG
ACCTGTCCGGTGCCCTGAATGAACTCCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGTGCCGGGGCAGG
ATCTCCTGTCATCTCACCTTGCTCCTGCCGAGAAAGTATCCATCATGGCTGATGCAATGCGGCGGCTGCATACGC
TTGATCCGGCTACCTGCCCATTCGACCACCAAGCGAAACATCGCATCGAGCGAGCACGTACTCGGATGGAAGCC
GGTCTTGTCGATCAGGATGATCTGGACGAAGAGCATCAGGGGCTCGCGCCAGCCGAACTGTTCGCCAGGCTCA
AGGCGCGCATGCCCGACGGCGAGGATCTCGTCGTGACACATGGCGATGCCTGCTTGCCGAATATCATGGTGGA
AAATGGCCGCTTTTCTGGATTCATCGACTGTGGCCGGCTGGGTGTGGCGGACCGCTATCAGGACATAGCGTTGG
CTACCCGTGATATTGCTGAAGAGCTTGGCGGCGAATGGGCTGACCGCTTCCTCGTGCTTTACGGTATCGCCGCTC
CCGATTCGCAGCGCATCGCCTTCTATCGCCTTCTTGACGAGTTCTTCTGAGCGATCGCAGAGCCATGGGGGCGG
TGAGAGCTCGCGGCGTCGGATCTCCGGTTTCAGTTTCAGCTACGCGACTTGTGTGTGTGTGTGTGTGTCAGTGCG
TGGTTACTCTGAGCTTCGGTATGTTTGGTGTGTACTGTTCAGTGTTTCATGGTGCTTCCTAGCAGTGTCTGTCGTG
TACTTTGCCTGTATGTTGAGCTCGGCTCAGTATGTTCTGGGAGTGAATAAATAAAAAAAAATTAACCAGATATT
GTCGCGTGTACTTAGTATACTAATCACCTGCTCCTGCTCTTGCTCTGTTTTATCACCTTTCGGGCTTGACTGTCTT
TGTGGAGGCCTCTCAGTCTGATCTGGAACTCTACCTAAGTTTTACTAGTCCCGGGAAGCTTCTGCAGTGCAGCGT
GACCCGGTCGTGCCCCTCACTAGAGATAATGAGCATTGCATGTCTAAGTTATAAAAAATTACCACATATTTTTTT
TGTCACACTTGTTTGAAGTGCAGTTTATCTATCTTTATACATATATTTAAACTTTACTCTACGAATAATATAATCT
ATAGTACTACAATAATATCAGTGTTTTAGAGAATCATATAAATGAACAGTTAGACATGGTCTAAAGGACAATTG
AGTATTTTGACAACAGGACTCTACAGTTTTATCTTTTTAGTGTGCATGTGTTCTCCTTTTTTTTTGCAAATAGCTT
CACCTATATAATACTTCATCCATTTTATTAGTACATCCATTTAGGGTTTAGGGTTAATGGTTTTTATAGACTAATT
TTTTTAGTACATCTATTTTATTCTATTTTAGCCTCTAAATTAAGAAAACTAAAACTCTATTTTAGTTTTTTTATTTA
ATAATTTAGATATAAAATAGAATAAAATAAAGTGACTAAAAATTAAACAAATACCCTTTAAGAAATTAAAAAA
ACTAAGGAAACATTTTTCTTGTTTCGAGTAGATAATGCCAGCCTGTTAAACGCCGTCGACGAGTCTAACGGACA
CCAACCAGCGAACCAGCAGCGTCGCGTCGGGCCAAGCGAAGCAGACGGCACGGCATCTCTGTCGCTGCCTCTG
GACCCCTCTCGAGAGTTCCGCTCCACCGTTGGACTTGCTCCGCTGTCGGCATCCAGAAATTGCGTGGCGGAGCG
GCAGACGTGAGCCGGCACGGCAGGCGGCCTCCTCCTCCTCTCACGGCACGGCAGCTACGGGGGATTCCTTTCCC
ACCGCTCCTTCGCTTTCCCTTCCTCGCCCGCCGTAATAAATAGACACCCCCTCCACACCCTCTTTCCCCAACCTC
GTGTTGTTCGGAGCGCACACACACACAACCAGATCTCCCCCAAATCCACCCGTCGGCACCTCCGCTTCAAGGTA
CGCCGCTCGTCCTCCCCCCCCCCCCCTCTCTACCTTCTCTAGATCGGCGTTCCGGTCCATGGTTAGGGCCCGGTA
GTTCTACTTCTGTTCATGTTTGTGTTAGATCCGTGTTTGTGTTAGATCCGTGCTGCTAGCGTTCGTACACGGATGC
GACCTGTACGTCAGACACGTTCTGATTGCTAACTTGCCAGTGTTTCTCTTTGGGGAATCCTGGGATGGCTCTAGC
CGTTCCGCAGACGGGATCGATTTCATGATTTTTTTTGTTTCGTTGCATAGGGTTTGGTTTGCCCTTTTCCTTTATTT
CAATATATGCCGTGCACTTGTTTGTCGGGTCATCTTTTCATGCTTTTTTTTGTCTTGGTTGTGATGATGTGGTCTG
GTTGGGCGGTCGTTCTAGATCGGAGTAGAATTCTGTTTCAAACTACCTGGTGGATTTATTAATTTTGGATCTGTA
TGTGTGTGCCATACATATTCATAGTTACGAATTGAAGATGATGGATGGAAATATCGATCTAGGATAGGTATACA
TGTTGATGCGGGTTTTACTGATGCATATACAGAGATGCTTTTTGTTCGCTTGGTTGTGATGATGTGGTGTGGTTG
GGCGGTCGTTCATTCGTTCTAGATCGGAGTAGAATACTGTTTCAAACTACCTGGTGTATTTATTAATTTTGGAAC
TGTATGTGTGTGTCATACATCTTCATAGTTACGAGTTTAAGATGGATGGAAATATCGATCTAGGATAGGTATAC
ATGTTGATGTGGGTTTTACTGATGCATATACATGATGGCATATGCAGCATCTATTCATATGCTCTAACCTTGAGT
ACCTATCTATTATAATAAACAAGTATGTTTTATAATTATTTTGATCTTGATATACTTGGATGATGGCATATGCAG
CAGCTATATGTGGATTTTTTTAGCCCTGCCTTCATACGCTATTTATTTGCTTGGTACTGTTTCTTTTGTCGATGCTC
ACCCTGTTGTTTGGTGTTACTTCTCTAGAGCGGCCGCATTTAAATCACTCTGTGGTCTCAAATGGACCGTATCCA
ACGGCTGCGAGCTCAAGCCGTCCATGGTGGTGAACCAGCCCAGGGTCGAGGTCGGGGGACCCGACATGAGGAC
CTTCTACACCCTCGTGATGGGAAGCCATCCGCAGTGGTGAATAAGCCAAGAGTTGATGTTGGGGGCACCGACCT
CAGGGTGTTCTTTACACTGGTAGGTATTAGTTGATCCAGATGCCCCAAGCCAACCATTCCACTAAGAATAACAT
ACAACAATAGGCTACTTCTGGCAAGTGCTGAGCTAAAGCCATCTGCGGTTGCAAGTAAACCACGAGTCGATGTC
GGTGGCGTTTGAATCAGATTCAGATTTCATTGCATCACAGAGATCCATCTTTACTCTACCGCTTGCTCTAACTTA
ACTTGTAATTGTTTTTTATCATGCAGGCCACCGACATCGACTCGTGGTTTACTTGCAACCGCAGATGGCTTTAGC
TCAGCACTTGCCAGAAGTAGCCTATTGTTGTATGTTATTCTTAGTGGAATGGTTGGCTTGGGGCATCTGGATCAA
CTAATACCTACCAGTGTAAAGAACACCCTGAGGTCGGTGCCCCCAACATCAACTCTTGGCTTATTCACCACTGC
GGATGGCTTCCCATCACGAGGGTGTAGAAGGTCCTCATGTCGGGTCCCCCGACCTCGACCCTGGGCTGGTTCAC
CACCATGGACGGCTTGAGCTCGCAGCCGTTGGATACGGTCGCTTTGAGACCACGAAGTGTTCGAATTAATTAAG
GGGACACCGTTGTCGGAGCAGTGCCAGTGAGCTGGGTGGCAACCTGCGGGACTCTGGTGAGGAAGAGCAAATG
GTAGAAAGCAGTAGCTGTGCTTCTGAACATTGTGTGTGTTTGTTTCCCTTGTTACCGCCAATCATCCTACCTGAT
GTGTCGTCTGGGGTTTCGTTTGAACCATGCTGGGTTTGGAGCTTTGTGATGCAAAGCAGAATGTCCTGATGCTTT
CTTTCTCCATAGAATAAATCTCTCCTGTTACCTAAAGGCCGGCCGATATCCATTCAAGAGGAGAGCTCATTTCCT
CTCCTCTCATCACCTTTGCCTAAAAACAGTATGAAAGTCAAGCTAAAGACTTTAAACTAGCTCACTTGGGAGGA
AGTCCCATGTCTATTCTTGTATATACTTTTTTCCCTTGTTATTTTTGATGTTTTGTGTAAGTATTTCAGGTTTTATA
GTCCACGGAAGTCAACGATCGAGTGATATAAATCGAGTCAAAGAATCTTAGCCGTGCGATAGAGTGACTTAAG
GCGAAGCATCCAGCTAATCCGAGTCCCACACAACCTATTCCTTGGAGCTCTAAACGGGAGAGTATGGACGACTT
CGTCAACTCCTATTTCACCAGATCACTAAGGTAACACACCACCCCCATTGTATATACCGTTATCTTTTATTTTTCT
TTGTTTGTTGTTTGTTTTAGTCTTGAATTCTCTCTCAGATTTTTAATACACAAGGGACTGTGTAATTTAAGTGTGG
GGGAGGGTTCAAGAAATATTTGACTTTATGTTCTCTTTTCTTATTTCAAATTTTGCATCATCTAAGGCATAAAAA
ACCATAAAAATTTAAAAACATTTATGAAAATGATTTCACCAAAACAGAGTGTCATGTAGATTGCATTGCATTTT
AGGATTGTATATAGAGTACTTGCATTTAGGATTTCTCATTTGCATAGGGGATAATGATGATTTCGTACCGTTAAG
CATGTTTTGATTCAATGAAGTAAGTTCAATGCCCAGGTTGTTAGTTCTCTGTTGCATAACCGATTGTTTCTCTGA
AGTAAGACTGCACCCTGCTTTGAATTTACAACTTGACTATACTTCTTTGATTCAAAACTCGCTTATCTGATGCCA
TTCCCTATCTATTAGAACCTGAACTGAATTTGCAATTATCATGTCTATGCATTGTTTGTGAACTCATGGCTACCA
TATACATACTTGTATTATCTTATTCACTTCACCACCCTCTGTTAATCTAAGTAGCTGACTCTCATTTTTAGAGCAG
TTTCCCATACCCTTAGCCTAGCCTTATTTCAAGCCAAAGATCATTTGTGTGTGATTGTGAGGTCTTTTTCGATTGA
GCTTGGTATAACGTGTCAGGTATGAGCCGACAAGAGCAGTCTTTCATGTAGACCTAGCCCACGCTTTTTGGGTT
AGCTAGGACTAGGTGGAGACTTGTATTTGGGATTGGGGTTGTGTATGGATTGAATGGGAAAGAGAATGAGAAT
GAAAAGAAAAGAAAAAAAGAGAGTAAGAGTTTAGAGTTCTTAAGGGGGATACAAAAAGTGTTAGTCTAGTAA
AGGGTTTGGAATTCAAAGAAGTTAGGCATTGTGATTTAAAGAAAAGGGTAAAACGACCTAAGATTAGAAGAAA
AGCCTTAAGTTTGTCAAAGTATAAAACCAAACCCACTAGACTCTTGAATCGTTTTAAAGAAGAACCCCTTAGAA
CTTGAACCAAAAAGAGAAAGAATGAGAAAAAGGGTAGAGTTAGGACATCTATGGGACTGGAACCATCTTATGA
ACTATCATACTATATCATTGGGTAGATGGGAGTCTGTTCTTGTTTGTGTCCATTGGCTTTTACCTTTAGCATTCTG
CTGACGCTCAATCTTTTCTTTGAGAGTCCCCTGTTACTCGACCAAATCATTAAAGGGACCATTTTTGTCTCTTAG
CCTAAGCCTGAAACCAAGTGAGTTCATAACCATTGCATTGCTTGATCCATTGTTCGTGCTTAGTGAATGTTAAAA
AGAATTGGTTGATGTGAATGCTTGAATAGATTAAGGCAGGTTGTTAGGTTGCTTTGTGACGAGTATGGCTAAAT
TTTTAAGTAAGGGTCTATCATTTTGCAACCTATAATTAGCTACCTGGACATTGAGCTTACCCGTTCTATATGCAT
GCTACGGGTTTTGAATCCCCACCTTCAAACCTCTCCTTCAACTTAAGATTTTTTGTTTGCTTGAGGGCAAGCAAG
AGATAAGTTTGGGGGAGTTGATAACACTGTAATTTACTCATTTTGGGCATGTATTTATCATCATTTAGCTTAGTG
ATCACTCATTGTTTCATATCATTTTGCATCACATTTAGCTTATTTTGCACACTTAGGCATTTAGGGTCATCTTGCA
TACATATTGCATGTTATGGTGTGTTTTCAGGTGCAAAATAAGTTTACAAGGAGTTTACTCGACCCTCTACCCTAT
TGCCCCAGAGGGGAACCAGAGGAGCAGCTACTCGACCAATTATTCATTGCCTCAGAGGGGAAGCAGCAGAGCA
GCTACTCGACCAGTTATTCGGCGCGCCTAGGGATAACAGGGTAATGTTCCATCCCAATGGCGCGCCGAGCTTGG
CGTAATCATGGTCATAGCTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGCGTTGCGCTCACTGCCCGC
TTTCCAGTCGGGAAACCTGTCGTGCCAGCTGCATTAATGAATCGGCCAACGCGCGGGGAGAGGCGGTTTGCGTA
TTGGGCGCTGTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCTCCCTTCGGGAA
GCGTGGCGCTTTCTCATAGCTCACGCTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTATGTAGGCGGTGCTACAG
AGTTCTTGAAGTGGTGGCCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAATGCTT
AATCAGTGAGGCACCTATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGAACCACGCTCACCGGCTC
CAGATTTATCAGCAATAAACCAGCCAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATTGGAAAACGTTCTTCGG
GGCGAAAACTCTCAAGGATCTTACCGCTGTTGAGATCCAGTTCGATGTAACCCACTCGTGCACCCAACTGATCT
TCAGCATCTTTTACTTTCACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAAGCATTTATCAGGGTTATT
GTCTCATGAGCGGATACATATTTGAATGTATTTAGAAAAATAAACAAATAGGGGTTCCGCGCACATTTCCCCGA
AAAGTGCCACCTGACGTCTAAGAAACCATTATTATCATGACATTAACCTATAAAAATAGGCGTATCACGAGGCC
CTTTTGTCTCGCGCGTTTCGGTGATGACGGTGAAAACCTCTGACACATGCAGCTCCCGGAGACTGTCACAGCTT
GTCTGTAAGCGGATGCCGGGAGCAGACAAGCCCGTCAGGGCGCGTCAGCGGGTGTTGGCGGGTGTCGGGGCTG
GCTTAACTATGCGGCATCAGAGCAGATTGTACTGAGAGTGCACCATATGCGGTGTGAAATACCGCACAGATGC
GTAAGGAGAAAATACCGCATCAGGCGCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAACGCCAGG
GTTTTCCCAGTCACGACGTTGTAAAACGACGGCCAGTGAATTGACGCGTATTGGGAT
SEQ ID NO: 12 (ScFT4, ScFT8, ScFT10 RNAi-hairpin-long expression vector, ALS marker,
12280 bp)
GGAACTAGGGATAACAGGGTAATGGCGCGCCGTTGTTTTAGTGTCCGAGGTTTGGTTTGGTGAAGTACATCTGA
ACTCTGAAGGTTCTTCGTGACATGGTTAGACGTGGTGTTGCAGTACTGTGATAAAACTTATCAGAATAAGTTTTC
GCTTCTAATTTGTTTTCTTAAATCATGTGTTGATCAGCCTATGAGTTGTTCATATCGTTGCCTTTCGTGTTGAACA
TTTGTTTTCTTGCCATACTAATGAGATTCATCTGCACAAACTGTACGAAGAGAATCAAGTTTAAACACTGCAGGT
CAACGGATCACCTATCAACATCCCAGCTAAAAACAGTAAAAAGGGGGAAAACGTGGGTGAGTTGAGTCTGTCT
TGTGGAAAAAACGTTTTAGTTTCTCCTGGAATTAACAATAAAAACAGTTGAACAAGATTGACTGTTCCTCCGGG
AGGGTTTGGAACATCGTTACAGATGTGAGCGAAAGGTGAGGAAACAGAGCGGAGGGCTTGGAGGTGACCTCGG
TAGTCGACGCCGGAGTTGAGCTTGATGACGACACCGTACTGGCGTACCAGGCCTAGTAGTGAACACCGGGCCT
GAAGCTGTCGCCGCCGCTGCTCATCTTGTGGGCTGTGCCCGGTGTCCCTGTTGCGGATTGCGGGTGGCAGCCTG
GCAGGTGGGTGCGACCCGTTTGGACTCCCTGATCTGGGCCCTTTGTGTCAGTACCGTCTGTACTCCGATGACATG
CACACCGTCGTCCACAGTCAAGTCCACAATCTCCCCTCTTTTTTTAACGGAATAGTTCAAAATCTCCTTGACGCA
CGCTATCGTGTACCAGCGCTCACTGGACACCACGTTTGTAATCCACGCCGACACGTCGGTCCCACGTCGACAGG
CCCCACCGTCCGGTCTGTAGCGTGTACGTATTCGGGCGACGGACGTGTCGTCGTCGTCTTGCGAGTCCCATTCCC
ATCACCATCTGAGCCACACATCCTCTGAACAAAAGCAGGGAGGCCTCTACGCACATCCCCCTTTCTCCCACTCC
TTGTCCGTGGCACCCACCCCAAACCCTCGCGCCGCCTCCGAGACAGCCGCCGCAACCATGGCCACCGCCTGCAG
GACCGTCTTCGGTACGCGCTCACTCCGCCCTCTGCCTTTGTTACTGCCACGTTTCTCTGAATGCTCTCTTGTGTGG
TGATTGCTGAGAGTGGTTTAGCTGGATCTAGAATTACACTCTGAAATCGTGTTCTGCCTGTGCTGATTACTTGCC
GTCCTTTGTAGCAGCAAAATATAGGGACATGGTAGTACGAAACGAAGATAGAACCTACACAGCAATACGAGAA
ATGTGTAATTTGGTGCTTAGCGGTATTTATTTAAGCACATGTTGGTGTTATAGGGCACTTGGATTCAGAAGTTTG
CTGTTAATTTAGGCACAGGCTTCATACTACATGGGTCAATAGTATAGGGATTCATATTATAGGCGATACTATAA
TAATTTGTTCGTCTGCAGAGCTTATTATTTGCCAAAATTAGATATTCCTATTCTGTTTTTGTTTGTGTGCTGTTAA
ATTGTTAACGCCTGAAGGAATAAATATAAATGACGAAATTTTGATGTTTATCTCTGCTCCTTTATTGTGACCATA
AGTCAAGATCAGATGCACTTGTTTTAAATATTGTTGTCTGAAGAAATAAGTACTGACAGTATTTTGATGCATTGA
TCTGCTTGTTTGTTGTAACAAAATTTAAAAATAAAGAGTTTCCTTTTTGTTGCTCTCCTTACCTCCTGATGGTATC
TAGTATCTACCAACTGACACTATATTGCTTCTCTTTACATACGTATCTTGCTCGATGCCTTCTCCCTAGTGTTGAC
CAGTGTTACTCACATAGTCTTTGCTCATTTCATTGTAATGCAGATACCAAGCGGCCCCTGCAGGATGGCCACCAC
CGCCGCCGCCGCTGCCGCCGCGCTAGCCGGCGCCACTACCGCTGCGCCCAAGGCGAGGCGCCGGGCGCACCTC
CTGGCCGCACGGCGCGCCCTCGCCGCGCCCATCAGGTGCTCAGCGGCGCCACCCGCCACGCTGACGGTGACGG
CTCCCCCGGCCACCCCGCTCCGGCCGTGGGGCCCCACCGATCCCCGCAAGGGCGCCGACATCCTCGTCGAGGCT
CTTGAGCGCTGCGGCGTCCGCGACGTCTTCGCCTACCCCGGCGGCGCGTCCATGGAGATCCACCAGGCACTCAC
CCGTTCCCCCGTCATCGCCAACCACCTCTTCCGCCACGAGCAAGGGGAGGCCTTCGCCGCCTCTGGCTTCGCGC
GCTCCTCGGGCCGCGTCGGCGTCTGCGTCGCCACCTCCGGCCCCGGCGCCACCAACCTAGTCTCCGCGCTCGCC
GACGCGCTGCTCGACTCCGTCCCCATGGTCGCCATCACGGGACAGGTTCCGCGGCGCATGATTGGCACCGACGC
CTTCCAGGAGACGCCCATCGTCGAGGTCACCCGCTCCATCACCAAACATAACTACCTGGTCCTCGACGTCGACG
ACATCCCCCGCGTCGTGCAGGAGGCTTTCTTCCTCGCCTCCTCCGGTCGCCCGGGACCGGTGCTTGTCGACATCC
CCAAGGACATCCAGCAGCAGATGGCCGTGCCGGTCTGGGACACGCCCATGAGTCTGCCTGGGTACATTGCGCG
CCTTCCCAAGCCTCCTGCGACTGAATTGCTTGAGCAGGTGCTGCGTCTTGTTGGTGAATCAAGGCGCCCTGTTCT
TTATGTTGGTGGTGGCTGCGCAGCATCTGGCGAGGAGTTGCGCCGCTTTGTGGAGATGACTGGAATCCCAGTCA
CAACTACTCTTATGGGCCTTGGCAATTTCCCTGGCGACGACCCACTGTCTCTGCGCATGCTTGGTATGCATGGCA
CGGTGTATGCAAATTATGCAGTGGATAAGGCGGATCTGTTGCTTGCATTTGGTGTGCGGTTTGATGATCGTGTG
ACAGGGAAGATTGAGGCTTTTGCAAGCAGGGCTAAGATTGTGCACATTGATATTGATCCCGCTGAGATTGGCAA
GAACAAGCAGCCACATGTGTCCATCTGTGCAGACGTTAAGCTTGCTTTGCAGGGCATGAATGCTCTTCTGGAAG
GAAGCACATCAAAGAAGAGCTTTGACTTTGGCTCATGGCAAGCTGAGTTGGATCAGCAGAAGAGAGAGTTCCC
CCTTGGGTATAAAACTTTTGATGACGAGATCCAGCCACAATATGCTATTCAGGTTCTTGATGAGCTGACAAAAG
GGGAGGCCATCATTGCCACAGGTGTTGGGCAGCACCAGATGTGGGCGGCACAGTACTACACTTACAAGCGGCC
AAGGCAGTGGTTGTCTTCAGCTGGTCTTGGGGCTATGGGATTTGGTTTGCCGGCTGCTGCTGGCGCTGCTGTGGC
CAACCCAGGTATCACTGTTGTTGACATCGACGGAGATGGTAGCTTCCTCATGAACATTCAGGAGCTAGCTATGA
TCCGAATTGAGAACCTCCCAGTGAAGGTCTTTGTGCTAAACAACCAGCACCTGGGGATGGTGGTGCAGTTGGAG
GACAGGTTCTATAAGGCCAATAGAGCACACACATACTTGGGAAACCCAGAGAATGAAAGTGAGATATATCCAG
ATTTCGTGACAATTGCCAAAGGGTTCAACATTCCAGCAGTCCGTGTGACAAAGAAGAGCGAAGTCCATGCAGC
AATCAAGAAGATGCTTGAGACTCCAGGGCCATACCTCTTGGATATAATCGTCCCGCACCAGGAGCATGTGTTGC
CTATGATCCCTAGTGGTGGGGCTTTCAAGGATATGATCCTGGATGGTGATGGCAGGACTGTGTATTGAGCGATC
GCAGAGCCATGGGGGCGGTGAGAGCTCGCGGCGTCGGATCTCCGGTTTCAGTTTCAGCTACGCGACTTGTGTGT
GTGTGTGTGTGTCAGTGCGTGGTTACTCTGAGCTTCGGTATGTTTGGTGTGTACTGTTCAGTGTTTCATGGTGCTT
CCTAGCAGTGTCTGTCGTGTACTTTGCCTGTATGTTGAGCTCGGCTCAGTATGTTCTGGGAGTGAATAAATAAAA
AAAAATTAACCAGATATTGTCGCGTGTACTTAGTATACTAATCACCTGCTCCTGCTCTTGCTCTGTTTTATCACCT
TTCGGGCTTGACTGTCTTTGTGGAGGCCTCTCAGTCTGATCTGGAACTCTACCTAAGTTTTACTAGTCCCGGGAA
GCTTCTGCAGTGCAGCGTGACCCGGTCGTGCCCCTCACTAGAGATAATGAGCATTGCATGTCTAAGTTATAAAA
AATTACCACATATTTTTTTTGTCACACTTGTTTGAAGTGCAGTTTATCTATCTTTATACATATATTTAAACTTTAC
TCTACGAATAATATAATCTATAGTACTACAATAATATCAGTGTTTTAGAGAATCATATAAATGAACAGTTAGAC
ATGGTCTAAAGGACAATTGAGTATTTTGACAACAGGACTCTACAGTTTTATCTTTTTAGTGTGCATGTGTTCTCC
TTTTTTTTTGCAAATAGCTTCACCTATATAATACTTCATCCATTTTATTAGTACATCCATTTAGGGTTTAGGGTTA
ATGGTTTTTATAGACTAATTTTTTTAGTACATCTATTTTATTCTATTTTAGCCTCTAAATTAAGAAAACTAAAACT
CTATTTTAGTTTTTTTATTTAATAATTTAGATATAAAATAGAATAAAATAAAGTGACTAAAAATTAAACAAATAC
CCTTTAAGAAATTAAAAAAACTAAGGAAACATTTTTCTTGTTTCGAGTAGATAATGCCAGCCTGTTAAACGCCG
TCGACGAGTCTAACGGACACCAACCAGCGAACCAGCAGCGTCGCGTCGGGCCAAGCGAAGCAGACGGCACGG
CATCTCTGTCGCTGCCTCTGGACCCCTCTCGAGAGTTCCGCTCCACCGTTGGACTTGCTCCGCTGTCGGCATCCA
GAAATTGCGTGGCGGAGCGGCAGACGTGAGCCGGCACGGCAGGCGGCCTCCTCCTCCTCTCACGGCACGGCAG
CTACGGGGGATTCCTTTCCCACCGCTCCTTCGCTTTCCCTTCCTCGCCCGCCGTAATAAATAGACACCCCCTCCA
CACCCTCTTTCCCCAACCTCGTGTTGTTCGGAGCGCACACACACACAACCAGATCTCCCCCAAATCCACCCGTCG
GCACCTCCGCTTCAAGGTACGCCGCTCGTCCTCCCCCCCCCCCCCTCTCTACCTTCTCTAGATCGGCGTTCCGGT
CCATGGTTAGGGCCCGGTAGTTCTACTTCTGTTCATGTTTGTGTTAGATCCGTGTTTGTGTTAGATCCGTGCTGCT
AGCGTTCGTACACGGATGCGACCTGTACGTCAGACACGTTCTGATTGCTAACTTGCCAGTGTTTCTCTTTGGGGA
ATCCTGGGATGGCTCTAGCCGTTCCGCAGACGGGATCGATTTCATGATTTTTTTTGTTTCGTTGCATAGGGTTTG
GTTTGCCCTTTTCCTTTATTTCAATATATGCCGTGCACTTGTTTGTCGGGTCATCTTTTCATGCTTTTTTTTGTCTT
GGTTGTGATGATGTGGTCTGGTTGGGCGGTCGTTCTAGATCGGAGTAGAATTCTGTTTCAAACTACCTGGTGGAT
TTATTAATTTTGGATCTGTATGTGTGTGCCATACATATTCATAGTTACGAATTGAAGATGATGGATGGAAATATC
GATCTAGGATAGGTATACATGTTGATGCGGGTTTTACTGATGCATATACAGAGATGCTTTTTGTTCGCTTGGTTG
TGATGATGTGGTGTGGTTGGGCGGTCGTTCATTCGTTCTAGATCGGAGTAGAATACTGTTTCAAACTACCTGGTG
TATTTATTAATTTTGGAACTGTATGTGTGTGTCATACATCTTCATAGTTACGAGTTTAAGATGGATGGAAATATC
GATCTAGGATAGGTATACATGTTGATGTGGGTTTTACTGATGCATATACATGATGGCATATGCAGCATCTATTCA
TATGCTCTAACCTTGAGTACCTATCTATTATAATAAACAAGTATGTTTTATAATTATTTTGATCTTGATATACTTG
GATGATGGCATATGCAGCAGCTATATGTGGATTTTTTTAGCCCTGCCTTCATACGCTATTTATTTGCTTGGTACT
GTTTCTTTTGTCGATGCTCACCCTGTTGTTTGGTGTTACTTCTCTAGAGCGGCCGCATTTAAATCACTCTGTGGTC
TCAAATGGACCGTATCCAACGGCTGCGAGCTCAAGCCGTCCATGGTGGTGAACCAGCCCAGGGTCGAGGTCGG
GGGACCCGACATGAGGACCTTCTACACCCTCGTGATGGGAAGCCATCCGCAGTGGTGAATAAGCCAAGAGTTG
ATGTTGGGGGCACCGACCTCAGGGTGTTCTTTACACTGGTAGGTATTAGTTGATCCAGATGCCCCAAGCCAACC
ATTCCACTAAGAATAACATACAACAATAGGCTACTTCTGGCAAGTGCTGAGCTAAAGCCATCTGCGGTTGCAAG
TAAACCACGAGTCGATGTCGGTGGCGTTTGAATCAGATTCAGATTTCATTGCATCACAGAGATCCATCTTTACTC
TACCGCTTGCTCTAACTTAACTTGTAATTGTTTTTTATCATGCAGGCCACCGACATCGACTCGTGGTTTACTTGCA
ACCGCAGATGGCTTTAGCTCAGCACTTGCCAGAAGTAGCCTATTGTTGTATGTTATTCTTAGTGGAATGGTTGGC
TTGGGGCATCTGGATCAACTAATACCTACCAGTGTAAAGAACACCCTGAGGTCGGTGCCCCCAACATCAACTCT
TGGCTTATTCACCACTGCGGATGGCTTCCCATCACGAGGGTGTAGAAGGTCCTCATGTCGGGTCCCCCGACCTC
GACCCTGGGCTGGTTCACCACCATGGACGGCTTGAGCTCGCAGCCGTTGGATACGGTCGCTTTGAGACCACGAA
GTGTTCGAATTAATTAAGGGGACACCGTTGTCGGAGCAGTGCCAGTGAGCTGGGTGGCAACCTGCGGGACTCTG
GTGAGGAAGAGCAAATGGTAGAAAGCAGTAGCTGTGCTTCTGAACATTGTGTGTGTTTGTTTCCCTTGTTACCG
CCAATCATCCTACCTGATGTGTCGTCTGGGGTTTCGTTTGAACCATGCTGGGTTTGGAGCTTTGTGATGCAAAGC
AGAATGTCCTGATGCTTTCTTTCTCCATAGAATAAATCTCTCCTGTTACCTAAAGGCCGGCCGATATCCATTCAA
GAGGAGAGCTCATTTCCTCTCCTCTCATCACCTTTGCCTAAAAACAGTATGAAAGTCAAGCTAAAGACTTTAAA
CTAGCTCACTTGGGAGGAAGTCCCATGTCTATTCTTGTATATACTTTTTTCCCTTGTTATTTTTGATGTTTTGTGT
AAGTATTTCAGGTTTTATAGTCCACGGAAGTCAACGATCGAGTGATATAAATCGAGTCAAAGAATCTTAGCCGT
GCGATAGAGTGACTTAAGGCGAAGCATCCAGCTAATCCGAGTCCCACACAACCTATTCCTTGGAGCTCTAAACG
GGAGAGTATGGACGACTTCGTCAACTCCTATTTCACCAGATCACTAAGGTAACACACCACCCCCATTGTATATA
CCGTTATCTTTTATTTTTCTTTGTTTGTTGTTTGTTTTAGTCTTGAATTCTCTCTCAGATTTTTAATACACAAGGGA
CTGTGTAATTTAAGTGTGGGGGAGGGTTCAAGAAATATTTGACTTTATGTTCTCTTTTCTTATTTCAAATTTTGCA
TCATCTAAGGCATAAAAAACCATAAAAATTTAAAAACATTTATGAAAATGATTTCACCAAAACAGAGTGTCATG
TAGATTGCATTGCATTTTAGGATTGTATATAGAGTACTTGCATTTAGGATTTCTCATTTGCATAGGGGATAATGA
TGATTTCGTACCGTTAAGCATGTTTTGATTCAATGAAGTAAGTTCAATGCCCAGGTTGTTAGTTCTCTGTTGCAT
AACCGATTGTTTCTCTGAAGTAAGACTGCACCCTGCTTTGAATTTACAACTTGACTATACTTCTTTGATTCAAAA
CTCGCTTATCTGATGCCATTCCCTATCTATTAGAACCTGAACTGAATTTGCAATTATCATGTCTATGCATTGTTTG
TGAACTCATGGCTACCATATACATACTTGTATTATCTTATTCACTTCACCACCCTCTGTTAATCTAAGTAGCTGA
CTCTCATTTTTAGAGCAGTTTCCCATACCCTTAGCCTAGCCTTATTTCAAGCCAAAGATCATTTGTGTGTGATTGT
GAGGTCTTTTTCGATTGAGCTTGGTATAACGTGTCAGGTATGAGCCGACAAGAGCAGTCTTTCATGTAGACCTA
GCCCACGCTTTTTGGGTTAGCTAGGACTAGGTGGAGACTTGTATTTGGGATTGGGGTTGTGTATGGATTGAATG
GGAAAGAGAATGAGAATGAAAAGAAAAGAAAAAAAGAGAGTAAGAGTTTAGAGTTCTTAAGGGGGATACAAA
AAGTGTTAGTCTAGTAAAGGGTTTGGAATTCAAAGAAGTTAGGCATTGTGATTTAAAGAAAAGGGTAAAACGA
CCTAAGATTAGAAGAAAAGCCTTAAGTTTGTCAAAGTATAAAACCAAACCCACTAGACTCTTGAATCGTTTTAA
AGAAGAACCCCTTAGAACTTGAACCAAAAAGAGAAAGAATGAGAAAAAGGGTAGAGTTAGGACATCTATGGG
ACTGGAACCATCTTATGAACTATCATACTATATCATTGGGTAGATGGGAGTCTGTTCTTGTTTGTGTCCATTGGC
TTTTACCTTTAGCATTCTGCTGACGCTCAATCTTTTCTTTGAGAGTCCCCTGTTACTCGACCAAATCATTAAAGGG
ACCATTTTTGTCTCTTAGCCTAAGCCTGAAACCAAGTGAGTTCATAACCATTGCATTGCTTGATCCATTGTTCGT
GCTTAGTGAATGTTAAAAAGAATTGGTTGATGTGAATGCTTGAATAGATTAAGGCAGGTTGTTAGGTTGCTTTG
TGACGAGTATGGCTAAATTTTTAAGTAAGGGTCTATCATTTTGCAACCTATAATTAGCTACCTGGACATTGAGCT
TACCCGTTCTATATGCATGCTACGGGTTTTGAATCCCCACCTTCAAACCTCTCCTTCAACTTAAGATTTTTTGTTT
GCTTGAGGGCAAGCAAGAGATAAGTTTGGGGGAGTTGATAACACTGTAATTTACTCATTTTGGGCATGTATTTA
TCATCATTTAGCTTAGTGATCACTCATTGTTTCATATCATTTTGCATCACATTTAGCTTATTTTGCACACTTAGGC
ATTTAGGGTCATCTTGCATACATATTGCATGTTATGGTGTGTTTTCAGGTGCAAAATAAGTTTACAAGGAGTTTA
CTCGACCCTCTACCCTATTGCCCCAGAGGGGAACCAGAGGAGCAGCTACTCGACCAATTATTCATTGCCTCAGA
GGGGAAGCAGCAGAGCAGCTACTCGACCAGTTATTCGGCGCGCCTAGGGATAACAGGGTAATGTTCCATCCCA
ATGGCGCGCCGAGCTTGGCGTAATCATGGTCATAGCTGTTTCCTGTGTGAAATTGTTATCCGCTCACAATTCCAC
ACAACATACGAGCCGGAAGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATGAATCGGCCAACGCGCGG
GGAGAGGCGGTTTGCGTATTGGGCGCTGTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAGGTATCTCAGTTCGGTGTAGGTCGTTC
GCTCCAAGCTGGGCTGTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCATGAGATTATCAAAAAGGA
TCTTCACCTAGATCCTTTTAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTCATCCATAGTTGCCTGAC
TCCCCGTCGTGTAGATAACTACGATACGGGAGGGCTTACCATCTGGCCCCAGTGCTGCAATGATACCGCGAGAA
CCACGCTCACCGGCTCCAGATTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTTACTGTC
ATGCCATCCGTAAGATGCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAAATAAACAAATAGGGGTT
CCGCGCACATTTCCCCGAAAAGTGCCACCTGACGTCTAAGAAACCATTATTATCATGACATTAACCTATAAAAA
TAGGCGTATCACGAGGCCCTTTTGTCTCGCGCGTTTCGGTGATGACGGTGAAAACCTCTGACACATGCAGCTCC
CGGAGACTGTCACAGCTTGTCTGTAAGCGGATGCCGGGAGCAGACAAGCCCGTCAGGGCGCGTCAGCGGGTGT
TGGCGGGTGTCGGGGCTGGCTTAACTATGCGGCATCAGAGCAGATTGTACTGAGAGTGCACCATATGCGGTGTG
AAATACCGCACAGATGCGTAAGGAGAAAATACCGCATCAGGCGCCATTCGCCATTCAGGCTGCGCAACTGTTG
GGAAGGGCGATCGGTGCGGGCCTCTTCGCTATTACGCCAGCTGGCGAAAGGGGGATGTGCTGCAAGGCGATTA
AGTTGGGTAACGCCAGGGTTTTCCCAGTCACGACGTTGTAAAACGACGGCCAGTGAATTGACGCGTATTGGGAT
SEQ ID NO: 13 (ScFT4 sgRNA expression vector, 13810 bp)
TCGCGCGTTTCGGTGATGACGGTGAAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGGCGGGTGTCGGGGCTGGCTTAACT
ATGCGGCATCAGAGCAGATTGTACTGAGAGTGCACCATATGCGGTGTGAAATACCGCACAGATGCGTAAGGAG
AAAATACCGCATCAGGCGCCATTCGCCATTCAGGCTGCGCAACTGTTGGGAAGGGCGATCGGTGCGGGCCTCTT
CGCTATTACGCCAGCTGGCGAAAGGGGGATGTGCTGCAAGGCGATTAAGTTGGGTAACGCCAGGGTTTTCCCA
GTCACGACGTTGTAAAACGACGGCCAGTGAATTGACGCGTATTGGGATTAACTATAACGGTCCTAAGGTAGCG
AGGCGCGCCGTTGTTTTAGTGTCCGAGGTTTGGTTTGGTGAAGTACATCTGAACTCTGAAGGTTCTTCGTGACAT
GGTTAGACGTGGTGTTGCAGTACTGTGATAAAACTTATCAGAATAAGTTTTCGCTTCTAATTTGTTTTCTTAAAT
CATGTGTTGATCAGCCTATGAGTTGTTCATATCGTTGCCTTTCGTGTTGAACATTTGTTTTCTTGCCATACTAATG
AGATTCATCTGCACAAACTGTACGAAGAGAATCAAGATATCGCCCGGGCTTTGTGAAAGTTGAATTACGGCATA
GCCGAAGGAATAACAGAATCGTTTCACACTTTCGTAACAAAGATCTTCTTATCATGTTTCAGACGATGGAGGCA
AGGCTGATCAAAGTGATCAAGCACATAAACGCATTTTTTTACCATGTTTCACTCCATAAGCGTCTGAGATTATCA
CAAGTCACGTCTAGTAGTTTGATGGTACACTAGTGACAATCAGTTCGTGCAGACAGAGCTCATACTTGACTACT
TGAGCGATTACAGGCGAAAGTGTGAAACGCATGTGATGTGGGCTGGGAGGAGGAGAATATATACTAATGGGCC
GTATCCTGATTTGGGCTGCGTCGGAAGGTGCAGCCCACGCGCGCCGTACCGCGCGGGTGGCGCTGCTACCCACT
TTAGTCCGTTGGATGGGGATCCGATGGTTTGCGCGGTGGCGTTGCGGGGGATGTTTAGTACCACATCGGAAACC
GAAAGACGATGGAACCAGCTTATAAACCCGCGCGCTGTAGTCAGCTTGCCGTAGCTGACCCTGAGGTGTTTTAG
AGCTAGAAATAGCAAGTTAAAATAAGGCTAGTCCGTTATCAACTTGAAAAAGTGGCACCGAGTCGGTGCTTTTT
TTTTAAGCTTTTTGTGAAAGTTGAATTACGGCATAGCCGAAGGAATAACAGAATCGTTTCACACTTTCGTAACA
AAGATCTTCTTATCATGTTTCAGACGATGGAGGCAAGGCTGATCAAAGTGATCAAGCACATAAACGCATTTTTT
TACCATGTTTCACTCCATAAGCGTCTGAGATTATCACAAGTCACGTCTAGTAGTTTGATGGTACACTAGTGACAA
TCAGTTCGTGCAGACAGAGCTCATACTTGACTACTTGAGCGATTACAGGCGAAAGTGTGAAACGCATGTGATGT
GGGCTGGGAGGAGGAGAATATATACTAATGGGCCGTATCCTGATTTGGGCTGCGTCGGAAGGTGCAGCCCACG
CGCGCCGTACCGCGCGGGTGGCGCTGCTACCCACTTTAGTCCGTTGGATGGGGATCCGATGGTTTGCGCGGTGG
CGTTGCGGGGGATGTTTAGTACCACATCGGAAACCGAAAGACGATGGAACCAGCTTATAAACCCGCGCGCTGT
AGTCAGCTTGGCTTGGGCTCGGAGCATCCGTTTTAGAGCTAGAAATAGCAAGTTAAAATAAGGCTAGTCCGTTA
TCAACTTGAAAAAGTGGCACCGAGTCGGTGCTTTTTTTTTGCGGCCGCCTGCAGGTCAACGGATCACCTATCAA
CATCCCAGCTAAAAACAGTAAAAAGGGGGAAAACGTGGGTGAGTTGAGTCTGTCTTGTGGAAAAAACGTTTTA
GTTTCTCCTGGAATTAACAATAAAAACAGTTGAACAAGATTGACTGTTCCTCCGGGAGGGTTTGGAACATCGTT
ACAGATGTGAGCGAAAGGTGAGGAAACAGAGCGGAGGGCTTGGAGGTGACCTCGGTAGTCGACGCCGGAGTT
GAGCTTGATGACGACACCGTACTGGCGTACCAGGCCTAGTAGTGAACACCGGGCCTGAAGCTGTCGCCGCCGCT
GCTCATCTTGTGGGCTGTGCCCGGTGTCCCTGTTGCGGATTGCGGGTGGCAGCCTGGCAGGTGGGTGCGACCCG
TTTGGACTCCCTGATCTGGGCCCTTTGTGTCAGTACCGTCTGTACTCCGATGACATGCACACCGTCGTCCACAGT
CAAGTCCACAATCTCCCCTCTTTTTTTAACGGAATAGTTCAAAATCTCCTTGACGCACGCTATCGTGTACCAGCG
CTCACTGGACACCACGTTTGTAATCCACGCCGACACGTCGGTCCCACGTCGACAGGCCCCACCGTCCGGTCTGT
AGCGTGTACGTATTCGGGCGACGGACGTGTCGTCGTCGTCTTGCGAGTCCCATTCCCATCACCATCTGAGCCAC
ACATCCTCTGAACAAAAGCAGGGAGGCCTCTACGCACATCCCCCTTTCTCCCACTCCTTGTCCGTGGCACCCACC
CCAAACCCTCGCGCCGCCTCCGAGACAGCCGCCGCAACCATGGCCACCGCCTGCAGGACCGTCTTCGGTACGCG
CTCACTCCGCCCTCTGCCTTTGTTACTGCCACGTTTCTCTGAATGCTCTCTTGTGTGGTGATTGCTGAGAGTGGTT
TAGCTGGATCTAGAATTACACTCTGAAATCGTGTTCTGCCTGTGCTGATTACTTGCCGTCCTTTGTAGCAGCAAA
ATATAGGGACATGGTAGTACGAAACGAAGATAGAACCTACACAGCAATACGAGAAATGTGTAATTTGGTGCTT
AGCGGTATTTATTTAAGCACATGTTGGTGTTATAGGGCACTTGGATTCAGAAGTTTGCTGTTAATTTAGGCACAG
GCTTCATACTACATGGGTCAATAGTATAGGGATTCATATTATAGGCGATACTATAATAATTTGTTCGTCTGCAGA
GCTTATTATTTGCCAAAATTAGATATTCCTATTCTGTTTTTGTTTGTGTGCTGTTAAATTGTTAACGCCTGAAGGA
ATAAATATAAATGACGAAATTTTGATGTTTATCTCTGCTCCTTTATTGTGACCATAAGTCAAGATCAGATGCACT
TGTTTTAAATATTGTTGTCTGAAGAAATAAGTACTGACAGTATTTTGATGCATTGATCTGCTTGTTTGTTGTAAC
AAAATTTAAAAATAAAGAGTTTCCTTTTTGTTGCTCTCCTTACCTCCTGATGGTATCTAGTATCTACCAACTGAC
ACTATATTGCTTCTCTTTACATACGTATCTTGCTCGATGCCTTCTCCCTAGTGTTGACCAGTGTTACTCACATAGT
CTTTGCTCATTTCATTGTAATGCAGATACCAAGCGGCCCCTGCAGGATGGGGATTGAACAAGATGGATTGCACG
CAGGTTCTCCGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGTGCCCTGAA
TGAACTCCAGGACGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTGTCATCTCACCTT
GCTCCTGCCGAGAAAGTATCCATCATGGCTGATGCAATGCGGCGGCTGCATACGCTTGATCCGGCTACCTGCCC
ATTCGACCACCAAGCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCGCGCATGCCCGACG
GCGAGGATCTCGTCGTGACACATGGCGATGCCTGCTTGCCGAATATCATGGTGGAAAATGGCCGCTTTTCTGGA
TTCATCGACTGTGGCCGGCTGGGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGAGCGATCGCAGAGCCATGGGGGCGGTGAGAGCTCGCGGCGTCG
GATCTCCGGTTTCAGTTTCAGCTACGCGACTTGTGTGTGTGTGTGTGTGTCAGTGCGTGGTTACTCTGAGCTTCG
GTATGTTTGGTGTGTACTGTTCAGTGTTTCATGGTGCTTCCTAGCAGTGTCTGTCGTGTACTTTGCCTGTATGTTG
AGCTCGGCTCAGTATGTTCTGGGAGTGAATAAATAAAAAAAAATTAACCAGATATTGTCGCGTGTACTTAGTAT
ACTAATCACCTGCTCCTGCTCTTGCTCTGTTTTATCACCTTTCGGGCTTGACTGTCTTTGTGGAGGCCTCTCAGTC
TGATCTGGAACTCTACCTAAGTTTTACTAGTCCCGGGAAGCTTCTGCAGTGCAGCGTGACCCGGTCGTGCCCCTC
ACTAGAGATAATGAGCATTGCATGTCTAAGTTATAAAAAATTACCACATATTTTTTTTGTCACACTTGTTTGAAG
TGCAGTTTATCTATCTTTATACATATATTTAAACTTTACTCTACGAATAATATAATCTATAGTACTACAATAATAT
CAGTGTTTTAGAGAATCATATAAATGAACAGTTAGACATGGTCTAAAGGACAATTGAGTATTTTGACAACAGGA
CTCTACAGTTTTATCTTTTTAGTGTGCATGTGTTCTCCTTTTTTTTTGCAAATAGCTTCACCTATATAATACTTCAT
CCATTTTATTAGTACATCCATTTAGGGTTTAGGGTTAATGGTTTTTATAGACTAATTTTTTTAGTACATCTATTTT
ATTCTATTTTAGCCTCTAAATTAAGAAAACTAAAACTCTATTTTAGTTTTTTTATTTAATAATTTAGATATAAAAT
AGAATAAAATAAAGTGACTAAAAATTAAACAAATACCCTTTAAGAAATTAAAAAAACTAAGGAAACATTTTTC
TTGTTTCGAGTAGATAATGCCAGCCTGTTAAACGCCGTCGACGAGTCTAACGGACACCAACCAGCGAACCAGCA
GCGTCGCGTCGGGCCAAGCGAAGCAGACGGCACGGCATCTCTGTCGCTGCCTCTGGACCCCTCTCGAGAGTTCC
GCTCCACCGTTGGACTTGCTCCGCTGTCGGCATCCAGAAATTGCGTGGCGGAGCGGCAGACGTGAGCCGGCACG
GCAGGCGGCCTCCTCCTCCTCTCACGGCACGGCAGCTACGGGGGATTCCTTTCCCACCGCTCCTTCGCTTTCCCT
TCCTCGCCCGCCGTAATAAATAGACACCCCCTCCACACCCTCTTTCCCCAACCTCGTGTTGTTCGGAGCGCACAC
ACACACAACCAGATCTCCCCCAAATCCACCCGTCGGCACCTCCGCTTCAAGGTACGCCGCTCGTCCTCCCCCCC
CCCCCCTCTCTACCTTCTCTAGATCGGCGTTCCGGTCCATGGTTAGGGCCCGGTAGTTCTACTTCTGTTCATGTTT
GTGTTAGATCCGTGTTTGTGTTAGATCCGTGCTGCTAGCGTTCGTACACGGATGCGACCTGTACGTCAGACACGT
TCTGATTGCTAACTTGCCAGTGTTTCTCTTTGGGGAATCCTGGGATGGCTCTAGCCGTTCCGCAGACGGGATCGA
TTTCATGATTTTTTTTGTTTCGTTGCATAGGGTTTGGTTTGCCCTTTTCCTTTATTTCAATATATGCCGTGCACTTG
TTTGTCGGGTCATCTTTTCATGCTTTTTTTTGTCTTGGTTGTGATGATGTGGTCTGGTTGGGCGGTCGTTCTAGAT
CGGAGTAGAATTCTGTTTCAAACTACCTGGTGGATTTATTAATTTTGGATCTGTATGTGTGTGCCATACATATTC
ATAGTTACGAATTGAAGATGATGGATGGAAATATCGATCTAGGATAGGTATACATGTTGATGCGGGTTTTACTG
ATGCATATACAGAGATGCTTTTTGTTCGCTTGGTTGTGATGATGTGGTGTGGTTGGGCGGTCGTTCATTCGTTCT
AGATCGGAGTAGAATACTGTTTCAAACTACCTGGTGTATTTATTAATTTTGGAACTGTATGTGTGTGTCATACAT
CTTCATAGTTACGAGTTTAAGATGGATGGAAATATCGATCTAGGATAGGTATACATGTTGATGTGGGTTTTACT
GATGCATATACATGATGGCATATGCAGCATCTATTCATATGCTCTAACCTTGAGTACCTATCTATTATAATAAAC
AAGTATGTTTTATAATTATTTTGATCTTGATATACTTGGATGATGGCATATGCAGCAGCTATATGTGGATTTTTTT
AGCCCTGCCTTCATACGCTATTTATTTGCTTGGTACTGTTTCTTTTGTCGATGCTCACCCTGTTGTTTGGTGTTACT
TCTGCAGGGCCGGCCATGGATCCTAAAAAGAAACGTAAGGTCGACAAGAAGTACAGCATTGGTCTGGACATCG
GGACGAATTCCGTTGGCTGGGCCGTGATCACCGATGAGTACAAGGTCCCTTCCAAGAAGTTCAAGGTTCTGGGG
AACACCGATCGGCACAGCATCAAGAAGAACCTCATTGGAGCCCTCCTGTTCGACTCAGGCGAGACCGCCGAAG
CAACAAGGCTCAAGAGAACCGCAAGGAGACGGTATACCAGAAGGAAGAACAGGATCTGCTACCTGCAGGAGA
TCTTCAGCAACGAAATGGCGAAGGTGGACGATTCGTTCTTCCACAGATTGGAAGAGAGTTTCCTCGTCGAGGAA
GACAAGAAGCACGAGAGGCATCCGATCTTCGGCAACATCGTCGACGAGGTTGCCTATCACGAGAAGTACCCCA
CCATCTACCATCTGCGGAAGAAGCTTGTGGACTCGACTGACAAGGCGGACCTTAGGTTGATCTACCTCGCTCTG
GCCCACATGATCAAGTTCAGGGGCCATTTCCTGATCGAGGGGGATCTGAACCCGGACAACAGCGATGTGGACA
AGTTGTTCATCCAGCTCGTCCAAACCTACAACCAGCTCTTTGAGGAGAACCCGATCAACGCTTCAGGCGTCGAC
GCCAAGGCGATCCTGTCTGCACGCCTGTCAAAGTCTCGCCGGCTTGAGAACTTGATCGCTCAACTCCCGGGCGA
GAAGAAGAACGGCTTGTTCGGGAATCTCATTGCACTTTCGTTGGGGCTCACCCCGAACTTCAAGAGCAACTTCG
ATCTCGCTGAGGACGCAAAGCTGCAGCTTTCCAAGGACACTTACGACGATGACCTGGATAACCTGTTGGCCCAA
ATCGGCGATCAGTACGCGGACTTGTTCCTCGCCGCGAAGAACTTGTCGGACGCGATCCTCCTGAGTGACATTCT
CCGCGTGAACACCGAGATCACCAAGGCCCCGCTCTCGGCGAGCATGATCAAGCGGTATGACGAGCACCATCAG
GATCTGACCCTGTTGAAGGCTTTGGTCCGGCAGCAACTCCCAGAGAAGTACAAGGAGATCTTCTTCGATCAGTC
CAAGAACGGCTACGCTGGCTACATCGACGGGGGGCATCGCAGGAGGAGTTCTACAAGTTCATCAAGCCAATC
CTGGAGAAGATGGATGGCACAGAGGAACTCCTGGTGAAGCTCAACAGGGAGGACCTGTTGCGGAAGCAAAGG
ACTTTCGATAACGGCAGCATCCCTCACCAGATCCATCTCGGGGAGCTGCACGCCATCCTGAGAAGGCAGGAAG
ACTTCTACCCCTTCCTTAAGGATAACCGGGAGAAGATCGAGAAGATCCTGACGTTCAGAATCCCGTACTACGTC
GGACCACTCGCCCGGGGTAATTCCAGATTCGCGTGGATGACCAGAAAGAGCGAGGAAACCATCACCCCTTGGA
ACTTCGAGGAAGTGGTCGATAAGGGCGCTTCCGCACAGAGCTTCATCGAGCGCATGACCAACTTCGACAAGAA
CCTGCCTAACGAGAAGGTCCTTCCCAAGCATTCCCTCCTGTACGAGTACTTCACTGTGTACAACGAACTCACGA
AGGTGAAGTACGTGACCGAGGGAATGCGCAAGCCCGCCTTCCTGAGCGGCGAGCAGAAGAAGGCGATCGTGG
ACCTGTTGTTCAAGACCAATCGGAAGGTCACAGTGAAGCAGCTCAAGGAGGACTACTTCAAGAAGATCGAATG
CTTCGATTCCGTTGAGATCAGCGGCGTGGAAGACAGGTTTAACGCCTCACTGGGGACTTACCACGATCTCCTGA
AGATCATCAAGGATAAGGACTTCTTGGACAACGAGGAGAATGAGGACATCCTCGAAGACATTGTCCTGACTCTT
ACGTTGTTCGAGGATAGGGAGATGATCGAGGAACGCTTGAAGACGTATGCCCATCTCTTCGATGACAAGGTGAT
GAAGCAGCTCAAGAGAAGAAGGTACACCGGATGGGGAAGGCTGTCCCGCAAGCTGATCAACGGCATCAGAGA
CAAGCAATCAGGGAAGACAATCCTTGACTTCTTGAAGTCTGATGGCTTCGCGAACAGGAACTTCATGCAGCTCA
TTCACGATGACTCACTGACCTTCAAGGAGGACATCCAGAAGGCTCAAGTGTCGGGACAAGGTGACAGTCTGCA
CGAGCATATCGCCAACCTTGCGGGATCTCCTGCCATCAAGAAGGGTATCCTGCAGACAGTCAAGGTTGTGGATG
AGCTTGTGAAGGTCATGGGACGGCATAAGCCCGAGAACATCGTTATCGAGATGGCCAGAGAGAATCAGACCAC
CCAGAAGGGTCAGAAGAACTCGAGGGAGCGCATGAAGCGCATCGAGGAAGGCATTAAGGAGCTGGGGAGTCA
GATCCTTAAGGAGCACCCGGTGGAGAACACGCAGTTGCAGAATGAGAAGCTCTATCTGTACTACCTGCAGAAT
GGCAGGGACATGTACGTGGACCAGGAGTTGGACATCAACCGCCTCTCGGATTACGACGTCGATCACATCGTTCC
TCAGTCCTTCCTTAAGGATGACAGCATTGACAACAAGGTTCTCACCAGGTCCGACAAGAACCGCGGGAAGTCCG
ATAACGTGCCCAGCGAGGAAGTCGTGAAGAAGATGAAGAACTACTGGAGGCAACTGCTGAATGCCAAGTTGAT
CACCCAGAGGAAGTTCGACAACCTCACTAAGGCCGAGCGCGGAGGTCTCAGCGAACTGGACAAGGCGGGCTTC
ATCAAGCGGCAACTGGTTGAGACTAGACAGATCACGAAGCACGTCGCGCAGATTCTCGATTCACGCATGAACA
CGAAGTACGATGAGAATGACAAGCTGATCCGGGAAGTGAAGGTCATCACCTTGAAGTCAAAGCTCGTTTCTGA
CTTCAGGAAGGATTTCCAGTTCTACAAGGTGCGCGAGATCAACAACTACCACCATGCTCATGACGCATACCTCA
ACGCTGTGGTCGGAACAGCACTGATCAAGAAGTACCCGAAGCTCGAGTCCGAATTCGTGTACGGTGACTACAA
GGTTTACGATGTGCGCAAGATGATCGCCAAGTCAGAGCAGGAAATTGGCAAGGCCACTGCGAAGTACTTCTTCT
ACTCCAACATCATGAACTTCTTCAAGACTGAGATCACGCTGGCTAATGGCGAAATCCGGAAGAGACCACTGATT
GAGACCAACGGCGAGACAGGGGAAATCGTCTGGGACAAGGGGAGGGATTTCGCCACAGTCCGCAAGGTTCTCT
CTATGCCTCAAGTGAACATCGTCAAGAAGACTGAAGTCCAGACGGGCGGGTTCTCAAAGGAGTCTATCCTGCCC
AAGCGGAACTCGGATAAGCTTATCGCCAGGAAGAAGGACTGGGATCCGAAGAAGTACGGAGGTTTCGACTCAC
CAACGGTGGCTTACTCTGTCCTGGTTGTGGCAAAGGTGGAGAAGGGAAAGTCGAAGAAGCTCAAGTCTGTCAA
GGAGCTGCTGGGTATCACCATTATGGAGAGGTCCAGCTTCGAGAAGAACCCGATCGACTTCCTCGAGGCGAAG
GGCTACAAGGAAGTGAAGAAGGACCTGATCATCAAGCTTCCGAAGTACAGTCTGTTCGAGTTGGAGAACGGCA
GGAAGCGCATGTTGGCTTCCGCAGGAGAGCTCCAGAAGGGTAACGAGCTTGCTTTGCCGTCCAAGTACGTGAA
CTTCCTCTATCTGGCATCCCACTACGAGAAGCTCAAGGGCAGCCCAGAGGATAACGAACAGAAGCAACTGTTTG
TGGAGCAACACAAGCATTACCTTGACGAGATCATCGAACAGATCTCGGAGTTCAGTAAGCGCGTCATCCTCGCC
GACGCGAATTTGGATAAGGTTCTCTCAGCCTACAACAAGCACCGGGACAAGCCTATCAGAGAGCAGGCGGAGA
ACATCATCCATCTCTTCACCCTGACAAACCTTGGGGCTCCCGCTGCCTTCAAGTACTTCGACACTACGATCGACC
GGAAGAGGTACACTTCTACGAAGGAGGTGCTGGATGCAACCCTTATCCACCAATCGATCACTGGCCTCTACGAG
ACGCGGATCGACTTGAGTCAGCTCGGTGGCGATAAGAGACCCGCAGCAACCAAGAAGGCAGGGCAAGCAAAG
AAGAAGAAGTGAGCGATCGCATATGAAGATGAAGATGAAATATTTGGTGTGTCAAATAAAAAGCTTGTGTGCT
TAAGTTTGTGTTTTTTTCTTGGCTTGTTGTGTTATGAATTTGTGGCTTTTTCTAATATTAAATGAATGTAAGATCT
CATTATAATGAATAAACAAATGTTTCTATAATCCATTGTGAATGTTTTGTTGGATCTCTTCTGCAGCATATAACT
ACTGTATGTGCTATGGTATGGACTATGGAATATGATTAAAGATAAGGGTACGTACCGCGATCGCATAACTTCGT
ATAGCATACATTATACGAAGTTATGAATTCGGCAATCAAGAGGTAAAGGATAATAATCCCAGCAGAAACCGTG
ATCAAAGAAACAATTCATTGCAATATAGACTAGGCCATGCATACGCAAATCCAAGCGTGCATCCACTGACTGCA
TAACATAAACCCCCAGGTACTACGACTAAGCCGAACTGATACAAAATGCACTAGCTATTGGATAATCAGATCCT
GGTCCATGCTGCTGTCATTCCATCGCAATCACCCTGGTAGCGACGACAGGATCATTTGCTTCCAGAACCGAACT
GTGCGCCTAGCTCGGCGCGCCTAACTATAACGGTCCTAAGGTAGCGAATCCCAATGGCGCGCCGAGCTTGGCTC
GAGCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCTGAAGCCAGT
TACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTAGAAAAACTCAT
CGAGCATCAAATGAAACTGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGGTCTGCGATTCCGACTCGT
CCAACATCAATACAACCTATTAATTTCCCCTCGTCAAAAATAAGGTTATCAAGTGAGAAATCACCATGAGTGAC
GACTGAATCCGGTGAGAATGGCAAAAGTTTATGCATTTCTTTCCAGACTTGTTCAACAGGCCAGCCATTACGCT
CGTCATCAAAATCACTCGCATCAACCAAACCGTTATTCATTCGTGATTGCGCCTGAGCGAGACGAAATACGCGA
TCGCTGTTAAAAGGACAATTACAAACAGGAATCGAATGCAACCGGCGCAGGAACACTGCCAGCGCATCAACAA
TATTTTCACCTGAATCAGGATATTCTTCTAATACCTGGAATGCTGTTTTCCCAGGGATCGCAGTGGTGAGTAACC
ATGCATCATCAGGAGTACGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTATACCCATATAAATCAGC
ATCCATGTTGGAATTTAATCGCGGCCTAGAGCAAGACGTTTCCCGTTGAATATGGCTCATACTCTTCCTTTTTCA
ATATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGACATTA
ACCTATAAAAATAGGCGTATCACGAGGCCCTTTCGTC
SEQ ID NO: 14 (ScFT8 sgRNA expression vector, 13810 bp)
TCGCGCGTTTCGGTGATGACGGTGAAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGGCGGGTGTCGGGGCTGGCTTAACT
ATGCGGCATCAGAGCAGATTGTACTGAGAGTGCACCATATGCGGTGTGAAATACCGCACAGATGCGTAAGGAG
AAAATACCGCATCAGGCGCCATTCGCCATTCAGGCTGCGCAACTGTTGGGAAGGGCGATCGGTGCGGGCCTCTT
CGCTATTACGCCAGCTGGCGAAAGGGGGATGTGCTGCAAGGCGATTAAGTTGGGTAACGCCAGGGTTTTCCCA
GTCACGACGTTGTAAAACGACGGCCAGTGAATTGACGCGTATTGGGATTAACTATAACGGTCCTAAGGTAGCG
AGGCGCGCCGTTGTTTTAGTGTCCGAGGTTTGGTTTGGTGAAGTACATCTGAACTCTGAAGGTTCTTCGTGACAT
GGTTAGACGTGGTGTTGCAGTACTGTGATAAAACTTATCAGAATAAGTTTTCGCTTCTAATTTGTTTTCTTAAAT
CATGTGTTGATCAGCCTATGAGTTGTTCATATCGTTGCCTTTCGTGTTGAACATTTGTTTTCTTGCCATACTAATG
AGATTCATCTGCACAAACTGTACGAAGAGAATCAAGATATCGCCCGGGCTTTGTGAAAGTTGAATTACGGCATA
GCCGAAGGAATAACAGAATCGTTTCACACTTTCGTAACAAAGATCTTCTTATCATGTTTCAGACGATGGAGGCA
AGGCTGATCAAAGTGATCAAGCACATAAACGCATTTTTTTACCATGTTTCACTCCATAAGCGTCTGAGATTATCA
CAAGTCACGTCTAGTAGTTTGATGGTACACTAGTGACAATCAGTTCGTGCAGACAGAGCTCATACTTGACTACT
TGAGCGATTACAGGCGAAAGTGTGAAACGCATGTGATGTGGGCTGGGAGGAGGAGAATATATACTAATGGGCC
GTATCCTGATTTGGGCTGCGTCGGAAGGTGCAGCCCACGCGCGCCGTACCGCGCGGGTGGCGCTGCTACCCACT
TTAGTCCGTTGGATGGGGATCCGATGGTTTGCGCGGTGGCGTTGCGGGGGATGTTTAGTACCACATCGGAAACC
GAAAGACGATGGAACCAGCTTATAAACCCGCGCGCTGTAGTCAGCTTGAGAAGTAGCCTATTGTTGTGTTTTAG
AGCTAGAAATAGCAAGTTAAAATAAGGCTAGTCCGTTATCAACTTGAAAAAGTGGCACCGAGTCGGTGCTTTTT
TTTTAAGCTTTTTGTGAAAGTTGAATTACGGCATAGCCGAAGGAATAACAGAATCGTTTCACACTTTCGTAACA
AAGATCTTCTTATCATGTTTCAGACGATGGAGGCAAGGCTGATCAAAGTGATCAAGCACATAAACGCATTTTTT
TACCATGTTTCACTCCATAAGCGTCTGAGATTATCACAAGTCACGTCTAGTAGTTTGATGGTACACTAGTGACAA
TCAGTTCGTGCAGACAGAGCTCATACTTGACTACTTGAGCGATTACAGGCGAAAGTGTGAAACGCATGTGATGT
GGGCTGGGAGGAGGAGAATATATACTAATGGGCCGTATCCTGATTTGGGCTGCGTCGGAAGGTGCAGCCCACG
CGCGCCGTACCGCGCGGGTGGCGCTGCTACCCACTTTAGTCCGTTGGATGGGGATCCGATGGTTTGCGCGGTGG
CGTTGCGGGGGATGTTTAGTACCACATCGGAAACCGAAAGACGATGGAACCAGCTTATAAACCCGCGCGCTGT
AGTCAGCTTGCCCAAGCAATCCATCACTGGTTTTAGAGCTAGAAATAGCAAGTTAAAATAAGGCTAGTCCGTTA
TCAACTTGAAAAAGTGGCACCGAGTCGGTGCTTTTTTTTTGCGGCCGCCTGCAGGTCAACGGATCACCTATCAA
CATCCCAGCTAAAAACAGTAAAAAGGGGGAAAACGTGGGTGAGTTGAGTCTGTCTTGTGGAAAAAACGTTTTA
GTTTCTCCTGGAATTAACAATAAAAACAGTTGAACAAGATTGACTGTTCCTCCGGGAGGGTTTGGAACATCGTT
ACAGATGTGAGCGAAAGGTGAGGAAACAGAGCGGAGGGCTTGGAGGTGACCTCGGTAGTCGACGCCGGAGTT
GAGCTTGATGACGACACCGTACTGGCGTACCAGGCCTAGTAGTGAACACCGGGCCTGAAGCTGTCGCCGCCGCT
GCTCATCTTGTGGGCTGTGCCCGGTGTCCCTGTTGCGGATTGCGGGTGGCAGCCTGGCAGGTGGGTGCGACCCG
TTTGGACTCCCTGATCTGGGCCCTTTGTGTCAGTACCGTCTGTACTCCGATGACATGCACACCGTCGTCCACAGT
CAAGTCCACAATCTCCCCTCTTTTTTTAACGGAATAGTTCAAAATCTCCTTGACGCACGCTATCGTGTACCAGCG
CTCACTGGACACCACGTTTGTAATCCACGCCGACACGTCGGTCCCACGTCGACAGGCCCCACCGTCCGGTCTGT
AGCGTGTACGTATTCGGGCGACGGACGTGTCGTCGTCGTCTTGCGAGTCCCATTCCCATCACCATCTGAGCCAC
ACATCCTCTGAACAAAAGCAGGGAGGCCTCTACGCACATCCCCCTTTCTCCCACTCCTTGTCCGTGGCACCCACC
CCAAACCCTCGCGCCGCCTCCGAGACAGCCGCCGCAACCATGGCCACCGCCTGCAGGACCGTCTTCGGTACGCG
CTCACTCCGCCCTCTGCCTTTGTTACTGCCACGTTTCTCTGAATGCTCTCTTGTGTGGTGATTGCTGAGAGTGGTT
TAGCTGGATCTAGAATTACACTCTGAAATCGTGTTCTGCCTGTGCTGATTACTTGCCGTCCTTTGTAGCAGCAAA
ATATAGGGACATGGTAGTACGAAACGAAGATAGAACCTACACAGCAATACGAGAAATGTGTAATTTGGTGCTT
AGCGGTATTTATTTAAGCACATGTTGGTGTTATAGGGCACTTGGATTCAGAAGTTTGCTGTTAATTTAGGCACAG
GCTTCATACTACATGGGTCAATAGTATAGGGATTCATATTATAGGCGATACTATAATAATTTGTTCGTCTGCAGA
GCTTATTATTTGCCAAAATTAGATATTCCTATTCTGTTTTTGTTTGTGTGCTGTTAAATTGTTAACGCCTGAAGGA
ATAAATATAAATGACGAAATTTTGATGTTTATCTCTGCTCCTTTATTGTGACCATAAGTCAAGATCAGATGCACT
TGTTTTAAATATTGTTGTCTGAAGAAATAAGTACTGACAGTATTTTGATGCATTGATCTGCTTGTTTGTTGTAAC
AAAATTTAAAAATAAAGAGTTTCCTTTTTGTTGCTCTCCTTACCTCCTGATGGTATCTAGTATCTACCAACTGAC
ACTATATTGCTTCTCTTTACATACGTATCTTGCTCGATGCCTTCTCCCTAGTGTTGACCAGTGTTACTCACATAGT
CTTTGCTCATTTCATTGTAATGCAGATACCAAGCGGCCCCTGCAGGATGGGGATTGAACAAGATGGATTGCACG
CAGGTTCTCCGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGTGCCCTGAA
TGAACTCCAGGACGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTGTCATCTCACCTT
GCTCCTGCCGAGAAAGTATCCATCATGGCTGATGCAATGCGGCGGCTGCATACGCTTGATCCGGCTACCTGCCC
ATTCGACCACCAAGCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCGCGCATGCCCGACG
GCGAGGATCTCGTCGTGACACATGGCGATGCCTGCTTGCCGAATATCATGGTGGAAAATGGCCGCTTTTCTGGA
TTCATCGACTGTGGCCGGCTGGGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGAGCGATCGCAGAGCCATGGGGGCGGTGAGAGCTCGCGGCGTCG
GATCTCCGGTTTCAGTTTCAGCTACGCGACTTGTGTGTGTGTGTGTGTGTCAGTGCGTGGTTACTCTGAGCTTCG
GTATGTTTGGTGTGTACTGTTCAGTGTTTCATGGTGCTTCCTAGCAGTGTCTGTCGTGTACTTTGCCTGTATGTTG
AGCTCGGCTCAGTATGTTCTGGGAGTGAATAAATAAAAAAAAATTAACCAGATATTGTCGCGTGTACTTAGTAT
ACTAATCACCTGCTCCTGCTCTTGCTCTGTTTTATCACCTTTCGGGCTTGACTGTCTTTGTGGAGGCCTCTCAGTC
TGATCTGGAACTCTACCTAAGTTTTACTAGTCCCGGGAAGCTTCTGCAGTGCAGCGTGACCCGGTCGTGCCCCTC
ACTAGAGATAATGAGCATTGCATGTCTAAGTTATAAAAAATTACCACATATTTTTTTTGTCACACTTGTTTGAAG
TGCAGTTTATCTATCTTTATACATATATTTAAACTTTACTCTACGAATAATATAATCTATAGTACTACAATAATAT
CAGTGTTTTAGAGAATCATATAAATGAACAGTTAGACATGGTCTAAAGGACAATTGAGTATTTTGACAACAGGA
CTCTACAGTTTTATCTTTTTAGTGTGCATGTGTTCTCCTTTTTTTTTGCAAATAGCTTCACCTATATAATACTTCAT
CCATTTTATTAGTACATCCATTTAGGGTTTAGGGTTAATGGTTTTTATAGACTAATTTTTTTAGTACATCTATTTT
ATTCTATTTTAGCCTCTAAATTAAGAAAACTAAAACTCTATTTTAGTTTTTTTATTTAATAATTTAGATATAAAAT
AGAATAAAATAAAGTGACTAAAAATTAAACAAATACCCTTTAAGAAATTAAAAAAACTAAGGAAACATTTTTC
TTGTTTCGAGTAGATAATGCCAGCCTGTTAAACGCCGTCGACGAGTCTAACGGACACCAACCAGCGAACCAGCA
GCGTCGCGTCGGGCCAAGCGAAGCAGACGGCACGGCATCTCTGTCGCTGCCTCTGGACCCCTCTCGAGAGTTCC
GCTCCACCGTTGGACTTGCTCCGCTGTCGGCATCCAGAAATTGCGTGGCGGAGCGGCAGACGTGAGCCGGCACG
GCAGGCGGCCTCCTCCTCCTCTCACGGCACGGCAGCTACGGGGGATTCCTTTCCCACCGCTCCTTCGCTTTCCCT
TCCTCGCCCGCCGTAATAAATAGACACCCCCTCCACACCCTCTTTCCCCAACCTCGTGTTGTTCGGAGCGCACAC
ACACACAACCAGATCTCCCCCAAATCCACCCGTCGGCACCTCCGCTTCAAGGTACGCCGCTCGTCCTCCCCCCC
CCCCCCTCTCTACCTTCTCTAGATCGGCGTTCCGGTCCATGGTTAGGGCCCGGTAGTTCTACTTCTGTTCATGTTT
GTGTTAGATCCGTGTTTGTGTTAGATCCGTGCTGCTAGCGTTCGTACACGGATGCGACCTGTACGTCAGACACGT
TCTGATTGCTAACTTGCCAGTGTTTCTCTTTGGGGAATCCTGGGATGGCTCTAGCCGTTCCGCAGACGGGATCGA
TTTCATGATTTTTTTTGTTTCGTTGCATAGGGTTTGGTTTGCCCTTTTCCTTTATTTCAATATATGCCGTGCACTTG
TTTGTCGGGTCATCTTTTCATGCTTTTTTTTGTCTTGGTTGTGATGATGTGGTCTGGTTGGGCGGTCGTTCTAGAT
CGGAGTAGAATTCTGTTTCAAACTACCTGGTGGATTTATTAATTTTGGATCTGTATGTGTGTGCCATACATATTC
ATAGTTACGAATTGAAGATGATGGATGGAAATATCGATCTAGGATAGGTATACATGTTGATGCGGGTTTTACTG
ATGCATATACAGAGATGCTTTTTGTTCGCTTGGTTGTGATGATGTGGTGTGGTTGGGCGGTCGTTCATTCGTTCT
AGATCGGAGTAGAATACTGTTTCAAACTACCTGGTGTATTTATTAATTTTGGAACTGTATGTGTGTGTCATACAT
CTTCATAGTTACGAGTTTAAGATGGATGGAAATATCGATCTAGGATAGGTATACATGTTGATGTGGGTTTTACT
GATGCATATACATGATGGCATATGCAGCATCTATTCATATGCTCTAACCTTGAGTACCTATCTATTATAATAAAC
AAGTATGTTTTATAATTATTTTGATCTTGATATACTTGGATGATGGCATATGCAGCAGCTATATGTGGATTTTTTT
AGCCCTGCCTTCATACGCTATTTATTTGCTTGGTACTGTTTCTTTTGTCGATGCTCACCCTGTTGTTTGGTGTTACT
TCTGCAGGGCCGGCCATGGATCCTAAAAAGAAACGTAAGGTCGACAAGAAGTACAGCATTGGTCTGGACATCG
GGACGAATTCCGTTGGCTGGGCCGTGATCACCGATGAGTACAAGGTCCCTTCCAAGAAGTTCAAGGTTCTGGGG
AACACCGATCGGCACAGCATCAAGAAGAACCTCATTGGAGCCCTCCTGTTCGACTCAGGCGAGACCGCCGAAG
CAACAAGGCTCAAGAGAACCGCAAGGAGACGGTATACCAGAAGGAAGAACAGGATCTGCTACCTGCAGGAGA
TCTTCAGCAACGAAATGGCGAAGGTGGACGATTCGTTCTTCCACAGATTGGAAGAGAGTTTCCTCGTCGAGGAA
GACAAGAAGCACGAGAGGCATCCGATCTTCGGCAACATCGTCGACGAGGTTGCCTATCACGAGAAGTACCCCA
CCATCTACCATCTGCGGAAGAAGCTTGTGGACTCGACTGACAAGGCGGACCTTAGGTTGATCTACCTCGCTCTG
GCCCACATGATCAAGTTCAGGGGCCATTTCCTGATCGAGGGGGATCTGAACCCGGACAACAGCGATGTGGACA
AGTTGTTCATCCAGCTCGTCCAAACCTACAACCAGCTCTTTGAGGAGAACCCGATCAACGCTTCAGGCGTCGAC
GCCAAGGCGATCCTGTCTGCACGCCTGTCAAAGTCTCGCCGGCTTGAGAACTTGATCGCTCAACTCCCGGGCGA
GAAGAAGAACGGCTTGTTCGGGAATCTCATTGCACTTTCGTTGGGGCTCACCCCGAACTTCAAGAGCAACTTCG
ATCTCGCTGAGGACGCAAAGCTGCAGCTTTCCAAGGACACTTACGACGATGACCTGGATAACCTGTTGGCCCAA
ATCGGCGATCAGTACGCGGACTTGTTCCTCGCCGCGAAGAACTTGTCGGACGCGATCCTCCTGAGTGACATTCT
CCGCGTGAACACCGAGATCACCAAGGCCCCGCTCTCGGCGAGCATGATCAAGCGGTATGACGAGCACCATCAG
GATCTGACCCTGTTGAAGGCTTTGGTCCGGCAGCAACTCCCAGAGAAGTACAAGGAGATCTTCTTCGATCAGTC
CAAGAACGGCTACGCTGGCTACATCGACGGCGGGGCATCGCAGGAGGAGTTCTACAAGTTCATCAAGCCAATC
CTGGAGAAGATGGATGGCACAGAGGAACTCCTGGTGAAGCTCAACAGGGAGGACCTGTTGCGGAAGCAAAGG
ACTTTCGATAACGGCAGCATCCCTCACCAGATCCATCTCGGGGAGCTGCACGCCATCCTGAGAAGGCAGGAAG
ACTTCTACCCCTTCCTTAAGGATAACCGGGAGAAGATCGAGAAGATCCTGACGTTCAGAATCCCGTACTACGTC
GGACCACTCGCCCGGGGTAATTCCAGATTCGCGTGGATGACCAGAAAGAGCGAGGAAACCATCACCCCTTGGA
ACTTCGAGGAAGTGGTCGATAAGGGCGCTTCCGCACAGAGCTTCATCGAGCGCATGACCAACTTCGACAAGAA
CCTGCCTAACGAGAAGGTCCTTCCCAAGCATTCCCTCCTGTACGAGTACTTCACTGTGTACAACGAACTCACGA
AGGTGAAGTACGTGACCGAGGGAATGCGCAAGCCCGCCTTCCTGAGCGGCGAGCAGAAGAAGGCGATCGTGG
ACCTGTTGTTCAAGACCAATCGGAAGGTCACAGTGAAGCAGCTCAAGGAGGACTACTTCAAGAAGATCGAATG
CTTCGATTCCGTTGAGATCAGCGGCGTGGAAGACAGGTTTAACGCCTCACTGGGGACTTACCACGATCTCCTGA
AGATCATCAAGGATAAGGACTTCTTGGACAACGAGGAGAATGAGGACATCCTCGAAGACATTGTCCTGACTCTT
ACGTTGTTCGAGGATAGGGAGATGATCGAGGAACGCTTGAAGACGTATGCCCATCTCTTCGATGACAAGGTGAT
GAAGCAGCTCAAGAGAAGAAGGTACACCGGATGGGGAAGGCTGTCCCGCAAGCTGATCAACGGCATCAGAGA
CAAGCAATCAGGGAAGACAATCCTTGACTTCTTGAAGTCTGATGGCTTCGCGAACAGGAACTTCATGCAGCTCA
TTCACGATGACTCACTGACCTTCAAGGAGGACATCCAGAAGGCTCAAGTGTCGGGACAAGGTGACAGTCTGCA
CGAGCATATCGCCAACCTTGCGGGATCTCCTGCCATCAAGAAGGGTATCCTGCAGACAGTCAAGGTTGTGGATG
AGCTTGTGAAGGTCATGGGACGGCATAAGCCCGAGAACATCGTTATCGAGATGGCCAGAGAGAATCAGACCAC
CCAGAAGGGTCAGAAGAACTCGAGGGAGCGCATGAAGCGCATCGAGGAAGGCATTAAGGAGCTGGGGAGTCA
GATCCTTAAGGAGCACCCGGTGGAGAACACGCAGTTGCAGAATGAGAAGCTCTATCTGTACTACCTGCAGAAT
GGCAGGGACATGTACGTGGACCAGGAGTTGGACATCAACCGCCTCTCGGATTACGACGTCGATCACATCGTTCC
TCAGTCCTTCCTTAAGGATGACAGCATTGACAACAAGGTTCTCACCAGGTCCGACAAGAACCGCGGGAAGTCCG
ATAACGTGCCCAGCGAGGAAGTCGTGAAGAAGATGAAGAACTACTGGAGGCAACTGCTGAATGCCAAGTTGAT
CACCCAGAGGAAGTTCGACAACCTCACTAAGGCCGAGCGCGGAGGTCTCAGCGAACTGGACAAGGCGGGCTTC
ATCAAGCGGCAACTGGTTGAGACTAGACAGATCACGAAGCACGTCGCGCAGATTCTCGATTCACGCATGAACA
CGAAGTACGATGAGAATGACAAGCTGATCCGGGAAGTGAAGGTCATCACCTTGAAGTCAAAGCTCGTTTCTGA
CTTCAGGAAGGATTTCCAGTTCTACAAGGTGCGCGAGATCAACAACTACCACCATGCTCATGACGCATACCTCA
ACGCTGTGGTCGGAACAGCACTGATCAAGAAGTACCCGAAGCTCGAGTCCGAATTCGTGTACGGTGACTACAA
GGTTTACGATGTGCGCAAGATGATCGCCAAGTCAGAGCAGGAAATTGGCAAGGCCACTGCGAAGTACTTCTTCT
ACTCCAACATCATGAACTTCTTCAAGACTGAGATCACGCTGGCTAATGGCGAAATCCGGAAGAGACCACTGATT
GAGACCAACGGCGAGACAGGGGAAATCGTCTGGGACAAGGGGAGGGATTTCGCCACAGTCCGCAAGGTTCTCT
CTATGCCTCAAGTGAACATCGTCAAGAAGACTGAAGTCCAGACGGGCGGGTTCTCAAAGGAGTCTATCCTGCCC
AAGCGGAACTCGGATAAGCTTATCGCCAGGAAGAAGGACTGGGATCCGAAGAAGTACGGAGGTTTCGACTCAC
CAACGGTGGCTTACTCTGTCCTGGTTGTGGCAAAGGTGGAGAAGGGAAAGTCGAAGAAGCTCAAGTCTGTCAA
GGAGCTGCTGGGTATCACCATTATGGAGAGGTCCAGCTTCGAGAAGAACCCGATCGACTTCCTCGAGGCGAAG
GGCTACAAGGAAGTGAAGAAGGACCTGATCATCAAGCTTCCGAAGTACAGTCTGTTCGAGTTGGAGAACGGCA
GGAAGCGCATGTTGGCTTCCGCAGGAGAGCTCCAGAAGGGTAACGAGCTTGCTTTGCCGTCCAAGTACGTGAA
CTTCCTCTATCTGGCATCCCACTACGAGAAGCTCAAGGGCAGCCCAGAGGATAACGAACAGAAGCAACTGTTTG
TGGAGCAACACAAGCATTACCTTGACGAGATCATCGAACAGATCTCGGAGTTCAGTAAGCGCGTCATCCTCGCC
GACGCGAATTTGGATAAGGTTCTCTCAGCCTACAACAAGCACCGGGACAAGCCTATCAGAGAGCAGGCGGAGA
ACATCATCCATCTCTTCACCCTGACAAACCTTGGGGCTCCCGCTGCCTTCAAGTACTTCGACACTACGATCGACC
GGAAGAGGTACACTTCTACGAAGGAGGTGCTGGATGCAACCCTTATCCACCAATCGATCACTGGCCTCTACGAG
ACGCGGATCGACTTGAGTCAGCTCGGTGGCGATAAGAGACCCGCAGCAACCAAGAAGGCAGGGCAAGCAAAG
AAGAAGAAGTGAGCGATCGCATATGAAGATGAAGATGAAATATTTGGTGTGTCAAATAAAAAGCTTGTGTGCT
TAAGTTTGTGTTTTTTTCTTGGCTTGTTGTGTTATGAATTTGTGGCTTTTTCTAATATTAAATGAATGTAAGATCT
CATTATAATGAATAAACAAATGTTTCTATAATCCATTGTGAATGTTTTGTTGGATCTCTTCTGCAGCATATAACT
ACTGTATGTGCTATGGTATGGACTATGGAATATGATTAAAGATAAGGGTACGTACCGCGATCGCATAACTTCGT
ATAGCATACATTATACGAAGTTATGAATTCGGCAATCAAGAGGTAAAGGATAATAATCCCAGCAGAAACCGTG
ATCAAAGAAACAATTCATTGCAATATAGACTAGGCCATGCATACGCAAATCCAAGCGTGCATCCACTGACTGCA
TAACATAAACCCCCAGGTACTACGACTAAGCCGAACTGATACAAAATGCACTAGCTATTGGATAATCAGATCCT
GGTCCATGCTGCTGTCATTCCATCGCAATCACCCTGGTAGCGACGACAGGATCATTTGCTTCCAGAACCGAACT
GTGCGCCTAGCTCGGCGCGCCTAACTATAACGGTCCTAAGGTAGCGAATCCCAATGGCGCGCCGAGCTTGGCTC
GAGCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCTGAAGCCAGT
TACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTAGAAAAACTCAT
CGAGCATCAAATGAAACTGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGGTCTGCGATTCCGACTCGT
CCAACATCAATACAACCTATTAATTTCCCCTCGTCAAAAATAAGGTTATCAAGTGAGAAATCACCATGAGTGAC
GACTGAATCCGGTGAGAATGGCAAAAGTTTATGCATTTCTTTCCAGACTTGTTCAACAGGCCAGCCATTACGCT
CGTCATCAAAATCACTCGCATCAACCAAACCGTTATTCATTCGTGATTGCGCCTGAGCGAGACGAAATACGCGA
TCGCTGTTAAAAGGACAATTACAAACAGGAATCGAATGCAACCGGCGCAGGAACACTGCCAGCGCATCAACAA
TATTTTCACCTGAATCAGGATATTCTTCTAATACCTGGAATGCTGTTTTCCCAGGGATCGCAGTGGTGAGTAACC
ATGCATCATCAGGAGTACGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTATACCCATATAAATCAGC
ATCCATGTTGGAATTTAATCGCGGCCTAGAGCAAGACGTTTCCCGTTGAATATGGCTCATACTCTTCCTTTTTCA
ATATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGACATTA
ACCTATAAAAATAGGCGTATCACGAGGCCCTTTCGTC
SEQ ID NO: 15 (ScFT10 sgRNA expression vector, 13810 bp)
TCGCGCGTTTCGGTGATGACGGTGAAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGGCGGGTGTCGGGGCTGGCTTAACT
ATGCGGCATCAGAGCAGATTGTACTGAGAGTGCACCATATGCGGTGTGAAATACCGCACAGATGCGTAAGGAG
AAAATACCGCATCAGGCGCCATTCGCCATTCAGGCTGCGCAACTGTTGGGAAGGGCGATCGGTGCGGGCCTCTT
CGCTATTACGCCAGCTGGCGAAAGGGGGATGTGCTGCAAGGCGATTAAGTTGGGTAACGCCAGGGTTTTCCCA
GTCACGACGTTGTAAAACGACGGCCAGTGAATTGACGCGTATTGGGATTAACTATAACGGTCCTAAGGTAGCG
AGGCGCGCCGTTGTTTTAGTGTCCGAGGTTTGGTTTGGTGAAGTACATCTGAACTCTGAAGGTTCTTCGTGACAT
GGTTAGACGTGGTGTTGCAGTACTGTGATAAAACTTATCAGAATAAGTTTTCGCTTCTAATTTGTTTTCTTAAAT
CATGTGTTGATCAGCCTATGAGTTGTTCATATCGTTGCCTTTCGTGTTGAACATTTGTTTTCTTGCCATACTAATG
AGATTCATCTGCACAAACTGTACGAAGAGAATCAAGATATCGCCCGGGCTTTGTGAAAGTTGAATTACGGCATA
GCCGAAGGAATAACAGAATCGTTTCACACTTTCGTAACAAAGATCTTCTTATCATGTTTCAGACGATGGAGGCA
AGGCTGATCAAAGTGATCAAGCACATAAACGCATTTTTTTACCATGTTTCACTCCATAAGCGTCTGAGATTATCA
CAAGTCACGTCTAGTAGTTTGATGGTACACTAGTGACAATCAGTTCGTGCAGACAGAGCTCATACTTGACTACT
TGAGCGATTACAGGCGAAAGTGTGAAACGCATGTGATGTGGGCTGGGAGGAGGAGAATATATACTAATGGGCC
GTATCCTGATTTGGGCTGCGTCGGAAGGTGCAGCCCACGCGCGCCGTACCGCGCGGGTGGCGCTGCTACCCACT
TTAGTCCGTTGGATGGGGATCCGATGGTTTGCGCGGTGGCGTTGCGGGGGATGTTTAGTACCACATCGGAAACC
GAAAGACGATGGAACCAGCTTATAAACCCGCGCGCTGTAGTCAGCTTGGTAAACCACGAGTCGATGTGTTTTAG
AGCTAGAAATAGCAAGTTAAAATAAGGCTAGTCCGTTATCAACTTGAAAAAGTGGCACCGAGTCGGTGCTTTTT
TTTTAAGCTTTTTGTGAAAGTTGAATTACGGCATAGCCGAAGGAATAACAGAATCGTTTCACACTTTCGTAACA
AAGATCTTCTTATCATGTTTCAGACGATGGAGGCAAGGCTGATCAAAGTGATCAAGCACATAAACGCATTTTTT
TACCATGTTTCACTCCATAAGCGTCTGAGATTATCACAAGTCACGTCTAGTAGTTTGATGGTACACTAGTGACAA
TCAGTTCGTGCAGACAGAGCTCATACTTGACTACTTGAGCGATTACAGGCGAAAGTGTGAAACGCATGTGATGT
GGGCTGGGAGGAGGAGAATATATACTAATGGGCCGTATCCTGATTTGGGCTGCGTCGGAAGGTGCAGCCCACG
CGCGCCGTACCGCGCGGGTGGCGCTGCTACCCACTTTAGTCCGTTGGATGGGGATCCGATGGTTTGCGCGGTGG
CGTTGCGGGGGATGTTTAGTACCACATCGGAAACCGAAAGACGATGGAACCAGCTTATAAACCCGCGCGCTGT
AGTCAGCTTGTCCAAGCCATCCATCACTAGTTTTAGAGCTAGAAATAGCAAGTTAAAATAAGGCTAGTCCGTTA
TCAACTTGAAAAAGTGGCACCGAGTCGGTGCTTTTTTTTTGCGGCCGCCTGCAGGTCAACGGATCACCTATCAA
CATCCCAGCTAAAAACAGTAAAAAGGGGGAAAACGTGGGTGAGTTGAGTCTGTCTTGTGGAAAAAACGTTTTA
GTTTCTCCTGGAATTAACAATAAAAACAGTTGAACAAGATTGACTGTTCCTCCGGGAGGGTTTGGAACATCGTT
ACAGATGTGAGCGAAAGGTGAGGAAACAGAGCGGAGGGCTTGGAGGTGACCTCGGTAGTCGACGCCGGAGTT
GAGCTTGATGACGACACCGTACTGGCGTACCAGGCCTAGTAGTGAACACCGGGCCTGAAGCTGTCGCCGCCGCT
GCTCATCTTGTGGGCTGTGCCCGGTGTCCCTGTTGCGGATTGCGGGTGGCAGCCTGGCAGGTGGGTGCGACCCG
TTTGGACTCCCTGATCTGGGCCCTTTGTGTCAGTACCGTCTGTACTCCGATGACATGCACACCGTCGTCCACAGT
CAAGTCCACAATCTCCCCTCTTTTTTTAACGGAATAGTTCAAAATCTCCTTGACGCACGCTATCGTGTACCAGCG
CTCACTGGACACCACGTTTGTAATCCACGCCGACACGTCGGTCCCACGTCGACAGGCCCCACCGTCCGGTCTGT
AGCGTGTACGTATTCGGGCGACGGACGTGTCGTCGTCGTCTTGCGAGTCCCATTCCCATCACCATCTGAGCCAC
ACATCCTCTGAACAAAAGCAGGGAGGCCTCTACGCACATCCCCCTTTCTCCCACTCCTTGTCCGTGGCACCCACC
CCAAACCCTCGCGCCGCCTCCGAGACAGCCGCCGCAACCATGGCCACCGCCTGCAGGACCGTCTTCGGTACGCG
CTCACTCCGCCCTCTGCCTTTGTTACTGCCACGTTTCTCTGAATGCTCTCTTGTGTGGTGATTGCTGAGAGTGGTT
TAGCTGGATCTAGAATTACACTCTGAAATCGTGTTCTGCCTGTGCTGATTACTTGCCGTCCTTTGTAGCAGCAAA
ATATAGGGACATGGTAGTACGAAACGAAGATAGAACCTACACAGCAATACGAGAAATGTGTAATTTGGTGCTT
AGCGGTATTTATTTAAGCACATGTTGGTGTTATAGGGCACTTGGATTCAGAAGTTTGCTGTTAATTTAGGCACAG
GCTTCATACTACATGGGTCAATAGTATAGGGATTCATATTATAGGCGATACTATAATAATTTGTTCGTCTGCAGA
GCTTATTATTTGCCAAAATTAGATATTCCTATTCTGTTTTTGTTTGTGTGCTGTTAAATTGTTAACGCCTGAAGGA
ATAAATATAAATGACGAAATTTTGATGTTTATCTCTGCTCCTTTATTGTGACCATAAGTCAAGATCAGATGCACT
TGTTTTAAATATTGTTGTCTGAAGAAATAAGTACTGACAGTATTTTGATGCATTGATCTGCTTGTTTGTTGTAAC
AAAATTTAAAAATAAAGAGTTTCCTTTTTGTTGCTCTCCTTACCTCCTGATGGTATCTAGTATCTACCAACTGAC
ACTATATTGCTTCTCTTTACATACGTATCTTGCTCGATGCCTTCTCCCTAGTGTTGACCAGTGTTACTCACATAGT
CTTTGCTCATTTCATTGTAATGCAGATACCAAGCGGCCCCTGCAGGATGGGGATTGAACAAGATGGATTGCACG
CAGGTTCTCCGGCCGCTTGGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGACCTGTCCGGTGCCCTGAA
TGAACTCCAGGACGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTGTCATCTCACCTT
GCTCCTGCCGAGAAAGTATCCATCATGGCTGATGCAATGCGGCGGCTGCATACGCTTGATCCGGCTACCTGCCC
ATTCGACCACCAAGCGAAACATCGCATCGAGCGAGCACGTACTCGGATGGAAGCCGGTCTTGTCGATCAGGAT
GATCTGGACGAAGAGCATCAGGGGCTCGCGCCAGCCGAACTGTTCGCCAGGCTCAAGGCGCGCATGCCCGACG
GCGAGGATCTCGTCGTGACACATGGCGATGCCTGCTTGCCGAATATCATGGTGGAAAATGGCCGCTTTTCTGGA
TTCATCGACTGTGGCCGGCTGGGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGAGCGATCGCAGAGCCATGGGGGCGGTGAGAGCTCGCGGCGTCG
GATCTCCGGTTTCAGTTTCAGCTACGCGACTTGTGTGTGTGTGTGTGTGTCAGTGCGTGGTTACTCTGAGCTTCG
GTATGTTTGGTGTGTACTGTTCAGTGTTTCATGGTGCTTCCTAGCAGTGTCTGTCGTGTACTTTGCCTGTATGTTG
AGCTCGGCTCAGTATGTTCTGGGAGTGAATAAATAAAAAAAAATTAACCAGATATTGTCGCGTGTACTTAGTAT
ACTAATCACCTGCTCCTGCTCTTGCTCTGTTTTATCACCTTTCGGGCTTGACTGTCTTTGTGGAGGCCTCTCAGTC
TGATCTGGAACTCTACCTAAGTTTTACTAGTCCCGGGAAGCTTCTGCAGTGCAGCGTGACCCGGTCGTGCCCCTC
ACTAGAGATAATGAGCATTGCATGTCTAAGTTATAAAAAATTACCACATATTTTTTTTGTCACACTTGTTTGAAG
TGCAGTTTATCTATCTTTATACATATATTTAAACTTTACTCTACGAATAATATAATCTATAGTACTACAATAATAT
CAGTGTTTTAGAGAATCATATAAATGAACAGTTAGACATGGTCTAAAGGACAATTGAGTATTTTGACAACAGGA
CTCTACAGTTTTATCTTTTTAGTGTGCATGTGTTCTCCTTTTTTTTTGCAAATAGCTTCACCTATATAATACTTCAT
CCATTTTATTAGTACATCCATTTAGGGTTTAGGGTTAATGGTTTTTATAGACTAATTTTTTTAGTACATCTATTTT
ATTCTATTTTAGCCTCTAAATTAAGAAAACTAAAACTCTATTTTAGTTTTTTTATTTAATAATTTAGATATAAAAT
AGAATAAAATAAAGTGACTAAAAATTAAACAAATACCCTTTAAGAAATTAAAAAAACTAAGGAAACATTTTTC
TTGTTTCGAGTAGATAATGCCAGCCTGTTAAACGCCGTCGACGAGTCTAACGGACACCAACCAGCGAACCAGCA
GCGTCGCGTCGGGCCAAGCGAAGCAGACGGCACGGCATCTCTGTCGCTGCCTCTGGACCCCTCTCGAGAGTTCC
GCTCCACCGTTGGACTTGCTCCGCTGTCGGCATCCAGAAATTGCGTGGCGGAGCGGCAGACGTGAGCCGGCACG
GCAGGCGGCCTCCTCCTCCTCTCACGGCACGGCAGCTACGGGGGATTCCTTTCCCACCGCTCCTTCGCTTTCCCT
TCCTCGCCCGCCGTAATAAATAGACACCCCCTCCACACCCTCTTTCCCCAACCTCGTGTTGTTCGGAGCGCACAC
ACACACAACCAGATCTCCCCCAAATCCACCCGTCGGCACCTCCGCTTCAAGGTACGCCGCTCGTCCTCCCCCCC
CCCCCCTCTCTACCTTCTCTAGATCGGCGTTCCGGTCCATGGTTAGGGCCCGGTAGTTCTACTTCTGTTCATGTTT
GTGTTAGATCCGTGTTTGTGTTAGATCCGTGCTGCTAGCGTTCGTACACGGATGCGACCTGTACGTCAGACACGT
TCTGATTGCTAACTTGCCAGTGTTTCTCTTTGGGGAATCCTGGGATGGCTCTAGCCGTTCCGCAGACGGGATCGA
TTTCATGATTTTTTTTGTTTCGTTGCATAGGGTTTGGTTTGCCCTTTTCCTTTATTTCAATATATGCCGTGCACTTG
TTTGTCGGGTCATCTTTTCATGCTTTTTTTTGTCTTGGTTGTGATGATGTGGTCTGGTTGGGCGGTCGTTCTAGAT
CGGAGTAGAATTCTGTTTCAAACTACCTGGTGGATTTATTAATTTTGGATCTGTATGTGTGTGCCATACATATTC
ATAGTTACGAATTGAAGATGATGGATGGAAATATCGATCTAGGATAGGTATACATGTTGATGCGGGTTTTACTG
ATGCATATACAGAGATGCTTTTTGTTCGCTTGGTTGTGATGATGTGGTGTGGTTGGGCGGTCGTTCATTCGTTCT
AGATCGGAGTAGAATACTGTTTCAAACTACCTGGTGTATTTATTAATTTTGGAACTGTATGTGTGTGTCATACAT
CTTCATAGTTACGAGTTTAAGATGGATGGAAATATCGATCTAGGATAGGTATACATGTTGATGTGGGTTTTACT
GATGCATATACATGATGGCATATGCAGCATCTATTCATATGCTCTAACCTTGAGTACCTATCTATTATAATAAAC
AAGTATGTTTTATAATTATTTTGATCTTGATATACTTGGATGATGGCATATGCAGCAGCTATATGTGGATTTTTTT
AGCCCTGCCTTCATACGCTATTTATTTGCTTGGTACTGTTTCTTTTGTCGATGCTCACCCTGTTGTTTGGTGTTACT
TCTGCAGGGCCGGCCATGGATCCTAAAAAGAAACGTAAGGTCGACAAGAAGTACAGCATTGGTCTGGACATCG
GGACGAATTCCGTTGGCTGGGCCGTGATCACCGATGAGTACAAGGTCCCTTCCAAGAAGTTCAAGGTTCTGGGG
AACACCGATCGGCACAGCATCAAGAAGAACCTCATTGGAGCCCTCCTGTTCGACTCAGGCGAGACCGCCGAAG
CAACAAGGCTCAAGAGAACCGCAAGGAGACGGTATACCAGAAGGAAGAACAGGATCTGCTACCTGCAGGAGA
TCTTCAGCAACGAAATGGCGAAGGTGGACGATTCGTTCTTCCACAGATTGGAAGAGAGTTTCCTCGTCGAGGAA
GACAAGAAGCACGAGAGGCATCCGATCTTCGGCAACATCGTCGACGAGGTTGCCTATCACGAGAAGTACCCCA
CCATCTACCATCTGCGGAAGAAGCTTGTGGACTCGACTGACAAGGCGGACCTTAGGTTGATCTACCTCGCTCTG
GCCCACATGATCAAGTTCAGGGGCCATTTCCTGATCGAGGGGGATCTGAACCCGGACAACAGCGATGTGGACA
AGTTGTTCATCCAGCTCGTCCAAACCTACAACCAGCTCTTTGAGGAGAACCCGATCAACGCTTCAGGCGTCGAC
GCCAAGGCGATCCTGTCTGCACGCCTGTCAAAGTCTCGCCGGCTTGAGAACTTGATCGCTCAACTCCCGGGCGA
GAAGAAGAACGGCTTGTTCGGGAATCTCATTGCACTTTCGTTGGGGCTCACCCCGAACTTCAAGAGCAACTTCG
ATCTCGCTGAGGACGCAAAGCTGCAGCTTTCCAAGGACACTTACGACGATGACCTGGATAACCTGTTGGCCCAA
ATCGGCGATCAGTACGCGGACTTGTTCCTCGCCGCGAAGAACTTGTCGGACGCGATCCTCCTGAGTGACATTCT
CCGCGTGAACACCGAGATCACCAAGGCCCCGCTCTCGGCGAGCATGATCAAGCGGTATGACGAGCACCATCAG
GATCTGACCCTGTTGAAGGCTTTGGTCCGGCAGCAACTCCCAGAGAAGTACAAGGAGATCTTCTTCGATCAGTC
CAAGAACGGCTACGCTGGCTACATCGACGGCGGGGCATCGCAGGAGGAGTTCTACAAGTTCATCAAGCCAATC
CTGGAGAAGATGGATGGCACAGAGGAACTCCTGGTGAAGCTCAACAGGGAGGACCTGTTGCGGAAGCAAAGG
ACTTTCGATAACGGCAGCATCCCTCACCAGATCCATCTCGGGGAGCTGCACGCCATCCTGAGAAGGCAGGAAG
ACTTCTACCCCTTCCTTAAGGATAACCGGGAGAAGATCGAGAAGATCCTGACGTTCAGAATCCCGTACTACGTC
GGACCACTCGCCCGGGGTAATTCCAGATTCGCGTGGATGACCAGAAAGAGCGAGGAAACCATCACCCCTTGGA
ACTTCGAGGAAGTGGTCGATAAGGGCGCTTCCGCACAGAGCTTCATCGAGCGCATGACCAACTTCGACAAGAA
CCTGCCTAACGAGAAGGTCCTTCCCAAGCATTCCCTCCTGTACGAGTACTTCACTGTGTACAACGAACTCACGA
AGGTGAAGTACGTGACCGAGGGAATGCGCAAGCCCGCCTTCCTGAGCGGCGAGCAGAAGAAGGCGATCGTGG
ACCTGTTGTTCAAGACCAATCGGAAGGTCACAGTGAAGCAGCTCAAGGAGGACTACTTCAAGAAGATCGAATG
CTTCGATTCCGTTGAGATCAGCGGCGTGGAAGACAGGTTTAACGCCTCACTGGGGACTTACCACGATCTCCTGA
AGATCATCAAGGATAAGGACTTCTTGGACAACGAGGAGAATGAGGACATCCTCGAAGACATTGTCCTGACTCTT
ACGTTGTTCGAGGATAGGGAGATGATCGAGGAACGCTTGAAGACGTATGCCCATCTCTTCGATGACAAGGTGAT
GAAGCAGCTCAAGAGAAGAAGGTACACCGGATGGGGAAGGCTGTCCCGCAAGCTGATCAACGGCATCAGAGA
CAAGCAATCAGGGAAGACAATCCTTGACTTCTTGAAGTCTGATGGCTTCGCGAACAGGAACTTCATGCAGCTCA
TTCACGATGACTCACTGACCTTCAAGGAGGACATCCAGAAGGCTCAAGTGTCGGGACAAGGTGACAGTCTGCA
CGAGCATATCGCCAACCTTGCGGGATCTCCTGCCATCAAGAAGGGTATCCTGCAGACAGTCAAGGTTGTGGATG
AGCTTGTGAAGGTCATGGGACGGCATAAGCCCGAGAACATCGTTATCGAGATGGCCAGAGAGAATCAGACCAC
CCAGAAGGGTCAGAAGAACTCGAGGGAGCGCATGAAGCGCATCGAGGAAGGCATTAAGGAGCTGGGGAGTCA
GATCCTTAAGGAGCACCCGGTGGAGAACACGCAGTTGCAGAATGAGAAGCTCTATCTGTACTACCTGCAGAAT
GGCAGGGACATGTACGTGGACCAGGAGTTGGACATCAACCGCCTCTCGGATTACGACGTCGATCACATCGTTCC
TCAGTCCTTCCTTAAGGATGACAGCATTGACAACAAGGTTCTCACCAGGTCCGACAAGAACCGCGGGAAGTCCG
ATAACGTGCCCAGCGAGGAAGTCGTGAAGAAGATGAAGAACTACTGGAGGCAACTGCTGAATGCCAAGTTGAT
CACCCAGAGGAAGTTCGACAACCTCACTAAGGCCGAGCGCGGAGGTCTCAGCGAACTGGACAAGGCGGGCTTC
ATCAAGCGGCAACTGGTTGAGACTAGACAGATCACGAAGCACGTCGCGCAGATTCTCGATTCACGCATGAACA
CGAAGTACGATGAGAATGACAAGCTGATCCGGGAAGTGAAGGTCATCACCTTGAAGTCAAAGCTCGTTTCTGA
CTTCAGGAAGGATTTCCAGTTCTACAAGGTGCGCGAGATCAACAACTACCACCATGCTCATGACGCATACCTCA
ACGCTGTGGTCGGAACAGCACTGATCAAGAAGTACCCGAAGCTCGAGTCCGAATTCGTGTACGGTGACTACAA
GGTTTACGATGTGCGCAAGATGATCGCCAAGTCAGAGCAGGAAATTGGCAAGGCCACTGCGAAGTACTTCTTCT
ACTCCAACATCATGAACTTCTTCAAGACTGAGATCACGCTGGCTAATGGCGAAATCCGGAAGAGACCACTGATT
GAGACCAACGGCGAGACAGGGGAAATCGTCTGGGACAAGGGGAGGGATTTCGCCACAGTCCGCAAGGTTCTCT
CTATGCCTCAAGTGAACATCGTCAAGAAGACTGAAGTCCAGACGGGCGGGTTCTCAAAGGAGTCTATCCTGCCC
AAGCGGAACTCGGATAAGCTTATCGCCAGGAAGAAGGACTGGGATCCGAAGAAGTACGGAGGTTTCGACTCAC
CAACGGTGGCTTACTCTGTCCTGGTTGTGGCAAAGGTGGAGAAGGGAAAGTCGAAGAAGCTCAAGTCTGTCAA
GGAGCTGCTGGGTATCACCATTATGGAGAGGTCCAGCTTCGAGAAGAACCCGATCGACTTCCTCGAGGCGAAG
GGCTACAAGGAAGTGAAGAAGGACCTGATCATCAAGCTTCCGAAGTACAGTCTGTTCGAGTTGGAGAACGGCA
GGAAGCGCATGTTGGCTTCCGCAGGAGAGCTCCAGAAGGGTAACGAGCTTGCTTTGCCGTCCAAGTACGTGAA
CTTCCTCTATCTGGCATCCCACTACGAGAAGCTCAAGGGCAGCCCAGAGGATAACGAACAGAAGCAACTGTTTG
TGGAGCAACACAAGCATTACCTTGACGAGATCATCGAACAGATCTCGGAGTTCAGTAAGCGCGTCATCCTCGCC
GACGCGAATTTGGATAAGGTTCTCTCAGCCTACAACAAGCACCGGGACAAGCCTATCAGAGAGCAGGCGGAGA
ACATCATCCATCTCTTCACCCTGACAAACCTTGGGGCTCCCGCTGCCTTCAAGTACTTCGACACTACGATCGACC
GGAAGAGGTACACTTCTACGAAGGAGGTGCTGGATGCAACCCTTATCCACCAATCGATCACTGGCCTCTACGAG
ACGCGGATCGACTTGAGTCAGCTCGGTGGCGATAAGAGACCCGCAGCAACCAAGAAGGCAGGGCAAGCAAAG
AAGAAGAAGTGAGCGATCGCATATGAAGATGAAGATGAAATATTTGGTGTGTCAAATAAAAAGCTTGTGTGCT
TAAGTTTGTGTTTTTTTCTTGGCTTGTTGTGTTATGAATTTGTGGCTTTTTCTAATATTAAATGAATGTAAGATCT
CATTATAATGAATAAACAAATGTTTCTATAATCCATTGTGAATGTTTTGTTGGATCTCTTCTGCAGCATATAACT
ACTGTATGTGCTATGGTATGGACTATGGAATATGATTAAAGATAAGGGTACGTACCGCGATCGCATAACTTCGT
ATAGCATACATTATACGAAGTTATGAATTCGGCAATCAAGAGGTAAAGGATAATAATCCCAGCAGAAACCGTG
ATCAAAGAAACAATTCATTGCAATATAGACTAGGCCATGCATACGCAAATCCAAGCGTGCATCCACTGACTGCA
TAACATAAACCCCCAGGTACTACGACTAAGCCGAACTGATACAAAATGCACTAGCTATTGGATAATCAGATCCT
GGTCCATGCTGCTGTCATTCCATCGCAATCACCCTGGTAGCGACGACAGGATCATTTGCTTCCAGAACCGAACT
GTGCGCCTAGCTCGGCGCGCCTAACTATAACGGTCCTAAGGTAGCGAATCCCAATGGCGCGCCGAGCTTGGCTC
GAGCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCTGAAGCCAGT
TACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGACAGTTAGAAAAACTCAT
CGAGCATCAAATGAAACTGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGGTCTGCGATTCCGACTCGT
CCAACATCAATACAACCTATTAATTTCCCCTCGTCAAAAATAAGGTTATCAAGTGAGAAATCACCATGAGTGAC
GACTGAATCCGGTGAGAATGGCAAAAGTTTATGCATTTCTTTCCAGACTTGTTCAACAGGCCAGCCATTACGCT
CGTCATCAAAATCACTCGCATCAACCAAACCGTTATTCATTCGTGATTGCGCCTGAGCGAGACGAAATACGCGA
TCGCTGTTAAAAGGACAATTACAAACAGGAATCGAATGCAACCGGCGCAGGAACACTGCCAGCGCATCAACAA
TATTTTCACCTGAATCAGGATATTCTTCTAATACCTGGAATGCTGTTTTCCCAGGGATCGCAGTGGTGAGTAACC
ATGCATCATCAGGAGTACGGATAAAATGCTTGATGGTCGGAAGAGGCATAAATTCCGTCAGCCAGTTTAGTCTG
ACCATCTCATCTGTAACATCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTATACCCATATAAATCAGC
ATCCATGTTGGAATTTAATCGCGGCCTAGAGCAAGACGTTTCCCGTTGAATATGGCTCATACTCTTCCTTTTTCA
ATATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGACATTA
ACCTATAAAAATAGGCGTATCACGAGGCCCTTTCGTC
SEQ ID NO: 16 (ScFT4, ScFT8, ScFT10 RNAi-hairpin-long, NPT marker, minimal cassette,
9025 bp)
CAGGGTAATGGCGCGCCGTTGTTTTAGTGTCCGAGGTTTGGTTTGGTGAAGTACATCTGAACTCTGAAGGTTCTT
CGTGACATGGTTAGACGTGGTGTTGCAGTACTGTGATAAAACTTATCAGAATAAGTTTTCGCTTCTAATTTGTTT
TCTTAAATCATGTGTTGATCAGCCTATGAGTTGTTCATATCGTTGCCTTTCGTGTTGAACATTTGTTTTCTTGCCA
TACTAATGAGATTCATCTGCACAAACTGTACGAAGAGAATCAAGTTTAAACACTGCAGGTCAACGGATCACCTA
TCAACATCCCAGCTAAAAACAGTAAAAAGGGGGAAAACGTGGGTGAGTTGAGTCTGTCTTGTGGAAAAAACGT
TTTAGTTTCTCCTGGAATTAACAATAAAAACAGTTGAACAAGATTGACTGTTCCTCCGGGAGGGTTTGGAACAT
CGTTACAGATGTGAGCGAAAGGTGAGGAAACAGAGCGGAGGGCTTGGAGGTGACCTCGGTAGTCGACGCCGG
AGTTGAGCTTGATGACGACACCGTACTGGCGTACCAGGCCTAGTAGTGAACACCGGGCCTGAAGCTGTCGCCGC
CGCTGCTCATCTTGTGGGCTGTGCCCGGTGTCCCTGTTGCGGATTGCGGGTGGCAGCCTGGCAGGTGGGTGCGA
CCCGTTTGGACTCCCTGATCTGGGCCCTTTGTGTCAGTACCGTCTGTACTCCGATGACATGCACACCGTCGTCCA
CAGTCAAGTCCACAATCTCCCCTCTTTTTTTAACGGAATAGTTCAAAATCTCCTTGACGCACGCTATCGTGTACC
AGCGCTCACTGGACACCACGTTTGTAATCCACGCCGACACGTCGGTCCCACGTCGACAGGCCCCACCGTCCGGT
CTGTAGCGTGTACGTATTCGGGCGACGGACGTGTCGTCGTCGTCTTGCGAGTCCCATTCCCATCACCATCTGAGC
CACACATCCTCTGAACAAAAGCAGGGAGGCCTCTACGCACATCCCCCTTTCTCCCACTCCTTGTCCGTGGCACCC
ACCCCAAACCCTCGCGCCGCCTCCGAGACAGCCGCCGCAACCATGGCCACCGCCTGCAGGACCGTCTTCGGTAC
GCGCTCACTCCGCCCTCTGCCTTTGTTACTGCCACGTTTCTCTGAATGCTCTCTTGTGTGGTGATTGCTGAGAGTG
GTTTAGCTGGATCTAGAATTACACTCTGAAATCGTGTTCTGCCTGTGCTGATTACTTGCCGTCCTTTGTAGCAGC
AAAATATAGGGACATGGTAGTACGAAACGAAGATAGAACCTACACAGCAATACGAGAAATGTGTAATTTGGTG
CTTAGCGGTATTTATTTAAGCACATGTTGGTGTTATAGGGCACTTGGATTCAGAAGTTTGCTGTTAATTTAGGCA
CAGGCTTCATACTACATGGGTCAATAGTATAGGGATTCATATTATAGGCGATACTATAATAATTTGTTCGTCTGC
AGAGCTTATTATTTGCCAAAATTAGATATTCCTATTCTGTTTTTGTTTGTGTGCTGTTAAATTGTTAACGCCTGAA
GGAATAAATATAAATGACGAAATTTTGATGTTTATCTCTGCTCCTTTATTGTGACCATAAGTCAAGATCAGATGC
ACTTGTTTTAAATATTGTTGTCTGAAGAAATAAGTACTGACAGTATTTTGATGCATTGATCTGCTTGTTTGTTGTA
ACAAAATTTAAAAATAAAGAGTTTCCTTTTTGTTGCTCTCCTTACCTCCTGATGGTATCTAGTATCTACCAACTG
ACACTATATTGCTTCTCTTTACATACGTATCTTGCTCGATGCCTTCTCCCTAGTGTTGACCAGTGTTACTCACATA
GTCTTTGCTCATTTCATTGTAATGCAGATACCAAGCGGCCCCTGCAGGATGGGGATTGAACAAGATGGATTGCA
CGCAGGTTCTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCGGTGCCCTG
AATGAACTCCAGGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTCCTGTCATCTCAC
CTTGCTCCTGCCGAGAAAGTATCCATCATGGCTGATGCAATGCGGCGGCTGCATACGCTTGATCCGGCTACCTG
CCCATTCGACCACCAAGCGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCTCAAGGCGCGCATGCCCG
ACGGCGAGGATCTCGTCGTGACACATGGCGATGCCTGCTTGCCGAATATCATGGTGGAAAATGGCCGCTTTTCT
GGATTCATCGACTGTGGCCGGCTGGGTGTGGCGGACCGCTATCAGGACATAGCGTTGGCTACCCGTGATATTGC
TGAAGAGCTTGGCGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAGCGATCGCAGAGCCATGGGGGCGGTGAGAGCTCGCGGCG
TCGGATCTCCGGTTTCAGTTTCAGCTACGCGACTTGTGTGTGTGTGTGTGTGTCAGTGCGTGGTTACTCTGAGCT
TCGGTATGTTTGGTGTGTACTGTTCAGTGTTTCATGGTGCTTCCTAGCAGTGTCTGTCGTGTACTTTGCCTGTATG
TTGAGCTCGGCTCAGTATGTTCTGGGAGTGAATAAATAAAAAAAAATTAACCAGATATTGTCGCGTGTACTTAG
TATACTAATCACCTGCTCCTGCTCTTGCTCTGTTTTATCACCTTTCGGGCTTGACTGTCTTTGTGGAGGCCTCTCA
GTCTGATCTGGAACTCTACCTAAGTTTTACTAGTCCCGGGAAGCTTCTGCAGTGCAGCGTGACCCGGTCGTGCCC
CTCACTAGAGATAATGAGCATTGCATGTCTAAGTTATAAAAAATTACCACATATTTTTTTTGTCACACTTGTTTG
AAGTGCAGTTTATCTATCTTTATACATATATTTAAACTTTACTCTACGAATAATATAATCTATAGTACTACAATA
ATATCAGTGTTTTAGAGAATCATATAAATGAACAGTTAGACATGGTCTAAAGGACAATTGAGTATTTTGACAAC
AGGACTCTACAGTTTTATCTTTTTAGTGTGCATGTGTTCTCCTTTTTTTTTGCAAATAGCTTCACCTATATAATAC
TTCATCCATTTTATTAGTACATCCATTTAGGGTTTAGGGTTAATGGTTTTTATAGACTAATTTTTTTAGTACATCT
ATTTTATTCTATTTTAGCCTCTAAATTAAGAAAACTAAAACTCTATTTTAGTTTTTTTATTTAATAATTTAGATAT
AAAATAGAATAAAATAAAGTGACTAAAAATTAAACAAATACCCTTTAAGAAATTAAAAAAACTAAGGAAACAT
TTTTCTTGTTTCGAGTAGATAATGCCAGCCTGTTAAACGCCGTCGACGAGTCTAACGGACACCAACCAGCGAAC
CAGCAGCGTCGCGTCGGGCCAAGCGAAGCAGACGGCACGGCATCTCTGTCGCTGCCTCTGGACCCCTCTCGAGA
GTTCCGCTCCACCGTTGGACTTGCTCCGCTGTCGGCATCCAGAAATTGCGTGGCGGAGCGGCAGACGTGAGCCG
GCACGGCAGGCGGCCTCCTCCTCCTCTCACGGCACGGCAGCTACGGGGGATTCCTTTCCCACCGCTCCTTCGCTT
TCCCTTCCTCGCCCGCCGTAATAAATAGACACCCCCTCCACACCCTCTTTCCCCAACCTCGTGTTGTTCGGAGCG
CACACACACACAACCAGATCTCCCCCAAATCCACCCGTCGGCACCTCCGCTTCAAGGTACGCCGCTCGTCCTCC
CCCCCCCCCCCTCTCTACCTTCTCTAGATCGGCGTTCCGGTCCATGGTTAGGGCCCGGTAGTTCTACTTCTGTTCA
TGTTTGTGTTAGATCCGTGTTTGTGTTAGATCCGTGCTGCTAGCGTTCGTACACGGATGCGACCTGTACGTCAGA
CACGTTCTGATTGCTAACTTGCCAGTGTTTCTCTTTGGGGAATCCTGGGATGGCTCTAGCCGTTCCGCAGACGGG
ATCGATTTCATGATTTTTTTTGTTTCGTTGCATAGGGTTTGGTTTGCCCTTTTCCTTTATTTCAATATATGCCGTGC
ACTTGTTTGTCGGGTCATCTTTTCATGCTTTTTTTTGTCTTGGTTGTGATGATGTGGTCTGGTTGGGCGGTCGTTC
TAGATCGGAGTAGAATTCTGTTTCAAACTACCTGGTGGATTTATTAATTTTGGATCTGTATGTGTGTGCCATACA
TATTCATAGTTACGAATTGAAGATGATGGATGGAAATATCGATCTAGGATAGGTATACATGTTGATGCGGGTTT
TACTGATGCATATACAGAGATGCTTTTTGTTCGCTTGGTTGTGATGATGTGGTGTGGTTGGGCGGTCGTTCATTC
GTTCTAGATCGGAGTAGAATACTGTTTCAAACTACCTGGTGTATTTATTAATTTTGGAACTGTATGTGTGTGTCA
TACATCTTCATAGTTACGAGTTTAAGATGGATGGAAATATCGATCTAGGATAGGTATACATGTTGATGTGGGTT
TTACTGATGCATATACATGATGGCATATGCAGCATCTATTCATATGCTCTAACCTTGAGTACCTATCTATTATAA
TAAACAAGTATGTTTTATAATTATTTTGATCTTGATATACTTGGATGATGGCATATGCAGCAGCTATATGTGGAT
TTTTTTAGCCCTGCCTTCATACGCTATTTATTTGCTTGGTACTGTTTCTTTTGTCGATGCTCACCCTGTTGTTTGGT
GTTACTTCTCTAGAGCGGCCGCATTTAAATCACTCTGTGGTCTCAAATGGCTGGTGGTTGGTAGGGTGGTGGGC
GACGTGCTGGACCCCTTCGTCCGGACCACCAACCTCAGGGTCAGCTACGGCACCAGGACCGTATCCAACGGCTG
CGAGCTCAAGCCGTCCATGGTGGTGAACCAGCCCAGGGTCGAGGTCGGGGGACCCGACATGAGGACCTTCTAC
ACCCTCGTGATGGTCATGTGATACGAGATGTGTTGGATTCATTTACACCAACTATACCCCTCAGAATAACCTAC
AACAATAGGCTACTTCTAGCAGGTGTTGAGCTGAAGCCATCCGCAGTGGTGAATAAGCCAAGAGTTGATGTTGG
GGGCACCGACCTCAGGGTGTTCTTTACACTGGTAGGTATTAGTTGATCCAGATGCCCCAAGCCCAAGCAATCCA
TCACTGAGGGGTCAGCAACCGATCCTTTGGTCATGGCTCATGTCATACAGGATGTGTTGGATCCCTTTACAACA
ACCATTCCACTAAGAATAACATACAACAATAGGCTACTTCTGGCAAGTGCTGAGCTAAAGCCATCTGCGGTTGC
AAGTAAACCACGAGTCGATGTCGGTGGCAATGACATGAGGGCTTTCTACACCCTGGGTTTGAATCAGATTCAGA
TTTCATTGCATCACAGAGATCCATCTTTACTCTACCGCTTGCTCTAACTTAACTTGTAATTGTTTTTTATCATGCA
GCCAGGGTGTAGAAAGCCCTCATGTCATTGCCACCGACATCGACTCGTGGTTTACTTGCAACCGCAGATGGCTT
TAGCTCAGCACTTGCCAGAAGTAGCCTATTGTTGTATGTTATTCTTAGTGGAATGGTTGTTGTAAAGGGATCCAA
CACATCCTGTATGACATGAGCCATGACCAAAGGATCGGTTGCTGACCCCTCAGTGATGGATTGCTTGGGCTTGG
GGCATCTGGATCAACTAATACCTACCAGTGTAAAGAACACCCTGAGGTCGGTGCCCCCAACATCAACTCTTGGC
TTATTCACCACTGCGGATGGCTTCAGCTCAACACCTGCTAGAAGTAGCCTATTGTTGTAGGTTATTCTGAGGGGT
ATAGTTGGTGTAAATGAATCCAACACATCTCGTATCACATGACCATCACGAGGGTGTAGAAGGTCCTCATGTCG
GGTCCCCCGACCTCGACCCTGGGCTGGTTCACCACCATGGACGGCTTGAGCTCGCAGCCGTTGGATACGGTCCT
GGTGCCGTAGCTGACCCTGAGGTTGGTGGTCCGGACGAAGGGGTCCAGCACGTCGCCCACCACCCTACCAACC
ACCAGCGCTTTGAGACCACGAAGTGTTCGAATTAATTAAGGGGACACCGTTGTCGGAGCAGTGCCAGTGAGCT
GGGTGGCAACCTGCGGGACTCTGGTGAGGAAGAGCAAATGGTAGAAAGCAGTAGCTGTGCTTCTGAACATTGT
GTGTGTTTGTTTCCCTTGTTACCGCCAATCATCCTACCTGATGTGTCGTCTGGGGTTTCGTTTGAACCATGCTGGG
TTTGGAGCTTTGTGATGCAAAGCAGAATGTCCTGATGCTTTCTTTCTCCATAGAATAAATCTCTCCTGTTACCTA
AAGGCCGGCCGATATCCATTCAAGAGGAGAGCTCATTTCCTCTCCTCTCATCACCTTTGCCTAAAAACAGTATG
AAAGTCAAGCTAAAGACTTTAAACTAGCTCACTTGGGAGGAAGTCCCATGTCTATTCTTGTATATACTTTTTTCC
CTTGTTATTTTTGATGTTTTGTGTAAGTATTTCAGGTTTTATAGTCCACGGAAGTCAACGATCGAGTGATATAAA
TCGAGTCAAAGAATCTTAGCCGTGCGATAGAGTGACTTAAGGCGAAGCATCCAGCTAATCCGAGTCCCACACA
ACCTATTCCTTGGAGCTCTAAACGGGAGAGTATGGACGACTTCGTCAACTCCTATTTCACCAGATCACTAAGGT
AACACACCACCCCCATTGTATATACCGTTATCTTTTATTTTTCTTTGTTTGTTGTTTGTTTTAGTCTTGAATTCTCT
CTCAGATTTTTAATACACAAGGGACTGTGTAATTTAAGTGTGGGGGAGGGTTCAAGAAATATTTGACTTTATGT
TCTCTTTTCTTATTTCAAATTTTGCATCATCTAAGGCATAAAAAACCATAAAAATTTAAAAACATTTATGAAAAT
GATTTCACCAAAACAGAGTGTCATGTAGATTGCATTGCATTTTAGGATTGTATATAGAGTACTTGCATTTAGGAT
TTCTCATTTGCATAGGGGATAATGATGATTTCGTACCGTTAAGCATGTTTTGATTCAATGAAGTAAGTTCAATGC
CCAGGTTGTTAGTTCTCTGTTGCATAACCGATTGTTTCTCTGAAGTAAGACTGCACCCTGCTTTGAATTTACAAC
TTGACTATACTTCTTTGATTCAAAACTCGCTTATCTGATGCCATTCCCTATCTATTAGAACCTGAACTGAATTTGC
AATTATCATGTCTATGCATTGTTTGTGAACTCATGGCTACCATATACATACTTGTATTATCTTATTCACTTCACCA
CCCTCTGTTAATCTAAGTAGCTGACTCTCATTTTTAGAGCAGTTTCCCATACCCTTAGCCTAGCCTTATTTCAAGC
CAAAGATCATTTGTGTGTGATTGTGAGGTCTTTTTCGATTGAGCTTGGTATAACGTGTCAGGTATGAGCCGACAA
GAGCAGTCTTTCATGTAGACCTAGCCCACGCTTTTTGGGTTAGCTAGGACTAGGTGGAGACTTGTATTTGGGATT
GGGGTTGTGTATGGATTGAATGGGAAAGAGAATGAGAATGAAAAGAAAAGAAAAAAAGAGAGTAAGAGTTTA
GAGTTCTTAAGGGGGATACAAAAAGTGTTAGTCTAGTAAAGGGTTTGGAATTCAAAGAAGTTAGGCATTGTGAT
TTAAAGAAAAGGGTAAAACGACCTAAGATTAGAAGAAAAGCCTTAAGTTTGTCAAAGTATAAAACCAAACCCA
CTAGACTCTTGAATCGTTTTAAAGAAGAACCCCTTAGAACTTGAACCAAAAAGAGAAAGAATGAGAAAAAGGG
TAGAGTTAGGACATCTATGGGACTGGAACCATCTTATGAACTATCATACTATATCATTGGGTAGATGGGAGTCT
GTTCTTGTTTGTGTCCATTGGCTTTTACCTTTAGCATTCTGCTGACGCTCAATCTTTTCTTTGAGAGTCCCCTGTTA
CTCGACCAAATCATTAAAGGGACCATTTTTGTCTCTTAGCCTAAGCCTGAAACCAAGTGAGTTCATAACCATTG
CATTGCTTGATCCATTGTTCGTGCTTAGTGAATGTTAAAAAGAATTGGTTGATGTGAATGCTTGAATAGATTAAG
GCAGGTTGTTAGGTTGCTTTGTGACGAGTATGGCTAAATTTTTAAGTAAGGGTCTATCATTTTGCAACCTATAAT
TAGCTACCTGGACATTGAGCTTACCCGTTCTATATGCATGCTACGGGTTTTGAATCCCCACCTTCAAACCTCTCC
TTCAACTTAAGATTTTTTGTTTGCTTGAGGGCAAGCAAGAGATAAGTTTGGGGGAGTTGATAACACTGTAATTT
ACTCATTTTGGGCATGTATTTATCATCATTTAGCTTAGTGATCACTCATTGTTTCATATCATTTTGCATCACATTT
AGCTTATTTTGCACACTTAGGCATTTAGGGTCATCTTGCATACATATTGCATGTTATGGTGTGTTTTCAGGTGCA
AAATAAGTTTACAAGGAGTTTACTCGACCCTCTACCCTATTGCCCCAGAGGGGAACCAGAGGAGCAGCTACTCG
ACCAATTATTCATTGCCTCAGAGGGGAAGCAGCAGAGCAGCTACTCGACCAGTTATTCGGCGCGCCTAGGGATA
A
SEQ ID NO: 17 (ScFT4, ScFT8, ScFT10 RNAi-hairpin-long, ALS marker, minimal cassette,
10153 bp)
CAGGGTAATGGCGCGCCGTTGTTTTAGTGTCCGAGGTTTGGTTTGGTGAAGTACATCTGAACTCTGAAGGTTCTT
CGTGACATGGTTAGACGTGGTGTTGCAGTACTGTGATAAAACTTATCAGAATAAGTTTTCGCTTCTAATTTGTTT
TCTTAAATCATGTGTTGATCAGCCTATGAGTTGTTCATATCGTTGCCTTTCGTGTTGAACATTTGTTTTCTTGCCA
TACTAATGAGATTCATCTGCACAAACTGTACGAAGAGAATCAAGTTTAAACACTGCAGGTCAACGGATCACCTA
TCAACATCCCAGCTAAAAACAGTAAAAAGGGGGAAAACGTGGGTGAGTTGAGTCTGTCTTGTGGAAAAAACGT
TTTAGTTTCTCCTGGAATTAACAATAAAAACAGTTGAACAAGATTGACTGTTCCTCCGGGAGGGTTTGGAACAT
CGTTACAGATGTGAGCGAAAGGTGAGGAAACAGAGCGGAGGGCTTGGAGGTGACCTCGGTAGTCGACGCCGG
AGTTGAGCTTGATGACGACACCGTACTGGCGTACCAGGCCTAGTAGTGAACACCGGGCCTGAAGCTGTCGCCGC
CGCTGCTCATCTTGTGGGCTGTGCCCGGTGTCCCTGTTGCGGATTGCGGGTGGCAGCCTGGCAGGTGGGTGCGA
CCCGTTTGGACTCCCTGATCTGGGCCCTTTGTGTCAGTACCGTCTGTACTCCGATGACATGCACACCGTCGTCCA
CAGTCAAGTCCACAATCTCCCCTCTTTTTTTAACGGAATAGTTCAAAATCTCCTTGACGCACGCTATCGTGTACC
AGCGCTCACTGGACACCACGTTTGTAATCCACGCCGACACGTCGGTCCCACGTCGACAGGCCCCACCGTCCGGT
CTGTAGCGTGTACGTATTCGGGCGACGGACGTGTCGTCGTCGTCTTGCGAGTCCCATTCCCATCACCATCTGAGC
CACACATCCTCTGAACAAAAGCAGGGAGGCCTCTACGCACATCCCCCTTTCTCCCACTCCTTGTCCGTGGCACCC
ACCCCAAACCCTCGCGCCGCCTCCGAGACAGCCGCCGCAACCATGGCCACCGCCTGCAGGACCGTCTTCGGTAC
GCGCTCACTCCGCCCTCTGCCTTTGTTACTGCCACGTTTCTCTGAATGCTCTCTTGTGTGGTGATTGCTGAGAGTG
GTTTAGCTGGATCTAGAATTACACTCTGAAATCGTGTTCTGCCTGTGCTGATTACTTGCCGTCCTTTGTAGCAGC
AAAATATAGGGACATGGTAGTACGAAACGAAGATAGAACCTACACAGCAATACGAGAAATGTGTAATTTGGTG
CTTAGCGGTATTTATTTAAGCACATGTTGGTGTTATAGGGCACTTGGATTCAGAAGTTTGCTGTTAATTTAGGCA
CAGGCTTCATACTACATGGGTCAATAGTATAGGGATTCATATTATAGGCGATACTATAATAATTTGTTCGTCTGC
AGAGCTTATTATTTGCCAAAATTAGATATTCCTATTCTGTTTTTGTTTGTGTGCTGTTAAATTGTTAACGCCTGAA
GGAATAAATATAAATGACGAAATTTTGATGTTTATCTCTGCTCCTTTATTGTGACCATAAGTCAAGATCAGATGC
ACTTGTTTTAAATATTGTTGTCTGAAGAAATAAGTACTGACAGTATTTTGATGCATTGATCTGCTTGTTTGTTGTA
ACAAAATTTAAAAATAAAGAGTTTCCTTTTTGTTGCTCTCCTTACCTCCTGATGGTATCTAGTATCTACCAACTG
ACACTATATTGCTTCTCTTTACATACGTATCTTGCTCGATGCCTTCTCCCTAGTGTTGACCAGTGTTACTCACATA
GTCTTTGCTCATTTCATTGTAATGCAGATACCAAGCGGCCCCTGCAGGATGGCCACCACCGCCGCCGCCGCTGC
CGCCGCGCTAGCCGGCGCCACTACCGCTGCGCCCAAGGCGAGGCGCCGGGCGCACCTCCTGGCCGCACGGCGC
GCCCTCGCCGCGCCCATCAGGTGCTCAGCGGCGCCACCCGCCACGCTGACGGTGACGGCTCCCCCGGCCACCCC
GCTCCGGCCGTGGGGCCCCACCGATCCCCGCAAGGGCGCCGACATCCTCGTCGAGGCTCTTGAGCGCTGCGGCG
TCCGCGACGTCTTCGCCTACCCCGGCGGCGCGTCCATGGAGATCCACCAGGCACTCACCCGTTCCCCCGTCATC
GCCAACCACCTCTTCCGCCACGAGCAAGGGGAGGCCTTCGCCGCCTCTGGCTTCGCGCGCTCCTCGGGCCGCGT
CGGCGTCTGCGTCGCCACCTCCGGCCCCGGCGCCACCAACCTAGTCTCCGCGCTCGCCGACGCGCTGCTCGACT
CCGTCCCCATGGTCGCCATCACGGGACAGGTTCCGCGGCGCATGATTGGCACCGACGCCTTCCAGGAGACGCCC
ATCGTCGAGGTCACCCGCTCCATCACCAAACATAACTACCTGGTCCTCGACGTCGACGACATCCCCCGCGTCGT
GCAGGAGGCTTTCTTCCTCGCCTCCTCCGGTCGCCCGGGACCGGTGCTTGTCGACATCCCCAAGGACATCCAGC
AGCAGATGGCCGTGCCGGTCTGGGACACGCCCATGAGTCTGCCTGGGTACATTGCGCGCCTTCCCAAGCCTCCT
GCGACTGAATTGCTTGAGCAGGTGCTGCGTCTTGTTGGTGAATCAAGGCGCCCTGTTCTTTATGTTGGTGGTGGC
TGCGCAGCATCTGGCGAGGAGTTGCGCCGCTTTGTGGAGATGACTGGAATCCCAGTCACAACTACTCTTATGGG
CCTTGGCAATTTCCCTGGCGACGACCCACTGTCTCTGCGCATGCTTGGTATGCATGGCACGGTGTATGCAAATTA
TGCAGTGGATAAGGCGGATCTGTTGCTTGCATTTGGTGTGCGGTTTGATGATCGTGTGACAGGGAAGATTGAGG
CTTTTGCAAGCAGGGCTAAGATTGTGCACATTGATATTGATCCCGCTGAGATTGGCAAGAACAAGCAGCCACAT
GTGTCCATCTGTGCAGACGTTAAGCTTGCTTTGCAGGGCATGAATGCTCTTCTGGAAGGAAGCACATCAAAGAA
GAGCTTTGACTTTGGCTCATGGCAAGCTGAGTTGGATCAGCAGAAGAGAGAGTTCCCCCTTGGGTATAAAACTT
TTGATGACGAGATCCAGCCACAATATGCTATTCAGGTTCTTGATGAGCTGACAAAAGGGGAGGCCATCATTGCC
ACAGGTGTTGGGCAGCACCAGATGTGGGCGGCACAGTACTACACTTACAAGCGGCCAAGGCAGTGGTTGTCTT
CAGCTGGTCTTGGGGCTATGGGATTTGGTTTGCCGGCTGCTGCTGGCGCTGCTGTGGCCAACCCAGGTATCACT
GTTGTTGACATCGACGGAGATGGTAGCTTCCTCATGAACATTCAGGAGCTAGCTATGATCCGAATTGAGAACCT
CCCAGTGAAGGTCTTTGTGCTAAACAACCAGCACCTGGGGATGGTGGTGCAGTTGGAGGACAGGTTCTATAAG
GCCAATAGAGCACACACATACTTGGGAAACCCAGAGAATGAAAGTGAGATATATCCAGATTTCGTGACAATTG
CCAAAGGGTTCAACATTCCAGCAGTCCGTGTGACAAAGAAGAGCGAAGTCCATGCAGCAATCAAGAAGATGCT
TGAGACTCCAGGGCCATACCTCTTGGATATAATCGTCCCGCACCAGGAGCATGTGTTGCCTATGATCCCTAGTG
GTGGGGCTTTCAAGGATATGATCCTGGATGGTGATGGCAGGACTGTGTATTGAGCGATCGCAGAGCCATGGGG
GCGGTGAGAGCTCGCGGCGTCGGATCTCCGGTTTCAGTTTCAGCTACGCGACTTGTGTGTGTGTGTGTGTGTCAG
TGCGTGGTTACTCTGAGCTTCGGTATGTTTGGTGTGTACTGTTCAGTGTTTCATGGTGCTTCCTAGCAGTGTCTGT
CGTGTACTTTGCCTGTATGTTGAGCTCGGCTCAGTATGTTCTGGGAGTGAATAAATAAAAAAAAATTAACCAGA
TATTGTCGCGTGTACTTAGTATACTAATCACCTGCTCCTGCTCTTGCTCTGTTTTATCACCTTTCGGGCTTGACTG
TCTTTGTGGAGGCCTCTCAGTCTGATCTGGAACTCTACCTAAGTTTTACTAGTCCCGGGAAGCTTCTGCAGTGCA
GCGTGACCCGGTCGTGCCCCTCACTAGAGATAATGAGCATTGCATGTCTAAGTTATAAAAAATTACCACATATT
TTTTTTGTCACACTTGTTTGAAGTGCAGTTTATCTATCTTTATACATATATTTAAACTTTACTCTACGAATAATAT
AATCTATAGTACTACAATAATATCAGTGTTTTAGAGAATCATATAAATGAACAGTTAGACATGGTCTAAAGGAC
AATTGAGTATTTTGACAACAGGACTCTACAGTTTTATCTTTTTAGTGTGCATGTGTTCTCCTTTTTTTTTGCAAAT
AGCTTCACCTATATAATACTTCATCCATTTTATTAGTACATCCATTTAGGGTTTAGGGTTAATGGTTTTTATAGAC
TAATTTTTTTAGTACATCTATTTTATTCTATTTTAGCCTCTAAATTAAGAAAACTAAAACTCTATTTTAGTTTTTTT
ATTTAATAATTTAGATATAAAATAGAATAAAATAAAGTGACTAAAAATTAAACAAATACCCTTTAAGAAATTAA
AAAAACTAAGGAAACATTTTTCTTGTTTCGAGTAGATAATGCCAGCCTGTTAAACGCCGTCGACGAGTCTAACG
GACACCAACCAGCGAACCAGCAGCGTCGCGTCGGGCCAAGCGAAGCAGACGGCACGGCATCTCTGTCGCTGCC
TCTGGACCCCTCTCGAGAGTTCCGCTCCACCGTTGGACTTGCTCCGCTGTCGGCATCCAGAAATTGCGTGGCGG
AGCGGCAGACGTGAGCCGGCACGGCAGGCGGCCTCCTCCTCCTCTCACGGCACGGCAGCTACGGGGGATTCCTT
TCCCACCGCTCCTTCGCTTTCCCTTCCTCGCCCGCCGTAATAAATAGACACCCCCTCCACACCCTCTTTCCCCAAC
CTCGTGTTGTTCGGAGCGCACACACACACAACCAGATCTCCCCCAAATCCACCCGTCGGCACCTCCGCTTCAAG
GTACGCCGCTCGTCCTCCCCCCCCCCCCCTCTCTACCTTCTCTAGATCGGCGTTCCGGTCCATGGTTAGGGCCCG
GTAGTTCTACTTCTGTTCATGTTTGTGTTAGATCCGTGTTTGTGTTAGATCCGTGCTGCTAGCGTTCGTACACGGA
TGCGACCTGTACGTCAGACACGTTCTGATTGCTAACTTGCCAGTGTTTCTCTTTGGGGAATCCTGGGATGGCTCT
AGCCGTTCCGCAGACGGGATCGATTTCATGATTTTTTTTGTTTCGTTGCATAGGGTTTGGTTTGCCCTTTTCCTTT
ATTTCAATATATGCCGTGCACTTGTTTGTCGGGTCATCTTTTCATGCTTTTTTTTGTCTTGGTTGTGATGATGTGG
TCTGGTTGGGCGGTCGTTCTAGATCGGAGTAGAATTCTGTTTCAAACTACCTGGTGGATTTATTAATTTTGGATC
TGTATGTGTGTGCCATACATATTCATAGTTACGAATTGAAGATGATGGATGGAAATATCGATCTAGGATAGGTA
TACATGTTGATGCGGGTTTTACTGATGCATATACAGAGATGCTTTTTGTTCGCTTGGTTGTGATGATGTGGTGTG
GTTGGGCGGTCGTTCATTCGTTCTAGATCGGAGTAGAATACTGTTTCAAACTACCTGGTGTATTTATTAATTTTG
GAACTGTATGTGTGTGTCATACATCTTCATAGTTACGAGTTTAAGATGGATGGAAATATCGATCTAGGATAGGT
ATACATGTTGATGTGGGTTTTACTGATGCATATACATGATGGCATATGCAGCATCTATTCATATGCTCTAACCTT
GAGTACCTATCTATTATAATAAACAAGTATGTTTTATAATTATTTTGATCTTGATATACTTGGATGATGGCATAT
GCAGCAGCTATATGTGGATTTTTTTAGCCCTGCCTTCATACGCTATTTATTTGCTTGGTACTGTTTCTTTTGTCGA
TGCTCACCCTGTTGTTTGGTGTTACTTCTCTAGAGCGGCCGCATTTAAATCACTCTGTGGTCTCAAATGGCTGGT
GGTTGGTAGGGTGGTGGGCGACGTGCTGGACCCCTTCGTCCGGACCACCAACCTCAGGGTCAGCTACGGCACCA
GGACCGTATCCAACGGCTGCGAGCTCAAGCCGTCCATGGTGGTGAACCAGCCCAGGGTCGAGGTCGGGGGACC
CGACATGAGGACCTTCTACACCCTCGTGATGGTCATGTGATACGAGATGTGTTGGATTCATTTACACCAACTAT
ACCCCTCAGAATAACCTACAACAATAGGCTACTTCTAGCAGGTGTTGAGCTGAAGCCATCCGCAGTGGTGAATA
AGCCAAGAGTTGATGTTGGGGGCACCGACCTCAGGGTGTTCTTTACACTGGTAGGTATTAGTTGATCCAGATGC
CCCAAGCCCAAGCAATCCATCACTGAGGGGTCAGCAACCGATCCTTTGGTCATGGCTCATGTCATACAGGATGT
GTTGGATCCCTTTACAACAACCATTCCACTAAGAATAACATACAACAATAGGCTACTTCTGGCAAGTGCTGAGC
TAAAGCCATCTGCGGTTGCAAGTAAACCACGAGTCGATGTCGGTGGCAATGACATGAGGGCTTTCTACACCCTG
GGTTTGAATCAGATTCAGATTTCATTGCATCACAGAGATCCATCTTTACTCTACCGCTTGCTCTAACTTAACTTG
TAATTGTTTTTTATCATGCAGCCAGGGTGTAGAAAGCCCTCATGTCATTGCCACCGACATCGACTCGTGGTTTAC
TTGCAACCGCAGATGGCTTTAGCTCAGCACTTGCCAGAAGTAGCCTATTGTTGTATGTTATTCTTAGTGGAATGG
TTGTTGTAAAGGGATCCAACACATCCTGTATGACATGAGCCATGACCAAAGGATCGGTTGCTGACCCCTCAGTG
ATGGATTGCTTGGGCTTGGGGCATCTGGATCAACTAATACCTACCAGTGTAAAGAACACCCTGAGGTCGGTGCC
CCCAACATCAACTCTTGGCTTATTCACCACTGCGGATGGCTTCAGCTCAACACCTGCTAGAAGTAGCCTATTGTT
GTAGGTTATTCTGAGGGGTATAGTTGGTGTAAATGAATCCAACACATCTCGTATCACATGACCATCACGAGGGT
GTAGAAGGTCCTCATGTCGGGTCCCCCGACCTCGACCCTGGGCTGGTTCACCACCATGGACGGCTTGAGCTCGC
AGCCGTTGGATACGGTCCTGGTGCCGTAGCTGACCCTGAGGTTGGTGGTCCGGACGAAGGGGTCCAGCACGTCG
CCCACCACCCTACCAACCACCAGCGCTTTGAGACCACGAAGTGTTCGAATTAATTAAGGGGACACCGTTGTCGG
AGCAGTGCCAGTGAGCTGGGTGGCAACCTGCGGGACTCTGGTGAGGAAGAGCAAATGGTAGAAAGCAGTAGCT
GTGCTTCTGAACATTGTGTGTGTTTGTTTCCCTTGTTACCGCCAATCATCCTACCTGATGTGTCGTCTGGGGTTTC
GTTTGAACCATGCTGGGTTTGGAGCTTTGTGATGCAAAGCAGAATGTCCTGATGCTTTCTTTCTCCATAGAATAA
ATCTCTCCTGTTACCTAAAGGCCGGCCGATATCCATTCAAGAGGAGAGCTCATTTCCTCTCCTCTCATCACCTTT
GCCTAAAAACAGTATGAAAGTCAAGCTAAAGACTTTAAACTAGCTCACTTGGGAGGAAGTCCCATGTCTATTCT
TGTATATACTTTTTTCCCTTGTTATTTTTGATGTTTTGTGTAAGTATTTCAGGTTTTATAGTCCACGGAAGTCAAC
GATCGAGTGATATAAATCGAGTCAAAGAATCTTAGCCGTGCGATAGAGTGACTTAAGGCGAAGCATCCAGCTA
ATCCGAGTCCCACACAACCTATTCCTTGGAGCTCTAAACGGGAGAGTATGGACGACTTCGTCAACTCCTATTTC
ACCAGATCACTAAGGTAACACACCACCCCCATTGTATATACCGTTATCTTTTATTTTTCTTTGTTTGTTGTTTGTT
TTAGTCTTGAATTCTCTCTCAGATTTTTAATACACAAGGGACTGTGTAATTTAAGTGTGGGGGAGGGTTCAAGA
AATATTTGACTTTATGTTCTCTTTTCTTATTTCAAATTTTGCATCATCTAAGGCATAAAAAACCATAAAAATTTAA
AAACATTTATGAAAATGATTTCACCAAAACAGAGTGTCATGTAGATTGCATTGCATTTTAGGATTGTATATAGA
GTACTTGCATTTAGGATTTCTCATTTGCATAGGGGATAATGATGATTTCGTACCGTTAAGCATGTTTTGATTCAA
TGAAGTAAGTTCAATGCCCAGGTTGTTAGTTCTCTGTTGCATAACCGATTGTTTCTCTGAAGTAAGACTGCACCC
TGCTTTGAATTTACAACTTGACTATACTTCTTTGATTCAAAACTCGCTTATCTGATGCCATTCCCTATCTATTAGA
ACCTGAACTGAATTTGCAATTATCATGTCTATGCATTGTTTGTGAACTCATGGCTACCATATACATACTTGTATT
ATCTTATTCACTTCACCACCCTCTGTTAATCTAAGTAGCTGACTCTCATTTTTAGAGCAGTTTCCCATACCCTTAG
CCTAGCCTTATTTCAAGCCAAAGATCATTTGTGTGTGATTGTGAGGTCTTTTTCGATTGAGCTTGGTATAACGTG
TCAGGTATGAGCCGACAAGAGCAGTCTTTCATGTAGACCTAGCCCACGCTTTTTGGGTTAGCTAGGACTAGGTG
GAGACTTGTATTTGGGATTGGGGTTGTGTATGGATTGAATGGGAAAGAGAATGAGAATGAAAAGAAAAGAAAA
AAAGAGAGTAAGAGTTTAGAGTTCTTAAGGGGGATACAAAAAGTGTTAGTCTAGTAAAGGGTTTGGAATTCAA
AGAAGTTAGGCATTGTGATTTAAAGAAAAGGGTAAAACGACCTAAGATTAGAAGAAAAGCCTTAAGTTTGTCA
AAGTATAAAACCAAACCCACTAGACTCTTGAATCGTTTTAAAGAAGAACCCCTTAGAACTTGAACCAAAAAGA
GAAAGAATGAGAAAAAGGGTAGAGTTAGGACATCTATGGGACTGGAACCATCTTATGAACTATCATACTATAT
CATTGGGTAGATGGGAGTCTGTTCTTGTTTGTGTCCATTGGCTTTTACCTTTAGCATTCTGCTGACGCTCAATCTT
TTCTTTGAGAGTCCCCTGTTACTCGACCAAATCATTAAAGGGACCATTTTTGTCTCTTAGCCTAAGCCTGAAACC
AAGTGAGTTCATAACCATTGCATTGCTTGATCCATTGTTCGTGCTTAGTGAATGTTAAAAAGAATTGGTTGATGT
GAATGCTTGAATAGATTAAGGCAGGTTGTTAGGTTGCTTTGTGACGAGTATGGCTAAATTTTTAAGTAAGGGTC
TATCATTTTGCAACCTATAATTAGCTACCTGGACATTGAGCTTACCCGTTCTATATGCATGCTACGGGTTTTGAA
TCCCCACCTTCAAACCTCTCCTTCAACTTAAGATTTTTTGTTTGCTTGAGGGCAAGCAAGAGATAAGTTTGGGGG
AGTTGATAACACTGTAATTTACTCATTTTGGGCATGTATTTATCATCATTTAGCTTAGTGATCACTCATTGTTTCA
TATCATTTTGCATCACATTTAGCTTATTTTGCACACTTAGGCATTTAGGGTCATCTTGCATACATATTGCATGTTA
TGGTGTGTTTTCAGGTGCAAAATAAGTTTACAAGGAGTTTACTCGACCCTCTACCCTATTGCCCCAGAGGGGAA
CCAGAGGAGCAGCTACTCGACCAATTATTCATTGCCTCAGAGGGGAAGCAGCAGAGCAGCTACTCGACCAGTT
ATTCGGCGCGCCTAGGGATAA
SEQ ID NO: 18 (ScFT4, ScFT8, ScFT10 RNAi-hairpin-short, NPT marker, minimal cassette,
8453 bp)
CAGGGTAATGGCGCGCCGTTGTTTTAGTGTCCGAGGTTTGGTTTGGTGAAGTACATCTGAACTCTGAAGGTTCTT
CGTGACATGGTTAGACGTGGTGTTGCAGTACTGTGATAAAACTTATCAGAATAAGTTTTCGCTTCTAATTTGTTT
TCTTAAATCATGTGTTGATCAGCCTATGAGTTGTTCATATCGTTGCCTTTCGTGTTGAACATTTGTTTTCTTGCCA
TACTAATGAGATTCATCTGCACAAACTGTACGAAGAGAATCAAGTTTAAACACTGCAGGTCAACGGATCACCTA
TCAACATCCCAGCTAAAAACAGTAAAAAGGGGGAAAACGTGGGTGAGTTGAGTCTGTCTTGTGGAAAAAACGT
TTTAGTTTCTCCTGGAATTAACAATAAAAACAGTTGAACAAGATTGACTGTTCCTCCGGGAGGGTTTGGAACAT
CGTTACAGATGTGAGCGAAAGGTGAGGAAACAGAGCGGAGGGCTTGGAGGTGACCTCGGTAGTCGACGCCGG
AGTTGAGCTTGATGACGACACCGTACTGGCGTACCAGGCCTAGTAGTGAACACCGGGCCTGAAGCTGTCGCCGC
CGCTGCTCATCTTGTGGGCTGTGCCCGGTGTCCCTGTTGCGGATTGCGGGTGGCAGCCTGGCAGGTGGGTGCGA
CCCGTTTGGACTCCCTGATCTGGGCCCTTTGTGTCAGTACCGTCTGTACTCCGATGACATGCACACCGTCGTCCA
CAGTCAAGTCCACAATCTCCCCTCTTTTTTTAACGGAATAGTTCAAAATCTCCTTGACGCACGCTATCGTGTACC
AGCGCTCACTGGACACCACGTTTGTAATCCACGCCGACACGTCGGTCCCACGTCGACAGGCCCCACCGTCCGGT
CTGTAGCGTGTACGTATTCGGGCGACGGACGTGTCGTCGTCGTCTTGCGAGTCCCATTCCCATCACCATCTGAGC
CACACATCCTCTGAACAAAAGCAGGGAGGCCTCTACGCACATCCCCCTTTCTCCCACTCCTTGTCCGTGGCACCC
ACCCCAAACCCTCGCGCCGCCTCCGAGACAGCCGCCGCAACCATGGCCACCGCCTGCAGGACCGTCTTCGGTAC
GCGCTCACTCCGCCCTCTGCCTTTGTTACTGCCACGTTTCTCTGAATGCTCTCTTGTGTGGTGATTGCTGAGAGTG
GTTTAGCTGGATCTAGAATTACACTCTGAAATCGTGTTCTGCCTGTGCTGATTACTTGCCGTCCTTTGTAGCAGC
AAAATATAGGGACATGGTAGTACGAAACGAAGATAGAACCTACACAGCAATACGAGAAATGTGTAATTTGGTG
CTTAGCGGTATTTATTTAAGCACATGTTGGTGTTATAGGGCACTTGGATTCAGAAGTTTGCTGTTAATTTAGGCA
CAGGCTTCATACTACATGGGTCAATAGTATAGGGATTCATATTATAGGCGATACTATAATAATTTGTTCGTCTGC
AGAGCTTATTATTTGCCAAAATTAGATATTCCTATTCTGTTTTTGTTTGTGTGCTGTTAAATTGTTAACGCCTGAA
GGAATAAATATAAATGACGAAATTTTGATGTTTATCTCTGCTCCTTTATTGTGACCATAAGTCAAGATCAGATGC
ACTTGTTTTAAATATTGTTGTCTGAAGAAATAAGTACTGACAGTATTTTGATGCATTGATCTGCTTGTTTGTTGTA
ACAAAATTTAAAAATAAAGAGTTTCCTTTTTGTTGCTCTCCTTACCTCCTGATGGTATCTAGTATCTACCAACTG
ACACTATATTGCTTCTCTTTACATACGTATCTTGCTCGATGCCTTCTCCCTAGTGTTGACCAGTGTTACTCACATA
GTCTTTGCTCATTTCATTGTAATGCAGATACCAAGCGGCCCCTGCAGGATGGGGATTGAACAAGATGGATTGCA
CGCAGGTTCTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCGGTGCCCTG
AATGAACTCCAGGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTCCTGTCATCTCAC
CTTGCTCCTGCCGAGAAAGTATCCATCATGGCTGATGCAATGCGGCGGCTGCATACGCTTGATCCGGCTACCTG
CCCATTCGACCACCAAGCGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCTCAAGGCGCGCATGCCCG
ACGGCGAGGATCTCGTCGTGACACATGGCGATGCCTGCTTGCCGAATATCATGGTGGAAAATGGCCGCTTTTCT
GGATTCATCGACTGTGGCCGGCTGGGTGTGGCGGACCGCTATCAGGACATAGCGTTGGCTACCCGTGATATTGC
TGAAGAGCTTGGCGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAGCGATCGCAGAGCCATGGGGGCGGTGAGAGCTCGCGGCG
TCGGATCTCCGGTTTCAGTTTCAGCTACGCGACTTGTGTGTGTGTGTGTGTGTCAGTGCGTGGTTACTCTGAGCT
TCGGTATGTTTGGTGTGTACTGTTCAGTGTTTCATGGTGCTTCCTAGCAGTGTCTGTCGTGTACTTTGCCTGTATG
TTGAGCTCGGCTCAGTATGTTCTGGGAGTGAATAAATAAAAAAAAATTAACCAGATATTGTCGCGTGTACTTAG
TATACTAATCACCTGCTCCTGCTCTTGCTCTGTTTTATCACCTTTCGGGCTTGACTGTCTTTGTGGAGGCCTCTCA
GTCTGATCTGGAACTCTACCTAAGTTTTACTAGTCCCGGGAAGCTTCTGCAGTGCAGCGTGACCCGGTCGTGCCC
CTCACTAGAGATAATGAGCATTGCATGTCTAAGTTATAAAAAATTACCACATATTTTTTTTGTCACACTTGTTTG
AAGTGCAGTTTATCTATCTTTATACATATATTTAAACTTTACTCTACGAATAATATAATCTATAGTACTACAATA
ATATCAGTGTTTTAGAGAATCATATAAATGAACAGTTAGACATGGTCTAAAGGACAATTGAGTATTTTGACAAC
AGGACTCTACAGTTTTATCTTTTTAGTGTGCATGTGTTCTCCTTTTTTTTTGCAAATAGCTTCACCTATATAATAC
TTCATCCATTTTATTAGTACATCCATTTAGGGTTTAGGGTTAATGGTTTTTATAGACTAATTTTTTTAGTACATCT
ATTTTATTCTATTTTAGCCTCTAAATTAAGAAAACTAAAACTCTATTTTAGTTTTTTTATTTAATAATTTAGATAT
AAAATAGAATAAAATAAAGTGACTAAAAATTAAACAAATACCCTTTAAGAAATTAAAAAAACTAAGGAAACAT
TTTTCTTGTTTCGAGTAGATAATGCCAGCCTGTTAAACGCCGTCGACGAGTCTAACGGACACCAACCAGCGAAC
CAGCAGCGTCGCGTCGGGCCAAGCGAAGCAGACGGCACGGCATCTCTGTCGCTGCCTCTGGACCCCTCTCGAGA
GTTCCGCTCCACCGTTGGACTTGCTCCGCTGTCGGCATCCAGAAATTGCGTGGCGGAGCGGCAGACGTGAGCCG
GCACGGCAGGCGGCCTCCTCCTCCTCTCACGGCACGGCAGCTACGGGGGATTCCTTTCCCACCGCTCCTTCGCTT
TCCCTTCCTCGCCCGCCGTAATAAATAGACACCCCCTCCACACCCTCTTTCCCCAACCTCGTGTTGTTCGGAGCG
CACACACACACAACCAGATCTCCCCCAAATCCACCCGTCGGCACCTCCGCTTCAAGGTACGCCGCTCGTCCTCC
CCCCCCCCCCCTCTCTACCTTCTCTAGATCGGCGTTCCGGTCCATGGTTAGGGCCCGGTAGTTCTACTTCTGTTCA
TGTTTGTGTTAGATCCGTGTTTGTGTTAGATCCGTGCTGCTAGCGTTCGTACACGGATGCGACCTGTACGTCAGA
CACGTTCTGATTGCTAACTTGCCAGTGTTTCTCTTTGGGGAATCCTGGGATGGCTCTAGCCGTTCCGCAGACGGG
ATCGATTTCATGATTTTTTTTGTTTCGTTGCATAGGGTTTGGTTTGCCCTTTTCCTTTATTTCAATATATGCCGTGC
ACTTGTTTGTCGGGTCATCTTTTCATGCTTTTTTTTGTCTTGGTTGTGATGATGTGGTCTGGTTGGGCGGTCGTTC
TAGATCGGAGTAGAATTCTGTTTCAAACTACCTGGTGGATTTATTAATTTTGGATCTGTATGTGTGTGCCATACA
TATTCATAGTTACGAATTGAAGATGATGGATGGAAATATCGATCTAGGATAGGTATACATGTTGATGCGGGTTT
TACTGATGCATATACAGAGATGCTTTTTGTTCGCTTGGTTGTGATGATGTGGTGTGGTTGGGCGGTCGTTCATTC
GTTCTAGATCGGAGTAGAATACTGTTTCAAACTACCTGGTGTATTTATTAATTTTGGAACTGTATGTGTGTGTCA
TACATCTTCATAGTTACGAGTTTAAGATGGATGGAAATATCGATCTAGGATAGGTATACATGTTGATGTGGGTT
TTACTGATGCATATACATGATGGCATATGCAGCATCTATTCATATGCTCTAACCTTGAGTACCTATCTATTATAA
TAAACAAGTATGTTTTATAATTATTTTGATCTTGATATACTTGGATGATGGCATATGCAGCAGCTATATGTGGAT
TTTTTTAGCCCTGCCTTCATACGCTATTTATTTGCTTGGTACTGTTTCTTTTGTCGATGCTCACCCTGTTGTTTGGT
GTTACTTCTCTAGAGCGGCCGCATTTAAATCACTCTGTGGTCTCAAATGGACCGTATCCAACGGCTGCGAGCTC
AAGCCGTCCATGGTGGTGAACCAGCCCAGGGTCGAGGTCGGGGGACCCGACATGAGGACCTTCTACACCCTCG
TGATGGGAAGCCATCCGCAGTGGTGAATAAGCCAAGAGTTGATGTTGGGGGCACCGACCTCAGGGTGTTCTTTA
CACTGGTAGGTATTAGTTGATCCAGATGCCCCAAGCCAACCATTCCACTAAGAATAACATACAACAATAGGCTA
CTTCTGGCAAGTGCTGAGCTAAAGCCATCTGCGGTTGCAAGTAAACCACGAGTCGATGTCGGTGGCGTTTGAAT
CAGATTCAGATTTCATTGCATCACAGAGATCCATCTTTACTCTACCGCTTGCTCTAACTTAACTTGTAATTGTTTT
TTATCATGCAGGCCACCGACATCGACTCGTGGTTTACTTGCAACCGCAGATGGCTTTAGCTCAGCACTTGCCAG
AAGTAGCCTATTGTTGTATGTTATTCTTAGTGGAATGGTTGGCTTGGGGCATCTGGATCAACTAATACCTACCAG
TGTAAAGAACACCCTGAGGTCGGTGCCCCCAACATCAACTCTTGGCTTATTCACCACTGCGGATGGCTTCCCAT
CACGAGGGTGTAGAAGGTCCTCATGTCGGGTCCCCCGACCTCGACCCTGGGCTGGTTCACCACCATGGACGGCT
TGAGCTCGCAGCCGTTGGATACGGTCGCTTTGAGACCACGAAGTGTTCGAATTAATTAAGGGGACACCGTTGTC
GGAGCAGTGCCAGTGAGCTGGGTGGCAACCTGCGGGACTCTGGTGAGGAAGAGCAAATGGTAGAAAGCAGTA
GCTGTGCTTCTGAACATTGTGTGTGTTTGTTTCCCTTGTTACCGCCAATCATCCTACCTGATGTGTCGTCTGGGGT
TTCGTTTGAACCATGCTGGGTTTGGAGCTTTGTGATGCAAAGCAGAATGTCCTGATGCTTTCTTTCTCCATAGAA
TAAATCTCTCCTGTTACCTAAAGGCCGGCCGATATCCATTCAAGAGGAGAGCTCATTTCCTCTCCTCTCATCACC
TTTGCCTAAAAACAGTATGAAAGTCAAGCTAAAGACTTTAAACTAGCTCACTTGGGAGGAAGTCCCATGTCTAT
TCTTGTATATACTTTTTTCCCTTGTTATTTTTGATGTTTTGTGTAAGTATTTCAGGTTTTATAGTCCACGGAAGTCA
ACGATCGAGTGATATAAATCGAGTCAAAGAATCTTAGCCGTGCGATAGAGTGACTTAAGGCGAAGCATCCAGC
TAATCCGAGTCCCACACAACCTATTCCTTGGAGCTCTAAACGGGAGAGTATGGACGACTTCGTCAACTCCTATT
TCACCAGATCACTAAGGTAACACACCACCCCCATTGTATATACCGTTATCTTTTATTTTTCTTTGTTTGTTGTTTG
TTTTAGTCTTGAATTCTCTCTCAGATTTTTAATACACAAGGGACTGTGTAATTTAAGTGTGGGGGAGGGTTCAAG
AAATATTTGACTTTATGTTCTCTTTTCTTATTTCAAATTTTGCATCATCTAAGGCATAAAAAACCATAAAAATTTA
AAAACATTTATGAAAATGATTTCACCAAAACAGAGTGTCATGTAGATTGCATTGCATTTTAGGATTGTATATAG
AGTACTTGCATTTAGGATTTCTCATTTGCATAGGGGATAATGATGATTTCGTACCGTTAAGCATGTTTTGATTCA
ATGAAGTAAGTTCAATGCCCAGGTTGTTAGTTCTCTGTTGCATAACCGATTGTTTCTCTGAAGTAAGACTGCACC
CTGCTTTGAATTTACAACTTGACTATACTTCTTTGATTCAAAACTCGCTTATCTGATGCCATTCCCTATCTATTAG
AACCTGAACTGAATTTGCAATTATCATGTCTATGCATTGTTTGTGAACTCATGGCTACCATATACATACTTGTAT
TATCTTATTCACTTCACCACCCTCTGTTAATCTAAGTAGCTGACTCTCATTTTTAGAGCAGTTTCCCATACCCTTA
GCCTAGCCTTATTTCAAGCCAAAGATCATTTGTGTGTGATTGTGAGGTCTTTTTCGATTGAGCTTGGTATAACGT
GTCAGGTATGAGCCGACAAGAGCAGTCTTTCATGTAGACCTAGCCCACGCTTTTTGGGTTAGCTAGGACTAGGT
GGAGACTTGTATTTGGGATTGGGGTTGTGTATGGATTGAATGGGAAAGAGAATGAGAATGAAAAGAAAAGAAA
AAAAGAGAGTAAGAGTTTAGAGTTCTTAAGGGGGATACAAAAAGTGTTAGTCTAGTAAAGGGTTTGGAATTCA
AAGAAGTTAGGCATTGTGATTTAAAGAAAAGGGTAAAACGACCTAAGATTAGAAGAAAAGCCTTAAGTTTGTC
AAAGTATAAAACCAAACCCACTAGACTCTTGAATCGTTTTAAAGAAGAACCCCTTAGAACTTGAACCAAAAAG
AGAAAGAATGAGAAAAAGGGTAGAGTTAGGACATCTATGGGACTGGAACCATCTTATGAACTATCATACTATA
TCATTGGGTAGATGGGAGTCTGTTCTTGTTTGTGTCCATTGGCTTTTACCTTTAGCATTCTGCTGACGCTCAATCT
TTTCTTTGAGAGTCCCCTGTTACTCGACCAAATCATTAAAGGGACCATTTTTGTCTCTTAGCCTAAGCCTGAAAC
CAAGTGAGTTCATAACCATTGCATTGCTTGATCCATTGTTCGTGCTTAGTGAATGTTAAAAAGAATTGGTTGATG
TGAATGCTTGAATAGATTAAGGCAGGTTGTTAGGTTGCTTTGTGACGAGTATGGCTAAATTTTTAAGTAAGGGT
CTATCATTTTGCAACCTATAATTAGCTACCTGGACATTGAGCTTACCCGTTCTATATGCATGCTACGGGTTTTGA
ATCCCCACCTTCAAACCTCTCCTTCAACTTAAGATTTTTTGTTTGCTTGAGGGCAAGCAAGAGATAAGTTTGGGG
GAGTTGATAACACTGTAATTTACTCATTTTGGGCATGTATTTATCATCATTTAGCTTAGTGATCACTCATTGTTTC
ATATCATTTTGCATCACATTTAGCTTATTTTGCACACTTAGGCATTTAGGGTCATCTTGCATACATATTGCATGTT
ATGGTGTGTTTTCAGGTGCAAAATAAGTTTACAAGGAGTTTACTCGACCCTCTACCCTATTGCCCCAGAGGGGA
ACCAGAGGAGCAGCTACTCGACCAATTATTCATTGCCTCAGAGGGGAAGCAGCAGAGCAGCTACTCGACCAGT
TATTCGGCGCGCCTAGGGATAA
SEQ ID NO: 19 (ScFT4, ScFT8, ScFT10 RNAi-hairpin-short, ALS marker, minimal cassette,
9581 bp)
CAGGGTAATGGCGCGCCGTTGTTTTAGTGTCCGAGGTTTGGTTTGGTGAAGTACATCTGAACTCTGAAGGTTCTT
CGTGACATGGTTAGACGTGGTGTTGCAGTACTGTGATAAAACTTATCAGAATAAGTTTTCGCTTCTAATTTGTTT
TCTTAAATCATGTGTTGATCAGCCTATGAGTTGTTCATATCGTTGCCTTTCGTGTTGAACATTTGTTTTCTTGCCA
TACTAATGAGATTCATCTGCACAAACTGTACGAAGAGAATCAAGTTTAAACACTGCAGGTCAACGGATCACCTA
TCAACATCCCAGCTAAAAACAGTAAAAAGGGGGAAAACGTGGGTGAGTTGAGTCTGTCTTGTGGAAAAAACGT
TTTAGTTTCTCCTGGAATTAACAATAAAAACAGTTGAACAAGATTGACTGTTCCTCCGGGAGGGTTTGGAACAT
CGTTACAGATGTGAGCGAAAGGTGAGGAAACAGAGCGGAGGGCTTGGAGGTGACCTCGGTAGTCGACGCCGG
AGTTGAGCTTGATGACGACACCGTACTGGCGTACCAGGCCTAGTAGTGAACACCGGGCCTGAAGCTGTCGCCGC
CGCTGCTCATCTTGTGGGCTGTGCCCGGTGTCCCTGTTGCGGATTGCGGGTGGCAGCCTGGCAGGTGGGTGCGA
CCCGTTTGGACTCCCTGATCTGGGCCCTTTGTGTCAGTACCGTCTGTACTCCGATGACATGCACACCGTCGTCCA
CAGTCAAGTCCACAATCTCCCCTCTTTTTTTAACGGAATAGTTCAAAATCTCCTTGACGCACGCTATCGTGTACC
AGCGCTCACTGGACACCACGTTTGTAATCCACGCCGACACGTCGGTCCCACGTCGACAGGCCCCACCGTCCGGT
CTGTAGCGTGTACGTATTCGGGCGACGGACGTGTCGTCGTCGTCTTGCGAGTCCCATTCCCATCACCATCTGAGC
CACACATCCTCTGAACAAAAGCAGGGAGGCCTCTACGCACATCCCCCTTTCTCCCACTCCTTGTCCGTGGCACCC
ACCCCAAACCCTCGCGCCGCCTCCGAGACAGCCGCCGCAACCATGGCCACCGCCTGCAGGACCGTCTTCGGTAC
GCGCTCACTCCGCCCTCTGCCTTTGTTACTGCCACGTTTCTCTGAATGCTCTCTTGTGTGGTGATTGCTGAGAGTG
GTTTAGCTGGATCTAGAATTACACTCTGAAATCGTGTTCTGCCTGTGCTGATTACTTGCCGTCCTTTGTAGCAGC
AAAATATAGGGACATGGTAGTACGAAACGAAGATAGAACCTACACAGCAATACGAGAAATGTGTAATTTGGTG
CTTAGCGGTATTTATTTAAGCACATGTTGGTGTTATAGGGCACTTGGATTCAGAAGTTTGCTGTTAATTTAGGCA
CAGGCTTCATACTACATGGGTCAATAGTATAGGGATTCATATTATAGGCGATACTATAATAATTTGTTCGTCTGC
AGAGCTTATTATTTGCCAAAATTAGATATTCCTATTCTGTTTTTGTTTGTGTGCTGTTAAATTGTTAACGCCTGAA
GGAATAAATATAAATGACGAAATTTTGATGTTTATCTCTGCTCCTTTATTGTGACCATAAGTCAAGATCAGATGC
ACTTGTTTTAAATATTGTTGTCTGAAGAAATAAGTACTGACAGTATTTTGATGCATTGATCTGCTTGTTTGTTGTA
ACAAAATTTAAAAATAAAGAGTTTCCTTTTTGTTGCTCTCCTTACCTCCTGATGGTATCTAGTATCTACCAACTG
ACACTATATTGCTTCTCTTTACATACGTATCTTGCTCGATGCCTTCTCCCTAGTGTTGACCAGTGTTACTCACATA
GTCTTTGCTCATTTCATTGTAATGCAGATACCAAGCGGCCCCTGCAGGATGGCCACCACCGCCGCCGCCGCTGC
CGCCGCGCTAGCCGGCGCCACTACCGCTGCGCCCAAGGCGAGGCGCCGGGCGCACCTCCTGGCCGCACGGCGC
GCCCTCGCCGCGCCCATCAGGTGCTCAGCGGCGCCACCCGCCACGCTGACGGTGACGGCTCCCCCGGCCACCCC
GCTCCGGCCGTGGGGCCCCACCGATCCCCGCAAGGGCGCCGACATCCTCGTCGAGGCTCTTGAGCGCTGCGGCG
TCCGCGACGTCTTCGCCTACCCCGGCGGCGCGTCCATGGAGATCCACCAGGCACTCACCCGTTCCCCCGTCATC
GCCAACCACCTCTTCCGCCACGAGCAAGGGGAGGCCTTCGCCGCCTCTGGCTTCGCGCGCTCCTCGGGCCGCGT
CGGCGTCTGCGTCGCCACCTCCGGCCCCGGCGCCACCAACCTAGTCTCCGCGCTCGCCGACGCGCTGCTCGACT
CCGTCCCCATGGTCGCCATCACGGGACAGGTTCCGCGGCGCATGATTGGCACCGACGCCTTCCAGGAGACGCCC
ATCGTCGAGGTCACCCGCTCCATCACCAAACATAACTACCTGGTCCTCGACGTCGACGACATCCCCCGCGTCGT
GCAGGAGGCTTTCTTCCTCGCCTCCTCCGGTCGCCCGGGACCGGTGCTTGTCGACATCCCCAAGGACATCCAGC
AGCAGATGGCCGTGCCGGTCTGGGACACGCCCATGAGTCTGCCTGGGTACATTGCGCGCCTTCCCAAGCCTCCT
GCGACTGAATTGCTTGAGCAGGTGCTGCGTCTTGTTGGTGAATCAAGGCGCCCTGTTCTTTATGTTGGTGGTGGC
TGCGCAGCATCTGGCGAGGAGTTGCGCCGCTTTGTGGAGATGACTGGAATCCCAGTCACAACTACTCTTATGGG
CCTTGGCAATTTCCCTGGCGACGACCCACTGTCTCTGCGCATGCTTGGTATGCATGGCACGGTGTATGCAAATTA
TGCAGTGGATAAGGCGGATCTGTTGCTTGCATTTGGTGTGCGGTTTGATGATCGTGTGACAGGGAAGATTGAGG
CTTTTGCAAGCAGGGCTAAGATTGTGCACATTGATATTGATCCCGCTGAGATTGGCAAGAACAAGCAGCCACAT
GTGTCCATCTGTGCAGACGTTAAGCTTGCTTTGCAGGGCATGAATGCTCTTCTGGAAGGAAGCACATCAAAGAA
GAGCTTTGACTTTGGCTCATGGCAAGCTGAGTTGGATCAGCAGAAGAGAGAGTTCCCCCTTGGGTATAAAACTT
TTGATGACGAGATCCAGCCACAATATGCTATTCAGGTTCTTGATGAGCTGACAAAAGGGGAGGCCATCATTGCC
ACAGGTGTTGGGCAGCACCAGATGTGGGCGGCACAGTACTACACTTACAAGCGGCCAAGGCAGTGGTTGTCTT
CAGCTGGTCTTGGGGCTATGGGATTTGGTTTGCCGGCTGCTGCTGGCGCTGCTGTGGCCAACCCAGGTATCACT
GTTGTTGACATCGACGGAGATGGTAGCTTCCTCATGAACATTCAGGAGCTAGCTATGATCCGAATTGAGAACCT
CCCAGTGAAGGTCTTTGTGCTAAACAACCAGCACCTGGGGATGGTGGTGCAGTTGGAGGACAGGTTCTATAAG
GCCAATAGAGCACACACATACTTGGGAAACCCAGAGAATGAAAGTGAGATATATCCAGATTTCGTGACAATTG
CCAAAGGGTTCAACATTCCAGCAGTCCGTGTGACAAAGAAGAGCGAAGTCCATGCAGCAATCAAGAAGATGCT
TGAGACTCCAGGGCCATACCTCTTGGATATAATCGTCCCGCACCAGGAGCATGTGTTGCCTATGATCCCTAGTG
GTGGGGCTTTCAAGGATATGATCCTGGATGGTGATGGCAGGACTGTGTATTGAGCGATCGCAGAGCCATGGGG
GCGGTGAGAGCTCGCGGCGTCGGATCTCCGGTTTCAGTTTCAGCTACGCGACTTGTGTGTGTGTGTGTGTGTCAG
TGCGTGGTTACTCTGAGCTTCGGTATGTTTGGTGTGTACTGTTCAGTGTTTCATGGTGCTTCCTAGCAGTGTCTGT
CGTGTACTTTGCCTGTATGTTGAGCTCGGCTCAGTATGTTCTGGGAGTGAATAAATAAAAAAAAATTAACCAGA
TATTGTCGCGTGTACTTAGTATACTAATCACCTGCTCCTGCTCTTGCTCTGTTTTATCACCTTTCGGGCTTGACTG
TCTTTGTGGAGGCCTCTCAGTCTGATCTGGAACTCTACCTAAGTTTTACTAGTCCCGGGAAGCTTCTGCAGTGCA
GCGTGACCCGGTCGTGCCCCTCACTAGAGATAATGAGCATTGCATGTCTAAGTTATAAAAAATTACCACATATT
TTTTTTGTCACACTTGTTTGAAGTGCAGTTTATCTATCTTTATACATATATTTAAACTTTACTCTACGAATAATAT
AATCTATAGTACTACAATAATATCAGTGTTTTAGAGAATCATATAAATGAACAGTTAGACATGGTCTAAAGGAC
AATTGAGTATTTTGACAACAGGACTCTACAGTTTTATCTTTTTAGTGTGCATGTGTTCTCCTTTTTTTTTGCAAAT
AGCTTCACCTATATAATACTTCATCCATTTTATTAGTACATCCATTTAGGGTTTAGGGTTAATGGTTTTTATAGAC
TAATTTTTTTAGTACATCTATTTTATTCTATTTTAGCCTCTAAATTAAGAAAACTAAAACTCTATTTTAGTTTTTTT
ATTTAATAATTTAGATATAAAATAGAATAAAATAAAGTGACTAAAAATTAAACAAATACCCTTTAAGAAATTAA
AAAAACTAAGGAAACATTTTTCTTGTTTCGAGTAGATAATGCCAGCCTGTTAAACGCCGTCGACGAGTCTAACG
GACACCAACCAGCGAACCAGCAGCGTCGCGTCGGGCCAAGCGAAGCAGACGGCACGGCATCTCTGTCGCTGCC
TCTGGACCCCTCTCGAGAGTTCCGCTCCACCGTTGGACTTGCTCCGCTGTCGGCATCCAGAAATTGCGTGGCGG
AGCGGCAGACGTGAGCCGGCACGGCAGGCGGCCTCCTCCTCCTCTCACGGCACGGCAGCTACGGGGGATTCCTT
TCCCACCGCTCCTTCGCTTTCCCTTCCTCGCCCGCCGTAATAAATAGACACCCCCTCCACACCCTCTTTCCCCAAC
CTCGTGTTGTTCGGAGCGCACACACACACAACCAGATCTCCCCCAAATCCACCCGTCGGCACCTCCGCTTCAAG
GTACGCCGCTCGTCCTCCCCCCCCCCCCCTCTCTACCTTCTCTAGATCGGCGTTCCGGTCCATGGTTAGGGCCCG
GTAGTTCTACTTCTGTTCATGTTTGTGTTAGATCCGTGTTTGTGTTAGATCCGTGCTGCTAGCGTTCGTACACGGA
TGCGACCTGTACGTCAGACACGTTCTGATTGCTAACTTGCCAGTGTTTCTCTTTGGGGAATCCTGGGATGGCTCT
AGCCGTTCCGCAGACGGGATCGATTTCATGATTTTTTTTGTTTCGTTGCATAGGGTTTGGTTTGCCCTTTTCCTTT
ATTTCAATATATGCCGTGCACTTGTTTGTCGGGTCATCTTTTCATGCTTTTTTTTGTCTTGGTTGTGATGATGTGG
TCTGGTTGGGCGGTCGTTCTAGATCGGAGTAGAATTCTGTTTCAAACTACCTGGTGGATTTATTAATTTTGGATC
TGTATGTGTGTGCCATACATATTCATAGTTACGAATTGAAGATGATGGATGGAAATATCGATCTAGGATAGGTA
TACATGTTGATGCGGGTTTTACTGATGCATATACAGAGATGCTTTTTGTTCGCTTGGTTGTGATGATGTGGTGTG
GTTGGGCGGTCGTTCATTCGTTCTAGATCGGAGTAGAATACTGTTTCAAACTACCTGGTGTATTTATTAATTTTG
GAACTGTATGTGTGTGTCATACATCTTCATAGTTACGAGTTTAAGATGGATGGAAATATCGATCTAGGATAGGT
ATACATGTTGATGTGGGTTTTACTGATGCATATACATGATGGCATATGCAGCATCTATTCATATGCTCTAACCTT
GAGTACCTATCTATTATAATAAACAAGTATGTTTTATAATTATTTTGATCTTGATATACTTGGATGATGGCATAT
GCAGCAGCTATATGTGGATTTTTTTAGCCCTGCCTTCATACGCTATTTATTTGCTTGGTACTGTTTCTTTTGTCGA
TGCTCACCCTGTTGTTTGGTGTTACTTCTCTAGAGCGGCCGCATTTAAATCACTCTGTGGTCTCAAATGGACCGT
ATCCAACGGCTGCGAGCTCAAGCCGTCCATGGTGGTGAACCAGCCCAGGGTCGAGGTCGGGGGACCCGACATG
AGGACCTTCTACACCCTCGTGATGGGAAGCCATCCGCAGTGGTGAATAAGCCAAGAGTTGATGTTGGGGGCAC
CGACCTCAGGGTGTTCTTTACACTGGTAGGTATTAGTTGATCCAGATGCCCCAAGCCAACCATTCCACTAAGAA
TAACATACAACAATAGGCTACTTCTGGCAAGTGCTGAGCTAAAGCCATCTGCGGTTGCAAGTAAACCACGAGTC
GATGTCGGTGGCGTTTGAATCAGATTCAGATTTCATTGCATCACAGAGATCCATCTTTACTCTACCGCTTGCTCT
AACTTAACTTGTAATTGTTTTTTATCATGCAGGCCACCGACATCGACTCGTGGTTTACTTGCAACCGCAGATGGC
TTTAGCTCAGCACTTGCCAGAAGTAGCCTATTGTTGTATGTTATTCTTAGTGGAATGGTTGGCTTGGGGCATCTG
GATCAACTAATACCTACCAGTGTAAAGAACACCCTGAGGTCGGTGCCCCCAACATCAACTCTTGGCTTATTCAC
CACTGCGGATGGCTTCCCATCACGAGGGTGTAGAAGGTCCTCATGTCGGGTCCCCCGACCTCGACCCTGGGCTG
GTTCACCACCATGGACGGCTTGAGCTCGCAGCCGTTGGATACGGTCGCTTTGAGACCACGAAGTGTTCGAATTA
ATTAAGGGGACACCGTTGTCGGAGCAGTGCCAGTGAGCTGGGTGGCAACCTGCGGGACTCTGGTGAGGAAGAG
CAAATGGTAGAAAGCAGTAGCTGTGCTTCTGAACATTGTGTGTGTTTGTTTCCCTTGTTACCGCCAATCATCCTA
CCTGATGTGTCGTCTGGGGTTTCGTTTGAACCATGCTGGGTTTGGAGCTTTGTGATGCAAAGCAGAATGTCCTGA
TGCTTTCTTTCTCCATAGAATAAATCTCTCCTGTTACCTAAAGGCCGGCCGATATCCATTCAAGAGGAGAGCTCA
TTTCCTCTCCTCTCATCACCTTTGCCTAAAAACAGTATGAAAGTCAAGCTAAAGACTTTAAACTAGCTCACTTGG
GAGGAAGTCCCATGTCTATTCTTGTATATACTTTTTTCCCTTGTTATTTTTGATGTTTTGTGTAAGTATTTCAGGTT
TTATAGTCCACGGAAGTCAACGATCGAGTGATATAAATCGAGTCAAAGAATCTTAGCCGTGCGATAGAGTGACT
TAAGGCGAAGCATCCAGCTAATCCGAGTCCCACACAACCTATTCCTTGGAGCTCTAAACGGGAGAGTATGGAC
GACTTCGTCAACTCCTATTTCACCAGATCACTAAGGTAACACACCACCCCCATTGTATATACCGTTATCTTTTAT
TTTTCTTTGTTTGTTGTTTGTTTTAGTCTTGAATTCTCTCTCAGATTTTTAATACACAAGGGACTGTGTAATTTAA
GTGTGGGGGAGGGTTCAAGAAATATTTGACTTTATGTTCTCTTTTCTTATTTCAAATTTTGCATCATCTAAGGCA
TAAAAAACCATAAAAATTTAAAAACATTTATGAAAATGATTTCACCAAAACAGAGTGTCATGTAGATTGCATTG
CATTTTAGGATTGTATATAGAGTACTTGCATTTAGGATTTCTCATTTGCATAGGGGATAATGATGATTTCGTACC
GTTAAGCATGTTTTGATTCAATGAAGTAAGTTCAATGCCCAGGTTGTTAGTTCTCTGTTGCATAACCGATTGTTT
CTCTGAAGTAAGACTGCACCCTGCTTTGAATTTACAACTTGACTATACTTCTTTGATTCAAAACTCGCTTATCTG
ATGCCATTCCCTATCTATTAGAACCTGAACTGAATTTGCAATTATCATGTCTATGCATTGTTTGTGAACTCATGG
CTACCATATACATACTTGTATTATCTTATTCACTTCACCACCCTCTGTTAATCTAAGTAGCTGACTCTCATTTTTA
GAGCAGTTTCCCATACCCTTAGCCTAGCCTTATTTCAAGCCAAAGATCATTTGTGTGTGATTGTGAGGTCTTTTT
CGATTGAGCTTGGTATAACGTGTCAGGTATGAGCCGACAAGAGCAGTCTTTCATGTAGACCTAGCCCACGCTTT
TTGGGTTAGCTAGGACTAGGTGGAGACTTGTATTTGGGATTGGGGTTGTGTATGGATTGAATGGGAAAGAGAAT
GAGAATGAAAAGAAAAGAAAAAAAGAGAGTAAGAGTTTAGAGTTCTTAAGGGGGATACAAAAAGTGTTAGTC
TAGTAAAGGGTTTGGAATTCAAAGAAGTTAGGCATTGTGATTTAAAGAAAAGGGTAAAACGACCTAAGATTAG
AAGAAAAGCCTTAAGTTTGTCAAAGTATAAAACCAAACCCACTAGACTCTTGAATCGTTTTAAAGAAGAACCCC
TTAGAACTTGAACCAAAAAGAGAAAGAATGAGAAAAAGGGTAGAGTTAGGACATCTATGGGACTGGAACCAT
CTTATGAACTATCATACTATATCATTGGGTAGATGGGAGTCTGTTCTTGTTTGTGTCCATTGGCTTTTACCTTTAG
CATTCTGCTGACGCTCAATCTTTTCTTTGAGAGTCCCCTGTTACTCGACCAAATCATTAAAGGGACCATTTTTGT
CTCTTAGCCTAAGCCTGAAACCAAGTGAGTTCATAACCATTGCATTGCTTGATCCATTGTTCGTGCTTAGTGAAT
GTTAAAAAGAATTGGTTGATGTGAATGCTTGAATAGATTAAGGCAGGTTGTTAGGTTGCTTTGTGACGAGTATG
GCTAAATTTTTAAGTAAGGGTCTATCATTTTGCAACCTATAATTAGCTACCTGGACATTGAGCTTACCCGTTCTA
TATGCATGCTACGGGTTTTGAATCCCCACCTTCAAACCTCTCCTTCAACTTAAGATTTTTTGTTTGCTTGAGGGCA
AGCAAGAGATAAGTTTGGGGGAGTTGATAACACTGTAATTTACTCATTTTGGGCATGTATTTATCATCATTTAGC
TTAGTGATCACTCATTGTTTCATATCATTTTGCATCACATTTAGCTTATTTTGCACACTTAGGCATTTAGGGTCAT
CTTGCATACATATTGCATGTTATGGTGTGTTTTCAGGTGCAAAATAAGTTTACAAGGAGTTTACTCGACCCTCTA
CCCTATTGCCCCAGAGGGGAACCAGAGGAGCAGCTACTCGACCAATTATTCATTGCCTCAGAGGGGAAGCAGC
AGAGCAGCTACTCGACCAGTTATTCGGCGCGCCTAGGGATAA
SEQ ID NO: 20 (ScFT4 sgRNA expression vector, minimal cassette, 11158 bp)
GGTAGCGAGGCGCGCCGTTGTTTTAGTGTCCGAGGTTTGGTTTGGTGAAGTACATCTGAACTCTGAAGGTTCTTC
GTGACATGGTTAGACGTGGTGTTGCAGTACTGTGATAAAACTTATCAGAATAAGTTTTCGCTTCTAATTTGTTTT
CTTAAATCATGTGTTGATCAGCCTATGAGTTGTTCATATCGTTGCCTTTCGTGTTGAACATTTGTTTTCTTGCCAT
ACTAATGAGATTCATCTGCACAAACTGTACGAAGAGAATCAAGATATCGCCCGGGCTTTGTGAAAGTTGAATTA
CGGCATAGCCGAAGGAATAACAGAATCGTTTCACACTTTCGTAACAAAGATCTTCTTATCATGTTTCAGACGAT
GGAGGCAAGGCTGATCAAAGTGATCAAGCACATAAACGCATTTTTTTACCATGTTTCACTCCATAAGCGTCTGA
GATTATCACAAGTCACGTCTAGTAGTTTGATGGTACACTAGTGACAATCAGTTCGTGCAGACAGAGCTCATACT
TGACTACTTGAGCGATTACAGGCGAAAGTGTGAAACGCATGTGATGTGGGCTGGGAGGAGGAGAATATATACT
AATGGGCCGTATCCTGATTTGGGCTGCGTCGGAAGGTGCAGCCCACGCGCGCCGTACCGCGCGGGTGGCGCTGC
TACCCACTTTAGTCCGTTGGATGGGGATCCGATGGTTTGCGCGGTGGCGTTGCGGGGGATGTTTAGTACCACAT
CGGAAACCGAAAGACGATGGAACCAGCTTATAAACCCGCGCGCTGTAGTCAGCTTGCCGTAGCTGACCCTGAG
GTGTTTTAGAGCTAGAAATAGCAAGTTAAAATAAGGCTAGTCCGTTATCAACTTGAAAAAGTGGCACCGAGTCG
GTGCTTTTTTTTTAAGCTTTTTGTGAAAGTTGAATTACGGCATAGCCGAAGGAATAACAGAATCGTTTCACACTT
TCGTAACAAAGATCTTCTTATCATGTTTCAGACGATGGAGGCAAGGCTGATCAAAGTGATCAAGCACATAAACG
CATTTTTTTACCATGTTTCACTCCATAAGCGTCTGAGATTATCACAAGTCACGTCTAGTAGTTTGATGGTACACT
AGTGACAATCAGTTCGTGCAGACAGAGCTCATACTTGACTACTTGAGCGATTACAGGCGAAAGTGTGAAACGC
ATGTGATGTGGGCTGGGAGGAGGAGAATATATACTAATGGGCCGTATCCTGATTTGGGCTGCGTCGGAAGGTG
CAGCCCACGCGCGCCGTACCGCGCGGGTGGCGCTGCTACCCACTTTAGTCCGTTGGATGGGGATCCGATGGTTT
GCGCGGTGGCGTTGCGGGGGATGTTTAGTACCACATCGGAAACCGAAAGACGATGGAACCAGCTTATAAACCC
GCGCGCTGTAGTCAGCTTGGCTTGGGCTCGGAGCATCCGTTTTAGAGCTAGAAATAGCAAGTTAAAATAAGGCT
AGTCCGTTATCAACTTGAAAAAGTGGCACCGAGTCGGTGCTTTTTTTTTGCGGCCGCCTGCAGGTCAACGGATC
ACCTATCAACATCCCAGCTAAAAACAGTAAAAAGGGGGAAAACGTGGGTGAGTTGAGTCTGTCTTGTGGAAAA
AACGTTTTAGTTTCTCCTGGAATTAACAATAAAAACAGTTGAACAAGATTGACTGTTCCTCCGGGAGGGTTTGG
AACATCGTTACAGATGTGAGCGAAAGGTGAGGAAACAGAGCGGAGGGCTTGGAGGTGACCTCGGTAGTCGACG
CCGGAGTTGAGCTTGATGACGACACCGTACTGGCGTACCAGGCCTAGTAGTGAACACCGGGCCTGAAGCTGTC
GCCGCCGCTGCTCATCTTGTGGGCTGTGCCCGGTGTCCCTGTTGCGGATTGCGGGTGGCAGCCTGGCAGGTGGG
TGCGACCCGTTTGGACTCCCTGATCTGGGCCCTTTGTGTCAGTACCGTCTGTACTCCGATGACATGCACACCGTC
GTCCACAGTCAAGTCCACAATCTCCCCTCTTTTTTTAACGGAATAGTTCAAAATCTCCTTGACGCACGCTATCGT
GTACCAGCGCTCACTGGACACCACGTTTGTAATCCACGCCGACACGTCGGTCCCACGTCGACAGGCCCCACCGT
CCGGTCTGTAGCGTGTACGTATTCGGGCGACGGACGTGTCGTCGTCGTCTTGCGAGTCCCATTCCCATCACCATC
TGAGCCACACATCCTCTGAACAAAAGCAGGGAGGCCTCTACGCACATCCCCCTTTCTCCCACTCCTTGTCCGTG
GCACCCACCCCAAACCCTCGCGCCGCCTCCGAGACAGCCGCCGCAACCATGGCCACCGCCTGCAGGACCGTCTT
CGGTACGCGCTCACTCCGCCCTCTGCCTTTGTTACTGCCACGTTTCTCTGAATGCTCTCTTGTGTGGTGATTGCTG
AGAGTGGTTTAGCTGGATCTAGAATTACACTCTGAAATCGTGTTCTGCCTGTGCTGATTACTTGCCGTCCTTTGT
AGCAGCAAAATATAGGGACATGGTAGTACGAAACGAAGATAGAACCTACACAGCAATACGAGAAATGTGTAA
TTTGGTGCTTAGCGGTATTTATTTAAGCACATGTTGGTGTTATAGGGCACTTGGATTCAGAAGTTTGCTGTTAAT
TTAGGCACAGGCTTCATACTACATGGGTCAATAGTATAGGGATTCATATTATAGGCGATACTATAATAATTTGTT
CGTCTGCAGAGCTTATTATTTGCCAAAATTAGATATTCCTATTCTGTTTTTGTTTGTGTGCTGTTAAATTGTTAAC
GCCTGAAGGAATAAATATAAATGACGAAATTTTGATGTTTATCTCTGCTCCTTTATTGTGACCATAAGTCAAGAT
CAGATGCACTTGTTTTAAATATTGTTGTCTGAAGAAATAAGTACTGACAGTATTTTGATGCATTGATCTGCTTGT
TTGTTGTAACAAAATTTAAAAATAAAGAGTTTCCTTTTTGTTGCTCTCCTTACCTCCTGATGGTATCTAGTATCTA
CCAACTGACACTATATTGCTTCTCTTTACATACGTATCTTGCTCGATGCCTTCTCCCTAGTGTTGACCAGTGTTAC
TCACATAGTCTTTGCTCATTTCATTGTAATGCAGATACCAAGCGGCCCCTGCAGGATGGGGATTGAACAAGATG
GATTGCACGCAGGTTCTCCGGCCGCTTGGGTGGAGAGGCTATTCGGCTATGACTGGGCACAACAGACAATCGGC
TGCTCTGATGCCGCCGTGTTCCGGCTGTCAGCGCAGGGGCGCCCGGTTCTTTTTGTCAAGACCGACCTGTCCGGT
GCCCTGAATGAACTCCAGGACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCGGTCTTGTCG
ATCAGGATGATCTGGACGAAGAGCATCAGGGGCTCGCGCCAGCCGAACTGTTCGCCAGGCTCAAGGCGCGCAT
GCCCGACGGCGAGGATCTCGTCGTGACACATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGCTCCCGATTCGCA
GCGCATCGCCTTCTATCGCCTTCTTGACGAGTTCTTCTGAGCGATCGCAGAGCCATGGGGGCGGTGAGAGCTCG
CGGCGTCGGATCTCCGGTTTCAGTTTCAGCTACGCGACTTGTGTGTGTGTGTGTGTGTCAGTGCGTGGTTACTCT
GAGCTTCGGTATGTTTGGTGTGTACTGTTCAGTGTTTCATGGTGCTTCCTAGCAGTGTCTGTCGTGTACTTTGCCT
GTATGTTGAGCTCGGCTCAGTATGTTCTGGGAGTGAATAAATAAAAAAAAATTAACCAGATATTGTCGCGTGTA
CTTAGTATACTAATCACCTGCTCCTGCTCTTGCTCTGTTTTATCACCTTTCGGGCTTGACTGTCTTTGTGGAGGCC
TCTCAGTCTGATCTGGAACTCTACCTAAGTTTTACTAGTCCCGGGAAGCTTCTGCAGTGCAGCGTGACCCGGTCG
TGCCCCTCACTAGAGATAATGAGCATTGCATGTCTAAGTTATAAAAAATTACCACATATTTTTTTTGTCACACTT
GTTTGAAGTGCAGTTTATCTATCTTTATACATATATTTAAACTTTACTCTACGAATAATATAATCTATAGTACTAC
AATAATATCAGTGTTTTAGAGAATCATATAAATGAACAGTTAGACATGGTCTAAAGGACAATTGAGTATTTTGA
CAACAGGACTCTACAGTTTTATCTTTTTAGTGTGCATGTGTTCTCCTTTTTTTTTGCAAATAGCTTCACCTATATA
ATACTTCATCCATTTTATTAGTACATCCATTTAGGGTTTAGGGTTAATGGTTTTTATAGACTAATTTTTTTAGTAC
ATCTATTTTATTCTATTTTAGCCTCTAAATTAAGAAAACTAAAACTCTATTTTAGTTTTTTTATTTAATAATTTAG
ATATAAAATAGAATAAAATAAAGTGACTAAAAATTAAACAAATACCCTTTAAGAAATTAAAAAAACTAAGGAA
ACATTTTTCTTGTTTCGAGTAGATAATGCCAGCCTGTTAAACGCCGTCGACGAGTCTAACGGACACCAACCAGC
GAACCAGCAGCGTCGCGTCGGGCCAAGCGAAGCAGACGGCACGGCATCTCTGTCGCTGCCTCTGGACCCCTCTC
GAGAGTTCCGCTCCACCGTTGGACTTGCTCCGCTGTCGGCATCCAGAAATTGCGTGGCGGAGCGGCAGACGTGA
GCCGGCACGGCAGGCGGCCTCCTCCTCCTCTCACGGCACGGCAGCTACGGGGGATTCCTTTCCCACCGCTCCTT
CGCTTTCCCTTCCTCGCCCGCCGTAATAAATAGACACCCCCTCCACACCCTCTTTCCCCAACCTCGTGTTGTTCG
GAGCGCACACACACACAACCAGATCTCCCCCAAATCCACCCGTCGGCACCTCCGCTTCAAGGTACGCCGCTCGT
CCTCCCCCCCCCCCCCTCTCTACCTTCTCTAGATCGGCGTTCCGGTCCATGGTTAGGGCCCGGTAGTTCTACTTCT
GTTCATGTTTGTGTTAGATCCGTGTTTGTGTTAGATCCGTGCTGCTAGCGTTCGTACACGGATGCGACCTGTACG
TCAGACACGTTCTGATTGCTAACTTGCCAGTGTTTCTCTTTGGGGAATCCTGGGATGGCTCTAGCCGTTCCGCAG
ACGGGATCGATTTCATGATTTTTTTTGTTTCGTTGCATAGGGTTTGGTTTGCCCTTTTCCTTTATTTCAATATATGC
CGTGCACTTGTTTGTCGGGTCATCTTTTCATGCTTTTTTTTGTCTTGGTTGTGATGATGTGGTCTGGTTGGGCGGT
CGTTCTAGATCGGAGTAGAATTCTGTTTCAAACTACCTGGTGGATTTATTAATTTTGGATCTGTATGTGTGTGCC
ATACATATTCATAGTTACGAATTGAAGATGATGGATGGAAATATCGATCTAGGATAGGTATACATGTTGATGCG
GGTTTTACTGATGCATATACAGAGATGCTTTTTGTTCGCTTGGTTGTGATGATGTGGTGTGGTTGGGCGGTCGTT
CATTCGTTCTAGATCGGAGTAGAATACTGTTTCAAACTACCTGGTGTATTTATTAATTTTGGAACTGTATGTGTG
TGTCATACATCTTCATAGTTACGAGTTTAAGATGGATGGAAATATCGATCTAGGATAGGTATACATGTTGATGT
GGGTTTTACTGATGCATATACATGATGGCATATGCAGCATCTATTCATATGCTCTAACCTTGAGTACCTATCTAT
TATAATAAACAAGTATGTTTTATAATTATTTTGATCTTGATATACTTGGATGATGGCATATGCAGCAGCTATATG
TGGATTTTTTTAGCCCTGCCTTCATACGCTATTTATTTGCTTGGTACTGTTTCTTTTGTCGATGCTCACCCTGTTGT
TTGGTGTTACTTCTGCAGGGCCGGCCATGGATCCTAAAAAGAAACGTAAGGTCGACAAGAAGTACAGCATTGG
TCTGGACATCGGGACGAATTCCGTTGGCTGGGCCGTGATCACCGATGAGTACAAGGTCCCTTCCAAGAAGTTCA
AGGTTCTGGGGAACACCGATCGGCACAGCATCAAGAAGAACCTCATTGGAGCCCTCCTGTTCGACTCAGGCGA
GACCGCCGAAGCAACAAGGCTCAAGAGAACCGCAAGGAGACGGTATACCAGAAGGAAGAACAGGATCTGCTA
CCTGCAGGAGATCTTCAGCAACGAAATGGCGAAGGTGGACGATTCGTTCTTCCACAGATTGGAAGAGAGTTTCC
TCGTCGAGGAAGACAAGAAGCACGAGAGGCATCCGATCTTCGGCAACATCGTCGACGAGGTTGCCTATCACGA
GAAGTACCCCACCATCTACCATCTGCGGAAGAAGCTTGTGGACTCGACTGACAAGGCGGACCTTAGGTTGATCT
ACCTCGCTCTGGCCCACATGATCAAGTTCAGGGGCCATTTCCTGATCGAGGGGGATCTGAACCCGGACAACAGC
GATGTGGACAAGTTGTTCATCCAGCTCGTCCAAACCTACAACCAGCTCTTTGAGGAGAACCCGATCAACGCTTC
AGGCGTCGACGCCAAGGCGATCCTGTCTGCACGCCTGTCAAAGTCTCGCCGGCTTGAGAACTTGATCGCTCAAC
TCCCGGGCGAGAAGAAGAACGGCTTGTTCGGGAATCTCATTGCACTTTCGTTGGGGCTCACCCCGAACTTCAAG
AGCAACTTCGATCTCGCTGAGGACGCAAAGCTGCAGCTTTCCAAGGACACTTACGACGATGACCTGGATAACCT
GTTGGCCCAAATCGGCGATCAGTACGCGGACTTGTTCCTCGCCGCGAAGAACTTGTCGGACGCGATCCTCCTGA
GTGACATTCTCCGCGTGAACACCGAGATCACCAAGGCCCCGCTCTCGGCGAGCATGATCAAGCGGTATGACGA
GCACCATCAGGATCTGACCCTGTTGAAGGCTTTGGTCCGGCAGCAACTCCCAGAGAAGTACAAGGAGATCTTCT
TCGATCAGTCCAAGAACGGCTACGCTGGCTACATCGACGGCGGGGCATCGCAGGAGGAGTTCTACAAGTTCAT
CAAGCCAATCCTGGAGAAGATGGATGGCACAGAGGAACTCCTGGTGAAGCTCAACAGGGAGGACCTGTTGCGG
AAGCAAAGGACTTTCGATAACGGCAGCATCCCTCACCAGATCCATCTCGGGGAGCTGCACGCCATCCTGAGAA
GGCAGGAAGACTTCTACCCCTTCCTTAAGGATAACCGGGAGAAGATCGAGAAGATCCTGACGTTCAGAATCCC
GTACTACGTCGGACCACTCGCCCGGGGTAATTCCAGATTCGCGTGGATGACCAGAAAGAGCGAGGAAACCATC
ACCCCTTGGAACTTCGAGGAAGTGGTCGATAAGGGCGCTTCCGCACAGAGCTTCATCGAGCGCATGACCAACTT
CGACAAGAACCTGCCTAACGAGAAGGTCCTTCCCAAGCATTCCCTCCTGTACGAGTACTTCACTGTGTACAACG
AACTCACGAAGGTGAAGTACGTGACCGAGGGAATGCGCAAGCCCGCCTTCCTGAGCGGCGAGCAGAAGAAGG
CGATCGTGGACCTGTTGTTCAAGACCAATCGGAAGGTCACAGTGAAGCAGCTCAAGGAGGACTACTTCAAGAA
GATCGAATGCTTCGATTCCGTTGAGATCAGCGGCGTGGAAGACAGGTTTAACGCCTCACTGGGGACTTACCACG
ATCTCCTGAAGATCATCAAGGATAAGGACTTCTTGGACAACGAGGAGAATGAGGACATCCTCGAAGACATTGT
CCTGACTCTTACGTTGTTCGAGGATAGGGAGATGATCGAGGAACGCTTGAAGACGTATGCCCATCTCTTCGATG
ACAAGGTGATGAAGCAGCTCAAGAGAAGAAGGTACACCGGATGGGGAAGGCTGTCCCGCAAGCTGATCAACG
GCATCAGAGACAAGCAATCAGGGAAGACAATCCTTGACTTCTTGAAGTCTGATGGCTTCGCGAACAGGAACTTC
ATGCAGCTCATTCACGATGACTCACTGACCTTCAAGGAGGACATCCAGAAGGCTCAAGTGTCGGGACAAGGTG
ACAGTCTGCACGAGCATATCGCCAACCTTGCGGGATCTCCTGCCATCAAGAAGGGTATCCTGCAGACAGTCAAG
GTTGTGGATGAGCTTGTGAAGGTCATGGGACGGCATAAGCCCGAGAACATCGTTATCGAGATGGCCAGAGAGA
ATCAGACCACCCAGAAGGGTCAGAAGAACTCGAGGGAGCGCATGAAGCGCATCGAGGAAGGCATTAAGGAGC
TGGGGAGTCAGATCCTTAAGGAGCACCCGGTGGAGAACACGCAGTTGCAGAATGAGAAGCTCTATCTGTACTA
CCTGCAGAATGGCAGGGACATGTACGTGGACCAGGAGTTGGACATCAACCGCCTCTCGGATTACGACGTCGAT
CACATCGTTCCTCAGTCCTTCCTTAAGGATGACAGCATTGACAACAAGGTTCTCACCAGGTCCGACAAGAACCG
CGGGAAGTCCGATAACGTGCCCAGCGAGGAAGTCGTGAAGAAGATGAAGAACTACTGGAGGCAACTGCTGAAT
GCCAAGTTGATCACCCAGAGGAAGTTCGACAACCTCACTAAGGCCGAGCGCGGAGGTCTCAGCGAACTGGACA
AGGCGGGCTTCATCAAGCGGCAACTGGTTGAGACTAGACAGATCACGAAGCACGTCGCGCAGATTCTCGATTC
ACGCATGAACACGAAGTACGATGAGAATGACAAGCTGATCCGGGAAGTGAAGGTCATCACCTTGAAGTCAAAG
CTCGTTTCTGACTTCAGGAAGGATTTCCAGTTCTACAAGGTGCGCGAGATCAACAACTACCACCATGCTCATGA
CGCATACCTCAACGCTGTGGTCGGAACAGCACTGATCAAGAAGTACCCGAAGCTCGAGTCCGAATTCGTGTACG
GTGACTACAAGGTTTACGATGTGCGCAAGATGATCGCCAAGTCAGAGCAGGAAATTGGCAAGGCCACTGCGAA
GTACTTCTTCTACTCCAACATCATGAACTTCTTCAAGACTGAGATCACGCTGGCTAATGGCGAAATCCGGAAGA
GACCACTGATTGAGACCAACGGCGAGACAGGGGAAATCGTCTGGGACAAGGGGAGGGATTTCGCCACAGTCCG
CAAGGTTCTCTCTATGCCTCAAGTGAACATCGTCAAGAAGACTGAAGTCCAGACGGGCGGGTTCTCAAAGGAGT
CTATCCTGCCCAAGCGGAACTCGGATAAGCTTATCGCCAGGAAGAAGGACTGGGATCCGAAGAAGTACGGAGG
TTTCGACTCACCAACGGTGGCTTACTCTGTCCTGGTTGTGGCAAAGGTGGAGAAGGGAAAGTCGAAGAAGCTCA
AGTCTGTCAAGGAGCTGCTGGGTATCACCATTATGGAGAGGTCCAGCTTCGAGAAGAACCCGATCGACTTCCTC
GAGGCGAAGGGCTACAAGGAAGTGAAGAAGGACCTGATCATCAAGCTTCCGAAGTACAGTCTGTTCGAGTTGG
AGAACGGCAGGAAGCGCATGTTGGCTTCCGCAGGAGAGCTCCAGAAGGGTAACGAGCTTGCTTTGCCGTCCAA
GTACGTGAACTTCCTCTATCTGGCATCCCACTACGAGAAGCTCAAGGGCAGCCCAGAGGATAACGAACAGAAG
CAACTGTTTGTGGAGCAACACAAGCATTACCTTGACGAGATCATCGAACAGATCTCGGAGTTCAGTAAGCGCGT
CATCCTCGCCGACGCGAATTTGGATAAGGTTCTCTCAGCCTACAACAAGCACCGGGACAAGCCTATCAGAGAGC
AGGCGGAGAACATCATCCATCTCTTCACCCTGACAAACCTTGGGGCTCCCGCTGCCTTCAAGTACTTCGACACT
ACGATCGACCGGAAGAGGTACACTTCTACGAAGGAGGTGCTGGATGCAACCCTTATCCACCAATCGATCACTG
GCCTCTACGAGACGCGGATCGACTTGAGTCAGCTCGGTGGCGATAAGAGACCCGCAGCAACCAAGAAGGCAGG
GCAAGCAAAGAAGAAGAAGTGAGCGATCGCATATGAAGATGAAGATGAAATATTTGGTGTGTCAAATAAAAA
GCTTGTGTGCTTAAGTTTGTGTTTTTTTCTTGGCTTGTTGTGTTATGAATTTGTGGCTTTTTCTAATATTAAATGAA
TGTAAGATCTCATTATAATGAATAAACAAATGTTTCTATAATCCATTGTGAATGTTTTGTTGGATCTCTTCTGCA
GCATATAACTACTGTATGTGCTATGGTATGGACTATGGAATATGATTAAAGATAAGGGTACGTACCGCGATCGC
ATAACTTCGTATAGCATACATTATACGAAGTTATGAATTCGGCAATCAAGAGGTAAAGGATAATAATCCCAGCA
GAAACCGTGATCAAAGAAACAATTCATTGCAATATAGACTAGGCCATGCATACGCAAATCCAAGCGTGCATCC
ACTGACTGCATAACATAAACCCCCAGGTACTACGACTAAGCCGAACTGATACAAAATGCACTAGCTATTGGATA
ATCAGATCCTGGTCCATGCTGCTGTCATTCCATCGCAATCACCCTGGTAGCGACGACAGGATCATTTGCTTCCAG
AACCGAACTGTGCGCCTAGCTCGGCGCGCCTAACTATAACGGTCCTAA
SEQ ID NO: 21 (ScFT8 sgRNA expression vector, minimal cassette, 11158 bp)
GGTAGCGAGGCGCGCCGTTGTTTTAGTGTCCGAGGTTTGGTTTGGTGAAGTACATCTGAACTCTGAAGGTTCTTC
GTGACATGGTTAGACGTGGTGTTGCAGTACTGTGATAAAACTTATCAGAATAAGTTTTCGCTTCTAATTTGTTTT
CTTAAATCATGTGTTGATCAGCCTATGAGTTGTTCATATCGTTGCCTTTCGTGTTGAACATTTGTTTTCTTGCCAT
ACTAATGAGATTCATCTGCACAAACTGTACGAAGAGAATCAAGATATCGCCCGGGCTTTGTGAAAGTTGAATTA
CGGCATAGCCGAAGGAATAACAGAATCGTTTCACACTTTCGTAACAAAGATCTTCTTATCATGTTTCAGACGAT
GGAGGCAAGGCTGATCAAAGTGATCAAGCACATAAACGCATTTTTTTACCATGTTTCACTCCATAAGCGTCTGA
GATTATCACAAGTCACGTCTAGTAGTTTGATGGTACACTAGTGACAATCAGTTCGTGCAGACAGAGCTCATACT
TGACTACTTGAGCGATTACAGGCGAAAGTGTGAAACGCATGTGATGTGGGCTGGGAGGAGGAGAATATATACT
AATGGGCCGTATCCTGATTTGGGCTGCGTCGGAAGGTGCAGCCCACGCGCGCCGTACCGCGCGGGTGGCGCTGC
TACCCACTTTAGTCCGTTGGATGGGGATCCGATGGTTTGCGCGGTGGCGTTGCGGGGGATGTTTAGTACCACAT
CGGAAACCGAAAGACGATGGAACCAGCTTATAAACCCGCGCGCTGTAGTCAGCTTGAGAAGTAGCCTATTGTT
GTGTTTTAGAGCTAGAAATAGCAAGTTAAAATAAGGCTAGTCCGTTATCAACTTGAAAAAGTGGCACCGAGTCG
GTGCTTTTTTTTTAAGCTTTTTGTGAAAGTTGAATTACGGCATAGCCGAAGGAATAACAGAATCGTTTCACACTT
TCGTAACAAAGATCTTCTTATCATGTTTCAGACGATGGAGGCAAGGCTGATCAAAGTGATCAAGCACATAAACG
CATTTTTTTACCATGTTTCACTCCATAAGCGTCTGAGATTATCACAAGTCACGTCTAGTAGTTTGATGGTACACT
AGTGACAATCAGTTCGTGCAGACAGAGCTCATACTTGACTACTTGAGCGATTACAGGCGAAAGTGTGAAACGC
ATGTGATGTGGGCTGGGAGGAGGAGAATATATACTAATGGGCCGTATCCTGATTTGGGCTGCGTCGGAAGGTG
CAGCCCACGCGCGCCGTACCGCGCGGGTGGCGCTGCTACCCACTTTAGTCCGTTGGATGGGGATCCGATGGTTT
GCGCGGTGGCGTTGCGGGGGATGTTTAGTACCACATCGGAAACCGAAAGACGATGGAACCAGCTTATAAACCC
GCGCGCTGTAGTCAGCTTGCCCAAGCAATCCATCACTGGTTTTAGAGCTAGAAATAGCAAGTTAAAATAAGGCT
AGTCCGTTATCAACTTGAAAAAGTGGCACCGAGTCGGTGCTTTTTTTTTGCGGCCGCCTGCAGGTCAACGGATC
ACCTATCAACATCCCAGCTAAAAACAGTAAAAAGGGGGAAAACGTGGGTGAGTTGAGTCTGTCTTGTGGAAAA
AACGTTTTAGTTTCTCCTGGAATTAACAATAAAAACAGTTGAACAAGATTGACTGTTCCTCCGGGAGGGTTTGG
AACATCGTTACAGATGTGAGCGAAAGGTGAGGAAACAGAGCGGAGGGCTTGGAGGTGACCTCGGTAGTCGACG
CCGGAGTTGAGCTTGATGACGACACCGTACTGGCGTACCAGGCCTAGTAGTGAACACCGGGCCTGAAGCTGTC
GCCGCCGCTGCTCATCTTGTGGGCTGTGCCCGGTGTCCCTGTTGCGGATTGCGGGTGGCAGCCTGGCAGGTGGG
TGCGACCCGTTTGGACTCCCTGATCTGGGCCCTTTGTGTCAGTACCGTCTGTACTCCGATGACATGCACACCGTC
GTCCACAGTCAAGTCCACAATCTCCCCTCTTTTTTTAACGGAATAGTTCAAAATCTCCTTGACGCACGCTATCGT
GTACCAGCGCTCACTGGACACCACGTTTGTAATCCACGCCGACACGTCGGTCCCACGTCGACAGGCCCCACCGT
CCGGTCTGTAGCGTGTACGTATTCGGGCGACGGACGTGTCGTCGTCGTCTTGCGAGTCCCATTCCCATCACCATC
TGAGCCACACATCCTCTGAACAAAAGCAGGGAGGCCTCTACGCACATCCCCCTTTCTCCCACTCCTTGTCCGTG
GCACCCACCCCAAACCCTCGCGCCGCCTCCGAGACAGCCGCCGCAACCATGGCCACCGCCTGCAGGACCGTCTT
CGGTACGCGCTCACTCCGCCCTCTGCCTTTGTTACTGCCACGTTTCTCTGAATGCTCTCTTGTGTGGTGATTGCTG
AGAGTGGTTTAGCTGGATCTAGAATTACACTCTGAAATCGTGTTCTGCCTGTGCTGATTACTTGCCGTCCTTTGT
AGCAGCAAAATATAGGGACATGGTAGTACGAAACGAAGATAGAACCTACACAGCAATACGAGAAATGTGTAA
TTTGGTGCTTAGCGGTATTTATTTAAGCACATGTTGGTGTTATAGGGCACTTGGATTCAGAAGTTTGCTGTTAAT
TTAGGCACAGGCTTCATACTACATGGGTCAATAGTATAGGGATTCATATTATAGGCGATACTATAATAATTTGTT
CGTCTGCAGAGCTTATTATTTGCCAAAATTAGATATTCCTATTCTGTTTTTGTTTGTGTGCTGTTAAATTGTTAAC
GCCTGAAGGAATAAATATAAATGACGAAATTTTGATGTTTATCTCTGCTCCTTTATTGTGACCATAAGTCAAGAT
CAGATGCACTTGTTTTAAATATTGTTGTCTGAAGAAATAAGTACTGACAGTATTTTGATGCATTGATCTGCTTGT
TTGTTGTAACAAAATTTAAAAATAAAGAGTTTCCTTTTTGTTGCTCTCCTTACCTCCTGATGGTATCTAGTATCTA
CCAACTGACACTATATTGCTTCTCTTTACATACGTATCTTGCTCGATGCCTTCTCCCTAGTGTTGACCAGTGTTAC
TCACATAGTCTTTGCTCATTTCATTGTAATGCAGATACCAAGCGGCCCCTGCAGGATGGGGATTGAACAAGATG
GATTGCACGCAGGTTCTCCGGCCGCTTGGGTGGAGAGGCTATTCGGCTATGACTGGGCACAACAGACAATCGGC
TGCTCTGATGCCGCCGTGTTCCGGCTGTCAGCGCAGGGGCGCCCGGTTCTTTTTGTCAAGACCGACCTGTCCGGT
GCCCTGAATGAACTCCAGGACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCGGTCTTGTCG
ATCAGGATGATCTGGACGAAGAGCATCAGGGGCTCGCGCCAGCCGAACTGTTCGCCAGGCTCAAGGCGCGCAT
GCCCGACGGCGAGGATCTCGTCGTGACACATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGCTCCCGATTCGCA
GCGCATCGCCTTCTATCGCCTTCTTGACGAGTTCTTCTGAGCGATCGCAGAGCCATGGGGGCGGTGAGAGCTCG
CGGCGTCGGATCTCCGGTTTCAGTTTCAGCTACGCGACTTGTGTGTGTGTGTGTGTGTCAGTGCGTGGTTACTCT
GAGCTTCGGTATGTTTGGTGTGTACTGTTCAGTGTTTCATGGTGCTTCCTAGCAGTGTCTGTCGTGTACTTTGCCT
GTATGTTGAGCTCGGCTCAGTATGTTCTGGGAGTGAATAAATAAAAAAAAATTAACCAGATATTGTCGCGTGTA
CTTAGTATACTAATCACCTGCTCCTGCTCTTGCTCTGTTTTATCACCTTTCGGGCTTGACTGTCTTTGTGGAGGCC
TCTCAGTCTGATCTGGAACTCTACCTAAGTTTTACTAGTCCCGGGAAGCTTCTGCAGTGCAGCGTGACCCGGTCG
TGCCCCTCACTAGAGATAATGAGCATTGCATGTCTAAGTTATAAAAAATTACCACATATTTTTTTTGTCACACTT
GTTTGAAGTGCAGTTTATCTATCTTTATACATATATTTAAACTTTACTCTACGAATAATATAATCTATAGTACTAC
AATAATATCAGTGTTTTAGAGAATCATATAAATGAACAGTTAGACATGGTCTAAAGGACAATTGAGTATTTTGA
CAACAGGACTCTACAGTTTTATCTTTTTAGTGTGCATGTGTTCTCCTTTTTTTTTGCAAATAGCTTCACCTATATA
ATACTTCATCCATTTTATTAGTACATCCATTTAGGGTTTAGGGTTAATGGTTTTTATAGACTAATTTTTTTAGTAC
ATCTATTTTATTCTATTTTAGCCTCTAAATTAAGAAAACTAAAACTCTATTTTAGTTTTTTTATTTAATAATTTAG
ATATAAAATAGAATAAAATAAAGTGACTAAAAATTAAACAAATACCCTTTAAGAAATTAAAAAAACTAAGGAA
ACATTTTTCTTGTTTCGAGTAGATAATGCCAGCCTGTTAAACGCCGTCGACGAGTCTAACGGACACCAACCAGC
GAACCAGCAGCGTCGCGTCGGGCCAAGCGAAGCAGACGGCACGGCATCTCTGTCGCTGCCTCTGGACCCCTCTC
GAGAGTTCCGCTCCACCGTTGGACTTGCTCCGCTGTCGGCATCCAGAAATTGCGTGGCGGAGCGGCAGACGTGA
GCCGGCACGGCAGGCGGCCTCCTCCTCCTCTCACGGCACGGCAGCTACGGGGGATTCCTTTCCCACCGCTCCTT
CGCTTTCCCTTCCTCGCCCGCCGTAATAAATAGACACCCCCTCCACACCCTCTTTCCCCAACCTCGTGTTGTTCG
GAGCGCACACACACACAACCAGATCTCCCCCAAATCCACCCGTCGGCACCTCCGCTTCAAGGTACGCCGCTCGT
CCTCCCCCCCCCCCCCTCTCTACCTTCTCTAGATCGGCGTTCCGGTCCATGGTTAGGGCCCGGTAGTTCTACTTCT
GTTCATGTTTGTGTTAGATCCGTGTTTGTGTTAGATCCGTGCTGCTAGCGTTCGTACACGGATGCGACCTGTACG
TCAGACACGTTCTGATTGCTAACTTGCCAGTGTTTCTCTTTGGGGAATCCTGGGATGGCTCTAGCCGTTCCGCAG
ACGGGATCGATTTCATGATTTTTTTTGTTTCGTTGCATAGGGTTTGGTTTGCCCTTTTCCTTTATTTCAATATATGC
CGTGCACTTGTTTGTCGGGTCATCTTTTCATGCTTTTTTTTGTCTTGGTTGTGATGATGTGGTCTGGTTGGGCGGT
CGTTCTAGATCGGAGTAGAATTCTGTTTCAAACTACCTGGTGGATTTATTAATTTTGGATCTGTATGTGTGTGCC
ATACATATTCATAGTTACGAATTGAAGATGATGGATGGAAATATCGATCTAGGATAGGTATACATGTTGATGCG
GGTTTTACTGATGCATATACAGAGATGCTTTTTGTTCGCTTGGTTGTGATGATGTGGTGTGGTTGGGCGGTCGTT
CATTCGTTCTAGATCGGAGTAGAATACTGTTTCAAACTACCTGGTGTATTTATTAATTTTGGAACTGTATGTGTG
TGTCATACATCTTCATAGTTACGAGTTTAAGATGGATGGAAATATCGATCTAGGATAGGTATACATGTTGATGT
GGGTTTTACTGATGCATATACATGATGGCATATGCAGCATCTATTCATATGCTCTAACCTTGAGTACCTATCTAT
TATAATAAACAAGTATGTTTTATAATTATTTTGATCTTGATATACTTGGATGATGGCATATGCAGCAGCTATATG
TGGATTTTTTTAGCCCTGCCTTCATACGCTATTTATTTGCTTGGTACTGTTTCTTTTGTCGATGCTCACCCTGTTGT
TTGGTGTTACTTCTGCAGGGCCGGCCATGGATCCTAAAAAGAAACGTAAGGTCGACAAGAAGTACAGCATTGG
TCTGGACATCGGGACGAATTCCGTTGGCTGGGCCGTGATCACCGATGAGTACAAGGTCCCTTCCAAGAAGTTCA
AGGTTCTGGGGAACACCGATCGGCACAGCATCAAGAAGAACCTCATTGGAGCCCTCCTGTTCGACTCAGGCGA
GACCGCCGAAGCAACAAGGCTCAAGAGAACCGCAAGGAGACGGTATACCAGAAGGAAGAACAGGATCTGCTA
CCTGCAGGAGATCTTCAGCAACGAAATGGCGAAGGTGGACGATTCGTTCTTCCACAGATTGGAAGAGAGTTTCC
TCGTCGAGGAAGACAAGAAGCACGAGAGGCATCCGATCTTCGGCAACATCGTCGACGAGGTTGCCTATCACGA
GAAGTACCCCACCATCTACCATCTGCGGAAGAAGCTTGTGGACTCGACTGACAAGGCGGACCTTAGGTTGATCT
ACCTCGCTCTGGCCCACATGATCAAGTTCAGGGGCCATTTCCTGATCGAGGGGGATCTGAACCCGGACAACAGC
GATGTGGACAAGTTGTTCATCCAGCTCGTCCAAACCTACAACCAGCTCTTTGAGGAGAACCCGATCAACGCTTC
AGGCGTCGACGCCAAGGCGATCCTGTCTGCACGCCTGTCAAAGTCTCGCCGGCTTGAGAACTTGATCGCTCAAC
TCCCGGGCGAGAAGAAGAACGGCTTGTTCGGGAATCTCATTGCACTTTCGTTGGGGCTCACCCCGAACTTCAAG
AGCAACTTCGATCTCGCTGAGGACGCAAAGCTGCAGCTTTCCAAGGACACTTACGACGATGACCTGGATAACCT
GTTGGCCCAAATCGGCGATCAGTACGCGGACTTGTTCCTCGCCGCGAAGAACTTGTCGGACGCGATCCTCCTGA
GTGACATTCTCCGCGTGAACACCGAGATCACCAAGGCCCCGCTCTCGGCGAGCATGATCAAGCGGTATGACGA
GCACCATCAGGATCTGACCCTGTTGAAGGCTTTGGTCCGGCAGCAACTCCCAGAGAAGTACAAGGAGATCTTCT
TCGATCAGTCCAAGAACGGCTACGCTGGCTACATCGACGGCGGGGCATCGCAGGAGGAGTTCTACAAGTTCAT
CAAGCCAATCCTGGAGAAGATGGATGGCACAGAGGAACTCCTGGTGAAGCTCAACAGGGAGGACCTGTTGCGG
AAGCAAAGGACTTTCGATAACGGCAGCATCCCTCACCAGATCCATCTCGGGGAGCTGCACGCCATCCTGAGAA
GGCAGGAAGACTTCTACCCCTTCCTTAAGGATAACCGGGAGAAGATCGAGAAGATCCTGACGTTCAGAATCCC
GTACTACGTCGGACCACTCGCCCGGGGTAATTCCAGATTCGCGTGGATGACCAGAAAGAGCGAGGAAACCATC
ACCCCTTGGAACTTCGAGGAAGTGGTCGATAAGGGCGCTTCCGCACAGAGCTTCATCGAGCGCATGACCAACTT
CGACAAGAACCTGCCTAACGAGAAGGTCCTTCCCAAGCATTCCCTCCTGTACGAGTACTTCACTGTGTACAACG
AACTCACGAAGGTGAAGTACGTGACCGAGGGAATGCGCAAGCCCGCCTTCCTGAGCGGCGAGCAGAAGAAGG
CGATCGTGGACCTGTTGTTCAAGACCAATCGGAAGGTCACAGTGAAGCAGCTCAAGGAGGACTACTTCAAGAA
GATCGAATGCTTCGATTCCGTTGAGATCAGCGGCGTGGAAGACAGGTTTAACGCCTCACTGGGGACTTACCACG
ATCTCCTGAAGATCATCAAGGATAAGGACTTCTTGGACAACGAGGAGAATGAGGACATCCTCGAAGACATTGT
CCTGACTCTTACGTTGTTCGAGGATAGGGAGATGATCGAGGAACGCTTGAAGACGTATGCCCATCTCTTCGATG
ACAAGGTGATGAAGCAGCTCAAGAGAAGAAGGTACACCGGATGGGGAAGGCTGTCCCGCAAGCTGATCAACG
GCATCAGAGACAAGCAATCAGGGAAGACAATCCTTGACTTCTTGAAGTCTGATGGCTTCGCGAACAGGAACTTC
ATGCAGCTCATTCACGATGACTCACTGACCTTCAAGGAGGACATCCAGAAGGCTCAAGTGTCGGGACAAGGTG
ACAGTCTGCACGAGCATATCGCCAACCTTGCGGGATCTCCTGCCATCAAGAAGGGTATCCTGCAGACAGTCAAG
GTTGTGGATGAGCTTGTGAAGGTCATGGGACGGCATAAGCCCGAGAACATCGTTATCGAGATGGCCAGAGAGA
ATCAGACCACCCAGAAGGGTCAGAAGAACTCGAGGGAGCGCATGAAGCGCATCGAGGAAGGCATTAAGGAGC
TGGGGAGTCAGATCCTTAAGGAGCACCCGGTGGAGAACACGCAGTTGCAGAATGAGAAGCTCTATCTGTACTA
CCTGCAGAATGGCAGGGACATGTACGTGGACCAGGAGTTGGACATCAACCGCCTCTCGGATTACGACGTCGAT
CACATCGTTCCTCAGTCCTTCCTTAAGGATGACAGCATTGACAACAAGGTTCTCACCAGGTCCGACAAGAACCG
CGGGAAGTCCGATAACGTGCCCAGCGAGGAAGTCGTGAAGAAGATGAAGAACTACTGGAGGCAACTGCTGAAT
GCCAAGTTGATCACCCAGAGGAAGTTCGACAACCTCACTAAGGCCGAGCGCGGAGGTCTCAGCGAACTGGACA
AGGCGGGCTTCATCAAGCGGCAACTGGTTGAGACTAGACAGATCACGAAGCACGTCGCGCAGATTCTCGATTC
ACGCATGAACACGAAGTACGATGAGAATGACAAGCTGATCCGGGAAGTGAAGGTCATCACCTTGAAGTCAAAG
CTCGTTTCTGACTTCAGGAAGGATTTCCAGTTCTACAAGGTGCGCGAGATCAACAACTACCACCATGCTCATGA
CGCATACCTCAACGCTGTGGTCGGAACAGCACTGATCAAGAAGTACCCGAAGCTCGAGTCCGAATTCGTGTACG
GTGACTACAAGGTTTACGATGTGCGCAAGATGATCGCCAAGTCAGAGCAGGAAATTGGCAAGGCCACTGCGAA
GTACTTCTTCTACTCCAACATCATGAACTTCTTCAAGACTGAGATCACGCTGGCTAATGGCGAAATCCGGAAGA
GACCACTGATTGAGACCAACGGCGAGACAGGGGAAATCGTCTGGGACAAGGGGAGGGATTTCGCCACAGTCCG
CAAGGTTCTCTCTATGCCTCAAGTGAACATCGTCAAGAAGACTGAAGTCCAGACGGGCGGGTTCTCAAAGGAGT
CTATCCTGCCCAAGCGGAACTCGGATAAGCTTATCGCCAGGAAGAAGGACTGGGATCCGAAGAAGTACGGAGG
TTTCGACTCACCAACGGTGGCTTACTCTGTCCTGGTTGTGGCAAAGGTGGAGAAGGGAAAGTCGAAGAAGCTCA
AGTCTGTCAAGGAGCTGCTGGGTATCACCATTATGGAGAGGTCCAGCTTCGAGAAGAACCCGATCGACTTCCTC
GAGGCGAAGGGCTACAAGGAAGTGAAGAAGGACCTGATCATCAAGCTTCCGAAGTACAGTCTGTTCGAGTTGG
AGAACGGCAGGAAGCGCATGTTGGCTTCCGCAGGAGAGCTCCAGAAGGGTAACGAGCTTGCTTTGCCGTCCAA
GTACGTGAACTTCCTCTATCTGGCATCCCACTACGAGAAGCTCAAGGGCAGCCCAGAGGATAACGAACAGAAG
CAACTGTTTGTGGAGCAACACAAGCATTACCTTGACGAGATCATCGAACAGATCTCGGAGTTCAGTAAGCGCGT
CATCCTCGCCGACGCGAATTTGGATAAGGTTCTCTCAGCCTACAACAAGCACCGGGACAAGCCTATCAGAGAGC
AGGCGGAGAACATCATCCATCTCTTCACCCTGACAAACCTTGGGGCTCCCGCTGCCTTCAAGTACTTCGACACT
ACGATCGACCGGAAGAGGTACACTTCTACGAAGGAGGTGCTGGATGCAACCCTTATCCACCAATCGATCACTG
GCCTCTACGAGACGCGGATCGACTTGAGTCAGCTCGGTGGCGATAAGAGACCCGCAGCAACCAAGAAGGCAGG
GCAAGCAAAGAAGAAGAAGTGAGCGATCGCATATGAAGATGAAGATGAAATATTTGGTGTGTCAAATAAAAA
GCTTGTGTGCTTAAGTTTGTGTTTTTTTCTTGGCTTGTTGTGTTATGAATTTGTGGCTTTTTCTAATATTAAATGAA
TGTAAGATCTCATTATAATGAATAAACAAATGTTTCTATAATCCATTGTGAATGTTTTGTTGGATCTCTTCTGCA
GCATATAACTACTGTATGTGCTATGGTATGGACTATGGAATATGATTAAAGATAAGGGTACGTACCGCGATCGC
ATAACTTCGTATAGCATACATTATACGAAGTTATGAATTCGGCAATCAAGAGGTAAAGGATAATAATCCCAGCA
GAAACCGTGATCAAAGAAACAATTCATTGCAATATAGACTAGGCCATGCATACGCAAATCCAAGCGTGCATCC
ACTGACTGCATAACATAAACCCCCAGGTACTACGACTAAGCCGAACTGATACAAAATGCACTAGCTATTGGATA
ATCAGATCCTGGTCCATGCTGCTGTCATTCCATCGCAATCACCCTGGTAGCGACGACAGGATCATTTGCTTCCAG
AACCGAACTGTGCGCCTAGCTCGGCGCGCCTAACTATAACGGTCCTAA
SEQ ID NO: 22 (ScFT10 sgRNA expression vector, minimal cassette, 11158 bp)
GGTAGCGAGGCGCGCCGTTGTTTTAGTGTCCGAGGTTTGGTTTGGTGAAGTACATCTGAACTCTGAAGGTTCTTC
GTGACATGGTTAGACGTGGTGTTGCAGTACTGTGATAAAACTTATCAGAATAAGTTTTCGCTTCTAATTTGTTTT
CTTAAATCATGTGTTGATCAGCCTATGAGTTGTTCATATCGTTGCCTTTCGTGTTGAACATTTGTTTTCTTGCCAT
ACTAATGAGATTCATCTGCACAAACTGTACGAAGAGAATCAAGATATCGCCCGGGCTTTGTGAAAGTTGAATTA
CGGCATAGCCGAAGGAATAACAGAATCGTTTCACACTTTCGTAACAAAGATCTTCTTATCATGTTTCAGACGAT
GGAGGCAAGGCTGATCAAAGTGATCAAGCACATAAACGCATTTTTTTACCATGTTTCACTCCATAAGCGTCTGA
GATTATCACAAGTCACGTCTAGTAGTTTGATGGTACACTAGTGACAATCAGTTCGTGCAGACAGAGCTCATACT
TGACTACTTGAGCGATTACAGGCGAAAGTGTGAAACGCATGTGATGTGGGCTGGGAGGAGGAGAATATATACT
AATGGGCCGTATCCTGATTTGGGCTGCGTCGGAAGGTGCAGCCCACGCGCGCCGTACCGCGCGGGTGGCGCTGC
TACCCACTTTAGTCCGTTGGATGGGGATCCGATGGTTTGCGCGGTGGCGTTGCGGGGGATGTTTAGTACCACAT
CGGAAACCGAAAGACGATGGAACCAGCTTATAAACCCGCGCGCTGTAGTCAGCTTGGTAAACCACGAGTCGAT
GTGTTTTAGAGCTAGAAATAGCAAGTTAAAATAAGGCTAGTCCGTTATCAACTTGAAAAAGTGGCACCGAGTCG
GTGCTTTTTTTTTAAGCTTTTTGTGAAAGTTGAATTACGGCATAGCCGAAGGAATAACAGAATCGTTTCACACTT
TCGTAACAAAGATCTTCTTATCATGTTTCAGACGATGGAGGCAAGGCTGATCAAAGTGATCAAGCACATAAACG
CATTTTTTTACCATGTTTCACTCCATAAGCGTCTGAGATTATCACAAGTCACGTCTAGTAGTTTGATGGTACACT
AGTGACAATCAGTTCGTGCAGACAGAGCTCATACTTGACTACTTGAGCGATTACAGGCGAAAGTGTGAAACGC
ATGTGATGTGGGCTGGGAGGAGGAGAATATATACTAATGGGCCGTATCCTGATTTGGGCTGCGTCGGAAGGTG
CAGCCCACGCGCGCCGTACCGCGCGGGTGGCGCTGCTACCCACTTTAGTCCGTTGGATGGGGATCCGATGGTTT
GCGCGGTGGCGTTGCGGGGGATGTTTAGTACCACATCGGAAACCGAAAGACGATGGAACCAGCTTATAAACCC
GCGCGCTGTAGTCAGCTTGTCCAAGCCATCCATCACTAGTTTTAGAGCTAGAAATAGCAAGTTAAAATAAGGCT
AGTCCGTTATCAACTTGAAAAAGTGGCACCGAGTCGGTGCTTTTTTTTTGCGGCCGCCTGCAGGTCAACGGATC
ACCTATCAACATCCCAGCTAAAAACAGTAAAAAGGGGGAAAACGTGGGTGAGTTGAGTCTGTCTTGTGGAAAA
AACGTTTTAGTTTCTCCTGGAATTAACAATAAAAACAGTTGAACAAGATTGACTGTTCCTCCGGGAGGGTTTGG
AACATCGTTACAGATGTGAGCGAAAGGTGAGGAAACAGAGCGGAGGGCTTGGAGGTGACCTCGGTAGTCGACG
CCGGAGTTGAGCTTGATGACGACACCGTACTGGCGTACCAGGCCTAGTAGTGAACACCGGGCCTGAAGCTGTC
GCCGCCGCTGCTCATCTTGTGGGCTGTGCCCGGTGTCCCTGTTGCGGATTGCGGGTGGCAGCCTGGCAGGTGGG
TGCGACCCGTTTGGACTCCCTGATCTGGGCCCTTTGTGTCAGTACCGTCTGTACTCCGATGACATGCACACCGTC
GTCCACAGTCAAGTCCACAATCTCCCCTCTTTTTTTAACGGAATAGTTCAAAATCTCCTTGACGCACGCTATCGT
GTACCAGCGCTCACTGGACACCACGTTTGTAATCCACGCCGACACGTCGGTCCCACGTCGACAGGCCCCACCGT
CCGGTCTGTAGCGTGTACGTATTCGGGCGACGGACGTGTCGTCGTCGTCTTGCGAGTCCCATTCCCATCACCATC
TGAGCCACACATCCTCTGAACAAAAGCAGGGAGGCCTCTACGCACATCCCCCTTTCTCCCACTCCTTGTCCGTG
GCACCCACCCCAAACCCTCGCGCCGCCTCCGAGACAGCCGCCGCAACCATGGCCACCGCCTGCAGGACCGTCTT
CGGTACGCGCTCACTCCGCCCTCTGCCTTTGTTACTGCCACGTTTCTCTGAATGCTCTCTTGTGTGGTGATTGCTG
AGAGTGGTTTAGCTGGATCTAGAATTACACTCTGAAATCGTGTTCTGCCTGTGCTGATTACTTGCCGTCCTTTGT
AGCAGCAAAATATAGGGACATGGTAGTACGAAACGAAGATAGAACCTACACAGCAATACGAGAAATGTGTAA
TTTGGTGCTTAGCGGTATTTATTTAAGCACATGTTGGTGTTATAGGGCACTTGGATTCAGAAGTTTGCTGTTAAT
TTAGGCACAGGCTTCATACTACATGGGTCAATAGTATAGGGATTCATATTATAGGCGATACTATAATAATTTGTT
CGTCTGCAGAGCTTATTATTTGCCAAAATTAGATATTCCTATTCTGTTTTTGTTTGTGTGCTGTTAAATTGTTAAC
GCCTGAAGGAATAAATATAAATGACGAAATTTTGATGTTTATCTCTGCTCCTTTATTGTGACCATAAGTCAAGAT
CAGATGCACTTGTTTTAAATATTGTTGTCTGAAGAAATAAGTACTGACAGTATTTTGATGCATTGATCTGCTTGT
TTGTTGTAACAAAATTTAAAAATAAAGAGTTTCCTTTTTGTTGCTCTCCTTACCTCCTGATGGTATCTAGTATCTA
CCAACTGACACTATATTGCTTCTCTTTACATACGTATCTTGCTCGATGCCTTCTCCCTAGTGTTGACCAGTGTTAC
TCACATAGTCTTTGCTCATTTCATTGTAATGCAGATACCAAGCGGCCCCTGCAGGATGGGGATTGAACAAGATG
GATTGCACGCAGGTTCTCCGGCCGCTTGGGTGGAGAGGCTATTCGGCTATGACTGGGCACAACAGACAATCGGC
TGCTCTGATGCCGCCGTGTTCCGGCTGTCAGCGCAGGGGCGCCCGGTTCTTTTTGTCAAGACCGACCTGTCCGGT
GCCCTGAATGAACTCCAGGACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCGGTCTTGTCG
ATCAGGATGATCTGGACGAAGAGCATCAGGGGCTCGCGCCAGCCGAACTGTTCGCCAGGCTCAAGGCGCGCAT
GCCCGACGGCGAGGATCTCGTCGTGACACATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGAT
ATTGCTGAAGAGCTTGGCGGCGAATGGGCTGACCGCTTCCTCGTGCTTTACGGTATCGCCGCTCCCGATTCGCA
GCGCATCGCCTTCTATCGCCTTCTTGACGAGTTCTTCTGAGCGATCGCAGAGCCATGGGGGCGGTGAGAGCTCG
CGGCGTCGGATCTCCGGTTTCAGTTTCAGCTACGCGACTTGTGTGTGTGTGTGTGTGTCAGTGCGTGGTTACTCT
GAGCTTCGGTATGTTTGGTGTGTACTGTTCAGTGTTTCATGGTGCTTCCTAGCAGTGTCTGTCGTGTACTTTGCCT
GTATGTTGAGCTCGGCTCAGTATGTTCTGGGAGTGAATAAATAAAAAAAAATTAACCAGATATTGTCGCGTGTA
CTTAGTATACTAATCACCTGCTCCTGCTCTTGCTCTGTTTTATCACCTTTCGGGCTTGACTGTCTTTGTGGAGGCC
TCTCAGTCTGATCTGGAACTCTACCTAAGTTTTACTAGTCCCGGGAAGCTTCTGCAGTGCAGCGTGACCCGGTCG
TGCCCCTCACTAGAGATAATGAGCATTGCATGTCTAAGTTATAAAAAATTACCACATATTTTTTTTGTCACACTT
GTTTGAAGTGCAGTTTATCTATCTTTATACATATATTTAAACTTTACTCTACGAATAATATAATCTATAGTACTAC
AATAATATCAGTGTTTTAGAGAATCATATAAATGAACAGTTAGACATGGTCTAAAGGACAATTGAGTATTTTGA
CAACAGGACTCTACAGTTTTATCTTTTTAGTGTGCATGTGTTCTCCTTTTTTTTTGCAAATAGCTTCACCTATATA
ATACTTCATCCATTTTATTAGTACATCCATTTAGGGTTTAGGGTTAATGGTTTTTATAGACTAATTTTTTTAGTAC
ATCTATTTTATTCTATTTTAGCCTCTAAATTAAGAAAACTAAAACTCTATTTTAGTTTTTTTATTTAATAATTTAG
ATATAAAATAGAATAAAATAAAGTGACTAAAAATTAAACAAATACCCTTTAAGAAATTAAAAAAACTAAGGAA
ACATTTTTCTTGTTTCGAGTAGATAATGCCAGCCTGTTAAACGCCGTCGACGAGTCTAACGGACACCAACCAGC
GAACCAGCAGCGTCGCGTCGGGCCAAGCGAAGCAGACGGCACGGCATCTCTGTCGCTGCCTCTGGACCCCTCTC
GAGAGTTCCGCTCCACCGTTGGACTTGCTCCGCTGTCGGCATCCAGAAATTGCGTGGCGGAGCGGCAGACGTGA
GCCGGCACGGCAGGCGGCCTCCTCCTCCTCTCACGGCACGGCAGCTACGGGGGATTCCTTTCCCACCGCTCCTT
CGCTTTCCCTTCCTCGCCCGCCGTAATAAATAGACACCCCCTCCACACCCTCTTTCCCCAACCTCGTGTTGTTCG
GAGCGCACACACACACAACCAGATCTCCCCCAAATCCACCCGTCGGCACCTCCGCTTCAAGGTACGCCGCTCGT
CCTCCCCCCCCCCCCCTCTCTACCTTCTCTAGATCGGCGTTCCGGTCCATGGTTAGGGCCCGGTAGTTCTACTTCT
GTTCATGTTTGTGTTAGATCCGTGTTTGTGTTAGATCCGTGCTGCTAGCGTTCGTACACGGATGCGACCTGTACG
TCAGACACGTTCTGATTGCTAACTTGCCAGTGTTTCTCTTTGGGGAATCCTGGGATGGCTCTAGCCGTTCCGCAG
ACGGGATCGATTTCATGATTTTTTTTGTTTCGTTGCATAGGGTTTGGTTTGCCCTTTTCCTTTATTTCAATATATGC
CGTGCACTTGTTTGTCGGGTCATCTTTTCATGCTTTTTTTTGTCTTGGTTGTGATGATGTGGTCTGGTTGGGCGGT
CGTTCTAGATCGGAGTAGAATTCTGTTTCAAACTACCTGGTGGATTTATTAATTTTGGATCTGTATGTGTGTGCC
ATACATATTCATAGTTACGAATTGAAGATGATGGATGGAAATATCGATCTAGGATAGGTATACATGTTGATGCG
GGTTTTACTGATGCATATACAGAGATGCTTTTTGTTCGCTTGGTTGTGATGATGTGGTGTGGTTGGGCGGTCGTT
CATTCGTTCTAGATCGGAGTAGAATACTGTTTCAAACTACCTGGTGTATTTATTAATTTTGGAACTGTATGTGTG
TGTCATACATCTTCATAGTTACGAGTTTAAGATGGATGGAAATATCGATCTAGGATAGGTATACATGTTGATGT
GGGTTTTACTGATGCATATACATGATGGCATATGCAGCATCTATTCATATGCTCTAACCTTGAGTACCTATCTAT
TATAATAAACAAGTATGTTTTATAATTATTTTGATCTTGATATACTTGGATGATGGCATATGCAGCAGCTATATG
TGGATTTTTTTAGCCCTGCCTTCATACGCTATTTATTTGCTTGGTACTGTTTCTTTTGTCGATGCTCACCCTGTTGT
TTGGTGTTACTTCTGCAGGGCCGGCCATGGATCCTAAAAAGAAACGTAAGGTCGACAAGAAGTACAGCATTGG
TCTGGACATCGGGACGAATTCCGTTGGCTGGGCCGTGATCACCGATGAGTACAAGGTCCCTTCCAAGAAGTTCA
AGGTTCTGGGGAACACCGATCGGCACAGCATCAAGAAGAACCTCATTGGAGCCCTCCTGTTCGACTCAGGCGA
GACCGCCGAAGCAACAAGGCTCAAGAGAACCGCAAGGAGACGGTATACCAGAAGGAAGAACAGGATCTGCTA
CCTGCAGGAGATCTTCAGCAACGAAATGGCGAAGGTGGACGATTCGTTCTTCCACAGATTGGAAGAGAGTTTCC
TCGTCGAGGAAGACAAGAAGCACGAGAGGCATCCGATCTTCGGCAACATCGTCGACGAGGTTGCCTATCACGA
GAAGTACCCCACCATCTACCATCTGCGGAAGAAGCTTGTGGACTCGACTGACAAGGCGGACCTTAGGTTGATCT
ACCTCGCTCTGGCCCACATGATCAAGTTCAGGGGCCATTTCCTGATCGAGGGGGATCTGAACCCGGACAACAGC
GATGTGGACAAGTTGTTCATCCAGCTCGTCCAAACCTACAACCAGCTCTTTGAGGAGAACCCGATCAACGCTTC
AGGCGTCGACGCCAAGGCGATCCTGTCTGCACGCCTGTCAAAGTCTCGCCGGCTTGAGAACTTGATCGCTCAAC
TCCCGGGCGAGAAGAAGAACGGCTTGTTCGGGAATCTCATTGCACTTTCGTTGGGGCTCACCCCGAACTTCAAG
AGCAACTTCGATCTCGCTGAGGACGCAAAGCTGCAGCTTTCCAAGGACACTTACGACGATGACCTGGATAACCT
GTTGGCCCAAATCGGCGATCAGTACGCGGACTTGTTCCTCGCCGCGAAGAACTTGTCGGACGCGATCCTCCTGA
GTGACATTCTCCGCGTGAACACCGAGATCACCAAGGCCCCGCTCTCGGCGAGCATGATCAAGCGGTATGACGA
GCACCATCAGGATCTGACCCTGTTGAAGGCTTTGGTCCGGCAGCAACTCCCAGAGAAGTACAAGGAGATCTTCT
TCGATCAGTCCAAGAACGGCTACGCTGGCTACATCGACGGCGGGGCATCGCAGGAGGAGTTCTACAAGTTCAT
CAAGCCAATCCTGGAGAAGATGGATGGCACAGAGGAACTCCTGGTGAAGCTCAACAGGGAGGACCTGTTGCGG
AAGCAAAGGACTTTCGATAACGGCAGCATCCCTCACCAGATCCATCTCGGGGAGCTGCACGCCATCCTGAGAA
GGCAGGAAGACTTCTACCCCTTCCTTAAGGATAACCGGGAGAAGATCGAGAAGATCCTGACGTTCAGAATCCC
GTACTACGTCGGACCACTCGCCCGGGGTAATTCCAGATTCGCGTGGATGACCAGAAAGAGCGAGGAAACCATC
ACCCCTTGGAACTTCGAGGAAGTGGTCGATAAGGGCGCTTCCGCACAGAGCTTCATCGAGCGCATGACCAACTT
CGACAAGAACCTGCCTAACGAGAAGGTCCTTCCCAAGCATTCCCTCCTGTACGAGTACTTCACTGTGTACAACG
AACTCACGAAGGTGAAGTACGTGACCGAGGGAATGCGCAAGCCCGCCTTCCTGAGCGGCGAGCAGAAGAAGG
CGATCGTGGACCTGTTGTTCAAGACCAATCGGAAGGTCACAGTGAAGCAGCTCAAGGAGGACTACTTCAAGAA
GATCGAATGCTTCGATTCCGTTGAGATCAGCGGCGTGGAAGACAGGTTTAACGCCTCACTGGGGACTTACCACG
ATCTCCTGAAGATCATCAAGGATAAGGACTTCTTGGACAACGAGGAGAATGAGGACATCCTCGAAGACATTGT
CCTGACTCTTACGTTGTTCGAGGATAGGGAGATGATCGAGGAACGCTTGAAGACGTATGCCCATCTCTTCGATG
ACAAGGTGATGAAGCAGCTCAAGAGAAGAAGGTACACCGGATGGGGAAGGCTGTCCCGCAAGCTGATCAACG
GCATCAGAGACAAGCAATCAGGGAAGACAATCCTTGACTTCTTGAAGTCTGATGGCTTCGCGAACAGGAACTTC
ATGCAGCTCATTCACGATGACTCACTGACCTTCAAGGAGGACATCCAGAAGGCTCAAGTGTCGGGACAAGGTG
ACAGTCTGCACGAGCATATCGCCAACCTTGCGGGATCTCCTGCCATCAAGAAGGGTATCCTGCAGACAGTCAAG
GTTGTGGATGAGCTTGTGAAGGTCATGGGACGGCATAAGCCCGAGAACATCGTTATCGAGATGGCCAGAGAGA
ATCAGACCACCCAGAAGGGTCAGAAGAACTCGAGGGAGCGCATGAAGCGCATCGAGGAAGGCATTAAGGAGC
TGGGGAGTCAGATCCTTAAGGAGCACCCGGTGGAGAACACGCAGTTGCAGAATGAGAAGCTCTATCTGTACTA
CCTGCAGAATGGCAGGGACATGTACGTGGACCAGGAGTTGGACATCAACCGCCTCTCGGATTACGACGTCGAT
CACATCGTTCCTCAGTCCTTCCTTAAGGATGACAGCATTGACAACAAGGTTCTCACCAGGTCCGACAAGAACCG
CGGGAAGTCCGATAACGTGCCCAGCGAGGAAGTCGTGAAGAAGATGAAGAACTACTGGAGGCAACTGCTGAAT
GCCAAGTTGATCACCCAGAGGAAGTTCGACAACCTCACTAAGGCCGAGCGCGGAGGTCTCAGCGAACTGGACA
AGGCGGGCTTCATCAAGCGGCAACTGGTTGAGACTAGACAGATCACGAAGCACGTCGCGCAGATTCTCGATTC
ACGCATGAACACGAAGTACGATGAGAATGACAAGCTGATCCGGGAAGTGAAGGTCATCACCTTGAAGTCAAAG
CTCGTTTCTGACTTCAGGAAGGATTTCCAGTTCTACAAGGTGCGCGAGATCAACAACTACCACCATGCTCATGA
CGCATACCTCAACGCTGTGGTCGGAACAGCACTGATCAAGAAGTACCCGAAGCTCGAGTCCGAATTCGTGTACG
GTGACTACAAGGTTTACGATGTGCGCAAGATGATCGCCAAGTCAGAGCAGGAAATTGGCAAGGCCACTGCGAA
GTACTTCTTCTACTCCAACATCATGAACTTCTTCAAGACTGAGATCACGCTGGCTAATGGCGAAATCCGGAAGA
GACCACTGATTGAGACCAACGGCGAGACAGGGGAAATCGTCTGGGACAAGGGGAGGGATTTCGCCACAGTCCG
CAAGGTTCTCTCTATGCCTCAAGTGAACATCGTCAAGAAGACTGAAGTCCAGACGGGCGGGTTCTCAAAGGAGT
CTATCCTGCCCAAGCGGAACTCGGATAAGCTTATCGCCAGGAAGAAGGACTGGGATCCGAAGAAGTACGGAGG
TTTCGACTCACCAACGGTGGCTTACTCTGTCCTGGTTGTGGCAAAGGTGGAGAAGGGAAAGTCGAAGAAGCTCA
AGTCTGTCAAGGAGCTGCTGGGTATCACCATTATGGAGAGGTCCAGCTTCGAGAAGAACCCGATCGACTTCCTC
GAGGCGAAGGGCTACAAGGAAGTGAAGAAGGACCTGATCATCAAGCTTCCGAAGTACAGTCTGTTCGAGTTGG
AGAACGGCAGGAAGCGCATGTTGGCTTCCGCAGGAGAGCTCCAGAAGGGTAACGAGCTTGCTTTGCCGTCCAA
GTACGTGAACTTCCTCTATCTGGCATCCCACTACGAGAAGCTCAAGGGCAGCCCAGAGGATAACGAACAGAAG
CAACTGTTTGTGGAGCAACACAAGCATTACCTTGACGAGATCATCGAACAGATCTCGGAGTTCAGTAAGCGCGT
CATCCTCGCCGACGCGAATTTGGATAAGGTTCTCTCAGCCTACAACAAGCACCGGGACAAGCCTATCAGAGAGC
AGGCGGAGAACATCATCCATCTCTTCACCCTGACAAACCTTGGGGCTCCCGCTGCCTTCAAGTACTTCGACACT
ACGATCGACCGGAAGAGGTACACTTCTACGAAGGAGGTGCTGGATGCAACCCTTATCCACCAATCGATCACTG
GCCTCTACGAGACGCGGATCGACTTGAGTCAGCTCGGTGGCGATAAGAGACCCGCAGCAACCAAGAAGGCAGG
GCAAGCAAAGAAGAAGAAGTGAGCGATCGCATATGAAGATGAAGATGAAATATTTGGTGTGTCAAATAAAAA
GCTTGTGTGCTTAAGTTTGTGTTTTTTTCTTGGCTTGTTGTGTTATGAATTTGTGGCTTTTTCTAATATTAAATGAA
TGTAAGATCTCATTATAATGAATAAACAAATGTTTCTATAATCCATTGTGAATGTTTTGTTGGATCTCTTCTGCA
GCATATAACTACTGTATGTGCTATGGTATGGACTATGGAATATGATTAAAGATAAGGGTACGTACCGCGATCGC
ATAACTTCGTATAGCATACATTATACGAAGTTATGAATTCGGCAATCAAGAGGTAAAGGATAATAATCCCAGCA
GAAACCGTGATCAAAGAAACAATTCATTGCAATATAGACTAGGCCATGCATACGCAAATCCAAGCGTGCATCC
ACTGACTGCATAACATAAACCCCCAGGTACTACGACTAAGCCGAACTGATACAAAATGCACTAGCTATTGGATA
ATCAGATCCTGGTCCATGCTGCTGTCATTCCATCGCAATCACCCTGGTAGCGACGACAGGATCATTTGCTTCCAG
AACCGAACTGTGCGCCTAGCTCGGCGCGCCTAACTATAACGGTCCTAA
SEQ ID NO: 23 (SbGhd7 sequence, 741 bp)
ATGTCAGGGCCAGCATGCGGTGTGTGCGGTGCAGCCGCCTGCTGCCGGCACCTCTTCCACACCGGCGACGAGAA
CGACGACTTCAACAGCCGGCGGGCCTTGTTCTCTGTGTTCCCTGCGGCGGTTCACCATCATGAGCCCAGCCCCA
GCAGCATGCAGCAGCAGCCTCCGGCGGGGTGCCTGCACGAGTTCCAGTTCTTTGGCCATCAGGACAACGATGAC
CACCAAGAAAGCATCGCCTGGCTCTTCGACCACCCGCCGCCACCTGCGCATGATGTCGACGACGACGACCGGTC
CCCAGCTGAGAACCAGCAGCCTCATCACCGGGCGTTTGACCCGTTTGGGACGGAGGGAAACGGGCTCACCTTTG
AGGTTGATGCCCGGCTGGGCCTCGGCAGCGGGGGCGCCGCCCGGCAAACAGCAGAGACAGCAGCAGCAAGCG
CCACCATCATGTCATTCTGTGGGAGCACATTCACAGACGCCGCAAGCTCGAGGCTCAAGGAGCCAACCCTGACT
GACGACAGTCAGCTGCAAATGCCGGTAGGTCAGTCAACGGAGAGGGAGGCTAAGTTGATGAGGTACAAGGAG
AAGAGGATGATGAGGTGTTATGAGAAGCAGATAAGATATGCATCCAGGAAAGCCTATGCGCAGGTGAGGCCCC
GGGTGAAAGGTCGCTTTGCCAAGGTAACCGAAGCCTGCTCCGCCACAGCAGACAATGTTGGCAACGACCACCT
GCTCTGA
SEQ ID NO: 24 (SbGhd7 expression vector, NPT marker, 11145 bp)
GGAACTAGGGATAACAGGGTAATGGCGCGCCGTTGTTTTAGTGTCCGAGGTTTGGTTTGGTGAAGTACATCTGA
ACTCTGAAGGTTCTTCGTGACATGGTTAGACGTGGTGTTGCAGTACTGTGATAAAACTTATCAGAATAAGTTTTC
GCTTCTAATTTGTTTTCTTAAATCATGTGTTGATCAGCCTATGAGTTGTTCATATCGTTGCCTTTCGTGTTGAACA
TTTGTTTTCTTGCCATACTAATGAGATTCATCTGCACAAACTGTACGAAGAGAATCAAGTTTAAACACTGCAGGT
CAACGGATCACCTATCAACATCCCAGCTAAAAACAGTAAAAAGGGGGAAAACGTGGGTGAGTTGAGTCTGTCT
TGTGGAAAAAACGTTTTAGTTTCTCCTGGAATTAACAATAAAAACAGTTGAACAAGATTGACTGTTCCTCCGGG
AGGGTTTGGAACATCGTTACAGATGTGAGCGAAAGGTGAGGAAACAGAGCGGAGGGCTTGGAGGTGACCTCGG
TAGTCGACGCCGGAGTTGAGCTTGATGACGACACCGTACTGGCGTACCAGGCCTAGTAGTGAACACCGGGCCT
GAAGCTGTCGCCGCCGCTGCTCATCTTGTGGGCTGTGCCCGGTGTCCCTGTTGCGGATTGCGGGTGGCAGCCTG
GCAGGTGGGTGCGACCCGTTTGGACTCCCTGATCTGGGCCCTTTGTGTCAGTACCGTCTGTACTCCGATGACATG
CACACCGTCGTCCACAGTCAAGTCCACAATCTCCCCTCTTTTTTTAACGGAATAGTTCAAAATCTCCTTGACGCA
CGCTATCGTGTACCAGCGCTCACTGGACACCACGTTTGTAATCCACGCCGACACGTCGGTCCCACGTCGACAGG
CCCCACCGTCCGGTCTGTAGCGTGTACGTATTCGGGCGACGGACGTGTCGTCGTCGTCTTGCGAGTCCCATTCCC
ATCACCATCTGAGCCACACATCCTCTGAACAAAAGCAGGGAGGCCTCTACGCACATCCCCCTTTCTCCCACTCC
TTGTCCGTGGCACCCACCCCAAACCCTCGCGCCGCCTCCGAGACAGCCGCCGCAACCATGGCCACCGCCTGCAG
GACCGTCTTCGGTACGCGCTCACTCCGCCCTCTGCCTTTGTTACTGCCACGTTTCTCTGAATGCTCTCTTGTGTGG
TGATTGCTGAGAGTGGTTTAGCTGGATCTAGAATTACACTCTGAAATCGTGTTCTGCCTGTGCTGATTACTTGCC
GTCCTTTGTAGCAGCAAAATATAGGGACATGGTAGTACGAAACGAAGATAGAACCTACACAGCAATACGAGAA
ATGTGTAATTTGGTGCTTAGCGGTATTTATTTAAGCACATGTTGGTGTTATAGGGCACTTGGATTCAGAAGTTTG
CTGTTAATTTAGGCACAGGCTTCATACTACATGGGTCAATAGTATAGGGATTCATATTATAGGCGATACTATAA
TAATTTGTTCGTCTGCAGAGCTTATTATTTGCCAAAATTAGATATTCCTATTCTGTTTTTGTTTGTGTGCTGTTAA
ATTGTTAACGCCTGAAGGAATAAATATAAATGACGAAATTTTGATGTTTATCTCTGCTCCTTTATTGTGACCATA
AGTCAAGATCAGATGCACTTGTTTTAAATATTGTTGTCTGAAGAAATAAGTACTGACAGTATTTTGATGCATTGA
TCTGCTTGTTTGTTGTAACAAAATTTAAAAATAAAGAGTTTCCTTTTTGTTGCTCTCCTTACCTCCTGATGGTATC
TAGTATCTACCAACTGACACTATATTGCTTCTCTTTACATACGTATCTTGCTCGATGCCTTCTCCCTAGTGTTGAC
CAGTGTTACTCACATAGTCTTTGCTCATTTCATTGTAATGCAGATACCAAGCGGCCCCTGCAGGATGGGGATTG
AACAAGATGGATTGCACGCAGGTTCTCCGGCCGCTTGGGTGGAGAGGCTATTCGGCTATGACTGGGCACAACA
GACAATCGGCTGCTCTGATGCCGCCGTGTTCCGGCTGTCAGCGCAGGGGCGCCCGGTTCTTTTTGTCAAGACCG
ACCTGTCCGGTGCCCTGAATGAACTCCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGTGCCGGGGCAGG
ATCTCCTGTCATCTCACCTTGCTCCTGCCGAGAAAGTATCCATCATGGCTGATGCAATGCGGCGGCTGCATACGC
TTGATCCGGCTACCTGCCCATTCGACCACCAAGCGAAACATCGCATCGAGCGAGCACGTACTCGGATGGAAGCC
GGTCTTGTCGATCAGGATGATCTGGACGAAGAGCATCAGGGGCTCGCGCCAGCCGAACTGTTCGCCAGGCTCA
AGGCGCGCATGCCCGACGGCGAGGATCTCGTCGTGACACATGGCGATGCCTGCTTGCCGAATATCATGGTGGA
AAATGGCCGCTTTTCTGGATTCATCGACTGTGGCCGGCTGGGTGTGGCGGACCGCTATCAGGACATAGCGTTGG
CTACCCGTGATATTGCTGAAGAGCTTGGCGGCGAATGGGCTGACCGCTTCCTCGTGCTTTACGGTATCGCCGCTC
CCGATTCGCAGCGCATCGCCTTCTATCGCCTTCTTGACGAGTTCTTCTGAGCGATCGCAGAGCCATGGGGGCGG
TGAGAGCTCGCGGCGTCGGATCTCCGGTTTCAGTTTCAGCTACGCGACTTGTGTGTGTGTGTGTGTGTCAGTGCG
TGGTTACTCTGAGCTTCGGTATGTTTGGTGTGTACTGTTCAGTGTTTCATGGTGCTTCCTAGCAGTGTCTGTCGTG
TACTTTGCCTGTATGTTGAGCTCGGCTCAGTATGTTCTGGGAGTGAATAAATAAAAAAAAATTAACCAGATATT
GTCGCGTGTACTTAGTATACTAATCACCTGCTCCTGCTCTTGCTCTGTTTTATCACCTTTCGGGCTTGACTGTCTT
TGTGGAGGCCTCTCAGTCTGATCTGGAACTCTACCTAAGTTTTACTAGTCCCGGGAAGCTTCTGCAGTGCAGCGT
GACCCGGTCGTGCCCCTCACTAGAGATAATGAGCATTGCATGTCTAAGTTATAAAAAATTACCACATATTTTTTT
TGTCACACTTGTTTGAAGTGCAGTTTATCTATCTTTATACATATATTTAAACTTTACTCTACGAATAATATAATCT
ATAGTACTACAATAATATCAGTGTTTTAGAGAATCATATAAATGAACAGTTAGACATGGTCTAAAGGACAATTG
AGTATTTTGACAACAGGACTCTACAGTTTTATCTTTTTAGTGTGCATGTGTTCTCCTTTTTTTTTGCAAATAGCTT
CACCTATATAATACTTCATCCATTTTATTAGTACATCCATTTAGGGTTTAGGGTTAATGGTTTTTATAGACTAATT
TTTTTAGTACATCTATTTTATTCTATTTTAGCCTCTAAATTAAGAAAACTAAAACTCTATTTTAGTTTTTTTATTTA
ATAATTTAGATATAAAATAGAATAAAATAAAGTGACTAAAAATTAAACAAATACCCTTTAAGAAATTAAAAAA
ACTAAGGAAACATTTTTCTTGTTTCGAGTAGATAATGCCAGCCTGTTAAACGCCGTCGACGAGTCTAACGGACA
CCAACCAGCGAACCAGCAGCGTCGCGTCGGGCCAAGCGAAGCAGACGGCACGGCATCTCTGTCGCTGCCTCTG
GACCCCTCTCGAGAGTTCCGCTCCACCGTTGGACTTGCTCCGCTGTCGGCATCCAGAAATTGCGTGGCGGAGCG
GCAGACGTGAGCCGGCACGGCAGGCGGCCTCCTCCTCCTCTCACGGCACGGCAGCTACGGGGGATTCCTTTCCC
ACCGCTCCTTCGCTTTCCCTTCCTCGCCCGCCGTAATAAATAGACACCCCCTCCACACCCTCTTTCCCCAACCTC
GTGTTGTTCGGAGCGCACACACACACAACCAGATCTCCCCCAAATCCACCCGTCGGCACCTCCGCTTCAAGGTA
CGCCGCTCGTCCTCCCCCCCCCCCCCTCTCTACCTTCTCTAGATCGGCGTTCCGGTCCATGGTTAGGGCCCGGTA
GTTCTACTTCTGTTCATGTTTGTGTTAGATCCGTGTTTGTGTTAGATCCGTGCTGCTAGCGTTCGTACACGGATGC
GACCTGTACGTCAGACACGTTCTGATTGCTAACTTGCCAGTGTTTCTCTTTGGGGAATCCTGGGATGGCTCTAGC
CGTTCCGCAGACGGGATCGATTTCATGATTTTTTTTGTTTCGTTGCATAGGGTTTGGTTTGCCCTTTTCCTTTATTT
CAATATATGCCGTGCACTTGTTTGTCGGGTCATCTTTTCATGCTTTTTTTTGTCTTGGTTGTGATGATGTGGTCTG
GTTGGGCGGTCGTTCTAGATCGGAGTAGAATTCTGTTTCAAACTACCTGGTGGATTTATTAATTTTGGATCTGTA
TGTGTGTGCCATACATATTCATAGTTACGAATTGAAGATGATGGATGGAAATATCGATCTAGGATAGGTATACA
TGTTGATGCGGGTTTTACTGATGCATATACAGAGATGCTTTTTGTTCGCTTGGTTGTGATGATGTGGTGTGGTTG
GGCGGTCGTTCATTCGTTCTAGATCGGAGTAGAATACTGTTTCAAACTACCTGGTGTATTTATTAATTTTGGAAC
TGTATGTGTGTGTCATACATCTTCATAGTTACGAGTTTAAGATGGATGGAAATATCGATCTAGGATAGGTATAC
ATGTTGATGTGGGTTTTACTGATGCATATACATGATGGCATATGCAGCATCTATTCATATGCTCTAACCTTGAGT
ACCTATCTATTATAATAAACAAGTATGTTTTATAATTATTTTGATCTTGATATACTTGGATGATGGCATATGCAG
CAGCTATATGTGGATTTTTTTAGCCCTGCCTTCATACGCTATTTATTTGCTTGGTACTGTTTCTTTTGTCGATGCTC
ACCCTGTTGTTTGGTGTTACTTCTCTAGAGCGGCCGCATTTAAATGCCGCCGCCATGTCAGGGCCAGCATGCGGT
GTGTGCGGTGCAGCCGCCTGCTGCCGGCACCTCTTCCACACCGGCGACGAGAACGACGACTTCAACAGCCGGC
GGGCCTTGTTCTCTGTGTTCCCTGCGGCGGTTCACCATCATGAGCCCAGCCCCAGCAGCATGCAGCAGCAGCCT
CCGGCGGGGTGCCTGCACGAGTTCCAGTTCTTTGGCCATCAGGACAACGATGACCACCAAGAAAGCATCGCCTG
GCTCTTCGACCACCCGCCGCCACCTGCGCATGATGTCGACGACGACGACCGGTCCCCAGCTGAGAACCAGCAGC
CTCATCACCGGGCGTTTGACCCGTTTGGGACGGAGGGAAACGGGCTCACCTTTGAGGTTGATGCCCGGCTGGGC
CTCGGCAGCGGGGGCGCCGCCCGGCAAACAGCAGAGACAGCAGCAGCAAGCGCCACCATCATGTCATTCTGTG
GGAGCACATTCACAGACGCCGCAAGCTCGAGGCTCAAGGAGCCAACCCTGACTGACGACAGTCAGCTGCAAAT
GCCGGTAGGTCAGTCAACGGAGAGGGAGGCTAAGTTGATGAGGTACAAGGAGAAGAGGATGATGAGGTGTTA
TGAGAAGCAGATAAGATATGCATCCAGGAAAGCCTATGCGCAGGTGAGGCCCCGGGTGAAAGGTCGCTTTGCC
AAGGTAACCGAAGCCTGCTCCGCCACAGCAGACAATGTTGGCAACGACCACCTGCTCTGATTCGAATTAATTAA
GGGGACACCGTTGTCGGAGCAGTGCCAGTGAGCTGGGTGGCAACCTGCGGGACTCTGGTGAGGAAGAGCAAAT
GGTAGAAAGCAGTAGCTGTGCTTCTGAACATTGTGTGTGTTTGTTTCCCTTGTTACCGCCAATCATCCTACCTGA
TGTGTCGTCTGGGGTTTCGTTTGAACCATGCTGGGTTTGGAGCTTTGTGATGCAAAGCAGAATGTCCTGATGCTT
TCTTTCTCCATAGAATAAATCTCTCCTGTTACCTAAAGGCCGGCCGATATCCATTCAAGAGGAGAGCTCATTTCC
TCTCCTCTCATCACCTTTGCCTAAAAACAGTATGAAAGTCAAGCTAAAGACTTTAAACTAGCTCACTTGGGAGG
AAGTCCCATGTCTATTCTTGTATATACTTTTTTCCCTTGTTATTTTTGATGTTTTGTGTAAGTATTTCAGGTTTTAT
AGTCCACGGAAGTCAACGATCGAGTGATATAAATCGAGTCAAAGAATCTTAGCCGTGCGATAGAGTGACTTAA
GGCGAAGCATCCAGCTAATCCGAGTCCCACACAACCTATTCCTTGGAGCTCTAAACGGGAGAGTATGGACGACT
TCGTCAACTCCTATTTCACCAGATCACTAAGGTAACACACCACCCCCATTGTATATACCGTTATCTTTTATTTTTC
TTTGTTTGTTGTTTGTTTTAGTCTTGAATTCTCTCTCAGATTTTTAATACACAAGGGACTGTGTAATTTAAGTGTG
GGGGAGGGTTCAAGAAATATTTGACTTTATGTTCTCTTTTCTTATTTCAAATTTTGCATCATCTAAGGCATAAAA
AACCATAAAAATTTAAAAACATTTATGAAAATGATTTCACCAAAACAGAGTGTCATGTAGATTGCATTGCATTT
TAGGATTGTATATAGAGTACTTGCATTTAGGATTTCTCATTTGCATAGGGGATAATGATGATTTCGTACCGTTAA
GCATGTTTTGATTCAATGAAGTAAGTTCAATGCCCAGGTTGTTAGTTCTCTGTTGCATAACCGATTGTTTCTCTG
AAGTAAGACTGCACCCTGCTTTGAATTTACAACTTGACTATACTTCTTTGATTCAAAACTCGCTTATCTGATGCC
ATTCCCTATCTATTAGAACCTGAACTGAATTTGCAATTATCATGTCTATGCATTGTTTGTGAACTCATGGCTACC
ATATACATACTTGTATTATCTTATTCACTTCACCACCCTCTGTTAATCTAAGTAGCTGACTCTCATTTTTAGAGCA
GTTTCCCATACCCTTAGCCTAGCCTTATTTCAAGCCAAAGATCATTTGTGTGTGATTGTGAGGTCTTTTTCGATTG
AGCTTGGTATAACGTGTCAGGTATGAGCCGACAAGAGCAGTCTTTCATGTAGACCTAGCCCACGCTTTTTGGGT
TAGCTAGGACTAGGTGGAGACTTGTATTTGGGATTGGGGTTGTGTATGGATTGAATGGGAAAGAGAATGAGAA
TGAAAAGAAAAGAAAAAAAGAGAGTAAGAGTTTAGAGTTCTTAAGGGGGATACAAAAAGTGTTAGTCTAGTA
AAGGGTTTGGAATTCAAAGAAGTTAGGCATTGTGATTTAAAGAAAAGGGTAAAACGACCTAAGATTAGAAGAA
AAGCCTTAAGTTTGTCAAAGTATAAAACCAAACCCACTAGACTCTTGAATCGTTTTAAAGAAGAACCCCTTAGA
ACTTGAACCAAAAAGAGAAAGAATGAGAAAAAGGGTAGAGTTAGGACATCTATGGGACTGGAACCATCTTATG
AACTATCATACTATATCATTGGGTAGATGGGAGTCTGTTCTTGTTTGTGTCCATTGGCTTTTACCTTTAGCATTCT
GCTGACGCTCAATCTTTTCTTTGAGAGTCCCCTGTTACTCGACCAAATCATTAAAGGGACCATTTTTGTCTCTTA
GCCTAAGCCTGAAACCAAGTGAGTTCATAACCATTGCATTGCTTGATCCATTGTTCGTGCTTAGTGAATGTTAAA
AAGAATTGGTTGATGTGAATGCTTGAATAGATTAAGGCAGGTTGTTAGGTTGCTTTGTGACGAGTATGGCTAAA
TTTTTAAGTAAGGGTCTATCATTTTGCAACCTATAATTAGCTACCTGGACATTGAGCTTACCCGTTCTATATGCA
TGCTACGGGTTTTGAATCCCCACCTTCAAACCTCTCCTTCAACTTAAGATTTTTTGTTTGCTTGAGGGCAAGCAA
GAGATAAGTTTGGGGGAGTTGATAACACTGTAATTTACTCATTTTGGGCATGTATTTATCATCATTTAGCTTAGT
GATCACTCATTGTTTCATATCATTTTGCATCACATTTAGCTTATTTTGCACACTTAGGCATTTAGGGTCATCTTGC
ATACATATTGCATGTTATGGTGTGTTTTCAGGTGCAAAATAAGTTTACAAGGAGTTTACTCGACCCTCTACCCTA
TTGCCCCAGAGGGGAACCAGAGGAGCAGCTACTCGACCAATTATTCATTGCCTCAGAGGGGAAGCAGCAGAGC
AGCTACTCGACCAGTTATTCGGCGCGCCTAGGGATAACAGGGTAATGTTCCATCCCAATGGCGCGCCGAGCTTG
GCGTAATCATGGTCATAGCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTGCGTTGCGCTCACTGCCCG
CTTTCCAGTCGGGAAACCTGTCGTGCCAGCTGCATTAATGAATCGGCCAACGCGCGGGGAGAGGCGGTTTGCGT
ATTGGGCGCTGTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGATACCTGTCCGCCTTTCTCCCTTCGGG
AAGCGTGGCGCTTTCTCATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATGTAGGCGGTGCTAC
AGAGTTCTTGAAGTGGTGGCCTAACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGAACCACGCTCACCGGCT
CCAGATTTATCAGCAATAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAACGATCAAG
GCGAGTTACATGATCCCCCATGTTGTGCAAAAAAGCGGTTAGCTCCTTCGGTCCTCCGATCGTTGTCAGAAGTA
AGTTGGCCGCAGTGTTATCACTCATGGTTATGGCAGCACTGCATAATTCTCTTACTGTCATGCCATCCGTAAGAT
GCTTTTCTGTGACTGGTGAGTACTCAACCAAGTCATTCTGAGAATAGTGTATGCGGCGACCGAGTTGCTCTTGCC
CGGCGTCAATACGGGATAATACCGCGCCACATAGCAGAACTTTAAAAGTGCTCATCATTGGAAAACGTTCTTCG
GGGCGAAAACTCTCAAGGATCTTACCGCTGTTGAGATCCAGTTCGATGTAACCCACTCGTGCACCCAACTGATC
TTCAGCATCTTTTACTTTCACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGAAGCATTTATCAGGGTTAT
TGTCTCATGAGCGGATACATATTTGAATGTATTTAGAAAAATAAACAAATAGGGGTTCCGCGCACATTTCCCCG
AAAAGTGCCACCTGACGTCTAAGAAACCATTATTATCATGACATTAACCTATAAAAATAGGCGTATCACGAGGC
CCTTTTGTCTCGCGCGTTTCGGTGATGACGGTGAAAACCTCTGACACATGCAGCTCCCGGAGACTGTCACAGCTT
GTCTGTAAGCGGATGCCGGGAGCAGACAAGCCCGTCAGGGCGCGTCAGCGGGTGTTGGCGGGTGTCGGGGCTG
GCTTAACTATGCGGCATCAGAGCAGATTGTACTGAGAGTGCACCATATGCGGTGTGAAATACCGCACAGATGC
GTAAGGAGAAAATACCGCATCAGGCGCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAACGCCAGG
GTTTTCCCAGTCACGACGTTGTAAAACGACGGCCAGTGAATTGACGCGTATTGGGAT
SEQ ID NO: 25 (SbGhd7 expression vector, minimal cassette, 8446 bp)
CAGGGTAATGGCGCGCCGTTGTTTTAGTGTCCGAGGTTTGGTTTGGTGAAGTACATCTGAACTCTGAAGGTTCTT
CGTGACATGGTTAGACGTGGTGTTGCAGTACTGTGATAAAACTTATCAGAATAAGTTTTCGCTTCTAATTTGTTT
TCTTAAATCATGTGTTGATCAGCCTATGAGTTGTTCATATCGTTGCCTTTCGTGTTGAACATTTGTTTTCTTGCCA
TACTAATGAGATTCATCTGCACAAACTGTACGAAGAGAATCAAGTTTAAACACTGCAGGTCAACGGATCACCTA
TCAACATCCCAGCTAAAAACAGTAAAAAGGGGGAAAACGTGGGTGAGTTGAGTCTGTCTTGTGGAAAAAACGT
TTTAGTTTCTCCTGGAATTAACAATAAAAACAGTTGAACAAGATTGACTGTTCCTCCGGGAGGGTTTGGAACAT
CGTTACAGATGTGAGCGAAAGGTGAGGAAACAGAGCGGAGGGCTTGGAGGTGACCTCGGTAGTCGACGCCGG
AGTTGAGCTTGATGACGACACCGTACTGGCGTACCAGGCCTAGTAGTGAACACCGGGCCTGAAGCTGTCGCCGC
CGCTGCTCATCTTGTGGGCTGTGCCCGGTGTCCCTGTTGCGGATTGCGGGTGGCAGCCTGGCAGGTGGGTGCGA
CCCGTTTGGACTCCCTGATCTGGGCCCTTTGTGTCAGTACCGTCTGTACTCCGATGACATGCACACCGTCGTCCA
CAGTCAAGTCCACAATCTCCCCTCTTTTTTTAACGGAATAGTTCAAAATCTCCTTGACGCACGCTATCGTGTACC
AGCGCTCACTGGACACCACGTTTGTAATCCACGCCGACACGTCGGTCCCACGTCGACAGGCCCCACCGTCCGGT
CTGTAGCGTGTACGTATTCGGGCGACGGACGTGTCGTCGTCGTCTTGCGAGTCCCATTCCCATCACCATCTGAGC
CACACATCCTCTGAACAAAAGCAGGGAGGCCTCTACGCACATCCCCCTTTCTCCCACTCCTTGTCCGTGGCACCC
ACCCCAAACCCTCGCGCCGCCTCCGAGACAGCCGCCGCAACCATGGCCACCGCCTGCAGGACCGTCTTCGGTAC
GCGCTCACTCCGCCCTCTGCCTTTGTTACTGCCACGTTTCTCTGAATGCTCTCTTGTGTGGTGATTGCTGAGAGTG
GTTTAGCTGGATCTAGAATTACACTCTGAAATCGTGTTCTGCCTGTGCTGATTACTTGCCGTCCTTTGTAGCAGC
AAAATATAGGGACATGGTAGTACGAAACGAAGATAGAACCTACACAGCAATACGAGAAATGTGTAATTTGGTG
CTTAGCGGTATTTATTTAAGCACATGTTGGTGTTATAGGGCACTTGGATTCAGAAGTTTGCTGTTAATTTAGGCA
CAGGCTTCATACTACATGGGTCAATAGTATAGGGATTCATATTATAGGCGATACTATAATAATTTGTTCGTCTGC
AGAGCTTATTATTTGCCAAAATTAGATATTCCTATTCTGTTTTTGTTTGTGTGCTGTTAAATTGTTAACGCCTGAA
GGAATAAATATAAATGACGAAATTTTGATGTTTATCTCTGCTCCTTTATTGTGACCATAAGTCAAGATCAGATGC
ACTTGTTTTAAATATTGTTGTCTGAAGAAATAAGTACTGACAGTATTTTGATGCATTGATCTGCTTGTTTGTTGTA
ACAAAATTTAAAAATAAAGAGTTTCCTTTTTGTTGCTCTCCTTACCTCCTGATGGTATCTAGTATCTACCAACTG
ACACTATATTGCTTCTCTTTACATACGTATCTTGCTCGATGCCTTCTCCCTAGTGTTGACCAGTGTTACTCACATA
GTCTTTGCTCATTTCATTGTAATGCAGATACCAAGCGGCCCCTGCAGGATGGGGATTGAACAAGATGGATTGCA
CGCAGGTTCTCCGGCCGCTTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCGACCTGTCCGGTGCCCTG
AATGAACTCCAGGACGAGGCAGCGCGGCTATCGTGGCTGGCCACGACGGGCGTTCCTTGCGCAGCTGTGCTCG
ACGTTGTCACTGAAGCGGGAAGGGACTGGCTGCTATTGGGCGAAGTGCCGGGGCAGGATCTCCTGTCATCTCAC
CTTGCTCCTGCCGAGAAAGTATCCATCATGGCTGATGCAATGCGGCGGCTGCATACGCTTGATCCGGCTACCTG
CCCATTCGACCACCAAGCGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCTCAAGGCGCGCATGCCCG
ACGGCGAGGATCTCGTCGTGACACATGGCGATGCCTGCTTGCCGAATATCATGGTGGAAAATGGCCGCTTTTCT
GGATTCATCGACTGTGGCCGGCTGGGTGTGGCGGACCGCTATCAGGACATAGCGTTGGCTACCCGTGATATTGC
TGAAGAGCTTGGCGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAGCGATCGCAGAGCCATGGGGGCGGTGAGAGCTCGCGGCG
TCGGATCTCCGGTTTCAGTTTCAGCTACGCGACTTGTGTGTGTGTGTGTGTGTCAGTGCGTGGTTACTCTGAGCT
TCGGTATGTTTGGTGTGTACTGTTCAGTGTTTCATGGTGCTTCCTAGCAGTGTCTGTCGTGTACTTTGCCTGTATG
TTGAGCTCGGCTCAGTATGTTCTGGGAGTGAATAAATAAAAAAAAATTAACCAGATATTGTCGCGTGTACTTAG
TATACTAATCACCTGCTCCTGCTCTTGCTCTGTTTTATCACCTTTCGGGCTTGACTGTCTTTGTGGAGGCCTCTCA
GTCTGATCTGGAACTCTACCTAAGTTTTACTAGTCCCGGGAAGCTTCTGCAGTGCAGCGTGACCCGGTCGTGCCC
CTCACTAGAGATAATGAGCATTGCATGTCTAAGTTATAAAAAATTACCACATATTTTTTTTGTCACACTTGTTTG
AAGTGCAGTTTATCTATCTTTATACATATATTTAAACTTTACTCTACGAATAATATAATCTATAGTACTACAATA
ATATCAGTGTTTTAGAGAATCATATAAATGAACAGTTAGACATGGTCTAAAGGACAATTGAGTATTTTGACAAC
AGGACTCTACAGTTTTATCTTTTTAGTGTGCATGTGTTCTCCTTTTTTTTTGCAAATAGCTTCACCTATATAATAC
TTCATCCATTTTATTAGTACATCCATTTAGGGTTTAGGGTTAATGGTTTTTATAGACTAATTTTTTTAGTACATCT
ATTTTATTCTATTTTAGCCTCTAAATTAAGAAAACTAAAACTCTATTTTAGTTTTTTTATTTAATAATTTAGATAT
AAAATAGAATAAAATAAAGTGACTAAAAATTAAACAAATACCCTTTAAGAAATTAAAAAAACTAAGGAAACAT
TTTTCTTGTTTCGAGTAGATAATGCCAGCCTGTTAAACGCCGTCGACGAGTCTAACGGACACCAACCAGCGAAC
CAGCAGCGTCGCGTCGGGCCAAGCGAAGCAGACGGCACGGCATCTCTGTCGCTGCCTCTGGACCCCTCTCGAGA
GTTCCGCTCCACCGTTGGACTTGCTCCGCTGTCGGCATCCAGAAATTGCGTGGCGGAGCGGCAGACGTGAGCCG
GCACGGCAGGCGGCCTCCTCCTCCTCTCACGGCACGGCAGCTACGGGGGATTCCTTTCCCACCGCTCCTTCGCTT
TCCCTTCCTCGCCCGCCGTAATAAATAGACACCCCCTCCACACCCTCTTTCCCCAACCTCGTGTTGTTCGGAGCG
CACACACACACAACCAGATCTCCCCCAAATCCACCCGTCGGCACCTCCGCTTCAAGGTACGCCGCTCGTCCTCC
CCCCCCCCCCCTCTCTACCTTCTCTAGATCGGCGTTCCGGTCCATGGTTAGGGCCCGGTAGTTCTACTTCTGTTCA
TGTTTGTGTTAGATCCGTGTTTGTGTTAGATCCGTGCTGCTAGCGTTCGTACACGGATGCGACCTGTACGTCAGA
CACGTTCTGATTGCTAACTTGCCAGTGTTTCTCTTTGGGGAATCCTGGGATGGCTCTAGCCGTTCCGCAGACGGG
ATCGATTTCATGATTTTTTTTGTTTCGTTGCATAGGGTTTGGTTTGCCCTTTTCCTTTATTTCAATATATGCCGTGC
ACTTGTTTGTCGGGTCATCTTTTCATGCTTTTTTTTGTCTTGGTTGTGATGATGTGGTCTGGTTGGGCGGTCGTTC
TAGATCGGAGTAGAATTCTGTTTCAAACTACCTGGTGGATTTATTAATTTTGGATCTGTATGTGTGTGCCATACA
TATTCATAGTTACGAATTGAAGATGATGGATGGAAATATCGATCTAGGATAGGTATACATGTTGATGCGGGTTT
TACTGATGCATATACAGAGATGCTTTTTGTTCGCTTGGTTGTGATGATGTGGTGTGGTTGGGCGGTCGTTCATTC
GTTCTAGATCGGAGTAGAATACTGTTTCAAACTACCTGGTGTATTTATTAATTTTGGAACTGTATGTGTGTGTCA
TACATCTTCATAGTTACGAGTTTAAGATGGATGGAAATATCGATCTAGGATAGGTATACATGTTGATGTGGGTT
TTACTGATGCATATACATGATGGCATATGCAGCATCTATTCATATGCTCTAACCTTGAGTACCTATCTATTATAA
TAAACAAGTATGTTTTATAATTATTTTGATCTTGATATACTTGGATGATGGCATATGCAGCAGCTATATGTGGAT
TTTTTTAGCCCTGCCTTCATACGCTATTTATTTGCTTGGTACTGTTTCTTTTGTCGATGCTCACCCTGTTGTTTGGT
GTTACTTCTCTAGAGCGGCCGCATTTAAATGCCGCCGCCATGTCAGGGCCAGCATGCGGTGTGTGCGGTGCAGC
CGCCTGCTGCCGGCACCTCTTCCACACCGGCGACGAGAACGACGACTTCAACAGCCGGCGGGCCTTGTTCTCTG
TGTTCCCTGCGGCGGTTCACCATCATGAGCCCAGCCCCAGCAGCATGCAGCAGCAGCCTCCGGCGGGGTGCCTG
CACGAGTTCCAGTTCTTTGGCCATCAGGACAACGATGACCACCAAGAAAGCATCGCCTGGCTCTTCGACCACCC
GCCGCCACCTGCGCATGATGTCGACGACGACGACCGGTCCCCAGCTGAGAACCAGCAGCCTCATCACCGGGCG
TTTGACCCGTTTGGGACGGAGGGAAACGGGCTCACCTTTGAGGTTGATGCCCGGCTGGGCCTCGGCAGCGGGG
GCGCCGCCCGGCAAACAGCAGAGACAGCAGCAGCAAGCGCCACCATCATGTCATTCTGTGGGAGCACATTCAC
AGACGCCGCAAGCTCGAGGCTCAAGGAGCCAACCCTGACTGACGACAGTCAGCTGCAAATGCCGGTAGGTCAG
TCAACGGAGAGGGAGGCTAAGTTGATGAGGTACAAGGAGAAGAGGATGATGAGGTGTTATGAGAAGCAGATA
AGATATGCATCCAGGAAAGCCTATGCGCAGGTGAGGCCCCGGGTGAAAGGTCGCTTTGCCAAGGTAACCGAAG
CCTGCTCCGCCACAGCAGACAATGTTGGCAACGACCACCTGCTCTGATTCGAATTAATTAAGGGGACACCGTTG
TCGGAGCAGTGCCAGTGAGCTGGGTGGCAACCTGCGGGACTCTGGTGAGGAAGAGCAAATGGTAGAAAGCAGT
AGCTGTGCTTCTGAACATTGTGTGTGTTTGTTTCCCTTGTTACCGCCAATCATCCTACCTGATGTGTCGTCTGGGG
TTTCGTTTGAACCATGCTGGGTTTGGAGCTTTGTGATGCAAAGCAGAATGTCCTGATGCTTTCTTTCTCCATAGA
ATAAATCTCTCCTGTTACCTAAAGGCCGGCCGATATCCATTCAAGAGGAGAGCTCATTTCCTCTCCTCTCATCAC
CTTTGCCTAAAAACAGTATGAAAGTCAAGCTAAAGACTTTAAACTAGCTCACTTGGGAGGAAGTCCCATGTCTA
TTCTTGTATATACTTTTTTCCCTTGTTATTTTTGATGTTTTGTGTAAGTATTTCAGGTTTTATAGTCCACGGAAGTC
AACGATCGAGTGATATAAATCGAGTCAAAGAATCTTAGCCGTGCGATAGAGTGACTTAAGGCGAAGCATCCAG
CTAATCCGAGTCCCACACAACCTATTCCTTGGAGCTCTAAACGGGAGAGTATGGACGACTTCGTCAACTCCTAT
TTCACCAGATCACTAAGGTAACACACCACCCCCATTGTATATACCGTTATCTTTTATTTTTCTTTGTTTGTTGTTT
GTTTTAGTCTTGAATTCTCTCTCAGATTTTTAATACACAAGGGACTGTGTAATTTAAGTGTGGGGGAGGGTTCAA
GAAATATTTGACTTTATGTTCTCTTTTCTTATTTCAAATTTTGCATCATCTAAGGCATAAAAAACCATAAAAATTT
AAAAACATTTATGAAAATGATTTCACCAAAACAGAGTGTCATGTAGATTGCATTGCATTTTAGGATTGTATATA
GAGTACTTGCATTTAGGATTTCTCATTTGCATAGGGGATAATGATGATTTCGTACCGTTAAGCATGTTTTGATTC
AATGAAGTAAGTTCAATGCCCAGGTTGTTAGTTCTCTGTTGCATAACCGATTGTTTCTCTGAAGTAAGACTGCAC
CCTGCTTTGAATTTACAACTTGACTATACTTCTTTGATTCAAAACTCGCTTATCTGATGCCATTCCCTATCTATTA
GAACCTGAACTGAATTTGCAATTATCATGTCTATGCATTGTTTGTGAACTCATGGCTACCATATACATACTTGTA
TTATCTTATTCACTTCACCACCCTCTGTTAATCTAAGTAGCTGACTCTCATTTTTAGAGCAGTTTCCCATACCCTT
AGCCTAGCCTTATTTCAAGCCAAAGATCATTTGTGTGTGATTGTGAGGTCTTTTTCGATTGAGCTTGGTATAACG
TGTCAGGTATGAGCCGACAAGAGCAGTCTTTCATGTAGACCTAGCCCACGCTTTTTGGGTTAGCTAGGACTAGG
TGGAGACTTGTATTTGGGATTGGGGTTGTGTATGGATTGAATGGGAAAGAGAATGAGAATGAAAAGAAAAGAA
AAAAAGAGAGTAAGAGTTTAGAGTTCTTAAGGGGGATACAAAAAGTGTTAGTCTAGTAAAGGGTTTGGAATTC
AAAGAAGTTAGGCATTGTGATTTAAAGAAAAGGGTAAAACGACCTAAGATTAGAAGAAAAGCCTTAAGTTTGT
CAAAGTATAAAACCAAACCCACTAGACTCTTGAATCGTTTTAAAGAAGAACCCCTTAGAACTTGAACCAAAAA
GAGAAAGAATGAGAAAAAGGGTAGAGTTAGGACATCTATGGGACTGGAACCATCTTATGAACTATCATACTAT
ATCATTGGGTAGATGGGAGTCTGTTCTTGTTTGTGTCCATTGGCTTTTACCTTTAGCATTCTGCTGACGCTCAATC
TTTTCTTTGAGAGTCCCCTGTTACTCGACCAAATCATTAAAGGGACCATTTTTGTCTCTTAGCCTAAGCCTGAAA
CCAAGTGAGTTCATAACCATTGCATTGCTTGATCCATTGTTCGTGCTTAGTGAATGTTAAAAAGAATTGGTTGAT
GTGAATGCTTGAATAGATTAAGGCAGGTTGTTAGGTTGCTTTGTGACGAGTATGGCTAAATTTTTAAGTAAGGG
TCTATCATTTTGCAACCTATAATTAGCTACCTGGACATTGAGCTTACCCGTTCTATATGCATGCTACGGGTTTTG
AATCCCCACCTTCAAACCTCTCCTTCAACTTAAGATTTTTTGTTTGCTTGAGGGCAAGCAAGAGATAAGTTTGGG
GGAGTTGATAACACTGTAATTTACTCATTTTGGGCATGTATTTATCATCATTTAGCTTAGTGATCACTCATTGTTT
CATATCATTTTGCATCACATTTAGCTTATTTTGCACACTTAGGCATTTAGGGTCATCTTGCATACATATTGCATGT
TATGGTGTGTTTTCAGGTGCAAAATAAGTTTACAAGGAGTTTACTCGACCCTCTACCCTATTGCCCCAGAGGGG
AACCAGAGGAGCAGCTACTCGACCAATTATTCATTGCCTCAGAGGGGAAGCAGCAGAGCAGCTACTCGACCAG
TTATTCGGCGCGCCTAGGGATAA
SEQ ID NO: 26 (Hairpin-loop sequence, rice sucrose synthase1 (RSuS1) gene, 95 bp)
GTTTGAATCAGATTCAGATTTCATTGCATCACAGAGATCCATCTTTACTCTACCGCTTGCTCTAACTTAACTTGT
AATTGTTTTTTATCATGCAG
SEQ ID NO: 27 (ScFT4g1: gRNA sequence, 20 bp)
GGCACCAGGACCGTATCCAA
SEQ ID NO: 28 (ScFT4g2: gRNA sequence, 20 bp)
GTGTAGAAGGTCCTCATGTC
SEQ ID NO: 29 (ScFT4g4: gRNA sequence, 20 bp)
GTAGCTGACCCTGAGGTTGG
SEQ ID NO: 30 (ScFT8g1: gRNA sequence, 20 bp)
AACAATAGGCTACTTCTAGC
SEQ ID NO: 31 (ScFT8g2: gRNA sequence, 20 bp)
GTTATTCTGAGGGGTATAGT
SEQ ID NO: 32 (ScFT8g5: gRNA sequence, 20 bp)
CCCTCAGTGATGGATTGCTT
SEQ ID NO: 33 (ScFT10g1: gRNA sequence, 20 bp)
GGTTTACTTGCAACCGCAGA
SEQ ID NO: 34 (ScFT10g2: gRNA sequence, 20 bp)
TTGCCACCGACATCGACTCG
SEQ ID NO: 35 (ScFT10g4: gRNA sequence, 20 bp)
AAACCACGAGTCGATGTCGG
SEQ ID NO: 36 (ScFT10g5: gRNA sequence, 20 bp)
GGTCATGGCTCATGTCATAC
SEQ ID NO: 37 (FT4 gene, 1-207, 496-557, 1374-1414, 2751-2980 nucleotides from: GenBank
Accession No.: MT666096.1: 1-207, 496-557, 1374-1414, 2751-2980 Saccharum hybrid
cultivar R570 FT4 (FT4) gene, complete cds, 539 bp)
ATGGCCGGCAGCGGCAGGGAAAGGGAGACGCTGGTGGTTGGTAGGGTGGTGGGCGACGTGCTGGACCCCTTCG
TCCGGACCACCAACCTCAGGGTCAGCTACGGCACCAGGACCGTATCCAACGGCTGCGAGCTCAAGCCGTCCAT
GGTGGTGAACCAGCCCAGGGTCGAGGTCGGGGGACCCGACATGAGGACCTTCTACACCCTCGTGATGGTCGAC
CCGGATGCTCCGAGCCCAAGCGACCCAAACCTTAGGGAGTATTTGCACTGGCTGGTCACGGATATTCCGGGAAC
TACTGGGGCAGCATTTGGGCAAGAGGTGATCTGCTACGAGAGTCCTCGGCCGACCATGGGGATCCACCGCTTCG
TGCTGGTGCTGTTCCAGCAGCTGGTGCGGCAGACGGTGTACGCCCCGGGGTGGCGCCAGAACTTCAACACCAG
GGACTTCGCCGAGCTCTACAACCTGGGCCCTCCCGTGGCCGCCGTCTACTTCAACTGCCAGCGTGAGGCCGGCT
CTGGGGGAAGGAGGATGTACTCATGA
SEQ ID NO: 38 (FT8 genc, 1000-1194, 1351-1412, 1578-1618, 2384-2619 nucleotides from:
MT723926.1: Saccharum hybrid cultivar R570 FT-like PEBP family member protein (ScFT8)
gene, complete cds, 534 bp)
ATGTCTCAGGTGGAACCGTTGGTTCTGGTTCATGTGATACGAGATGTGTTGGATTCATTTACACCAACTATACCC
CTCAGAATAACCTACAACAATAGGCTACTTCTAGCAGGTGTTGAGCTGAAGCCATCCGCAGTGGTGAATAAGCC
AAGAGTTGATGTTGGGGGCACCGACCTCAGGGTGTTCTTTACACTGGTATTAGTTGATCCAGATGCCCCAAGCC
CAAGCAATCCATCACTGAGGGAATATTTGCACTGGATGGTGATAGATATTCCAGGAACAACTGCAGCCAACTTT
GGTCAGGAGCTCATGTTTTACGAAAGGCCAGAACCGAGATCTGGTATACACCGCATGGTATTTGTGCTGTTCCG
GCAACTTGGTAGGGGGACAGTTTTTGCACCAGACATGCGACATAACTTCAACTGCAAGAACTTTGCACGTCAGT
ACCACCTGGACATTGTGGCTGCCACATATTTCAACTGTCAGAGGGAAGCAGGATCTGGGGGCAGAAGGTTCAG
GCCCGAAAGTTCGTAA
SEQ ID NO: 39 (FT10 gene, 1281-1475, 1680-1741, 1828-1868, 2565-2794 nucleotides from:
MT723928.1: Saccharum hybrid cultivar R570 FT-like PEBP family member protein (ScFT10)
gene, complete cds, 529 bp)
ATGTCAGCAACCGATCCTTTGGTCATGGCTCATGTCATACAGGATGTGTTGGATCCCTTTACACCAACCATTCCA
CTAAGAATAACATACAACAATAGGCTACTTCTGGCAAGTGCTGAGCTAAAGCCATCTGCGGTTGCAAGTAAACC
ACGAGTCGATGTCGGTGGCAATGACATGAGGGCTTTCTACACCCTGGTACTGATTGACCCGGACGCTCCAAGTC
CAAGCCATCCATCACTAAGGGAGTACTTGCACTGGATGGTGACAGATATTCCTGAAACAACTAGTGTCAACTTT
GGCaCAAGAGCTAGTATTTTATGAGAGACCGGACCCAAGATCTGGCATCCACAGGCTCGTATTTGTGCTGTTCC
GTCAACTTGGCAGGGGGACAGTTTTTGCACCAGAAATGCGCCACCACTTCAACTGCAGAAGCTTTGCACGGCAA
TATCACCTCAGCGTTGCCACTGCTACATATTTCAACTGTCAAAGGGAAGGTGGATCGGGTGGAAGAAGGTTTAG
GGAAGAGTAG

What is claimed:

1. An engineered sugarcane or energycane plant having delayed flowering, wherein the engineered sugarcane or energycane plant has decreased expression of one or more of the FT4 gene, the FT8 gene, and the FT10 gene.

2. The engineered sugarcane or energycane plant of claim 1, wherein the engineered sugarcane or energycane plant has decreased expression of the FT4 gene, the FT8 gene, and the FT10 gene.

3. The engineered sugarcane or energycane plant of claim 1 or 2, wherein the engineered sugarcane or energycane plant has loss of function mutation in the FT4 gene, the FT8 gene, and/or the FT10 gene.

4. The engineered sugarcane or energycane plant of claim 3, wherein the loss of function mutation is a CRISPR-induced loss of function mutation.

5. The engineered sugarcane or energycane plant of claim 1 or 2, wherein the engineered sugarcane or energycane plant expresses a one or more RNAi constructs targeting one or more of the FT4 gene, the FT8 gene, and the FT10 gene.

6. The engineered sugarcane or energycane plant of claim 5, wherein the engineered sugarcane or energycane plant expresses a single RNAi construct targeting one or more of the FT4 gene, the FT8 gene, and the FT10 gene.

7. The engineered sugarcane or energycane plant of claim 6, wherein the engineered sugarcane or energycane plant expresses a single RNAi construct targeting the FT4 gene, the FT8 gene, and the FT10 gene.

8. A method for delaying flowering in a sugarcane or energycane plant comprising introducing into the sugarcane or energycane plant one or more CRISPR constructs for introducing a loss of function mutation into one or more of the FT4 gene, the FT8 gene, and the FT10 gene, or expressing in the sugarcane or energycane plant one or more RNAi constructs targeting one or more of the FT4 gene, the FT8 gene, and the FT10 gene.

9. A method for generating a sugarcane or energycane plant having delayed flowering comprising:
(a) transforming a plant cell with
(i) one or more CRISPR constructs for introducing loss of function mutations into one or more of the FT4 gene, the FT8 gene, and the FT10 gene, or
(ii) one or more expression vectors encoding one or more RNAi constructs targeting one or more of the FT4 gene, the FT8 gene, and the FT10 gene;
(b) producing a regenerant plant from the transformed plant cell.

10. The method of claim 9, wherein the method comprises transforming a plurality of plant cells, producing a plurality of regenerant plants from the plurality of transformed plant cells, and selecting regenerant plants with delayed flowering.

11. The method of claim 9, wherein the method further comprising asexually propagating the regenerant plant of produce a population of sugarcane or energycane plant having delayed flowering.

12. The method of claim 9, wherein transforming the plant cell further comprises introducing a genetic marker into the plant cell.

13. A method of decreasing expression of the FT4 gene, the FT8 gene, and/or the FT10 gene in a sugarcane or energycane plant comprising introducing into the plant or a progenitor of the plant one or more expression vectors encoding one or more CRISPR constructs for introducing loss of function mutations into one or more of the FT4 gene, the FT8 gene, and the FT10 gene, or one or more expression vectors encoding one or more RNAi constructs targeting one or more of the FT4 gene, the FT8 gene, and the FT10 gene.

14. The method of claim 13, wherein the progenitor of the plant is a plant cell, wherein the plant cell is transformed with one or more expression vectors encoding one or more CRISPR constructs for introducing loss of function mutations into one or more of the FT4 gene, the FT8 gene, and the FT10 gene, or one or more expression vectors encoding one or more RNAi constructs targeting one or more of the FT4 gene, the FT8 gene, and the FT10 gene.

15. The method of any one of claims 9-12, wherein the cell is an embryogenic callus cell, a cell in embryogenic callus, cell in a leaf or stem, plant tissue culture cell, immature embryo, a cell in an immature embryo, a friable tissue cell, a cell in a friable tissue, or a protoplast.

16. The method of any one of claims 9-12 and 15, wherein transforming the plant cell comprises: electroporation, microprojectile bombardment, or Agrobacterium tumefaciens-mediated transformation.

17. The method of any one of claims 13-14, wherein the one or more expression vectors are introduced into the plant or a progenitor of the plant by electroporation, microprojectile bombardment, or Agrobacterium tumefaciens-mediated transformation.

18. A nucleic acid for decreasing expression of the FT4 gene, the FT8 gene, or the FT10 gene in a sugarcane or energycane cell or plant comprising: a CRISPR construct for introducing a loss of function mutation into one or more of the FT4 gene, the FT8 gene, and the FT10 gene.

19. The nucleic acid of claim 18, wherein the nucleic acid comprises: SEQ ID NOs: 3-8, 13, 14, 15, 20, 21, and/or 22.

20. A nucleic acid for decreasing expression of the FT4 gene, the FT8 gene, or the FT10 gene in a sugarcane or energycane cell plant comprising: an RNAi construct targeting one or more of the FT4 gene, the FT8 gene, and the FT10 gene.

21. The nucleic acid of claim 20, wherein the RNAi construct comprises a hairpin, wherein a stem portion of the hairpin contains one or more regions of complementarity to a portion of one or more of a FT4 mRNA, a FT8 mRNA and a FT10 mRNA.

22. The nucleic acid of claim 21, wherein a loop portion of the hairpin comprises a sequence from an intron.

23. The nucleic acid sequence of any one of claims 20-22, wherein the RNAi construct targets the FT4 gene, the FT8 gene, and the FT10 gene.

24. The nucleic acid sequence of claim 23, wherein the RNAi construct contains the stem portion of the hairpin comprises a region complementary to a portion of the FT4 mRNA, a region complementary to a portion of the FT8 gene, and a region complementary to a portion of the FT10 gene.

25. The method of claim 24, wherein the nucleic acid comprises SEQ ID NOs: 1, 2, 9, 10, 11, 12, 16, 17, 18 and/or 19.
